(19) United States
(12) Patent Application Publication
(10) Pub. No.: US 2025/0195461 A1
(43) Pub. Date: Jun. 19, 2025

(54) Fibrate Compositions for Treating Inflammation and Neuroinflammation

(71) Applicant: TRx Biosciences Limited, Cambridge, Cambridgeshire (GB)

(72) Inventors: Brew John, St. Albans (GB); Daniel Gooding, Cambridge (GB); Robin M. Bannister, Saffron Walden (GB)

(73) Assignee: TRx Biosciences Limited, Cambridge, Cambridgeshire, EN (GB)

(21) Appl. No.: 18/847,243

(22) PCT Filed: Sep. 14, 2023
(86) PCT No.: PCT/EP2023/056491

§ 371 Date: Sep. 14, 2024

Related U.S. Application Data
(60) Provisional application No. 63/362,005, filed on Mar. 28, 2022.
(60) Provisional application No. 63/269,327, filed on Mar. 14, 2022.

(51) Int. Cl. A61K 31/216; A61K 9/48; A61P 29/00

(52) U.S. Cl. CPC A61K 31/216; A61K 9/4858; A61P 29/00

(57) ABSTRACT

The present specification discloses pharmaceutical composition disclosed herein comprises one or more fibrates, one or more glycerolipids, and one or more digestion enhancers. The disclosed glycerolipids comprise one or more hard fats and one or more liquid fats. The disclosed digestion enhancers comprise one or more bile acids, one or more phospholipids, one or more free C₁₄₋₂₄ fatty acids, one or more free C₁₄₋₂₄ fatty acid surfactants, or any combination thereof. The present specification also discloses methods and procedures to formulate the disclosed one or more fibrates into the disclosed pharmaceutical compositions. The present specification further discloses methods and uses of the disclosed pharmaceutical compositions in the treatment of an inflammation and/or neuroinflammation.

This application is a 35 U.S.C. § 371 U.S. national stage patent application which claims the benefit of priority and is entitled to the filing date of International Patent Application PCT/EP2023/056491, filed Mar. 14, 2023, an international patent application which claims the benefit of priority and is entitled to the filing date pursuant to 35 U.S.C. § 119(e) of U.S. Provisional Patent Application 63/362,005, filed Mar. 28, 2022, and U.S. Provisional Patent Application 63/269,327, filed Mar. 14, 2022, the content of each of which is hereby incorporated by reference in its entirety.

Inflammation involves the activation of the immune system in response to harmful stimuli, such as, e.g., a pathogen, infection, irritant, or damage to cells. As a stereotyped response, inflammation is a mechanism of innate immunity, as compared to adaptive immunity, which is specific for each pathogen. Inflammation can be classified as either acute or chronic. Generally speaking, acute inflammation is mediated by granulocytes, while chronic inflammation is mediated by mononuclear cells such as monocytes and lymphocytes.

Acute inflammation is an initial protective response of the body to remove an injurious stimulus by maintaining tissue integrity and contributing to tissue repair. It is a part of the body's natural defense system against injury and disease, and in the absence of acute inflammation, wounds and infections would never heal and progressive destruction of the tissue would compromise the survival of the organism.

The process of acute inflammation is initiated by cells already present in all tissues, mainly resident macrophages, dendritic cells, histiocytes, Kupffer cells, mastocytes, vascular endothelial cells, and vascular smooth muscle cells. At the onset of a harmful stimulus, these cells undergo activation and release inflammatory mediating and sensitizing molecules, such as, e.g., pro-inflammatory cytokines, pro-inflammatory prostaglandins, leukotrienes, histamine, serotonin, neutral proteases, bradykinin and nitric oxide. These inflammatory molecules modulate a complex series of biological events involving cellular and acellular components of the local vascular system, the immune system, and the injured tissue site to propagate and mature the inflammatory response. These events are responsible for eliciting an acute inflammatory response, typically characterized by 1) vasodilatation which increases blood flow into the tissue thereby causing erythema (redness and warmth), which may extend beyond this site (the flare response); 2) blood vessel permeability which increases plasma leakage into the tissue thereby causing edema (swelling); 3) alter the excitability of certain sensory neurons causing hypersensitivity and pain; 4) stimulate the release of inflammation inducing molecules such as, e.g., neuropeptides like substance P (SP) and calcitonin gene-related peptide (CGRP), prostaglandins, and amino acids like glutamate, from the peripheral nerve endings; and 5) increase migration of leukocytes, mainly granulocytes, from the blood vessels into the tissue. An acute inflammatory response requires constant stimulation to be sustained and must be actively terminated when no longer needed. Hence, acute inflammation ceases once the injurious stimulus has been removed.

However, severe or prolonged noxious stimulation results in a chronic inflammatory response that leads to a progressive shift in the type of cells present at the site of tissue injury. Chronic inflammation may be characterized as the simultaneous destruction and healing of tissue from the inflammatory process, with the net result of provoking injury rather than mediating repair. As such, chronic inflammation is a disease. As an inflammatory response can occur anywhere in the body, chronic inflammation has been implicated in the pathophysiology of a wide range of seemingly unrelated disorders which underlay a large and varied group of human diseases. For example, chronic inflammation is involved in diseases as diverse as cardiovascular diseases, cancers, allergies, obesity, diabetes, digestive system diseases, degenerative diseases, auto-immune disorders, and Alzheimer's disease.

Attempts to treat chronic inflammation have met with limited success. This is due, in part, to the fact that the etiology of chronic inflammation is a complex response based in part on the various inflammation inducing molecules and the multitude of inflammation mediating and sensitizing molecules that appear to elicit inflammation via redundant mechanism. In addition, besides blocking pro-inflammatory molecules, many anti-inflammatory drugs, also inhibit regulatory loops that release endogenous anti-inflammatory molecules. For example, NSAIDs reduce inflammation by blocking the enzymatic activity of cyclooxygenase, a key enzyme that catalyzes the conversion of arachidonic acid to prostaglandins and leukotrienes. Thus, NSAIDs reduce inflammation by preventing the synthesis of all prostaglandins. However, NSAIDs not only prevents the synthesis of proinflammatory prostaglandins, but these compounds also prevent the synthesis of anti-inflammatory prostaglandins. Hence, NSAIDs have limited success as they block endogenous anti-inflammatory response, which in some instances may prolong chronic inflammation. Therefore, compounds, compositions, uses, and methods preferentially inhibiting pro-inflammatory responses would be highly desirable for the treatment of chronic inflammation.

Neuroinflammation is an inflammatory response of the nervous tissue and can be both acute and chronic. Acute neuroinflammatory responses are a well-established defense against harmful conditions, such as infections, toxins, and neuronal cell injury. With respect to the peripheral nervous system (PNS), a neuroinflammatory response is handled in a manner similar to an inflammatory response. The central nervous system (CNS) is considered an immunologically privileged site since the blood-brain barrier (BBB) typically prevents peripheral immune cells from entering the CNS. Instead, in response to an inflammatory trigger, resident glia cells, called microglia, become activated to destroy infectious agents before they damage neural tissue, and as such, are main form of active immune defense in the CNS. However, when there is a disruption in the equilibrium of anti-inflammatory and proinflammatory signaling, microglia become chronically activated, leading to overproduction of proinflammatory factors (i.e., cytokines) and a progression of neurodegenerative changes (e.g., atrophy and loss of function of neurons). Activation of chronic neuroinflammation further triggers the infiltration of immune cells from the periphery across the BBB, accelerating neuroinflammation and the neurodegenerative process. Much research has focused on the central role of neuroinflammation in the pathogenesis of many conditions relating to the CNS, including e.g., traumatic brain injury, stroke, Alzheimer's disease, post-operative cognitive decline/perioperative neurocognitive disorder and now even long-term cognitive side effects from SARS-CoV-2. Attempts to treat neuroinflammation have however been met with limited success which is largely because NSAIDs have typically been formulated for minimal blood brain barrier penetration. Therefore, compounds, compositions, uses, and methods that can successfully deliver an anti-inflammatory agent to the brain would be highly desirable for the treatment of neuroinflammation.

The present specification provides an alternative approach where pharmaceutical compositions disclosed herein are formulated to rely on physiological lipid digestion and absorption systems to achieve absorption and enhanced efficacy and increase bioavailability of one or more fibrates to better facilitate treatment of both inflammation and neuroinflammation.

### SUMMARY

Aspects of the present specification disclose, in part, a pharmaceutic composition for use in treating an inflammation, the pharmaceutic composition comprising a) one or more fibrates, b) one or more glycerolipids, and c) one or more digestion enhancers. A fibrate disclosed herein can be a bezafibrate, a ciprofibrate, a clinofibrate, a clofibrate, a clofibride, an etofibrate, a fenofibrate, a fenofibric acid, a gemfibrozil, a nafenopin, a ronifibrate, and/or a simfibrate. A glycerolipid disclosed herein includes hard fats and liquid fats. A hard fat is a glycerolipid that is solid at 18° C. and includes triglycerides. A liquid fat is a glycerolipid that is liquid at 18° C. and also includes partially hydrolyzed glycerolipids and monoglycerides. Digestion enhancers disclosed herein include one or more bile acids, one or more phospholipids, one or more free C₁₄₋₂₄ fatty acid surfactants, one or more fatty acid salts, one or more fatty acid derivatives with a polyhydroxylated head group, one or more steroidal surfactants, or any combination thereof. A composition disclosed herein may further comprise one or more pharmaceutically acceptable stabilizing agents.

Other aspects of the present specification disclose, in part, a pharmaceutic composition for use in treating a neuroinflammation, the pharmaceutic composition comprising a) one or more fibrates, b) one or more glycerolipids, and c) one or more digestion enhancers. A fibrate disclosed herein can be a bezafibrate, a ciprofibrate, a clinofibrate, a clofibrate, a clofibride, an etofibrate, a fenofibrate, a fenofibric acid, a gemfibrozil, a nafenopin, a ronifibrate, and/or a simfibrate. A glycerolipid disclosed herein includes hard fats and liquid fats. A hard fat is a glycerolipid that is solid at 18° C. and includes triglycerides. A liquid fat is a glycerolipid that is liquid at 18° C. and also includes partially hydrolyzed glycerolipids and monoglycerides. Digestion enhancers disclosed herein include one or more bile acids, one or more phospholipids, one or more free C₁₄₋₂₄ fatty acid surfactants, one or more fatty acid salts, one or more fatty acid derivatives with a polyhydroxylated head group, one or more steroidal surfactants, or any combination thereof. A composition disclosed herein may further comprise one or more pharmaceutically-acceptable stabilizing agents.

Other aspects of the present specification disclose a method of treating an individual with an inflammation, the method comprising the step of administering to the individual in need thereof a composition disclosed herein, wherein administration results in a reduction in a symptom associated with the inflammation, thereby treating the individual.

Other aspects of the present specification disclose a method of treating an individual with a neuroinflammation, the method comprising the step of administering to the individual in need thereof a composition disclosed herein, wherein administration results in a reduction in a symptom associated with the neuroinflammation, thereby treating the individual.

Other aspects of the present specification disclose a use of a composition disclosed herein in the manufacture of a medicament for the treatment of an inflammation.

Other aspects of the present specification disclose a use of a composition disclosed herein in the manufacture of a medicament for the treatment of a neuroinflammation.

### BRIEF DESCRIPTION OF THE DRAWINGS

The accompanying drawings, which are incorporated in and constitute a part of this specification, illustrate aspects of the disclosed subject matter in at least one of its exemplary embodiments, which are further defined in detail in the following description. Features, elements, and aspects of the disclosure are referenced by numerals with like numerals in different drawings representing the same, equivalent, or similar features, elements, or aspects, in accordance with one or more embodiments. The drawings are not necessarily to scale, emphasis instead being placed upon illustrating the principles herein described and provided by exemplary embodiments of the invention. In such drawings:

FIGS. 1A-1H show representative PXRD spectra analyzing a disclosed pharmaceutical composition comprising fenofibrate with FIG. 1A showing a representative PXRD spectra of a GELCURE® 43/01 standard; FIG. 1B showing a representative PXRD spectra of a cholic acid standard; FIG. 1C showing a representative PXRD spectra of a fenofibrate standard; FIG. 1D showing a representative PXRD spectra of a Vehicle standard; FIG. 1E showing a representative PXRD spectra of a disclosed pharmaceutical composition comprising fenofibrate standard; FIG. 1F showing a representative PXRD spectra of a fenofibrate standard superimposed over a representative PXRD spectra of a disclosed pharmaceutical composition comprising fenofibrate standard with asterisks above peak indicating relevant peak overlap; FIG. 1G showing a representative PXRD spectra of a cholic acid standard superimposed over a representative PXRD spectra of a Vehicle standard with asterisks above peak indicating relevant peak overlap; and FIG. 1H showing a representative PXRD spectra of a cholic acid standard superimposed over a disclosed pharmaceutical composition comprising fenofibrate standard with asterisks above peak indicating relevant peak overlap;

FIGS. 2A-2B show representative UHPLC tracings of fenofibric acid levels in blood and brain with FIG. 2A shows a UHPLC tracing of fenofibric acid levels in blood after oral administration of 30 mg/kg of disclosed pharmaceutical compositions comprising fenofibrate; and FIG. 2B shows a UHPLC tracing of fenofibric acid levels in brain after oral administration of 30 mg/kg of disclosed pharmaceutical compositions comprising fenofibrate;

FIGS. 3A-3B show representative UHPLC tracings of fenofibric acid levels in blood and brain with FIG. 3A shows a UHPLC tracing of fenofibric acid levels in blood after oral administration of 30 mg/kg of disclosed pharmaceutical compositions comprising fenofibrate; and FIG. 3B shows a UHPLC tracing of fenofibric acid levels in brain after oral administration of 30 mg/kg of disclosed pharmaceutical compositions comprising fenofibrate;

FIG. 4 show representative UHPLC tracings of fenofibric acid levels in blood after oral administration of 3 mg/kg, 10 mg/kg, 30 mg/kg and 100 mg/kg of disclosed pharmaceutical compositions comprising fenofibrate;

FIGS. 5A-5F show bar graphs of blood cytokine levels in animals undergoing 5 days of an LPS challenge with treatment occurring only on Day 1 of the LPS challenge with FIG. 5A showing IL-1 levels; FIG. 5B showing IL-6 levels; FIG. 5C showing TNFα levels; FIG. 5D showing MCP-1 levels; FIG. 5E showing IL-10 levels; and FIG. 5F showing MCP-2 levels (bars represent the SEM,*=p<0.05, **=p<0.01; ***=p<0.001 versus vehicle control);

FIGS. 6A-6F show bar graphs of blood cytokine levels in animals undergoing 5 days of pre-treatment before 5 days of an LPS challenge with FIG. 6A showing IL-1p levels; FIG. 6B showing IL-6 levels; FIG. 6C showing TNFα levels; FIG. 6D showing MCP-1 levels; FIG. 6E showing IL-10 levels; and FIG. 6F showing MCP-2 levels (bars represent the SEM, *=p<0.05, **=p<0.01; ***=p<0.001);

FIGS. 7A-7F show bar graphs of brain cytokine levels in animals undergoing 5 days of an LPS challenge with treatment occurring only on Day 1 prior to an initial LPS challenge with FIG. 7A showing IL-1p levels; FIG. 7B showing IL-6 levels; FIG. 7C showing TNFα levels; FIG. 7D showing MCP-1 levels; FIG. 7E showing IL-10 levels; and FIG. 7F showing MCP-2 levels (bars represent the SEM,*=p<0.05, **=p<0.01; ***=p<0.001 versus vehicle control);

FIGS. 8A-8F show bar graphs of brain cytokine levels in animals undergoing 5 days of pre-treatment before 5 days of an LPS challenge with FIG. 8A showing IL-1p levels; FIG. 8B showing IL-6 levels; FIG. 8C showing TNFα levels; FIG. 8D showing MCP-1 levels; FIG. 8E showing IL-10 levels; and FIG. 8F showing MCP-2 levels (bars the SEM,*=p<0.05, **=p<0.01; ***=p<0.001 versus vehicle control);

FIGS. 9A-9D show bar graphs representing percent levels of IBA-1+ cells and CD-68⁺ cells per high power field (HPF, ×400) in hippocampal and cortex slices in the brain with FIG. 9A showing percent levels of cells present in a hippocampal slice stained with anti-IBA-1 antibodies; FIG. 9B showing percent levels of cells present in a hippocampal slice stained with anti-CD-68 antibodies; FIG. 9C showing percent levels of cells present in a cortical slice stained with anti-IBA-1 antibodies; FIG. 9D showing percent levels of cells present in a cortical slice stained with anti-CD-68 antibodies (bars represent the SEM,*=p<0.05, **=p<0.01; ***=p<0.001 versus vehicle control, #=p<0.05, ##=p<0.01; ###=p<0.001 versus CMF2); and

FIG. 10 is a bar graph showing fenofibric acid levels in cerebral spinal fluid (CSF) acid after oral administration of 70 mg, 140 mg, and 125 mg of disclosed pharmaceutical compositions comprising fenofibrate (bars represent the minimum and maximum concentrations measured; *=mean; and the box represents Q1 (the upper box) and Q3 (the lower box) with the line dividing the Q1 and Q3 boxes representing the median).

### DESCRIPTION

Fibrates are fibric acid derivatives and a class of amphipathic carboxylic acids. Fibrates activate peroxisome proliferator-activated receptors (PPARs), especially PPARα. PPARs are a class of ligand-activated nuclear receptors that modulate carbohydrate (or glucose) and fat metabolism, adipose tissue differentiation, endothelial function, and inflammation. PPAR antagonism mediate lipid level modifying properties, such as, e.g., lowering triglycerides and LDL as well as increase levels of HDL by increasing lipolysis, activating lipoprotein lipase, and reducing apoprotein C-III. As such, fibrates are considered hypolipidemic agents and have been used for a range of metabolic disorders, mainly hypercholesterolemia (high cholesterol). PPAR agonism also leads to a phenotypic shift from M1/TH1 proinflammatory to M2/TH2 anti-inflammatory and PPAR receptor agonism acts in complementary fashion to COX Inhibition to allow complete resolution of inflammation.

Fenofibrate is one example of a fibrate. This compound is fibric acid derivative comprising fenofibric acid linked to an isopropyl ester. In vivo, the isopropyl ester of fenofibrate is completely hydrolyzed by liver carboxylesterase 1 to produce fenofibric acid. As such, fenofibrate can be considered a prodrug of fenofibric acid. Interestingly, both fenofibrate and fenofibric acid exhibit complimentary pharmacologies that allow complete resolution of inflammation. For example, the primary anti-inflammatory effects of fenofibric acid involves its PPARα agonistic activity mediated though multiple mechanisms. On the other hand, fenofibrate mediates most of its anti-inflammatory activity through inhibition of selective cyclooxygenase 2 (COX2) activity as well as agonism of cannabinoid receptor type 2 (CB2).

As such, one advantage of utilizing the combined activity of fenofibrate and fenofibric acid to treat inflammation is their ability to influence three independent signaling pathways that mediate an inflammatory response. In addition, unlike the traditional NSAIDs and selective COX2 inhibitors, fenofibrate and fenofibric acid are devoid of gastric erosion potential and are similarly devoid of cardiac liability. Thus, the ability to harness the activity of fenofibrate and fenofibric acid to completely resolving inflammation would be of great benefit.

One obstacle in taking advantage of these three complimentary pharmacologies in resolving inflammation is the speed in which fenofibrate is converted into fenofibric acid. Its rapid hydrolysis severely limits the therapeutic effect this compound can exert. Thus, formulations which can slow, but not prevent, its conversion to fenofibric acid would be advantageous in eliciting the anti-inflammatory effects mediated by PPARα and CB2 activation and COX2 inhibition. The disclosed pharmaceutical compositions achieve such a retardation of fenofibrate hydrolysis to fenofibric acid. In addition, to increasing the therapeutic effect of fenofibrate in the blood, the disclosed pharmaceutical compositions surprisingly enable the delivery of both of fenofibrate and fenofibric acid to the brain, thereby allowing the ability of these fibrates to treat neuroinflammation.

The majority of the digestion of dietary organic macromolecules and almost all absorption of the resulting breakdown products, occurs in the small intestine. The luminal wall of the small intestine is lined with many projections called villi, each of which comprises intestinal cells called enterocytes. Enterocytes not only secrete enzymes that digest proteins (polypeptides), carbohydrates (polysaccharides), and fats (lipids) but these cells also absorbed the amino acids, monosaccharides, and fatty acid breakdown products. Interestingly, however, enterocytes process these breakdown products differently with amino acids and monosaccharides being taken up by capillaries and transported systemically by the blood system and fatty acids being taken up by blind ended lymphatic vessels called lacteal and then transported systemically by the lymphatic system. The disclosed pharmaceutical compositions take advantage of this differential processing by formulating fibrates that are preferentially processed by enterocytes in a manner where these compounds are taken up and transported into the lymphatic system.

Dietary lipids typically consumed by a mammal comprise 90% triglycerides as well as small amounts of cholesterol esters and phospholipids. Unlike proteins and carbohydrates, dietary lipids are hydrophobic molecules that cannot dissolve in the fluids present in the lumen of the small intestine and instead aggregate together to form fat globules. Pancreatic lipase is a water-soluble enzyme that cleaves ester bonds and breaks down lipid triglycerides into fatty acids and glycerol. However, due to its hydrophilic character lipase remain in the intestinal fluids, are unable to dissolve into fat globules and as such can only access and cleave triglycerides located at the surface where fat globules and intestinal fluids interface. In order to increase efficiency and cleavage rate at which lipase can breakdown lipids in the small intestine, the liver produces a fluid called bile. Bile contains amphipathic molecules such as bile salts including sodium cholate and sodium chenodeoxycholate, phospholipids including lecithin, as well as the hydrophobic steroid cholesterol. When bile is excreted into the small intestine it mixes with the fat globules in a manner where bile salts and phospholipids intercalate with and break apart these structures into smaller units called emulsion droplets as well as recruits an amphipathic molecule called coliapse. Colipase is a protein co-enzyme that binds lipase to the emulsion droplets and stabilizes the enzyme in its active conformation, As such, emulsification greatly increases the surface area of which lipases can act upon triglycerides as well as increases its efficiency and catalytic rate thereby enabling sufficient amounts of lipid digestion to occur in the small intestine.

As lipid triglyceride digestion proceeds, the resulting free fatty acids, which are also hydrophobic and insoluble in the intestinal fluids, associate with the phospholipids from the bile to form tiny droplets called mixed micelles having a phospholipid and bile salt composition which encapsulates the free fatty acids. Mixed micelles, which are about 200 to 500 times smaller in size than emulsion droplets, fuse with the membranes of enterocytes where the free fatty acids enter the cytosol of these cells. Once inside the enterocytes, the free fatty acids are transported to the lumen of smooth endoplasmic reticulum and are transformed back into triglycerides are assembled with cholesterol and phospholipids into spherical lipid structures. These lipid structures are transported to the rough endoplasmic reticulum where apoprotein Apo B-48 is attached to the surface to form large lipoproteins called chylomicrons. Chylomicrons are then packaged is the Golgi apparatus and exit the basolateral side of the enterocytes via exocytosis. Since chylomicrons are too large to be taken up by blood capillaries, these lipoproteins enter the lymphatic system via the lacteal in a process that depends on Apo B-48. The chylomicrons then circulate through the lymph vessels and drain into the blood system via the thoracic duct bypassing the liver circulation. Once chylomicrons are in the blood system, these lipoproteins travel to various extrahepatic tissues where their triglycerides are hydrolyzed by the activity of the lipoprotein lipase, allowing the released free fatty acids and glycerol to be absorbed by the tissues. When a large portion of the triglycerides has been hydrolyzed, chylomicron remnants are formed and are taken up by the liver, thereby also transferring dietary fat to this organ.

The present specification discloses pharmaceutical compositions formulated for oral delivery in a manner where the fibrate present in the pharmaceutical composition is preferential taken up into the lymphatic system. The pharmaceutical compositions disclosed herein comprises one or more fibrates, one or more glycerolipids, and one or more digestion enhancers. A digestion enhancer disclosed herein increases the solubility of the one or more fibrates in the glycerolipid matrix, in conjunction with the glycerolipids, increases absorption of these compounds into the lymphatic system and increases availability of these compounds to their therapeutic target.

Natural digestion processes rely upon the secretion of bile onto the ingested gut contents and rely upon their mixing with the gut contents to initiate emulsification and access to other digestion processes such as the activity of lipases. These processes must be completed for the drug to be absorbed. A full gall bladder response in relation to the ingestion of lipids will secrete up to 50-60 mL of bile into the duodenum. The present invention relies upon the intimate mixing of digestion enhancers with the fibrate in the preparation of a pharmaceutical composition disclosed herein that can then be taken orally by the patient. The fibrate is then presented to the gut lumen in a form that is immediately ready for uptake. This process is highly efficient as the digestion enhancers are intimately mixed with the formulation lipid excipients. Without wishing to be limited by any theory, the pharmaceutical compositions disclosed herein employ components that are the products of triglyceride digestion to mimic the conditions created by a high fat meal. There formulations enable the one or more fibrates contained therein to be bundled along with the free fatty acids into micelles and absorbed by the enterocytes which then package the one or more fibrates into chylomicrons. The fibrate loaded chylomicrons are then transported by the lymphatic system to their target cells where the fibrates are taken up by these cells to exert their beneficial effects. In essence, the chylomicrons are being co-opted as a drug delivery system for the one or more fibrates contain in a pharmaceutical composition disclosed herein. By controlling the components and amounts of the one or more glycerolipids and one or more digestion enhancers, the disclosed pharmaceutical compositions provide a more consistent and predictable bioavailability of the one or more fibrates disclosed herein that could ever be achieved by reliance on a high fat meal.

The pharmaceutical compositions disclosed herein are advantageous for several reasons all of which ultimately increase the bioavailability and efficacy of the one or more fibrates contained therein. For example, a pharmaceutical composition disclosed herein delivers its fibrates via the lymphatic system. The use of the lymphatic system to deliver fibrates is beneficial for several reasons. First, the lymphatic system avoids pre-systemic metabolism that reduces the bioavailability of many fibrates administered using a traditional oral delivery approach. Also called first pass effect or first-pass metabolism, a fibrate absorbed by the digestive system must first enter the hepatic portal system before reaching systemic circulation. While in the hepatic portal system, a fibrate can be metabolized by hepatic enzymes of the liver which reduce the amount of fibrate that enters systemic circulation. As such, delivery of fibrates via the lymphatic system acts as a bypass to the hepatic portal system for fibrates susceptible to hepatic metabolism. The lymphatic uptake system represents an attractive opportunity for the preferential delivery of drugs. However, small water-soluble molecules that obey the rules for paracellular absorption (such as Lipinski's Rules) are not ideal substrates for lymphatic uptake. Additional highly lipophilic molecules which are often poorly and variably bioavailable even when absorbed by lymphatic uptake. This invention, uses a wide family of digestion enhancers in a lipidic delivery system, to allow both the small molecule Lipinski compliant drugs and the highly lipophilic drugs to be effectively delivered through the lymphatic uptake route.

Additionally, the lymphatic route of administration can aid delivery of fibrates directly to its target cells, thereby increasing its bioavailability and decreasing its clearance rate. For example, fibrate loaded chylomicrons transported into the lymphatic system would drain into the thoracic artery and circulate throughout the body via the arterial system until reaching a capillary bed where the fibrate loaded chylomicrons extracavates into the surrounding tissue to be taken up by target cell. Fibrate loaded chylomicrons not taken up by target cells and cleared from the extracellular environment by interstitial fluid where they are taken up by lacteals, transported by the lymphatic system, and drained into the thoracic artery where the fibrate loaded chylomicrons would once again be systemically recirculated throughout the body. Such lymphatic-based administration results in more fibrate being delivered to the various systems, organs, and tissues due to the avoidance of the hepatic portal system discussed above. Additionally, since more fibrate enters the general circulatory system, its elimination, i.e., metabolism and excretion, is prolonged, thereby decreasing the clearance rate of this compound which effectively increases its half-life.

Another advantage of the disclosed pharmaceutical compositions is the use of chylomicrons to selectively biodistribute the one or more fibrates contained therein to immune cells such as, e.g., macrophages and dendritic cells. For example, there are several processes whereby macrophages can take up chylomicrons. First, macrophages circulating in the lymph and blood secrete lipoprotein lipase and chylomicrons are substrates for this enzymatic activity resulting in the uptake of chylomicron proteins and lipids, and by extension any fibrates contained within the chylomicrons. Additionally, the exogenous lipoprotein metabolism pathway through a series of processing events converts chylomicrons to LDL particles which become oxidized by ROS to create oxidized LDL particles. FAT/CD36 scavenger receptor located on membrane of macrophages bind to and endocytose these oxidized LDL particles including any fibrates contained therein. Ultimately, these fibrate-loaded macrophages will be directed to cells undergoing pathologic distress where the fibrates can be delivered to these distressed cells.

Additionally, when processed in the small intestine into micelles, components of the disclosed pharmaceutical compositions are believed to mimic pathogen-associated molecular patterns (PAMPs). Absorption of these micelles by gut-associated lymphoid tissue (GALT) of the small intestine result in the subsequent the subsequent uptake by immune cells like macrophages and dendritic cells by a pattern recognition receptor-mediated process. It is also suspected that since these micelles share structural similarities to chylomicrons, these micelles can also be taken up via the macrophage lipoprotein lipase process discussed above.

The disclosed pharmaceutical compositions are also advantageous because the components used for its formulation mimic the signals that trigger bile and pancreatic lipoprotein lipase secretion, enhance the rate of micellular formation, and increase the enterocyte absorption rate of micelle above that achieved with the use of the digestion enhancers in the formulation alone. Such characteristics increase the speed and amount of fibrate entering the lymphatic system and thus its bioavailability.

Besides the preferential uptake into the lymphatic system and selective biodistribution to immune cells, the disclosed pharmaceutical compositions have several additional advantages. For example, fibrates are poorly water soluble or hydrophobic therapeutic compounds that heretofore have proven difficult to formulate in a therapeutically effective manner due to, e.g., their insolubility or instability in solution resulting in precipitation. Additionally, it has surprisingly be found that significantly higher concentrations of fibrates can be formulated in a pharmaceutical composition disclosed herein relative to currently known formulations. In addition, a disclosed pharmaceutical composition comprising one or more fibrates dramatically increase the dissolution rate and bioavailability of these compounds.

Yet another advantage of the disclosed pharmaceutical compositions is enhanced specificity for the cellular targets of the one or more fibrates. Chylomicrons comprise membrane-bound proteins which function as ligands which associate with their cognate receptors located on the membrane surface of cells. One such ligand of these large lipoprotein particles are proteins that interact with lipid transport proteins. As fibrate-loaded chylomicrons are shed for absorption, cells that express high levels of lipid transport proteins on the cellular membrane preferentially bind and internalize these lipoprotein particles. For example, the brain has high levels of lipid transport proteins on the cellular membrane allowing passage across the blood brain barrier, and other epithelial tissue such as the choroid plexus. This mechanism thereby increases the efficacy of these compounds. Other cells that express high levels of lipid transport proteins on the cellular membrane include, immune, heart, adipose, hepatic, and cancer cells. Heart cells express high levels of lipid transport proteins on their surface and we may therefore anticipate an increased cardiotoxicity for therapeutic compounds delivered through the lymphatic pathway. However, cardiotoxicity of therapeutic compounds is commonly caused through a high Cₘₐₓ concentrations of free compound, resulting in GPCR or ion channel related pathologies. Surprisingly therefore lipid delivery of therapeutic compounds can result in lower-than-expected cardiotoxicity as these targets are protected from high concentrations of free compounds, as they are only slowly released from the chylomicron phase. Thus, the use of a pharmaceutical composition disclosed herein can also be associated with reduce cardiac side effects and diminished toxicity. In addition, upon ingestion, therapeutic compounds remain embedded in the oily/fatty excipients, limiting opportunity for the compounds to become solubilized in the aqueous gut contents, thereby avoiding contact toxicities such as gastric erosion and other localized damage or harm. As well as avoiding first pass metabolism and local toxicities, the enhanced uptake through the lymphatic pathway also minimizes the contact and availability of free therapeutic compound further down the alimentary canal. In this way interaction and disruption of the gut microbiome can be minimized. This is a particular benefit in using the technology with antimicrobial agents. Furthermore, due to the anatomy of the lymphatic system, the lungs can be effectively targeted with appropriate medications. For example, anti-inflammatory, antifibrotic, antimicrobial and bronchodilatory medications can be considered ling targeting through lymphatic delivery, using this technology.

Furthermore, the disclosed pharmaceutical compositions are unlike current surfactant-based formulations, such as, e.g., macroemulsion, microemulsion, self-emulsifying drug delivery systems (SEDDS), self-microemulsifying drug delivery systems (SMEDDS), self-nanoemulsifying drug delivery systems (SNEDDS), solid-lipid nanoparticle (SLN), liposomes and lipoplexes. The disclosed pharmaceutical compositions are not emulsions or self-emulsifying compositions, and as such avoid the side-effects associated with these formulations like destabilizing the membranes lining in the stomach causing irritation. Additionally, the disclosed pharmaceutical compositions avoid the manufacturing issues associated with current surfactant-based formulations, such as, e.g., formulation handling issues and no predictive in vitro testing. The disclosed pharmaceutical compositions also avoid the problems associated with current surfactant-based formulations include, e.g., in vivo drug precipitation, limited lymphatic uptake, and lack of and oxidation of unsaturated fatty acids. Surprisingly, by mimicking the microenvironment of a mixed micelle, the disclosed pharmaceutical compositions completely bypass the dissolution phase of drug uptake resulting in significantly greater bioavailability of one or more fibrates contained therein. Unlike the self-emulsifying systems above the formulations disclosed herein are designed to avoid the formation of stable emulsions and when added to water, do not undergo spontaneous emulsification (exhibit the Ouzo Effect). Rather, when added to water these materials are clearly immiscible.

### Pharmaceutical Composition

Aspects of the present specification disclose, in part, a composition. A composition disclosed herein is generally administered as a pharmaceutical acceptable composition. As used herein, the term “pharmaceutically acceptable” refers any molecular entity or composition useful in preparing a pharmaceutical composition that is generally safe, non-toxic, and neither biologically nor otherwise undesirable and includes that which is acceptable for veterinary use as well as human pharmaceutical use. As used herein, the term “pharmaceutically acceptable composition” is synonymous with “pharmaceutical composition” and means the combination of one or more fibrates disclosed herein that are combined with one or more glycerolipids, one or more digestion enhancers, and other components disclosed herein to form the product that is administered to an individual. A pharmaceutical composition disclosed herein is useful for medical and veterinary applications. A pharmaceutical composition may be administered to an individual alone, or in combination with other supplementary active ingredients, agents, drugs, or hormones.

The present specification discloses pharmaceutical compositions useful to formulate one or more fibrates. In some embodiments, a pharmaceutical composition disclosed herein comprises a) one or more fibrates, b) one or more glycerolipids, and c) one or more digestion enhancers. In some embodiments, a pharmaceutical composition disclosed herein comprises a) one or more fibrates, b) one or more glycerolipids, c) one or more bile acids and/or one or more bile salts, one or more phospholipids, one or more free C₁₄₋₂₄ fatty acid surfactants, or any combination thereof. In some embodiments, a pharmaceutical composition disclosed herein comprises a) one or more fibrates, b) one or more glycerolipids, c) one or more bile acids and/or one or more bile salts; and d) one or more phospholipids. In some embodiments, a pharmaceutical composition disclosed herein comprises a) one or more fibrates, b) one or more glycerolipids, c) one or more bile acids and/or one or more bile salts; d) one or more phospholipids; and e) one or more free C₁₄₋₂₄ fatty acid surfactants.

In some embodiments, a pharmaceutical composition disclosed herein comprises a) one or more fibrates, b) one or more triglycerides, one or more partially hydrolyzed glycerolipids including a mixture of mono-, di- and triglycerides, or a combination of one or more triglycerides and one or more partially hydrolyzed glycerolipids, and c) one or more digestion enhancers. In some embodiments, a pharmaceutical composition disclosed herein comprises a) one or more fibrates, b) one or more triglycerides, one or more partially hydrolyzed glycerolipids including a mixture of mono-, di- and triglycerides, or a combination of one or more triglycerides and one or more partially hydrolyzed glycerolipids, c) one or more bile acids and/or one or more bile salts, one or more phospholipids, one or more free C₁₄₋₂₄ fatty acid surfactants, or any combination thereof. In some embodiments, a pharmaceutical composition disclosed herein comprises a) one or more fibrates, b) one or more triglycerides, one or more partially hydrolyzed glycerolipids including a mixture of mono-, di- and triglycerides, or a combination of one or more triglycerides and one or more partially hydrolyzed glycerolipids, c) one or more bile acids and/or one or more bile salts; and d) one or more phospholipids. In some embodiments, a pharmaceutical composition disclosed herein comprises a) one or more fibrates, b) one or more triglycerides, one or more partially hydrolyzed glycerolipids including a mixture of mono-, di- and triglycerides, or a combination of one or more triglycerides and one or more partially hydrolyzed glycerolipids, c) one or more bile acids and/or one or more bile salts; d) one or more phospholipids; and e) one or more free C₁₄₋₂₄ fatty acid surfactants.

In some embodiments, a pharmaceutical composition disclosed herein comprises a) one or more fibrates, b) one or more triglycerides, one or more monoglycerides, or a combination of one or more triglycerides and one or more monoglycerides, and c) one or more digestion enhancers. In some embodiments, a pharmaceutical composition disclosed herein comprises a) one or more fibrates, b) one or more triglycerides, one or more monoglycerides, or a combination of one or more triglycerides and one or more monoglycerides, c) one or more bile acids and/or one or more bile salts, one or more phospholipids, one or more free C₁₄₋₂₄ fatty acid surfactants, or any combination thereof. In some embodiments, a pharmaceutical composition disclosed herein comprises a) one or more fibrates, b) one or more triglycerides, one or more monoglycerides, or a combination of one or more triglycerides and one or more monoglycerides, c) one or more bile acids and/or one or more bile salts; and d) one or more phospholipids. In some embodiments, a pharmaceutical composition disclosed herein comprises a) one or more fibrates, b) one or more triglycerides, one or more monoglycerides, or a combination of one or more triglycerides and one or more monoglycerides, c) one or more bile acids and/or one or more bile salts; d) one or more phospholipids; and e) one or more free C₁₄₋₂₄ fatty acid surfactants.

A pharmaceutical composition disclosed herein is formulated as an anhydrous solid, a solid dispersion, or a molecular dispersion. As such, the formulations of the disclosed pharmaceutical compositions lack any water. In addition, as discussed above, the disclosed pharmaceutical compositions are not emulsions or self-emulsifying compositions. As such, a pharmaceutical composition disclosed herein will maintain its hydrophobic lipid characteristics when in an aqueous environment, behaving just like fats and oils and requiring the lipid digestive process to be broken down for absorption. Only when exposed to pancreatic juices from the small intestine will emulsification occur. One reason is that the one or more glycerolipids and the one or more digestion enhancers used are not sufficiently amphiphilic to initiate emulsification and require the action of bile secreted by the gall bladder during the digestion process in order for these components to contribute to the formation of micellar structures. Another reason is that the bile acid, fatty acid surfactants, phospholipids, and any other digestion enhancer are all individually and in combination below the critical micellar concentration necessary for emulsification to occur.

### Fibrates

Aspects of the present specification disclose, in part, a fibrate. Examples of a suitable fibrate include, without limitation, a bezafibrate, a ciprofibrate, a clinofibrate, a clofibrate, a clofibride, an etofibrate, a fenofibrate, a fenofibric acid, a gemfibrozil, a nafenopin, a ronifibrate, and a simfibrate. Fibrates are higher lipophilic or hydrophobic, with bezafibrate having a log P of 4.0, ciprofibrate having a log P of 3.6, clinofibrate having a log P of 7.5, clofibrate having a log P of 3.4, clofibride having a log P of 2.8, etofibrate having a log P of 3.5, fenofibrate having a log P of 5.3, fenofibric acid having a log P of 4.4, gemfibrozil having a log P of 4.4, nafenopin having a log P of 4.9, ronifibrate having a log P of 3.9, and simfibrate having a log P of 6.2.

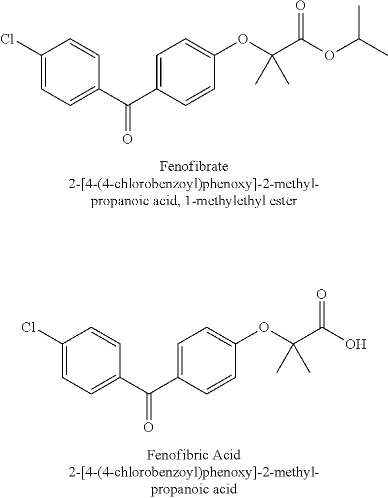 [SMILES: CC(C)OC(=O)C(C)(C)Oc1ccc(C(=O)c2ccc(Cl)cc2)cc1 ; CC(C)(Oc1ccc(C(=O)c2ccc(Cl)cc2)cc1)C(=O)O]

With respect to the other fibrates, bezafibrate, ciprofibrate, and gemfibrozil, these compounds exist in acid form whereas, like fenofibrate, clofibrate, ronifibrate, and simfibrate are fibric acid derivative esters. Besides being a PPARα agonist, bezafibrate may also have some agonistic activity on PPARγ and PPARδ.

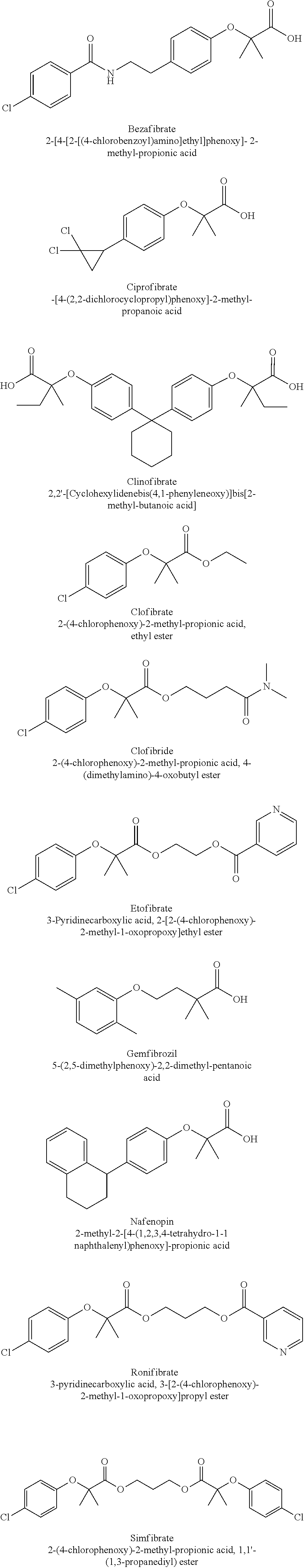

In one embodiment, a pharmaceutical composition disclosed herein comprises one or more fibrates in an amount of, e.g., about 0.05%, about 0.1%, about 1%, about 2.5%, about 5%, about 7.5%, about 10%, about 12.5%, about 15%, about 17.5%, about 20%, about 22.5%, about 25%, about 30%, about 35%, about 40%, about 45%, or about 50% by weight. In one embodiment, a pharmaceutical composition disclosed herein comprises one or more fibrates in an amount of, e.g., at least 0.05%, at least 0.1%, at least 1%, at least 2.5%, at least 5%, at least 7.5%, at least 10%, at least 12.5%, at least 15%, at least 17.5%, at least 20%, at least 22.5%, at least 25%, at least 30%, at least 35%, at least 40%, at least 45%, or at least 50% by weight. In one embodiment, a pharmaceutical composition disclosed herein comprises one or more fibrates in an amount of, e.g., at most 0.05%, at most 0.1%, at most 1%, at most 2.5%, at most 5%, at most 7.5%, at most 10%, at most 12.5%, at most 15%, at most 17.5%, at most 20%, at most 22.5%, at most 25%, at most 30%, at most 35%, at most 40%, at most 45%, or at most 50% by weight.

In one embodiment, a pharmaceutical composition disclosed herein comprises one or more fibrates disclosed herein in an amount of, e.g., about 0.05% to about 1%, about 0.05% to about 2.5%, about 0.05% to about 5%, about 0.05% to about 7.5%, about 0.05% to about 10%, about 0.05% to about 12.5%, about 0.05% to about 15%, about 0.05% to about 17.5%, about 0.05% to about 20%, about 0.05% to about 22.5%, about 0.05% to about 25%, about 0.05% to about 30%, about 0.05% to about 40%, about 0.05% to about 50%, about 0.1% to about 1%, about 0.1% to about 2.5%, about 0.1% to about 5%, about 0.1% to about 7.5%, about 0.1% to about 10%, about 0.1% to about 12.5%, about 0.1% to about 15%, about 0.1% to about 17.5%, about 0.1% to about 20%, about 0.1% to about 22.5%, about 0.1% to about 25%, about 0.1% to about 30%, about 0.1% to about 40%, about 0.1% to about 50%, about 1% to about 2.5%, about 1% to about 5%, about 1% to about 7.5%, about 1% to about 10%, about 1% to about 12.5%, about 1% to about 15%, about 1% to about 17.5%, about 1% to about 20%, about 1% to about 22.5%, about 1% to about 25%, about 1% to about 30%, about 1% to about 40%, about 1% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50% by weight.

In one embodiment, a pharmaceutical composition disclosed herein comprises one or more fibrates in a concentration of, e.g., about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 60 mg/mL, about 65 mg/mL, about 70 mg/mL, about 75 mg/mL, about 100 mg/mL, about 125 mg/mL, about 150 mg/mL, about 175 mg/mL, about 200 mg/mL, about 225 mg/mL, about 250 mg/mL, about 275 mg/mL, or about 300 mg/mL. In one embodiment, a pharmaceutical composition disclosed herein comprises one or more fibrates in a concentration of, e.g., at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 60 mg/mL, at least 65 mg/mL, at least 70 mg/mL, at least 75 mg/mL, at least 100 mg/mL, at least 125 mg/mL, at least 150 mg/mL, at least 175 mg/mL, at least 200 mg/mL, at least 225 mg/mL, at least 250 mg/mL, at least 275 mg/mL, or at least 300 mg/mL. In one embodiment, a pharmaceutical composition disclosed herein comprises one or more fibrates in a concentration of, e.g., at most 10 mg/mL, at most 15 mg/mL, at most 20 mg/mL, at most 25 mg/mL, at most 30 mg/mL, at most 35 mg/mL, at most 40 mg/mL, at most 45 mg/mL, at most 50 mg/mL, at most 60 mg/mL, at most 65 mg/mL, at most 70 mg/mL, at most 75 mg/mL, at most 100 mg/mL, at most 125 mg/mL, at most 150 mg/mL, at most 175 mg/mL, at most 200 mg/mL, at most 225 mg/mL, at most 250 mg/mL, at most 275 mg/mL, or at most 300 mg/mL.

In one embodiment, a pharmaceutical composition disclosed herein comprises one or more fibrates in a concentration of, e.g., about 10 mg/mL to about 25 mg/mL, about 10 mg/mL to about 50 mg/mL, about 10 mg/mL to about 75 mg/mL, about 10 mg/mL to about 100 mg/mL, about 10 mg/mL to about 125 mg/mL, about 10 mg/mL to about 150 mg/mL, about 10 mg/mL to about 200 mg/mL, about 10 mg/mL to about 250 mg/mL, about 10 mg/mL to about 300 mg/mL, about 25 mg/mL to about 50 mg/mL, about 25 mg/mL to about 75 mg/mL, about 25 mg/mL to about 100 mg/mL, about 25 mg/mL to about 125 mg/mL, about 25 mg/mL to about 150 mg/mL, about 25 mg/mL to about 200 mg/mL, about 25 mg/mL to about 250 mg/mL, about 25 mg/mL to about 300 mg/mL, about 50 mg/mL to about 100 mg/mL, about 50 mg/mL to about 150 mg/mL, about 50 mg/mL to about 200 mg/mL, about 50 mg/mL to about 250 mg/mL, about 50 mg/mL to about 300 mg/mL, about 75 mg/mL to about 100 mg/mL, about 75 mg/mL to about 150 mg/mL, about 75 mg/mL to about 200 mg/mL, about 75 mg/mL to about 250 mg/mL, about 75 mg/mL to about 300 mg/mL, about 100 mg/mL to about 150 mg/mL, about 100 mg/mL to about 200 mg/mL, about 100 mg/mL to about 250 mg/mL, about 100 mg/mL to about 300 mg/mL, about 125 mg/mL to about 150 mg/mL, about 125 mg/mL to about 200 mg/mL, about 125 mg/mL to about 250 mg/mL, about 125 mg/mL to about 300 mg/mL, about 150 mg/mL to about 200 mg/mL, about 150 mg/mL to about 250 mg/mL, about 150 mg/mL to about 300 mg/mL, about 200 mg/mL to about 250 mg/mL, about 200 mg/mL to about 300 mg/mL, or about 250 mg/mL to about 300 mg/mL.

### Glycerolipids

A pharmaceutical composition disclosed herein may comprises one or more glycerolipids. Glycerolipids are composed mainly of mono-, di-, and tri-substituted glycerols and are hydrophobic molecules having an HLB of less than 4. One group of glycerolipids is the glycerides, where one, two, or all three hydroxyl groups of glycerol are each esterified using a fatty acid to produce monoglycerides, diglycerides, and triglycerides, respectively. In these compounds, each hydroxyl groups of glycerol may be esterified by the same fatty acid or different fatty acids. In some embodiments, a monoglyceride disclosed herein may include a saturated or unsaturated fatty acid having a carbon length of C₁₂-C₂₄. In some embodiments, a diglyceride disclosed herein may include one saturated or unsaturated fatty acid having a carbon length of C₁₂-C₂₄, or two saturated or unsaturated fatty acids each having a carbon length of C₁₂-C₂₄. In some embodiments, a triglyceride disclosed herein may include one saturated or unsaturated fatty acid having a carbon length of C₁₂-C₂₄, two saturated or unsaturated fatty acids each having a carbon length of C₁₂-C₂₄, or three saturated or unsaturated fatty acids each having a carbon length of C₁₂-C₂₄.

Two types of glycerolipids are used in formulating one or more fibrates disclosed herein to produce a pharmaceutical composition disclosed herein. One type is hard fats, namely glycerolipids that are solid at 18° C. A disclosed hard fat or glycerolipid that is solid at 18° C. has several purposes. During the formulation process of a pharmaceutical composition disclosed herein, a hard fat complexes with a fibrate disclosed herein, stabilizes it in a glycerolipid matrix that prevents the compound from precipitating out. Additionally, during administration of a pharmaceutical composition disclosed herein, a hard fat disclosed herein triggers the lipid digestion process stimulating the release of bile from the gallbladder to enhance the emulsification of the administered pharmaceutical composition. Furthermore, the hard fats present in the pharmaceutical composition serve as substrates for pancreatic lipase which breaks down these hard fats into glycerol and free fatty acids. The presence of these digested lipid molecules triggers their absorption, along with the associated fibrate, by the enterocytes lining the lumen of the duodenum of the small intestine. Once internalized, the enterocytes subsequent process and distribute the free fatty acid/fibrate mixture into the lymphatic system. A disclosed hard fat or glycerolipid that is solid at 18° C. do not have emulsion forming properties since these lipids does not exhibit a critical micelle concentration. As such, another purpose of a disclosed hard fat or glycerolipid that is solid at 18° C. is to prevent formulary components that do possess a critical micelle concentration from initiating emulsification by diluting these components to below their critical micelle concentration and providing an anhydrous environment that reduces the necessary interaction of these components to initiate emulsification.

The other type of glycerolipid used in formulating one or more fibrates disclosed herein is liquid fats or oils, namely glycerolipids that are liquid at 18° C. The primary purposes of a disclosed liquid fat or glycerolipid that is liquid at 18° C. is as a solvent that facilitates dissolvement of a fibrate disclosed herein as well as a stabilizing agent that prevents a disclosed fibrate from precipitating out of the glycerolipid matrix. A disclosed liquid fat or glycerolipid that is liquid at 18° C. do not have emulsion forming properties since these lipids does not exhibit a critical micelle concentration. As such, another purpose of a disclosed liquid fat or glycerolipid that is liquid at 18° C. is to prevent formulary components that do possess a critical micelle concentration from initiating emulsification by diluting these components to below their critical micelle concentration and providing an anhydrous environment that reduces the necessary interaction of these components to initiate emulsification.

In some embodiments, a pharmaceutical composition disclosed herein may include one or more glycerolipids in an amount of, e.g., about 20% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, about 80% by weight, about 85% by weight, about 90% by weight, or about 95% by weight. In some embodiments, a pharmaceutical composition disclosed herein may include one or more glycerolipids in an amount of, e.g., at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, at least 75% by weight, at least 80% by weight, at least 85% by weight, at least 90% by weight, or at least 95% by weight. In some embodiments, a pharmaceutical composition disclosed herein may include one or more glycerolipids in an amount of, e.g., at most 20% by weight, at most 25% by weight, at most 30% by weight, at most 35% by weight, at most 40% by weight, at most 45% by weight, at most 50% by weight, at most 55% by weight, at most 60% by weight, at most 65% by weight, at most 70% by weight, at most 75% by weight, at most 80% by weight, at most 85% by weight, at most 90% by weight, at most 95% by weight, or at most 99% by weight.

In some embodiments, a pharmaceutical composition disclosed herein may include one or more glycerolipids in an amount of, e.g., about 20% to about 30%, about 20% to about 40%, about 20% to about 50%, about 20% to about 55%, about 20% to about 60%, about 20% to about 70%, about 20% to about 80%, about 20% to about 90%, about 25% to about 30%, about 25% to about 40%, about 25% to about 50%, about 25% to about 55%, about 25% to about 60%, about 25% to about 70%, about 25% to about 80%, about 25% to about 90%, about 30% to about 40%, about 30% to about 50%, about 30% to about 55%, about 30% to about 60%, about 30% to about 70%, about 30% to about 80%, about 30% to about 90%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 55% to about 60%, about 55% to about 65%, about 55% to about 70%, about 55% to about 75%, about 55% to about 80%, about 55% to about 85%, about 55% to about 90%, about 55% to about 95%, about 60% to about 65%, about 60% to about 70%, about 60% to about 75%, about 60% to about 80%, about 60% to about 85%, about 60% to about 90%, about 60% to about 95%, about 65% to about 70%, about 65% to about 75%, about 65% to about 80%, about 65% to about 85%, about 65% to about 90%, about 65% to about 95%, about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 80% to about 99%, about 85% to about 90%, about 85% to about 95%, about 85% to about 99%, about 90% to about 95%, or about 90% to about 99% by weight.

In some embodiments, pharmaceutical composition disclosed herein may include one or more hard fats or glycerolipids that are solid at 18° C. comprising, or consisting essentially of or consisting of a mixture of saturated C₁₀-C₁₈ triglycerides, a mixture of saturated C₁₀-C₂₀ triglycerides, a mixture of saturated C₁₀-C₂₂ triglycerides, a mixture of saturated C₁₀-C₂₄ triglycerides, a mixture of saturated C₁₂-C₁₈ triglycerides, a mixture of saturated C₁₂-C₂₀ triglycerides, a mixture of saturated C₁₂-C₂₂ triglycerides, a mixture of saturated C₁₂-C₂₄ triglycerides, a mixture of saturated C₁₄-C₁₈ triglycerides, a mixture of saturated C₁₄-C₂₀ triglycerides, a mixture of saturated C₁₄-C₂₂ triglycerides, a mixture of saturated C₁₄-C₂₄ triglycerides, a mixture of saturated C₁₆-C₁₈ triglycerides, a mixture of saturated C₁₆-C₂₀ triglycerides, a mixture of saturated C₁₆-C₂₂ triglycerides, a mixture of saturated C₁₆-C₂₄ triglycerides, a mixture of saturated C₁₈-C₂₀ triglycerides, a mixture of saturated C₁₈-C₂₂ triglycerides, a mixture of saturated C₁₈-C₂₄ triglycerides, a mixture of saturated C₂₀-C₂₂ triglycerides, or a mixture of saturated C₂₂-C₂₄ triglycerides.

In some embodiments, pharmaceutical composition disclosed herein may include one or more hard fats or glycerolipids that are solid at 18° C. comprising, or consisting essentially of or consisting of a mixture of unsaturated C₁₀-C₁₈ triglycerides, a mixture of unsaturated C₁₀-C₂₀ triglycerides, a mixture of unsaturated C₁₀-C₂₂ triglycerides, a mixture of unsaturated C₁₀-C₂₄ triglycerides, a mixture of unsaturated C₁₂-C₁₈ triglycerides, a mixture of unsaturated C₁₂-C₂₀ triglycerides, a mixture of unsaturated C₁₂-C₂₂ triglycerides, a mixture of unsaturated C₁₂-C₂₄ triglycerides, a mixture of unsaturated C₁₄-C₁₈ triglycerides, a mixture of unsaturated C₁₄-C₂₀ triglycerides, a mixture of unsaturated C₁₄-C₂₂ triglycerides, a mixture of unsaturated C₁₄-C₂₄ triglycerides, a mixture of unsaturated C₁₆-C₁₈ triglycerides, a mixture of unsaturated C₁₆-C₂₀ triglycerides, a mixture of unsaturated C₁₆-C₂₂ triglycerides, a mixture of unsaturated C₁₆-C₂₄ triglycerides, a mixture of unsaturated C₁₈-C₂₀ triglycerides, a mixture of unsaturated C₁₈-C₂₂ triglycerides, a mixture of unsaturated C₁₈-C₂₄ triglycerides, a mixture of unsaturated C₂₀-C₂₂ triglycerides, or a mixture of unsaturated C₂₂-C₂₄ triglycerides.

In some embodiments, pharmaceutical composition disclosed herein may include one or more hard fats or glycerolipids that are solid at 18° C. comprising, or consisting essentially of or consisting of a mixture of saturated and unsaturated C₁₀-C₁₈ triglycerides, a mixture of saturated and unsaturated C₁₀-C₂₀ triglycerides, a mixture of saturated and unsaturated C₁₀-C₂₂ triglycerides, a mixture of saturated and unsaturated C₁₀-C₂₄ triglycerides, a mixture of saturated and unsaturated C₁₂-C₁₈ triglycerides, a mixture of saturated and unsaturated C₁₂-C₂₀ triglycerides, a mixture of saturated and unsaturated C₁₂-C₂₂ triglycerides, a mixture of saturated and unsaturated C₁₂-C₂₄ triglycerides, a mixture of saturated and unsaturated C₁₄-C₁₈ triglycerides, a mixture of saturated and unsaturated C₁₄-C₂₀ triglycerides, a mixture of saturated and unsaturated C₁₄-C₂₂ triglycerides, a mixture of saturated and unsaturated C₁₄-C₂₄ triglycerides, a mixture of saturated and unsaturated C₁₆-C₁₈ triglycerides, a mixture of saturated and unsaturated C₁₆-C₂₀ triglycerides, a mixture of saturated and unsaturated C₁₆-C₂₂ triglycerides, a mixture of saturated and unsaturated C₁₆-C₂₄ triglycerides, a mixture of saturated and unsaturated C₁₈-C₂₀ triglycerides, a mixture of saturated and unsaturated C₁₈-C₂₂ triglycerides, a mixture of saturated and unsaturated C₁₈-C₂₄ triglycerides, a mixture of saturated and unsaturated C₂₀-C₂₂ triglycerides, or a mixture of saturated and unsaturated C₂₂-C₂₄ triglycerides.

In some embodiments, a pharmaceutical composition disclosed herein may include one or more hard fats or glycerolipids that are solid at 18° C. comprising, or consisting essentially of or consisting of a mixture of triglycerides having a melting point of, e.g., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 43° C., about 43° C., about 44° C., about 45° C., about 45° C., about 47° C., about 48° C., about 49° C., or about 50° C. In some embodiments, a pharmaceutical composition disclosed herein may include one or more hard fats or glycerolipids that are solid at 18° C. comprising, or consisting essentially of or consisting of a mixture of triglycerides having a melting point of, e.g., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 43° C., at least 43° C., at least 44° C., at least 45° C., at least 45° C., at least 47° C., at least 48° C., at least 49° C., or at least 50° C. In some embodiments, a pharmaceutical composition disclosed herein may include one or more hard fats or glycerolipids that are solid at 18° C. comprising, or consisting essentially of or consisting of a mixture of triglycerides having a melting point of, e.g., at most 25° C., at most 26° C., at most 27° C., at most 28° C., at most 29° C., at most 30° C., at most 31° C., at most 32° C., at most 33° C., at most 34° C., at most 35° C., at most 36° C., at most 37° C., at most 38° C., at most 39° C., at most 40° C., at most 41° C., at most 43° C., at most 43° C., at most 44° C., at most 45° C., at most 45° C., at most 47° C., at most 48° C., at most 49° C., or at most 50° C.

In some embodiment, a pharmaceutical composition disclosed herein may include one or more hard fats or glycerolipids that are solid at 18° C. comprising, or consisting essentially of or consisting of a mixture of triglycerides having a melting point of, e.g., about 25° C. to about 37° C., about 25° C. to about 38° C., about 25° C. to about 39° C., about 25° C. to about 40° C., about 25° C. to about 41° C., about 25° C. to about 42° C., about 25° C. to about 43° C., about 25° C. to about 44° C., about 25° C. to about 45° C., about 25° C. to about 46° C., about 25° C. to about 47° C., about 25° C. to about 48° C., about 25° C. to about 49° C., about 25° C. to about 50° C., about 28° C. to about 37° C., about 28° C. to about 38° C., about 28° C. to about 39° C., about 28° C. to about 40° C., about 28° C. to about 41° C., about 28° C. to about 42° C., about 28° C. to about 43° C., about 28° C. to about 44° C., about 28° C. to about 45° C., about 28° C. to about 46° C., about 28° C. to about 47° C., about 28° C. to about 48° C., about 28° C. to about 49° C., about 28° C. to about 50° C., about 30° C. to about 37° C., about 30° C. to about 38° C., about 30° C. to about 39° C., about 30° C. to about 40° C., about 30° C. to about 41° C., about 30° C. to about 42° C., about 30° C. to about 43° C., about 30° C. to about 44° C., about 30° C. to about 45° C., about 30° C. to about 46° C., about 30° C. to about 47° C., about 30° C. to about 48° C., about 30° C. to about 49° C., about 30° C. to about 50° C., about 32° C. to about 44° C., about 32° C. to about 45° C., about 32° C. to about 46° C., about 32° C. to about 47° C., about 32° C. to about 48° C., about 32° C. to about 49° C., about 32° C. to about 50° C., about 34° C. to about 44° C., about 34° C. to about 45° C., about 34° C. to about 46° C., about 34° C. to about 47° C., about 34° C. to about 48° C., about 34° C. to about 49° C., about 34° C. to about 50° C., about 36° C. to about 44° C., about 36° C. to about 45° C., about 36° C. to about 46° C., about 36° C. to about 47° C., about 36° C. to about 48° C., about 36° C. to about 49° C., about 36° C. to about 50° C., about 38° C. to about 44° C., about 38° C. to about 45° C., about 38° C. to about 46° C., about 38° C. to about 47° C., about 38° C. to about 48° C., about 38° C. to about 49° C., about 38° C. to about 50° C., about 40° C. to about 44° C., about 40° C. to about 45° C., about 40° C. to about 46° C., about 40° C. to about 47° C., about 40° C. to about 48° C., about 40° C. to about 49° C., about 40° C. to about 50° C., about 42° C. to about 44° C., about 42° C. to about 45° C., about 42° C. to about 46° C., about 42° C. to about 47° C., about 42° C. to about 48° C., about 42° C. to about 49° C., or about 42° C. to about 50° C.

In some embodiments, a pharmaceutical composition disclosed herein may include one or more hard fats or glycerolipids that are solid at 18° C. comprising, or consisting essentially of or consisting of a mixture of triglycerides in an amount of, e.g., about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, or about 75% by weight. In some embodiments, a pharmaceutical composition disclosed herein may include one or more hard fats or glycerolipids that are solid at 18° C. comprising, or consisting essentially of or consisting of a mixture of triglycerides in an amount of, e.g., at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, or at least 75% by weight. In some embodiments, a pharmaceutical composition disclosed herein may include one or more hard fats or glycerolipids that are solid at 18° C. comprising, or consisting essentially of or consisting of a mixture of triglycerides in an amount of, e.g., at most 10% by weight, at most 15% by weight, at most 20% by weight, at most 25% by weight, at most 30% by weight, at most 35% by weight, at most 40% by weight, at most 45% by weight, at most 50% by weight, at most 55% by weight, at most 60% by weight, at most 65% by weight, at most 70% by weight, or at most 75% by weight.

In some embodiments, a pharmaceutical composition disclosed herein may include one or more hard fats or glycerolipids that are solid at 18° C. comprising, or consisting essentially of or consisting of a mixture of triglycerides in an amount of, e.g., about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 30% to about 35%, about 30% to about 40%, about 30% to about 45%, about 30% to about 50%, about 30% to about 55%, about 30% to about 60%, about 30% to about 65%, about 30% to about 70%, about 35% to about 40%, about 35% to about 45%, about 35% to about 50%, about 35% to about 55%, about 35% to about 60%, about 35% to about 65%, about 35% to about 70%, about 40% to about 45%, about 40% to about 50%, about 40% to about 55%, about 40% to about 60%, about 40% to about 65%, about 40% to about 70%, about 45% to about 50%, about 45% to about 55%, about 45% to about 60%, about 45% to about 65%, about 45% to about 70%, about 50% to about 55%, about 50% to about 60%, about 50% to about 65%, about 50% to about 70%, about 55% to about 60%, about 55% to about 65%, about 55% to about 70%, about 60% to about 65%, about 60% to about 70%, or about 65% to about 70% by weight.

In some embodiments, a pharmaceutical composition disclosed herein may include one or more liquid fats or glycerolipids that are liquid at 18° C. comprising, or consisting essentially of or consisting of a mixture of mono-, di- and triglycerides. In some embodiments, pharmaceutical composition disclosed herein may include one or more liquid fats or glycerolipids that are liquid at 18° C. comprising, or consisting essentially of or consisting of a mixture of unsaturated C₁₀-C₁₈ monoglycerides, C₁₀-C₁₈ diglycerides, and C₁₀-C₁₈ triglycerides, a mixture of unsaturated C₁₀-C₂₀ monoglycerides, C₁₀-C₂₀ diglycerides, and C₁₀-C₂₀ triglycerides, a mixture of unsaturated C₁₀-C₂₂ monoglycerides, C₁₀-C₂₂ diglycerides, and C₁₀-C₂₂ triglycerides, a mixture of unsaturated C₁₀-C₂₄ monoglycerides, C₁₀-C₂₄ diglycerides, and C₁₀-C₂₄ triglycerides, a mixture of unsaturated C₁₂-C₁₈ monoglycerides, C₁₂-C₁₈ diglycerides, and C₁₂-C₁₈ triglycerides, a mixture of unsaturated C₁₂-C₂₀ monoglycerides, C₁₂-C₂₀ diglycerides, and C₁₂-C₂₀ triglycerides, a mixture of unsaturated C₁₂-C₂₂ monoglycerides, C₁₂-C₂₂ diglycerides, and C₁₂-C₂₂ triglycerides, a mixture of unsaturated C₁₂-C₂₄ monoglycerides, C₁₂-C₂₄ diglycerides, and C₁₂-C₂₄ triglycerides, a mixture of unsaturated C₁₄-C₁₈ monoglycerides, C₁₄-C₁₈ diglycerides, and C₁₄-C₁₈ triglycerides, a mixture of unsaturated C₁₄-C₂₀ monoglycerides, C₁₄-C₂₀ diglycerides, and C₁₄-C₂₀ triglycerides, a mixture of unsaturated C₁₄-C₂₂ monoglycerides, C₁₄-C₂₂ diglycerides, and C₁₄-C₂₂ triglycerides, a mixture of unsaturated C₁₄-C₂₄ monoglycerides, C₁₄-C₂₄ diglycerides, and C₁₄-C₂₄ triglycerides, a mixture of unsaturated C₁₆-C₁₈ monoglycerides, C₁₆-C₁₈ diglycerides, and C₁₆-C₁₈ triglycerides, a mixture of unsaturated C₁₆-C₂₀ monoglycerides, C₁₆-C₂₀ diglycerides, and C₁₆-C₂₀ triglycerides, a mixture of unsaturated C₁₆-C₂₂ monoglycerides, C₁₆-C₂₂ diglycerides, and C₁₆-C₂₂ triglycerides, a mixture of unsaturated C₁₆-C₂₄ monoglycerides, C₁₆-C₂₄ diglycerides, and C₁₆-C₂₄ triglycerides, a mixture of unsaturated C₁₈-C₂₀ monoglycerides, C₁₈-C₂₀ diglycerides, and C₁₈-C₂₀ triglycerides, a mixture of unsaturated C₁₈-C₂₂ monoglycerides, C₁₈-C₂₂ diglycerides, and C₁₈-C₂₂ triglycerides, a mixture of unsaturated C₁₈-C₂₄ monoglycerides, C₁₈-C₂₄ diglycerides, and C₁₈-C₂₄ triglycerides, a mixture of unsaturated C₂₀-C₂₂ monoglycerides, C₂₀-C₂₂ diglycerides, C₂₀-C₂₂ triglycerides, or a mixture of unsaturated C₂₂-C₂₄ monoglycerides, C₂₂-C₂₄ diglycerides, and C₂₂-C₂₄ triglycerides.

In some embodiments, a pharmaceutical composition disclosed herein may include one or more liquid fats or glycerolipids that are liquid at 18° C. comprising, or consisting essentially of or consisting of a mixture of saturated C₁₀-C₁₈ monoglycerides, C₁₀-C₁₈ diglycerides, and C₁₀-C₁₈ triglycerides, a mixture of saturated C₁₀-C₂₀ monoglycerides, C₁₀-C₂₀ diglycerides, and C₁₀-C₂₀ triglycerides, a mixture of saturated C₁₀-C₂₂ monoglycerides, C₁₀-C₂₂ diglycerides, and C₁₀-C₂₂ triglycerides, a mixture of saturated C₁₀-C₂₄ monoglycerides, C₁₀-C₂₄ diglycerides, and C₁₀-C₂₄ triglycerides, a mixture of saturated C₁₂-C₁₈ monoglycerides, C₁₂-C₁₈ diglycerides, and C₁₂-C₁₈ triglycerides, a mixture of saturated C₁₂-C₂₀ monoglycerides, C₁₂-C₂₀ diglycerides, and C₁₂-C₂₀ triglycerides, a mixture of saturated C₁₂-C₂₂ monoglycerides, C₁₂-C₂₂ diglycerides, and C₁₂-C₂₂ triglycerides, a mixture of saturated C₁₂-C₂₄ monoglycerides, C₁₂-C₂₄ diglycerides, and C₁₂-C₂₄ triglycerides, a mixture of saturated C₁₄-C₁₈ monoglycerides, C₁₄-C₁₈ diglycerides, and C₁₄-C₁₈ triglycerides, a mixture of saturated C₁₄-C₂₀ monoglycerides, C₁₄-C₂₀ diglycerides, and C₁₄-C₂₀ triglycerides, a mixture of saturated C₁₄-C₂₂ monoglycerides, C₁₄-C₂₂ diglycerides, and C₁₄-C₂₂ triglycerides, a mixture of saturated C₁₄-C₂₄ monoglycerides, C₁₄-C₂₄ diglycerides, and C₁₄-C₂₄ triglycerides, a mixture of saturated C₁₆-C₁₈ monoglycerides, C₁₆-C₁₈ diglycerides, and C₁₆-C₁₈ triglycerides, a mixture of saturated C₁₆-C₂₀ monoglycerides, C₁₆-C₂₀ diglycerides, and C₁₆-C₂₀ triglycerides, a mixture of saturated C₁₆-C₂₂ monoglycerides, C₁₆-C₂₂ diglycerides, and C₁₆-C₂₂ triglycerides, a mixture of saturated C₁₆-C₂₄ monoglycerides, C₁₆-C₂₄ diglycerides, and C₁₆-C₂₄ triglycerides, a mixture of saturated C₁₈-C₂₀ monoglycerides, C₁₈-C₂₀ diglycerides, and C₁₈-C₂₀ triglycerides, a mixture of saturated C₁₈-C₂₂ monoglycerides, C₁₈-C₂₂ diglycerides, and C₁₈-C₂₂ triglycerides, a mixture of saturated C₁₈-C₂₄ monoglycerides, C₁₈-C₂₄ diglycerides, and C₁₈-C₂₄ triglycerides, a mixture of saturated C₂₀-C₂₂ monoglycerides, C₂₀-C₂₂ diglycerides, C₂₀-C₂₂ triglycerides, or a mixture of saturated C₂₂-C₂₄ monoglycerides, C₂₂-C₂₄ diglycerides, and C₂₂-C₂₄ triglycerides.

In some embodiments, a pharmaceutical composition disclosed herein may include one or more liquid fats or glycerolipids that are liquid at 18° C. comprising, or consisting essentially of or consisting of a mixture of saturated and unsaturated C₁₀-C₁₈ monoglycerides, C₁₀-C₁₈ diglycerides, and C₁₀-C₁₈ triglycerides, a mixture of saturated and unsaturated C₁₀-C₂₀ monoglycerides, C₁₀-C₂₀ diglycerides, and C₁₀-C₂₀ triglycerides, a mixture of saturated and unsaturated C₁₀-C₂₂ monoglycerides, C₁₀-C₂₂ diglycerides, and C₁₀-C₂₂ triglycerides, a mixture of saturated and unsaturated C₁₀-C₂₄ monoglycerides, C₁₀-C₂₄ diglycerides, and C₁₀-C₂₄ triglycerides, a mixture of saturated and unsaturated C₁₂-C₁₈ monoglycerides, C₁₂-C₁₈ diglycerides, and C₁₂-C₁₈ triglycerides, a mixture of saturated and unsaturated C₁₂-C₂₀ monoglycerides, C₁₂-C₂₀ diglycerides, and C₁₂-C₂₀ triglycerides, a mixture of saturated and unsaturated C₁₂-C₂₂ monoglycerides, C₁₂-C₂₂ diglycerides, and C₁₂-C₂₂ triglycerides, a mixture of saturated and unsaturated C₁₂-C₂₄ monoglycerides, C₁₂-C₂₄ diglycerides, and C₁₂-C₂₄ triglycerides, a mixture of saturated and unsaturated C₁₄-C₁₈ monoglycerides, C₁₄-C₁₈ diglycerides, and C₁₄-C₁₈ triglycerides, a mixture of saturated and unsaturated C₁₄-C₂₀ monoglycerides, C₁₄-C₂₀ diglycerides, and C₁₄-C₂₀ triglycerides, a mixture of saturated and unsaturated C₁₄-C₂₂ monoglycerides, C₁₄-C₂₂ diglycerides, and C₁₄-C₂₂ triglycerides, a mixture of saturated and unsaturated C₁₄-C₂₄ monoglycerides, C₁₄-C₂₄ diglycerides, and C₁₄-C₂₄ triglycerides, a mixture of saturated and unsaturated C₁₆-C₁₈ monoglycerides, C₁₆-C₁₈ diglycerides, and C₁₆-C₁₈ triglycerides, a mixture of saturated and unsaturated C₁₆-C₂₀ monoglycerides, C₁₆-C₂₀ diglycerides, and C₁₆-C₂₀ triglycerides, a mixture of saturated and unsaturated C₁₆-C₂₂ monoglycerides, C₁₆-C₂₂ diglycerides, and C₁₆-C₂₂ triglycerides, a mixture of saturated and unsaturated C₁₆-C₂₄ monoglycerides, C₁₆-C₂₄ diglycerides, and C₁₆-C₂₄ triglycerides, a mixture of saturated and unsaturated C₁₈-C₂₀ monoglycerides, C₁₈-C₂₀ diglycerides, and C₁₈-C₂₀ triglycerides, a mixture of saturated and unsaturated C₁₈-C₂₂ monoglycerides, C₁₈-C₂₂ diglycerides, and C₁₈-C₂₂ triglycerides, a mixture of saturated and unsaturated C₁₈-C₂₄ monoglycerides, C₁₈-C₂₄ diglycerides, and C₁₈-C₂₄ triglycerides, a mixture of saturated and unsaturated C₂₀-C₂₂ monoglycerides, C₂₀-C₂₂ diglycerides, C₂₀-C₂₂ triglycerides, or a mixture of saturated and unsaturated C₂₂-C₂₄ monoglycerides, C₂₂-C₂₄ diglycerides, and C₂₂-C₂₄ triglycerides.

In some embodiments, a pharmaceutical composition disclosed herein may include one or more liquid fats or glycerolipids that are liquid at 18° C. comprising, or consisting essentially of or consisting of a mixture of mono-, di- and triglycerides having a melting point of, e.g., at most 15° C., at most 16° C., at most 17° C., at most 18° C., at most 19° C., or at most 20° C. In some embodiments, a pharmaceutical composition disclosed herein may include one or more liquid fats or glycerolipids that are liquid at 18° C. comprising, or consisting essentially of or consisting of a mixture of mono-, di- and triglycerides having a melting point between, e.g., about 0° C. to about 5° C., about 0° C. to about 10° C., about 0° C. to about 15° C., about 0° C. to about 20° C., about 0° C. to about 22° C., about 0° C. to about 25° C., about 5° C. to about 10° C., about 5° C. to about 15° C., about 5° C. to about 20° C., about 5° C. to about 22° C., about 5° C. to about 25° C., about 10° C. to about 15° C., about 10° C. to about 20° C., about 10° C. to about 22° C., about 10° C. to about 25° C., about 15° C. to about 20° C., about 15° C. to about 22° C., or about 15° C. to about 25° C.

In some embodiments, a pharmaceutical composition disclosed herein may include one or more liquid fats or glycerolipids that are liquid at 18° C. comprising or consisting essentially of or consisting of a mixture of mono-, di-, and/or triglycerides in an amount of, e.g., about 10% by weight, about 15% by weight, about 20% by weight, about 25% by weight, about 30% by weight, about 35% by weight, about 40% by weight, about 45% by weight, about 50% by weight, about 55% by weight, about 60% by weight, about 65% by weight, about 70% by weight, or about 75% by weight. In some embodiments, a pharmaceutical composition disclosed herein may include one or more liquid fats or glycerolipids that are liquid at 18° C. comprising or consisting essentially of or consisting of one or more monoglycerides in an amount of, e.g. at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, or at least 75% by weight. In some embodiments, a pharmaceutical composition disclosed herein may include one or more liquid fats or glycerolipids that are liquid at 18° C. comprising or consisting essentially of or consisting of one or more monoglycerides in an amount of, e.g., at most 10% by weight, at most 15% by weight, at most 20% by weight, at most 25% by weight, at most 30% by weight, at most 35% by weight, at most 40% by weight, at most 45% by weight, at most 50% by weight, at most 55% by weight, at most 60% by weight, at most 65% by weight, at most 70% by weight, or at most 75% by weight.

In some embodiments, a pharmaceutical composition disclosed herein may include one or more liquid fats or glycerolipids that are liquid at 18° C. comprising or consisting essentially of or consisting of a mixture of mono-, di-, and/or triglycerides in an amount of, e.g., about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 30% to about 35%, about 30% to about 40%, about 30% to about 45%, about 30% to about 50%, about 30% to about 55%, about 30% to about 60%, about 30% to about 65%, about 30% to about 70%, about 35% to about 40%, about 35% to about 45%, about 35% to about 50%, about 35% to about 55%, about 35% to about 60%, about 35% to about 65%, about 35% to about 70%, about 40% to about 45%, about 40% to about 50%, about 40% to about 55%, about 40% to about 60%, about 40% to about 65%, about 40% to about 70%, about 45% to about 50%, about 45% to about 55%, about 45% to about 60%, about 45% to about 65%, about 45% to about 70%, about 50% to about 55%, about 50% to about 60%, about 50% to about 65%, about 50% to about 70%, about 55% to about 60%, about 55% to about 65%, about 55% to about 70%, about 60% to about 65%, about 60% to about 70%, or about 65% to about 70% by weight.

In some embodiments, pharmaceutical composition disclosed herein may include one or more liquid fats or glycerolipids that are liquid at 18° C. comprising, or consisting essentially of or consisting of one or more monoglycerides. A monoglyceride includes, without limitation, glycerol monomyristoleate, glycerol monopalmitoleate, glycerol monosapienate, glycerol monooleate, glycerol monoelaidate, glycerol monovaccenate, glycerol monolinoleate, glycerol monolinoelaidate, glycerol monolinolenate, glycerol monostearidonate, glycerol monoeicosenoate, glycerol monomeadate, glycerol monoarachidonate, glycerol monoeicosapentaenoate, glycerol monoerucate, glycerol monodocosahexaenoate, and glycerol mononervonate.

In some embodiments, pharmaceutical composition disclosed herein may include one or more liquid fats or glycerolipids that are liquid at 18° C. comprising, or consisting essentially of or consisting of unsaturated C₁₀-C₁₈ monoglycerides, unsaturated C₁₀-C₂₀ monoglycerides, unsaturated C₁₀-C₂₂ monoglycerides, unsaturated C₁₀-C₂₄ monoglycerides, unsaturated C₁₂-C₁₈ monoglycerides, unsaturated C₁₂-C₂₀ monoglycerides, unsaturated C₁₂-C₂₂ monoglycerides, unsaturated C₁₂-C₂₄ monoglycerides, unsaturated C₁₄-C₁₈ monoglycerides, unsaturated C₁₄-C₂₀ monoglycerides, unsaturated C₁₄-C₂₂ monoglycerides, unsaturated C₁₄-C₂₄ monoglycerides, unsaturated C₁₆-C₁₈ monoglycerides, unsaturated C₁₆-C₂₀ monoglycerides, unsaturated C₁₆-C₂₂ monoglycerides, unsaturated C₁₆-C₂₄ monoglycerides, unsaturated C₁₈-C₂₀ monoglycerides, unsaturated C₁₈-C₂₂ monoglycerides, unsaturated C₁₈-C₂₄ monoglycerides, unsaturated C₂₀-C₂₂ monoglycerides, or unsaturated C₂₂-C₂₄ monoglycerides.

In some embodiments, pharmaceutical composition disclosed herein may include one or more liquid fats or glycerolipids that are liquid at 18° C. comprising, or consisting essentially of or consisting of saturated C₁₀-C₁₈ monoglycerides, saturated C₁₀-C₂₀ monoglycerides, saturated C₁₀-C₂₂ monoglycerides, saturated C₁₀-C₂₄ monoglycerides, saturated C₁₂-C₁₈ monoglycerides, saturated C₁₂-C₂₀ monoglycerides, saturated C₁₂-C₂₂ monoglycerides, saturated C₁₂-C₂₄ monoglycerides, saturated C₁₄-C₁₈ monoglycerides, saturated C₁₄-C₂₀ monoglycerides, saturated C₁₄-C₂₂ monoglycerides, saturated C₁₄-C₂₄ monoglycerides, saturated C₁₆-C₁₈ monoglycerides, saturated C₁₆-C₂₀ monoglycerides, saturated C₁₆-C₂₂ monoglycerides, saturated C₁₆-C₂₄ monoglycerides, saturated C₁₈-C₂₀ monoglycerides, saturated C₁₈-C₂₂ monoglycerides, saturated C₁₈-C₂₄ monoglycerides, saturated C₂₀-C₂₂ monoglycerides, or saturated C₂₂-C₂₄ monoglycerides.

In some embodiments, a pharmaceutical composition disclosed herein may include one or more liquid fats or glycerolipids that are liquid at 18° C. comprising, or consisting essentially of or consisting of a mixture of saturated and unsaturated C₁₀-C₁₈ monoglycerides, a mixture of saturated and unsaturated C₁₀-C₂₀ monoglycerides, a mixture of saturated and unsaturated C₁₀-C₂₂ monoglycerides, a mixture of saturated and unsaturated C₁₀-C₂₄ monoglycerides, a mixture of saturated and unsaturated C₁₂-C₁₈ monoglycerides, a mixture of saturated and unsaturated C₁₂-C₂₀ monoglycerides, a mixture of saturated and unsaturated C₁₂-C₂₂ monoglycerides, a mixture of saturated and unsaturated C₁₂-C₂₄ monoglycerides, a mixture of saturated and unsaturated C₁₄-C₁₈ monoglycerides, a mixture of saturated and unsaturated C₁₄-C₂₀ monoglycerides, a mixture of saturated and unsaturated C₁₄-C₂₂ monoglycerides, a mixture of saturated and unsaturated C₁₄-C₂₄ monoglycerides, a mixture of saturated and unsaturated C₁₆-C₁₈ monoglycerides, a mixture of saturated and unsaturated C₁₆-C₂₀ monoglycerides, a mixture of saturated and unsaturated C₁₆-C₂₂ monoglycerides, a mixture of saturated and unsaturated C₁₆-C₂₄ monoglycerides, a mixture of saturated and unsaturated C₁₈-C₂₀ monoglycerides, a mixture of saturated and unsaturated C₁₈-C₂₂ monoglycerides, a mixture of saturated and unsaturated C₁₈-C₂₄ monoglycerides, a mixture of saturated and unsaturated C₂₀-C₂₂ monoglycerides, or a mixture of saturated and unsaturated C₂₂-C₂₄ monoglycerides.

In some embodiments, a pharmaceutical composition disclosed herein may include one or more liquid fats or glycerolipids that are liquid at 18° C. comprising, or consisting essentially of or consisting of a monoiglyceride having a melting point of, e.g., at most 15° C., at most 16° C., at most 17° C., at most 18° C., at most 19° C., or at most 20° C. In some embodiments, a pharmaceutical composition disclosed herein may include one or more liquid fats or glycerolipids that are liquid at 18° C. comprising, or consisting essentially of or consisting of a monoglyceride having a melting point between, e.g., about 0° C. to about 5° C., about 0° C. to about 10° C., about 0° C. to about 15° C., about 0° C. to about 20° C., about 0° C. to about 22° C., about 0° C. to about 25° C., about 5° C. to about 10° C., about 5° C. to about 15° C., about 5° C. to about 20° C., about 5° C. to about 22° C., about 5° C. to about 25° C., about 10° C. to about 15° C., about 10° C. to about 20° C., about 10° C. to about 22° C., about 10° C. to about 25° C., about 15° C. to about 20° C., about 15° C. to about 22° C., or about 15° C. to about 25° C.

In some embodiments, a pharmaceutical composition disclosed herein may include one or more liquid fats or glycerolipids that are liquid at 18° C. comprising or consisting essentially of or consisting of one or more monoglycerides in an amount of, e.g., about 10% by weight, about 15% by weight, about 20% by weight, about 25% by weight, about 30% by weight, about 35% by weight, about 40% by weight, about 45% by weight, about 50% by weight, about 55% by weight, or about 60% by weight. In some embodiments, a pharmaceutical composition disclosed herein may include one or more liquid fats or glycerolipids that are liquid at 18° C. comprising or consisting essentially of or consisting of one or more monoglycerides in an amount of, e.g. at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, or at least 60% by weight. In some embodiments, a pharmaceutical composition disclosed herein may include one or more liquid fats or glycerolipids that are liquid at 18° C. comprising or consisting essentially of or consisting of one or more monoglycerides in an amount of, e.g., at most 10% by weight, at most 15% by weight, at most 20% by weight, at most 25% by weight, at most 30% by weight, at most 35% by weight, at most 40% by weight, at most 45% by weight, at most 50% by weight, at most 55% by weight, or at most 60% by weight.

In some embodiments, a pharmaceutical composition disclosed herein may include one or more liquid fats or glycerolipids that are liquid at 18° C. comprising or consisting essentially of or consisting of one or more monoglycerides in an amount of, e.g., about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, about 55% to about 60% by weight.

Commercially available hard fats or glycerolipids that are solid at 18° C. include, without limitation, Cocoa butter, mixtures of saturated C₁₀-C₁₈ triglycerides having a melting point around 33° C. (GELUCIRE® 33/01), mixtures of saturated C₁₀-C₁₈ triglycerides having a melting point around 39° C. (GELUCIRE® 39/01), and mixtures of saturated C₁₀-C₁₈ triglycerides having a melting point around 43° C. (GELUCIRE® 43/01), Commercially available liquid fats or glycerolipids that are liquid at 18° C. include, without limitation, a hydrolyzed corn oil including glycerol monolinoleate (MAISINE™ 35-1, MAISINE™ CC). In some embodiments, a hydrolyzed corn oil including glycerol monolinoleate (MAISINE™ 35-1, MAISINE™ CC) comprises about 32% to 52% monoglycerides including glycerol monolinoleate, about 40% to 50% diglycerides, and about 5% to 30% triglycerides.

A pharmaceutical composition disclosed herein comprises any ratio of hard fats or glycerolipids that are solid at 18° C. to liquid fats or glycerolipids that are liquid at 18° C. that stabilizes one or more fibrates disclosed herein in a manner that prevents precipitation of the one or more fibrates. In some embodiments, a pharmaceutical composition disclosed herein comprises a hard fats or glycerolipids that are solid at 18° C. to a liquid fats or glycerolipids that are liquid at 18° C. ratio of, e.g., about 5:1, about 4:1, about 3:1, about 2:1, or about 1:1. In some embodiments, a pharmaceutical composition disclosed herein comprises a hard fats or glycerolipids that are solid at 18° C. to a liquid fats or glycerolipids that are liquid at 18° C. ratio of, e.g., about 5:1 to about 4:1, about 5:1 to about 3:1, about 5:1 to about 2:1, about 5:1 to about 1:1, about 4:1 to about 3:1, about 4:1 to about 2:1, about 4:1 to about 1:1, about 3:1 to about 2:1, about 3:1 to about 1:1, or about 2:1 to about 1:1.

In some embodiments, a pharmaceutical composition disclosed herein comprises a hard fats or glycerolipids that are solid at 18° C. to a liquid fats or glycerolipids that are liquid at 18° C. ratio of, e.g., about 1:5, about 1:4, about 1:3, or about 1:2. In some embodiments, a pharmaceutical composition disclosed herein comprises a hard fats or glycerolipids that are solid at 18° C. to a liquid fats or glycerolipids that are liquid at 18° C. ratio of, e.g., about 1:5 to about 1:4, about 1:5 to about 1:3, about 1:5 to about 1:2, about 1:5 to about 1:1, about 1:4 to about 1:3, about 1:4 to about 1:2, about 1:4 to about 1:1, about 1:3 to about 1:2, about 1:3 to about 1:1, or about 1:2 to about 1:1.

### Digestion Enhancers

A pharmaceutical composition disclosed herein may comprises one or more digestion enhancers. The primary purposes of the one or more digestion enhancers are to enhance solubility of a fibrate disclosed herein with the glycerolipid admixture, to enhance absorption of a fibrate disclosed herein thereby improving the pharmacokinetics of the compound, and/or to improve availability and facilitate selected bio-distribution of a fibrate disclosed herein thereby improving the pharmacodynamics of the compound. These improved properties are achieved by the one or more digestion enhancers by creating a pre-lipid digestion formulation of a fibrate disclosed herein which facilitates and enhances the processing of the one or more glycerolipids disclosed herein when a pharmaceutical composition disclosed herein enters the duodenum region of the small intestine. Such glycerolipid processing enables the one or more fibrates contained in the pharmaceutical composition to be absorbed by enterocytes along with the digested glycerolipids and subsequently processed and transported into the lymphatic system. In some embodiments, a pharmaceutical composition disclosed comprises one or more digestion enhancers that includes one or more bile acids. In some embodiments, a pharmaceutical composition disclosed comprises one or more digestion enhancers that includes cholic acid. In some embodiments, a pharmaceutical composition disclosed comprises one or more digestion enhancers that includes one or more C₁₄-C₂₄ free fatty acids. In some embodiments, a pharmaceutical composition disclosed comprises one or more digestion enhancers that includes one or more C₁₄-C₂₀ free fatty acids. In some embodiments, a pharmaceutical composition disclosed comprises one or more digestion enhancers that includes an oleic acid, a steric acid, or a linoleic acid. In some embodiments, a pharmaceutical composition disclosed comprises one or more digestion enhancers that includes one or more C₁₄-C₂₄ free fatty acid surfactants. In some embodiments, a pharmaceutical composition disclosed comprises one or more digestion enhancers that includes one or more C₁₄-C₂₀ free fatty acid surfactants. In some embodiments, a pharmaceutical composition disclosed comprises one or more digestion enhancers that includes a sodium oleate, a sodium stearate, and/or a sodium linoleate.

In some embodiments, a pharmaceutical composition disclosed comprises one or more digestion enhancers that includes 1) one or more bile acids disclosed herein; and 2) one or more C₁₄-C₂₄ free fatty acids disclosed herein. In some embodiments, a pharmaceutical composition disclosed comprises one or more digestion enhancers that includes 1) a cholic acid disclosed herein; and 2) one or more C₁₄-C₂₀ free fatty acids disclosed herein. In some embodiments, a pharmaceutical composition disclosed comprises one or more digestion enhancers that includes 1) a cholic acid disclosed herein; and 2) an oleic acid, a steric acid, or a linoleic acid.

In some embodiments, a pharmaceutical composition disclosed comprises one or more digestion enhancers that includes 1) one or more bile acids disclosed herein; 2) one or more C₁₄-C₂₄ free fatty acids disclosed herein; and 3) one or more C₁₄-C₂₄ free fatty acid surfactants disclosed herein. In some embodiments, a pharmaceutical composition disclosed comprises one or more digestion enhancers that includes 1) a cholic acid disclosed herein; 2) one or more C₁₄-C₂₀ free fatty acids disclosed herein; and 3) one or more C₁₄-C₂₀ free fatty acid surfactants disclosed herein. In some embodiments, a pharmaceutical composition disclosed comprises one or more digestion enhancers that includes 1) a cholic acid disclosed herein; 2) an oleic acid, a steric acid, and/or a linoleic acid; and 3) a sodium oleate, a sodium stearate, and/or a sodium linoleate.

In some embodiments, a pharmaceutical composition disclosed comprises one or more digestion enhancers that includes 1) one or more bile acids disclosed herein; and 2) one or more phospholipids disclosed herein. In some embodiments, a pharmaceutical composition disclosed comprises one or more digestion enhancers that includes 1) a cholic acid disclosed herein; and 2) one or more phospholipids disclosed herein.

In some embodiments, a pharmaceutical composition disclosed comprises one or more digestion enhancers that includes 1) one or more C₁₄-C₂₄ free fatty acids disclosed herein; and 2) one or more phospholipids disclosed herein. In some embodiments, a pharmaceutical composition disclosed comprises one or more digestion enhancers that includes 1) one or more C₁₄-C₂₀ free fatty acids disclosed herein; and 2) one or more phospholipids disclosed herein. In some embodiments, a pharmaceutical composition disclosed comprises one or more digestion enhancers that includes 1) an oleic acid, a steric acid, and/or a linoleic acid; and 2) one or more phospholipids disclosed herein.

In some embodiments, a pharmaceutical composition disclosed comprises one or more digestion enhancers that includes 1) one or more bile acids disclosed herein; 2) one or more C₁₄-C₂₄ free fatty acids disclosed herein; and 3) one or more phospholipids disclosed herein. In some embodiments, a pharmaceutical composition disclosed comprises one or more digestion enhancers that includes 1) one or more bile acids disclosed herein; 2) one or more C₁₄-C₂₀ free fatty acids disclosed herein; and 3) one or more phospholipids disclosed herein. In some embodiments, a pharmaceutical composition disclosed comprises one or more digestion enhancers that includes 1) a cholic acid disclosed herein; 2) an oleic acid, a steric acid, and/or a linoleic acid; and 3) one or more phospholipids disclosed herein.

In some embodiments, a pharmaceutical composition disclosed comprises one or more digestion enhancers that includes 1) one or more bile acids disclosed herein; 2) one or more phospholipids disclosed herein; and 3) one or more C₁₄-C₂₄ free fatty acid surfactants disclosed herein. In some embodiments, a pharmaceutical composition disclosed comprises one or more digestion enhancers that includes 1) a cholic acid disclosed herein; 2) one or more phospholipids disclosed herein; and 3) one or more C₁₄-C₂₀ free fatty acid surfactants disclosed herein. In some embodiments, a pharmaceutical composition disclosed comprises one or more digestion enhancers that includes 1) a cholic acid disclosed herein; 2) one or more phospholipids disclosed herein; and 3) a sodium C₁₄-C₂₀ free fatty acid disclosed herein. In some embodiments, a pharmaceutical composition disclosed comprises one or more digestion enhancers that includes 1) a cholic acid disclosed herein; 2) one or more phospholipids disclosed herein; and 3) a sodium oleate, a sodium stearate, and/or a sodium linoleate.

In some embodiments, a pharmaceutical composition disclosed comprises one or more digestion enhancers that includes 1) one or more C₁₄-C₂₄ free fatty acids disclosed herein; 2) one or more phospholipids disclosed herein; and 3) one or more C₁₄-C₂₄ free fatty acid surfactants disclosed herein. In some embodiments, a pharmaceutical composition disclosed comprises one or more digestion enhancers that includes 1) one or more C₁₄-C₂₀ free fatty acids disclosed herein; 2) one or more phospholipids disclosed herein; and 3) one or more C₁₄-C₂₀ free fatty acid surfactants disclosed herein. In some embodiments, a pharmaceutical composition disclosed comprises one or more digestion enhancers that includes 1) an oleic acid, a steric acid, and/or a linoleic acid; 2) one or more phospholipids disclosed herein; and 3) one or more sodium C₁₄-C₂₀ free fatty acid surfactants disclosed herein. In some embodiments, a pharmaceutical composition disclosed comprises one or more digestion enhancers that includes 1) an oleic acid, a steric acid, and/or a linoleic acid; 2) one or more phospholipids disclosed herein; and 3) a sodium oleate, a sodium stearate, and/or a sodium linoleate.

In some embodiments, a pharmaceutical composition disclosed comprises one or more digestion enhancers that includes 1) one or more bile acids disclosed herein; 2) one or more C₁₄-C₂₄ free fatty acids disclosed herein; 3) one or more phospholipids disclosed herein; and 4) one or more C₁₄-C₂₄ free fatty acid surfactants disclosed herein. In some embodiments, a pharmaceutical composition disclosed comprises one or more digestion enhancers that includes 1) one or more bile acids disclosed herein; 2) one or more C₁₄-C₂₀ free fatty acids disclosed herein; 3) one or more phospholipids disclosed herein; and 4) one or more C₁₄-C₂₀ free fatty acid surfactants disclosed herein. In some embodiments, a pharmaceutical composition disclosed comprises one or more digestion enhancers that includes 1) a cholic acid disclosed herein; 2) an oleic acid, a steric acid, and/or a linoleic acid; 3) one or more phospholipids disclosed herein; and 4) one or more sodium C₁₄-C₂₀ free fatty acid surfactants disclosed herein. In some embodiments, a pharmaceutical composition disclosed comprises one or more digestion enhancers that includes 1) a cholic acid disclosed herein; 2) an oleic acid, a steric acid, and/or a linoleic acid; 3) one or more phospholipids disclosed herein; and 4) a sodium oleate, a sodium stearate, and/or a sodium linoleate.

In some embodiments, a pharmaceutical composition disclosed herein may comprising or consisting essentially of or consisting of one or more digestion enhancers in an amount of, e.g., about 1% by weight, about 2.5% by weight, about 5% by weight, about 7.5% by weight, about 10% by weight, about 12.5% by weight, about 15% by weight, about 17.5% by weight, about 20% by weight, about 22.5% by weight, about 25% by weight, about 30% by weight, about 35% by weight, about 40% by weight, about 45% by weight, about 50% by weight, about 55% by weight, about 60% by weight, about 65% by weight, about 70% by weight, about 75% by weight, or about 80% by weight. In some embodiments, a pharmaceutical composition disclosed herein may comprising or consisting essentially of or consisting of one or more digestion enhancers in an amount of, e.g., at least 1% by weight, at least 2.5% by weight, at least 5% by weight, at least 7.5% by weight, at least 10% by weight, at least 12.5% by weight, at least 15% by weight, at least 17.5% by weight, at least 20% by weight, at least 22.5% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, at least 75% by weight, or at least 75% by weight. In some embodiments, a pharmaceutical composition disclosed herein may comprising or consisting essentially of or consisting of one or more digestion enhancers in an amount of, e.g., at most 1% by weight, at most 2.5% by weight, at most 5% by weight, at most 7.5% by weight, at most 10% by weight, at most 12.5% by weight, at most 15% by weight, at most 17.5% by weight, at most 20% by weight, at most 22.5% by weight, at most 25% by weight, at most 30% by weight, at most 35% by weight, at most 40% by weight, at most 45% by weight, at most 50% by weight, at most 55% by weight, at most 60% by weight, at most 65% by weight, at most 70% by weight, at most 75% by weight, or at most 80% by weight.

In some embodiments, a pharmaceutical composition disclosed herein may comprising or consisting essentially of or consisting of one or more digestion enhancers in an amount of, e.g., about 1% to about 2.5% by weight, about 1% to about 5% by weight, about 1% to about 10% by weight, about 1% to about 15% by weight, about 1% to about 20% by weight, about 1% to about 25% by weight, about 2.5% to about 5% by weight, about 2.5% to about 10% by weight, about 2.5% to about 15% by weight, about 2.5% to about 20% by weight, about 2.5% to about 25% by weight, about 5% to about 10% by weight, about 5% to about 15% by weight, about 5% to about 20% by weight, about 5% to about 25% by weight, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 30% to about 35%, about 30% to about 40%, about 30% to about 45%, about 30% to about 50%, about 30% to about 55%, about 30% to about 60%, about 30% to about 65%, about 30% to about 70%, about 35% to about 40%, about 35% to about 45%, about 35% to about 50%, about 35% to about 55%, about 35% to about 60%, about 35% to about 65%, about 35% to about 70%, about 40% to about 45%, about 40% to about 50%, about 40% to about 55%, about 40% to about 60%, about 40% to about 65%, about 40% to about 70%, about 40% to about 75%, about 40% to about 80%, about 45% to about 50%, about 45% to about 55%, about 45% to about 60%, about 45% to about 65%, about 45% to about 70%, about 45% to about 75%, about 45% to about 80%, about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70% by weight, about 65% to about 75%, about 65% to about 80%, about 70% to about 75%, about 70% to about 80%, or about 75% to about 80%.

### Bile Acids

A pharmaceutical composition disclosed herein may comprises one or more bile acids. The primary purpose of bile acids are to enhance solubility of a fibrate disclosed herein within the glycerolipid admixture, to enhance absorption of a fibrate disclosed herein thereby improving the pharmacokinetics of the compound, and/or to improve availability and facilitate selected bio-distribution of a fibrate disclosed herein thereby improving the pharmacodynamics of the compound. In addition, as disclosed in Example 2, a bile acid disclosed herein improves the solubility of a fibrate disclosed herein within one or more glycerolipid and/or one or more free C₁₄₋₂₄ fatty acids disclosed herein. The improved solubility properties are achieved by the bile acids by preventing the recrystallization of a fibrate during solidification when the molton pharmaceutical composition cools to room temperature (18° C. to 20° C.). The improved pharmacokinetic properties are achieved by the bile acids by breaking apart the lipid component of a pharmaceutical composition disclosed herein via its surfactant properties into smaller lipid structures that mimic emulsion droplets, thereby facilitating the emulsification process. The “emulsion droplets” recruit coliapse and create a greater surface area for which pancreatic lipase can digest the hard fat glycerolipids present there within which ultimately enhances enterocyte absorption and subsequent chylomicron formation. Bile acids are involved in signaling for the initiation of chylomicron formation. Hence the inclusion of bile acids in the lipid formulation will maximise this signaling pathway and the production of chylomicrons, As a result, the improved pharmacodynamic properties provided by bile acids are produced by increasing the availability of a fibrate by increasing its content within chylomicrons prior to entering the circulatory system and subsequently facilitating the delivery of the fibrate to compartments such as the brain passing across membranes such as the blood-brain barrier and the choroid plexus.

Amphipathic molecules having an HLB of greater than 12, bile acids have a specific chemical structure, different from ordinary aliphatic surfactants, due to the presence of a large, rigid, and planar hydrophobic moiety of a steroid nucleus carrying 2-4 hydroxyl groups. Specifically, bile acids comprise the following basic components: (1) 4 rings, (2) a 5-/8-carbon side chain that ends with a carboxylic acid, and (3) a number of hydroxyl groups (whose position/number changes among the various salts). The rings are ascribed the letters A, B, C, and D based on their distance from the side chain with the —COOH group, the D ring being the most distant (as well as being 1 C smaller than the other rings), as discussed below. Beta hydroxyl groups face up/out, alpha groups down, and every bile acid has a 3-hydroxyl group that originates from their cholesterol precursor. The chemical structure of bile salts results in this emulsification pathway being useful in accordance with the teachings of the disclosure, and accordingly, synthetic surfactants will not work.

Examples of bile acids include, without limitation, chenodeoxycholic acid, cholic acid, dafachronic acid, deoxycholic acid, glycocholic acid, glycohenodeoxycholic acid, lithocholic acid, taurochenodeoxycholic acid, taurocholic acid, and any stereoisomer thereof. cholic acid and chenodeoxycholic acid are referred to as primary bile acids while deoxycholic acid (which is converted from cholic acid) and lithocholic acid (which is converted from chenodeoxycholic acid) are referred to as secondary bile acids. Taurocholic acid and glycocholic acid (derivatives of cholic acid) and taurochenodeoxycholic acid and glycochenodeoxycholic acid (derivatives of chenodeoxycholic acid) are the major bile acids that serve as the basis for the bile salts found in bile.

There is a direct correlation between the amount of a bile acid present in a pharmaceutical composition disclosed herein and the improved properties observed. As such, the more bile acid present in a pharmaceutical composition disclosed herein the greater the improvement in solubility, absorption, and availability of the therapeutic composition. In addition, the upper limit of bile acid including in a pharmaceutical composition disclosed herein is not limited to the solution point of a bile acid. As such, a pharmaceutical composition disclosed herein can include supersaturating amounts of a bile acid. As such, the upper limit of bile acid that can be included in a pharmaceutical composition disclosed herein is its critical micellar concentration (CMC). Besides the above-mentioned improved properties, an additional advantage of supersaturating amounts of a bile acid is the presence of the resulting nanoparticle formation of crystalline bile acid in a pharmaceutical composition disclosed herein. Without wishing to be limited by a one theory, bile acid nanoparticles can serve as a reservoir that upon exposure to the alkaline environment of the small intestine dissolve and form bile salts which in turn further enhances the emulsification process of the pharmaceutical composition disclosed herein.

The amount of a bile acid useful in a pharmaceutical composition disclosed herein is an amount below its CMC. In some embodiments, the amount of a bile acid useful in a pharmaceutical composition disclosed herein is an amount below its CMC and one that is supersaturating. In some embodiments, a pharmaceutical composition disclosed herein may comprising or consisting essentially of or consisting of one or more bile acids in an amount of, e.g., about 0.1% by weight, about 0.5% by weight, about 1.0% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, or about 10.0% by weight. In some embodiments, a pharmaceutical composition disclosed herein may comprising or consisting essentially of or consisting of one or more bile acids in an amount of, e.g., at least 0.1% by weight, at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, or at least 10.0% by weight. In some embodiments, a pharmaceutical composition disclosed herein may comprising or consisting essentially of or consisting of one or more bile acids in an amount of, e.g., at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, or at most 10.0% by weight.

In some embodiments, a pharmaceutical composition disclosed herein may comprising or consisting essentially of or consisting of one or more bile acids in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.

In some embodiments, a pharmaceutical composition disclosed herein may comprising or consisting essentially of or consisting of one or more bile acids in a concentration of, e.g., at most 0.01 mM, at most 0.025 mM, at most 0.05 mM, at most 0.075 mM, at most 0.1 mM, at most 0.25 mM, at most 0.5 mM, at most 0.75 mM, at most 1 mM, or at most 5 mM. In some embodiments, a pharmaceutical composition disclosed herein may comprising or consisting essentially of or consisting of one or more bile acids in a concentration of, e.g., about 0.01 mM to about 0.05 mM, about 0.01 mM to about 0.1 mM, about 0.01 mM to about 0.5 mM, about 0.01 mM to about 1 mM, about 0.01 mM to about 5 mM, about 0.05 mM to about 0.1 mM, about 0.05 mM to about 0.5 mM, about 0.05 mM to about 1 mM, about 0.05 mM to about 5 mM, about 0.1 mM to about 0.5 mM, about 0.1 mM to about 1 mM, about 0.1 mM to about 5 mM, or about 1 mM to about 5 mM.

### C₁₄₋₂₄ Fatty Acids

A pharmaceutical composition disclosed herein may comprises one or more free C₁₄₋₂₄ fatty acids. A fatty acid comprises a carboxylic acid with a long unbranched hydrocarbon chain which may be either saturated or unsaturated and are hydrophobic molecules having an HLB of less than 4. The primary purpose of free C₁₄₋₂₄ fatty acids are to enhance solubility of a fibrate disclosed herein within the glycerolipid admixture and to enhance absorption of a fibrate disclosed herein thereby improving the pharmacokinetics of the compound. The improved solubility properties are achieved by the free C₁₄₋₂₄ fatty acids due to their properties of being a solvent that facilitates dissolvement of a fibrate disclosed herein. The improved absorption properties are achieved by the free C₁₄₋₂₄ fatty acids by facilitating and increasing the formation of micelles by breaking up larger emulsion droplets, thereby mimicking the lipid digestion products of triglycerides, namely free fatty acids. As such, a C₁₄₋₂₄ fatty acid disclosed herein increases the uptake of micelles comprising one or more fibrates into enterocytes. In addition, as disclosed in Example 2, a free C₁₄₋₂₄ fatty acids disclosed herein improves the solubility of a bile salt disclosed herein.

In some embodiments, pharmaceutical composition disclosed herein may include one or more free C₁₄₋₂₄ fatty acids comprising, or consisting essentially of or consisting of unsaturated free C₁₄-C₁₆ fatty acids, unsaturated free C₁₄-C₁₈ fatty acids, unsaturated free C₁₄-C₂₀ fatty acids, unsaturated free C₁₄-C₂₂ fatty acids, unsaturated free C₁₄-C₂₄ fatty acids, unsaturated free C₁₆-C₁₈ fatty acids, unsaturated free C₁₆-C₂₀ fatty acids, unsaturated free C₁₆-C₂₂ fatty acids, unsaturated free C₁₆-C₂₄ fatty acids, unsaturated free C₁₈-C₂₀ fatty acids, unsaturated free C₁₈-C₂₂ fatty acids, unsaturated free C₁₈-C₂₄ fatty acids, unsaturated free C₂₀-C₂₂ fatty acids, or unsaturated free C₂₂-C₂₄ fatty acids. In some embodiments, pharmaceutical composition disclosed herein may include one or more free C₁₄₋₂₄ fatty acids comprising, or consisting essentially of or consisting of ω-3 unsaturated free C₁₈-C₂₂ fatty acids, w-5 unsaturated free C₁₈-C₂₂ fatty acids, ω-6 unsaturated free C₁₈-C₂₂ fatty acids, ω-7 unsaturated free C₁₈-C₂₂ fatty acids, ω-9 unsaturated free C₁₈-C₂₂ fatty acids, ω-10 unsaturated free C₁₈-C₂₂ fatty acids, ω-11 unsaturated free C₁₈-C₂₂ fatty acids, or ω-12 unsaturated free C₁₈-C₂₂ fatty acids.

In some embodiments, pharmaceutical composition disclosed herein may include one or more free C₁₄₋₂₄ fatty acids comprising, or consisting essentially of or consisting of saturated free C₁₄-C₁₆ fatty acids, saturated free C₁₄-C₁₈ fatty acids, saturated free C₁₄-C₂₀ fatty acids, saturated free C₁₄-C₂₂ fatty acids, saturated free C₁₄-C₂₄ fatty acids, saturated free C₁₆-C₁₈ fatty acids, saturated free C₁₆-C₂₀ fatty acids, saturated free C₁₆-C₂₂ fatty acids, saturated free C₁₆-C₂₄ fatty acids, saturated free C₁₈-C₂₀ fatty acids, saturated free C₁₈-C₂₂ fatty acids, saturated free C₁₈-C₂₄ fatty acids, saturated free C₂₀-C₂₂ fatty acids, or saturated free C₂₂-C₂₄ fatty acids.

In some embodiments, a pharmaceutical composition disclosed herein may include one or more free C₁₄₋₂₄ fatty acids comprising, or consisting essentially of or consisting of a mixture of saturated and unsaturated free C₁₄-C₁₆ fatty acids, a mixture of saturated and unsaturated free C₁₄-C₁₈ fatty acids, a mixture of saturated and unsaturated free C₁₄-C₂₀ fatty acids, a mixture of saturated and unsaturated free C₁₄-C₂₂ fatty acids, a mixture of saturated and unsaturated free C₁₄-C₂₄ fatty acids, a mixture of saturated and unsaturated free C₁₆-C₁₈ fatty acids, a mixture of saturated and unsaturated free C₁₆-C₂₀ fatty acids, a mixture of saturated and unsaturated free C₁₆-C₂₂ fatty acids, a mixture of saturated and unsaturated free C₁₆-C₂₄ fatty acids, a mixture of saturated and unsaturated free C₁₈-C₂₀ fatty acids, a mixture of saturated and unsaturated free C₁₈-C₂₂ fatty acids, a mixture of saturated and unsaturated free C₁₈-C₂₄ fatty acids, a mixture of saturated and unsaturated free C₂₀-C₂₂ fatty acids, or a mixture of saturated and unsaturated free C₂₂-C₂₄ fatty acids.

Non-limiting examples of a free C₁₄₋₂₄ fatty acids include palmitic acid (hexadecenoic acid), palmitolinolenic acid, palmitidonic acid, palmitovaccenic acid, palmitoleic acid, sapienic acid, 4-Hexadecenoic acid, stearic acid (octadecenoic acid), α-linolenic acid, stearidonic acid, α-eleostearic acid, β-eleostearic acid, pumicic acid, 7,10,13-octadecatrienoic acid, 12-octadecenoic acid, linoleic acid, linolelaidic acid. γ-linolenic acid, calendic acid, pinolenic acid, vaccinic acid, ruminic acid, oleic acid, elaidic acid, petroselinic acid, arachidic acid (eicosanoic acid), dihomo-α-linolenic acid, eicosic acidtraenoic acid, eicosapentaenoic acid, 9,12,15-eicosatrienoic acid, β-eicosic acidtraenoic acid, dihomo-linoleic acid, dihomo-γ-linolenic acid, arachidonic acid, paullinic acid, 7,10,13-eicosatrienoic acid, gondoic acid, 8,11-eicosadienoic acid, meadic acid, gadoleic acid, 8-eicosenoic acid, behenic acid (docosanoic acid), clupanodonic acid, docosahexaenoic acid, adrenic acid, osbondic acid, erucic acid, lignoceric acid (tetracosanic acid), 9,12,15,18,21-Tetracosapentaenoic acid, 6,9,12,15,18,21-Tetracosahexaenoic acid, and nervonic acid.

In some embodiments, a pharmaceutical composition disclosed herein may comprising or consisting essentially of or consisting of one or more free C₁₄₋₂₄ fatty acids in an amount of, e.g., about 1% by weight, about 2.5% by weight, about 5% by weight, about 7.5% by weight, about 10% by weight, about 12.5% by weight, about 15% by weight, about 17.5% by weight, about 20% by weight, about 22.5% by weight, about 25% by weight, about 30% by weight, about 35% by weight, about 40% by weight, about 50% by weight, about 60% by weight, about 70% by weight, or about 75% by weight. In some embodiments, a pharmaceutical composition disclosed herein may comprising or consisting essentially of or consisting of one or more free C₁₄₋₂₄ fatty acids in an amount of, e.g., at least 1% by weight, at least 2.5% by weight, at least 5% by weight, at least 7.5% by weight, at least 10% by weight, at least 12.5% by weight, at least 15% by weight, at least 17.5% by weight, at least 20% by weight, at least 22.5% by weight, at least 25% by weight, at least 30% by weight, at least 35% by weight, at least 40% by weight, at least 50% by weight, at least 60% by weight, at least 70% by weight, or at least 75% by weight. In some embodiments, a pharmaceutical composition disclosed herein may comprising or consisting essentially of or consisting of one or more free C₁₄₋₂₄ fatty acids in an amount of, e.g., at most 1% by weight, at most 2.5% by weight, at most 5% by weight, at most 7.5% by weight, at most 10% by weight, at most 12.5% by weight, at most 15% by weight, at most 17.5% by weight, at most 20% by weight, at most 22.5% by weight, at most 25% by weight, at most 30% by weight, at most 35% by weight, at most 40% by weight, at most 50% by weight, at most 60% by weight, at most 70% by weight, or at most 75% by weight.

In some embodiments, a pharmaceutical composition disclosed herein may comprising or consisting essentially of or consisting of one or more free C₁₄₋₂₄ fatty acids in an amount of, e.g., about 1% to about 2.5% by weight, about 1% to about 5% by weight, about 1% to about 10% by weight, about 1% to about 15% by weight, about 1% to about 20% by weight, about 1% to about 25% by weight, about 2.5% to about 5% by weight, about 2.5% to about 10% by weight, about 2.5% to about 15% by weight, about 2.5% to about 20% by weight, about 2.5% to about 25% by weight, about 2.5% to about 30% by weight, about 5% to about 10% by weight, about 5% to about 15% by weight, about 5% to about 20% by weight, about 5% to about 25% by weight, about 5% to about 30% by weight, about 10% to about 15% by weight, about 10% to about 20% by weight, about 10% to about 25% by weight, about 10% to about 30% by weight, about 10% to about 40% by weight, about 10% to about 45% by weight, about 10% to about 50% by weight, about 10% to about 60% by weight, about 10% to about 70% by weight, about 15% to about 20% by weight, about 15% to about 25% by weight, about 15% to about 30% by weight, about 15% to about 40% by weight, about 15% to about 45% by weight, about 15% to about 50% by weight, about 15% to about 60% by weight, about 15% to about 70% by weight, about 20% to about 25% by weight, about 20% to about 30% by weight, about 20% to about 40% by weight, about 20% to about 45% by weight, about 20% to about 50% by weight, about 20% to about 60% by weight, about 20% to about 70% by weight, about 30% to about 40% by weight, about 30% to about 50% by weight, about 30% to about 60% by weight, about 30% to about 70% by weight, about 30% to about 75% by weight, about 35% to about 40% by weight, about 35% to about 50% by weight, about 35% to about 60% by weight, about 35% to about 70% by weight, about 35% to about 75% by weight, about 40% to about 50% by weight, about 40% to about 60% by weight, about 40% to about 70% by weight, about 40% to about 75% by weight, about 50% to about 60% by weight, about 50% to about 70% by weight, or about 60% to about 70% by weight.

In some embodiments, a pharmaceutical composition disclosed herein may comprising or consisting essentially of or consisting of one or more free C₁₄₋₂₄ fatty acids in a concentration of, e.g., at most 0.01 mM, at most 0.025 mM, at most 0.05 mM, at most 0.075 mM, at most 0.1 mM, at most 0.25 mM, at most 0.5 mM, at most 0.75 mM, at most 1 mM, at most 1.25 mM, at most 1.5 mM, at most 1.75 mM, or at most 2 mM. In some embodiments, a pharmaceutical composition disclosed herein may comprising or consisting essentially of or consisting of one or more free C₁₄₋₂₄ fatty acids in a concentration of, e.g., about 0.01 mM to about 0.05 mM, about 0.01 mM to about 0.1 mM, about 0.01 mM to about 0.5 mM, about 0.01 mM to about 1 mM, about 0.01 mM to about 1.25 mM, about 0.01 mM to about 1.5 mM, about 0.01 mM to about 1.75 mM, about 0.01 mM to about 2 mM, about 0.05 mM to about 0.1 mM, about 0.05 mM to about 0.5 mM, about 0.05 mM to about 1 mM, about 0.05 mM to about 1.25 mM, about 0.05 mM to about 1.5 mM, about 0.05 mM to about 1.75 mM, about 0.05 mM to about 2 mM, about 0.1 mM to about 0.5 mM, about 0.1 mM to about 1 mM, about 0.1 mM to about 1.25 mM, about 0.1 mM to about 1.5 mM, about 0.1 mM to about 1.75 mM, about 0.1 mM to about 2 mM, about 0.5 mM to about 1 mM, about 0.5 mM to about 1.25 mM, about 0.5 mM to about 1.5 mM, about 0.5 mM to about 1.75 mM, about 0.5 mM to about 2 mM, about 1 mM to about 1.25 mM, about 1 mM to about 1.5 mM, about 1 mM to about 1.75 mM, or about 15 mM to about 2 mM.

### Phospholipids

A pharmaceutical composition disclosed herein may comprises one or more phospholipids. Like bile acids disclosed herein, phospholipids disclosed herein to break apart the lipid component of a pharmaceutical composition disclosed herein via its surfactant properties into smaller lipid structures that mimic emulsion droplets, thereby facilitating the emulsification process. The “emulsion droplets” recruit coliapse and create a greater surface area for which pancreatic lipase can digest the hard fat glycerolipids present therewithin. In addition phospholipids disclosed herein, along with free fatty acid surfactants disclosed herein, facilitate formation of micelles by associating with the lipid digestion products of triglycerides, and then enhancing association of the triglyceride digestion products with fatty acid transporters, thereby enhancing absorption lipid molecules and the associated fibrate into enterocytes.

The structure of the phospholipid generally comprises a hydrophobic tail of one or more fatty acids and a hydrophilic head containing phosphoric acid functional group and is amphipathic in nature and having an HLB of greater than 12. Phospholipids include, without limitation, phosphoglycerides and phosphosphingolipids. Phosphoglycerides have a general structure comprising a glycerol backbone with two fatty acids esterified to the first and second hydroxyl groups of glycerol and a phosphoric acid group esterified to the third hydroxyl group of glycerol. An alcohol group is esterified to the phosphoric acid group of a phosphoglyceride. Phosphoglycerides always have two fatty acids usually with one fatty acid being saturated and the other being unsaturated. Phosphoglycerides are generally typed according to the particular alcohol group present on the phosphortic acid group, such as, e.g., ethanolamine, choline, serine, or inositol. Non-limiting examples of phosphoglycerides include a phosphatidic acid (phosphatidate) (PA), a phosphatidylethanolamine (PE), a phosphatidylcholine (PC), a phosphatidylserine (PS), a cardiolipin, and a phosphoinositide including phosphatidylinositol (PI), phosphatidylinositol phosphate (PIP), phosphatidylinositol bisphosphate (PIP2), and phosphatidylinositol triphosphate (PIP3).

Although structurally different, phosphosphingolipids also have a polar head and two nonpolar tails. Phosphosphingolipids have a general structure comprising a long-chain amino alcohol sphingosine backbone with a fatty acid forming an amide linkage to the amino group of to the sphingosine backbone and a phosphoric acid group esterified to the hydroxyl group of the sphingosine backbone. Non-limiting examples of phosphosphingolipids include a ceramide phosphorylethanolamine (Cer-PE), and ceramide phosphorylcholine (Cer-PC), and ceramide phosphorylglycerol (Cer-PG).

In addition to its amphipathic nature, a phospholipid can be a zwitterionic phospholipids. A zwitterionic phospholipid is a fully ionized molecule that contains an equal number of positively charged and negatively charged functional groups and is electrically neutral. Non-limiting examples of a zwitterionic phospholipid include phosphatidylethanolamine (PE), phosphatidylcholine (PC), ceramide phosphorylethanolamine (Cer-PE), and ceramide phosphorylcholine (Cer-PC).

Phospholipids disclosed herein include lecthins. Lecthins are amphiphilic mixtures of glycerophospholipids. In some embodiments, a lecthin comprises a mixture of phosphatidylcholine, phosphatidylethanolamine, phosphatidylinositol, phosphatidylserine, and phosphatidic acid. In some embodiments, a lecthin comprises 19% to 21% phosphatidylcholine, 8% to 20% phosphatidylethanolamine, 20% to 21% phosphatidylinositol, and 5% to 11% phospholipids comprising phosphatidylserine, and phosphatidic acid.

In some embodiments, a pharmaceutical composition disclosed herein may comprising or consisting essentially of or consisting of one or more phospholipids in an amount of, e.g., about 0.1% by weight, about 0.5% by weight, about 1.0% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, or about 10.0% by weight. In some embodiments, a pharmaceutical composition disclosed herein may comprising or consisting essentially of or consisting of one or more phospholipids in an amount of, e.g., at least 0.1% by weight, at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, or at least 10.0% by weight. In some embodiments, a pharmaceutical composition disclosed herein may comprising or consisting essentially of or consisting of one or more phospholipids in an amount of, e.g., at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, or at most 10.0% by weight.

In some embodiments, a pharmaceutical composition disclosed herein may comprising or consisting essentially of or consisting of one or more phospholipids in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.

### C₁₄₋₂₄ Fatty Acid Surfactants

A pharmaceutical composition disclosed herein may comprises one or more free C₁₄₋₂₄ fatty acid surfactants. A fatty acid surfactant comprises a carboxylic acid with a long unbranched hydrocarbon chain which may be either saturated or unsaturated associated with an alkali metal and are hydrophobic molecules amphipathic molecules having a HLB of greater than 12. The primary purpose of free C₁₄₋₂₄ fatty acid surfactants are to enhance solubility of a fibrate disclosed herein with the glycerolipid admixture and to enhance absorption of a fibrate disclosed herein thereby improving the pharmacokinetics of the compound. The improved solubility properties are achieved by the free C₁₄₋₂₄ fatty acid surfactants through the interaction of its carboxylic acid functional group with sodium ions present on a fibrate which neutralizes the charge and facilitating the compounds interaction with the hydrophobic glycolipid admixture. The improved absorption properties are achieved by the free C₁₄₋₂₄ fatty acid surfactants by facilitating and increasing the formation of mixed micelles by breaking up larger emulsion droplets, thereby mimicking the lipid digestion products of triglycerides, namely free fatty acids. As such, a C₁₄₋₂₄ fatty acid surfactant disclosed herein increases the uptake of micelles comprising one or more fibrates into enterocytes. A free C₁₄₋₂₄ fatty acid surfactant disclosed herein is a free C₁₄₋₂₄ fatty acid that is associated with an alkali metal, such as, e.g., lithium (Li), sodium (Na), potassium (K), rubidium (Rb), cesium (Cs), and francium (Fr), or alkaline earth metal, such as, e.g., beryllium (Be), magnesium (Mg), calcium (Ca), strontium (Sr), barium (Ba), and radium (Ra).

In some embodiments, pharmaceutical composition disclosed herein may include one or more free C₁₄₋₂₄ fatty acid surfactants comprising, or consisting essentially of or consisting of unsaturated free C₁₄-C₁₆ fatty acid surfactants, unsaturated free C₁₄-C₁₈ fatty acid surfactants, unsaturated free C₁₄-C₂₀ fatty acid surfactants, unsaturated free C₁₄-C₂₂ fatty acid surfactants, unsaturated free C₁₄-C₂₄ fatty acid surfactants, unsaturated free C₁₆-C₁₈ fatty acid surfactants, unsaturated free C₁₆-C₂₀ fatty acid surfactants, unsaturated free C₁₆-C₂₂ fatty acid surfactants, unsaturated free C₁₆-C₂₄ fatty acid surfactants, unsaturated free C₁₈-C₂₀ fatty acid surfactants, unsaturated free C₁₈-C₂₂ fatty acid surfactants, unsaturated free C₁₈-C₂₄ fatty acid surfactants, unsaturated free C₂₀-C₂₂ fatty acid surfactants, or unsaturated free C₂₂-C₂₄ fatty acid surfactants. In some embodiments, pharmaceutical composition disclosed herein may include one or more free C₁₄₋₂₄ fatty acid surfactants comprising, or consisting essentially of or consisting of ω-3 unsaturated free C₁₈-C₂₂ fatty acid surfactants, ω-5 unsaturated free C₁₈-C₂₂ fatty acid surfactants, ω-6 unsaturated free C₁₈-C₂₂ fatty acid surfactants, ω-7 unsaturated free C₁₈-C₂₂ fatty acid surfactants, ω-9 unsaturated free C₁₈-C₂₂ fatty acid surfactants, ω-10 unsaturated free C₁₈-C₂₂ fatty acid surfactants, ω-11 unsaturated free C₁₈-C₂₂ fatty acid surfactants, or ω-12 unsaturated free C₁₈-C₂₂ fatty acid surfactants.

In some embodiments, pharmaceutical composition disclosed herein may include one or more free C₁₄₋₂₄ fatty acid surfactants comprising, or consisting essentially of or consisting of saturated free C₁₄-C₁₆ fatty acid surfactants, saturated free C₁₄-C₁₈ fatty acid surfactants, saturated free C₁₄-C₂₀ fatty acid surfactants, saturated free C₁₄-C₂₂ fatty acid surfactants, saturated free C₁₄-C₂₄ fatty acid surfactants, saturated free C₁₆-C₁₈ fatty acid surfactants, saturated free C₁₆-C₂₀ fatty acid surfactants, saturated free C₁₆-C₂₂ fatty acid surfactants, saturated free C₁₆-C₂₄ fatty acid surfactants, saturated free C₁₈-C₂₀ fatty acid surfactants, saturated free C₁₈-C₂₂ fatty acid surfactants, saturated free C₁₈-C₂₄ fatty acid surfactants, saturated free C₂₀-C₂₂ fatty acid surfactants, or saturated free C₂₂-C₂₄ fatty acid surfactants.

In some embodiments, a pharmaceutical composition disclosed herein may include one or more free C₁₄₋₂₄ fatty acid surfactants comprising, or consisting essentially of or consisting of a mixture of saturated and unsaturated free C₁₄-C₁₆ fatty acid surfactants, a mixture of saturated and unsaturated free C₁₄-C₁₈ fatty acid surfactants, a mixture of saturated and unsaturated free C₁₄-C₂₀ fatty acid surfactants, a mixture of saturated and unsaturated free C₁₄-C₂₂ fatty acid surfactants, a mixture of saturated and unsaturated free C₁₄-C₂₄ fatty acid surfactants, a mixture of saturated and unsaturated free C₁₆-C₁₈ fatty acid surfactants, a mixture of saturated and unsaturated free C₁₆-C₂₀ fatty acid surfactants, a mixture of saturated and unsaturated free C₁₆-C₂₂ fatty acid surfactants, a mixture of saturated and unsaturated free C₁₆-C₂₄ fatty acid surfactants, a mixture of saturated and unsaturated free C₁₈-C₂₀ fatty acid surfactants, a mixture of saturated and unsaturated free C₁₈-C₂₂ fatty acid surfactants, a mixture of saturated and unsaturated free C₁₈-C₂₄ fatty acid surfactants, a mixture of saturated and unsaturated free C₂₀-C₂₂ fatty acid surfactants, or a mixture of saturated and unsaturated free C₂₂-C₂₄ fatty acid surfactants.

Non-limiting examples of a free C₁₄₋₂₄ fatty acid surfactant include sodium palmitate (hexadecanoate), sodium palmitolinolenate, sodium palmitidonate, sodium palmitovaccenate, sodium palmitoleate, sodium sapienate, sodium 4-Hexadecenoate, sodium stearate (octadecenoate), sodium α-linolenate, sodium stearidonate, sodium α-eleostearate, sodium β-eleostearate, sodium pumicate, sodium 7,10,13-octadecatrienoate, sodium 12-octadecenoate, sodium linoleate, sodium linolelaidate. Sodium y-linolenate, sodium calendate, sodium pinolenate, sodium vaccinate, sodium ruminate, sodium oleate, sodium elaidate, sodium petroselinate, sodium arachidate (eicosanoate), sodium dihomo-α-linolenate, sodium eicosatetraenoate, sodium eicosapentaenoate, sodium 9,12,15-eicosatrienoate, sodium p-eicosatetraenoate, sodium dihomo-linoleate, sodium dihomo-γ-linolenate, sodium arachidonate, sodium paullinate, sodium 7,10,13-eicosatrienoate, sodium gondoate, Sodium 8,11-eicosadienoate, sodium meadate, sodium gadoleate, sodium 8-eicosenoate, sodium behenate (docosanoate), sodium clupanodonate, sodium docosahexaenoate, sodium adrenate, sodium osbondate, sodium erucate, sodium lignocerate (tetracosanate), sodium 9,12,15,18,21-Tetracosapentaenoate, sodium 6,9,12,15,18,21-Tetracosahexaenoate, and sodium nervonate.

The lower limit of one or more free C₁₄₋₂₄ fatty acid surfactants that can be included in a pharmaceutical composition disclosed herein is an amount sufficient to solubilize a fibrate and confer its improved absorption properties. The upper limit of one or more free C₁₄₋₂₄ fatty acid surfactants that can be included in a pharmaceutical composition disclosed herein is its micellar concentration (CMC).

The amount of one or more free C₁₄₋₂₄ fatty acid surfactants useful in a pharmaceutical composition disclosed herein is an amount below its CMC. In some embodiments, a pharmaceutical composition disclosed herein may comprising or consisting essentially of or consisting of one or more free C₁₄₋₂₄ fatty acid surfactants in an amount of, e.g., about 0.1% by weight, about 0.5% by weight, about 1.0% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, or about 10.0% by weight. In some embodiments, a pharmaceutical composition disclosed herein may comprising or consisting essentially of or consisting of one or more free C₁₄₋₂₄ fatty acid surfactants in an amount of, e.g., at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, or at least 10.0% by weight. In some embodiments, a pharmaceutical composition disclosed herein may comprising or consisting essentially of or consisting of one or more free C₁₄₋₂₄ fatty acid surfactants in an amount of, e.g., at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, or at most 10.0% by weight.

In some embodiments, a pharmaceutical composition disclosed herein may comprising or consisting essentially of or consisting of one or more free C₁₄₋₂₄ fatty acid surfactants in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.

In some embodiments, a pharmaceutical composition disclosed herein may comprising or consisting essentially of or consisting of one or more free C₁₄₋₂₄ fatty acid surfactants in a concentration of, e.g., at most 5 μM, at most 10 μM, at most 15 μM, at most 20 μM, at most 25 μM, at most μM, or at most 35 μM. In some embodiments, a pharmaceutical composition disclosed herein may comprising or consisting essentially of or consisting of one or more free C₁₄₋₂₄ fatty acid surfactants in a concentration of, e.g., about 1 μM to about 5 μM, about 1 μM to about 10 μM, about 1 μM to about 15 μM, about 1 μM to about 20 μM, about 1 μM to about 25 μM, about 1 μM to about 30 μM, about 1 μM to about 35 μM, about 5 μM to about 10 μM, about 5 μM to about 15 μM, about 5 μM to about 20 μM, about 5 μM to about 25 μM, about 5 μM to about 30 μM, about 5 μM to about 35 μM, about 10 μM to about 15 μM, about 10 μM to about 20 μM, about 10 μM to about 25 μM, about 10 μM to about 30 μM, about 10 μM to about 35 μM, about 15 μM to about 20 μM, about 15 μM to about 25 μM, about 15 μM to about 30 μM, about 15 μM to about 35 μM, about 20 μM to about 25 μM, about 20 μM to about 30 μM, about 20 μM to about 35 μM, about 25 μM to about 30 μM, about 25 μM to about 35 μM, or about 30 μM to about 35 μM.

### Curcumin

Aspects of the present specification disclose, in part, a curcumin. In some embodiments, a pharmaceutical composition disclosed herein may include a curcumin. Curcumin is a pigment of phenolic nature extracted from Curcuma longa. Although a pharmacologically bioactive molecule, curcumin is able to facilitate gall bladder contraction, making this compound useful as a digestion enhancer disclosed herein. Gall bladder contraction is an important process in the absorption of fat and thus provide an important benefit increase the formation of mixed micelles by breaking up larger emulsion droplets, thereby increasing the uptake of micelles comprising one or more fibrates into enterocytes.

The amount of a curcumin useful in a pharmaceutical composition disclosed herein is a therapeutically effective amount or an amount effective in facilitating gall bladder contraction. In some embodiments, a pharmaceutical composition disclosed herein may comprising or consisting essentially of or consisting of curcumin in an amount of, e.g., about 0.1% by weight, about 0.5% by weight, about 1.0% by weight, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, or about 10.0% by weight. In some embodiments, a pharmaceutical composition disclosed herein may comprising or consisting essentially of or consisting of curcumin in an amount of, e.g., at least 0.1% by weight, at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, or at least 10.0% by weight. In some embodiments, a pharmaceutical composition disclosed herein may comprising or consisting essentially of or consisting of curcumin in an amount of, e.g., at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, or at most 10.0% by weight.

In some embodiments, a pharmaceutical composition disclosed herein may comprising or consisting essentially of or consisting of curcumin in an amount of, e.g., about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.

### Glycol Polymers

Aspects of the present specification disclose, in part, a glycol polymer. In some embodiments, a pharmaceutical composition disclosed herein may include one or more glycol polymers.

A pharmaceutical composition disclosed herein may comprise a stabilizing agent in an amount sufficient to stabilize the free acid or base present in a fibrate disclosed herein. In other aspects of this embodiment, a pharmaceutical composition disclosed herein may comprise a stabilizing agent in an amount of, e.g., less than about 40% by weight, less than about 35% by weight, less than about 30% by weight, less than about 25% by weight, less than about 20% by weight, less than about 19% by weight, less than about 18% by weight, less than about 17% by weight, less than about 16% by weight, less than about 15% by weight, less than about 14% by weight, less than about 13% by weight, less than about 12% by weight, less than about 11% by weight, less than about 10% by weight, less than about 9% by weight, less than about 8% by weight, less than about 7% by weight, less than about 6% by weight, less than about 5% by weight, less than about 4% by weight, less than about 3% by weight, less than about 2% by weight, or less than about 1%. In other aspects of this embodiment, a pharmaceutical composition disclosed herein may comprise a stabilizing agent in an amount of, e.g., about 1% to about 5% by weight, about 1% to about 7% by weight, about 1% to about 10% by weight, about 1% to about 12% by weight, about 1% to about 15% by weight, about 1% to about 18% by weight, about 1% to about 20% by weight, about 2% to about 5% by weight, about 2% to about 7% by weight, about 2% to about 10% by weight, about 2% to about 12% by weight, about 2% to about 15% by weight, about 2% to about 18% by weight, about 2% to about 20% by weight, about 3% to about 5% by weight, about 3% to about 7% by weight, about 3% to about 10% by weight, about 3% to about 12% by weight, about 3% to about 15% by weight, about 3% to about 18% by weight, about 3% to about 20% by weight, about 4% to about 5% by weight, about 4% to about 7% by weight, about 4% to about 10% by weight, about 4% to about 12% by weight, about 4% to about 15% by weight, about 4% to about 18% by weight, about 4% to about 20% by weight, about 5% to about 7% by weight, about 5% to about 10% by weight, about 5% to about 12% by weight, about 5% to about 15% by weight, about 5% to about 18% by weight, about 5% to about 20% by weight, about 6% to about 7% by weight, about 6% to about 10% by weight, about 6% to about 12% by weight, about 6% to about 15% by weight, about 6% to about 18% by weight, about 6% to about 20% by weight, about 7% to about 10% by weight, about 7% to about 12% by weight, about 7% to about 15% by weight, about 7% to about 18% by weight, about 7% to about 20% by weight, about 8% to about 10% by weight, about 8% to about 12% by weight, about 8% to about 15% by weight, about 8% to about 18% by weight, about 8% to about 20% by weight, about 9% to about 10% by weight, about 9% to about 12% by weight, about 9% to about 15% by weight, about 9% to about 18% by weight, about 9% to about 20% by weight, about 10% to about 12% by weight, about 10% to about 15% by weight, about 10% to about 18% by weight, or about 10% to about 20% by weight.

A stability agent as disclosed herein is not a solvent as it is used in an amount that does not result in substantial dissolving of a solute. As such, the amount stability agent used in a solid solution composition disclosed herein results in no more than 85% dissolution of a fibrate disclosed herein. In aspects of this embodiment, the amount stability agent used in a solid solution composition disclosed herein results in. e.g., no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, or no more than 5% dissolution of a fibrate disclosed herein.

In an embodiment, a glycol polymer may comprise a pharmaceutically acceptable PEG polymer. PEG polymers, also known as polyethylene oxide (PEO) polymers or polyoxyethylene (POE) polymers, are prepared by polymerization of ethylene oxide and are commercially available over a wide range of molecular weights from 100 g/mol to 10,000,000 g/mol. PEG polymers with a low molecular mass are liquids or low-melting solids, whereas PEG polymers of a higher molecular mass are solids. In an aspect of this embodiment, a PEG polymer used as a stability agent is a liquid PEG polymer. In aspects of this embodiment, a PEG polymer has a molecular weight of, e.g., no more than 100 g/mol, no more than 200 g/mol, no more than 300 g/mol, no more than 400 g/mol, no more than 500 g/mol, no more than 600 g/mol, no more than 700 g/mol, no more than 800 g/mol, no more than 900 g/mol, or no more than 1000 g/mol.

A PEG polymer include, without limitation, PEG 100, PEG 200, PEG 300, PEG 400, PEG 500, PEG 600, PEG 700, PEG 800, PEG 900, PEG 1000, PEG 1100, PEG 1200, PEG 1300, PEG 1400, PEG 1500, PEG 1600, PEG 1700, PEG 1800, PEG 1900, PEG 2000, PEG 2100, PEG 2200, PEG 2300, PEG 2400, PEG 2500, PEG 2600, PEG 2700, PEG 2800, PEG 2900, PEG 3000, PEG 3250, PEG 3350, PEG 3500, PEG 3750, PEG 4000, PEG 4250, PEG 4500, PEG 4750, PEG 5000, PEG 5500, PEG 6000, PEG 6500, PEG 7000, PEG 7500, PEG 8000, PEG 8500, PEG 9000, PEG 9500, PEG 10,000, PEG 11,000, PEG 12,000, PEG 13,000, PEG 14,000, PEG 15,000, PEG 16,000, PEG 17,000, PEG 18,000, PEG 19,000, or PEG 20,000.

In another embodiment, a glycol polymer may comprise a pharmaceutically acceptable polypropylene glycol (PPG) polymer. PPG polymers, also known as polypropylene oxide (PPO) polymers or polyoxypropylene (POP) polymers, are prepared by polymerization of propylene oxide and are commercially available over a wide range of molecular weights from 100 g/mol to 10,000,000 g/mol. PPG polymers with a low molecular mass are liquids or low-melting solids, whereas PPG polymers of a higher molecular mass are solids. In an aspect of this embodiment, a PPG polymer used as a stability agent is a liquid PPG polymer. In aspects of this embodiment, a PPG polymer has a molecular weight of, e.g., no more than 100 g/mol, no more than 200 g/mol, no more than 300 g/mol, no more than 400 g/mol, no more than 500 g/mol, no more than 600 g/mol, no more than 700 g/mol, no more than 800 g/mol, no more than 900 g/mol, or no more than 1000 g/mol.

A PPG polymer include, without limitation, PPG 100, PPG 200, PPG 300, PPG 400, PPG 500, PPG 600, PPG 700, PPG 800, PPG 900, PPG 1000, PPG 1100, PPG 1200, PPG 1300, PPG 1400, PPG 1500, PPG 1600, PPG 1700, PPG 1800, PPG 1900, PPG 2000, PPG 2100, PPG 2200, PPG 2300, PPG 2400, PPG 2500, PPG 2600, PPG 2700, PPG 2800, PPG 2900, PPG 3000, PPG 3250, PPG 3350, PPG 3500, PPG 3750, PPG 4000, PPG 4250, PPG 4500, PPG 4750, PPG 5000, PPG 5500, PPG 6000, PPG 6500, PPG 7000, PPG 7500, PPG 8000, PPG 8500, PPG 9000, PPG 9500, PPG 10,000, PPG 11,000, PPG 12,000, PPG 13,000, PPG 14,000, PPG 15,000, PPG 16,000, PPG 17,000, PPG 18,000, PPG 19,000, or PPG 20,000.

In some embodiments, a pharmaceutical composition disclosed herein does not include a glycol polymer. In aspects of these embodiments, a pharmaceutical composition disclosed herein does not include a PEG polymer. In other aspects of these embodiments, a pharmaceutical composition disclosed herein does not include a PGG polymer. In yet other aspects of these embodiments, a pharmaceutical composition disclosed herein does not include both a PEG polymer and a PGG polymer.

### Release Effects

In an embodiment, a substantial amount of a fibrate present in a pharmaceutical composition disclosed herein is delivered to or enters a lipid digestion and/or absorption pathway. In aspects of this embodiment, about 10%, about 20%, about 30%, about 40%, about 50%, about 60%, about 70%, about 80% about 90%, or about 95% of a fibrate present in a pharmaceutical composition disclosed herein is delivered to or enters a lipid digestion and/or absorption pathway. In other aspects of this embodiment, at least 10%, at least 20%, at least 30%, at least 40%, at least 50%, at least 60%, at least 70%, at least 80% at least 90%, or at least 95% of a fibrate present in a pharmaceutical composition disclosed herein is delivered to or enters a lipid digestion and/or absorption pathway. In yet other aspects of this embodiment, at most 10%, at most 20%, at most 30%, at most 40%, at most 50%, at most 60%, at most 70%, at most 80% at most 90%, or at most 95% of a fibrate present in a pharmaceutical composition disclosed herein is delivered to or enters a lipid digestion and/or absorption pathway.

In yet other aspects of this embodiment, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%, of a fibrate present in a pharmaceutical composition disclosed herein is delivered to or enters a lipid digestion and/or absorption pathway.

In an embodiment, an insubstantial amount of a fibrate present in a pharmaceutical composition disclosed herein is absorbed by blood capillaries and enters directly into the blood. In aspects of this embodiment, at most 1%, at most 5%, at most 10%, at most 20%, at most 30%, at most 40%, at most 50%, at most 60%, at most 70%, at most 80% at most 90%, or at most 95% of a fibrate present in a pharmaceutical composition disclosed herein is absorbed by blood capillaries and enters directly into the blood. In aspects of this embodiment, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 95%, about 10% to about 20%, about 10% to about 30%, about 10% to about 40%, about 10% to about 50%, about 10% to about 60%, about 10% to about 70%, about 10% to about 80%, about 10% to about 90%, about 10% to about 95%, about 20% to about 30%, about 20% to about 40%, about 20% to about 50%, about 20% to about 60%, about 20% to about 70%, about 20% to about 80%, about 20% to about 90%, about 20% to about 95%, about 30% to about 40%, about 30% to about 50%, about 30% to about 60%, about 30% to about 70%, about 30% to about 80%, about 30% to about 90%, about 30% to about 95%, about 40% to about 50%, about 40% to about 60%, about 40% to about 70%, about 40% to about 80%, about 40% to about 90%, about 40% to about 95%, about 50% to about 60%, about 50% to about 70%, about 50% to about 80%, about 50% to about 90%, about 50% to about 95%, about 60% to about 70%, about 60% to about 80%, about 60% to about 90%, about 60% to about 95%, about 70% to about 80%, about 70% to about 90%, about 70% to about 95%, about 80% to about 90%, about 80% to about 95%, or about 90% to about 95%, of a fibrate present in a pharmaceutical composition disclosed herein is absorbed by blood capillaries and enters directly into the blood.

### Formulating Procedure

The inclusion of one or more digestion enhancers to one or more fibrates to a pharmaceutical composition disclosed herein is applicable to any fibrate administered by a route of administration where uptake of the compound is achieved by absorption through the gastrointestinal tract, such as, e.g., oral delivery. However, formulation of a pharmaceutical composition disclosed herein is dependent on the solubility of a fibrate in the one or more glycerolipid used in formulating the pharmaceutical composition. As such, formulation of any one particular fibrate disclosed herein is achieved by the process described below which produces pharmaceutical compositions where one or more fibrates remain stably incorporated in the glycerolipid mixture.

In some embodiments, a selected fibrate can be formulated using 1) glycerolipids including at least one liquid fat (glycerolipid that is liquid at 18° C.) and at least one hard fat (glycerolipid that is solid at 18° C.) and 2) one or more digestion enhancers. In some embodiments, the one or more liquid and hard fats are first heated and the one or more digestion enhancers are solubilized in the glycerolipid admixture. Upon completion of solubility of the digestion enhancers, and if appropriate, the temperature of the admixture can be adjusted and a selected fibrate is then dissolved in this heated admixture to incorporate the compound. In some embodiments, the one or more liquid fats are first heated and the one or more digestion enhancers are solubilized in the liquid fats. Upon completion of solubility of the digestion enhancers, and if appropriate, the temperature of the admixture can be adjusted and a selected fibrate is then dissolved in this heated admixture to incorporate the compound. Upon complete dissolution of the fibrate, one or more hard fats are then added to this heated admixture. In some embodiments, one or more digestion enhancers are first heated and a selected fibrate is then dissolved to this heated admixture to incorporate the compound. Upon completion dissolution of the fibrate, and if appropriate, the temperature of the admixture can be adjusted and one or more liquid fats are then added and incorporated into this admixture. Upon completion of solubility of the one or more liquid fats, one or more hard fats are then added and incorporated into this admixture. The initial heating step in all procedures is performed at a temperature sufficient to dissolve the one or more digestion enhancers and selected fibrate and can be empirically determined based on the melting point of the selected components. Generally, this temperature range is about 60° C. to about 170° C. Any subsequent adjustment to the heat when one or more liquid fats and/or one or more hart fats are being added to the admixture is performed at a temperature sufficient to melt the hard fats and can be empirically determined based on the melting point of the hard fat used in the formulation. Generally, this temperature range is about 40° C. to about 60° C.

This incorporated mixture is then allowed to cool to room temperature, at which time stirring is ceased and the mixture is transferred to suitable containers where it will solidify. Once cooled, the pharmaceutical composition can be optionally stability tested by reheating the composition to a temperature sufficient to cause it to melt. The reheating step is performed at a temperature sufficient to melt the glycerolipid components and can be empirically determined based on the melting point of the hard fat used in the formulation. Generally, this temperature range is 40° C. to 50° C. The selection of the one or more digestion enhancers is generally not a critical component in this process, as a bile acid, free fatty acid, phospholipid, or free fatty acid surfactant can all be used in any combination to achieve a pharmaceutical composition disclosed herein. As shown in Example 2, the preparative methods all include a complete dissolution phase that upon cooling nano-crystallization of some components occurs and this is described in the XRPD spectra.

Example 3 exemplifies fibrates formulated according to this process. These examples also reveal two formulary subgroups. In one subgroup, the presence of one or more digestion enhancers does not appreciable enhance the solubility of the fibrate in a glycerolipid matrix. Instead, the presence of digestion enhancers serves to improve bioavailability of the fibrate.

### Methods and Uses

Aspects of the present specification disclose, in part, a method of treating an individual with a chronic inflammation. In one embodiment, the method comprises the step of administering to an individual in need thereof a pharmaceutical composition disclosed herein, wherein administration reduces a symptom associated with the chronic inflammation, thereby treating the individual. Other aspects of the present specification disclose, in part, a method of treating an individual with a neuroinflammation. In one embodiment, the method comprises the step of administering to an individual in need thereof a pharmaceutical composition disclosed herein, wherein administration reduces a symptom associated with the neuroinflammation, thereby treating the individual.

Aspects of the present specification disclose, in part, a pharmaceutical composition disclosed herein for use in the treatment of chronic inflammation. Other aspects of the present specification disclose, in part, a pharmaceutical composition disclosed herein for use in the treatment of a neuroinflammation.

Aspects of the present specification disclose, in part, use of a pharmaceutical composition disclosed herein for the treatment of chronic inflammation. Other aspects of the present specification disclose, in part, use of a pharmaceutical composition disclosed herein for the treatment of a neuroinflammation.

As a pharmaceutical composition disclosed herein relies on the digestive process of the gastrointestinal tract, oral administration is the preferred route of administration.

Aspects of the present specification disclose, in part, treating an individual suffering from a chronic inflammation and/or a neuroinflammation. As used herein, the term “treating,” refers to reducing or eliminating in an individual a clinical symptom of a chronic inflammation and/or a neuroinflammation; or delaying or preventing in an individual the onset of a clinical symptom of a chronic inflammation and/or a neuroinflammation. For example, the term “treating” can mean reducing a symptom of a condition characterized by a chronic inflammation and/or a neuroinflammation by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%.

The actual symptoms associated with chronic inflammation and/or a neuroinflammation are well known and can be determined by a person of ordinary skill in the art by taking into account factors, including, without limitation, the location of the chronic inflammation and/or a neuroinflammation, the cause of the chronic inflammation and/or a neuroinflammation, the severity of the chronic inflammation, and/or the tissue or organ affected by the chronic inflammation and/or a neuroinflammation. Those of skill in the art will know the appropriate symptoms or indicators associated with a specific type of chronic inflammation and/or a neuroinflammation and will know how to determine if an individual is a candidate for treatment as disclosed herein.

Chronic inflammation symptoms include, without limitation, edema, hyperemia, erythema, bruising, tenderness, stiffness, swollenness, fever, chills, stuffy nose, stuffy head, breathing problems, fluid retention, blood clots, loss of appetite, increased heart rate, formation of granulomas, fibrinous, pus, non-viscous serous fluid, or ulcer and pain. The actual symptoms associated with a chronic inflammation are well known and can be determined by a person of ordinary skill in the art by taking into account factors, including, without limitation, the location of the inflammation, the cause of the inflammation, the severity of the inflammation, the tissue or organ affected, and the associated disorder.

Specific patterns of chronic inflammation are seen during particular situations that arise in the body, such as when inflammation occurs on an epithelial surface, or pyogenic bacteria are involved. For example, granulomatous inflammation is an inflammation resulting from the formation of granulomas arising from a limited but diverse number of diseases, include, without limitation, tuberculosis, leprosy, sarcoidosis, and syphilis. Purulent inflammation is an inflammation resulting in large amount of pus, which consists of neutrophils, dead cells, and fluid. Infection by pyogenic bacteria such as staphylococci is characteristic of this kind of inflammation. Serous inflammation is an inflammation resulting from copious effusion of non-viscous serous fluid, commonly produced by mesothelial cells of serous membranes, but may be derived from blood plasma. Skin blisters exemplify this pattern of inflammation. Ulcerative inflammation is an inflammation resulting from the necrotic loss of tissue from the epithelial surface, exposing lower layers and forming an ulcer.

A chronic inflammation symptom can be associated with a large, unrelated group of disorders which underlay a variety of diseases and disorders. The immune system is often involved with chronic inflammatory disorders, demonstrated in both allergic reactions and some myopathies, with many immune system disorders resulting in abnormal inflammation. Non-immune diseases with etiological origins in chronic inflammatory processes include cancer, atherosclerosis, and ischaemic heart disease. Non-limiting examples of disorders exhibiting chronic inflammation as a symptom include, without limitation, acne, acid reflux/heartburn, age related macular degeneration (AMD), allergy, allergic rhinitis, Alzheimer's disease, amyotrophic lateral sclerosis, anemia, appendicitis, arteritis, arthritis, asthma. atherosclerosis, autoimmune disorders, balanitis, blepharitis, bronchiolitis, bronchitis, a bullous pemphigoid, burn, bursitis, cancer, cardiac arrest, carditis, celiac disease, cellulitis, cervicitis, cholangitis, cholecystitis, chorioamnionitis, chronic obstructive pulmonary disease (COPD), cirrhosis, colitis, congestive heart failure, conjunctivitis, cyclophosphamide-induced cystitis, cystic fibrosis, cystitis, common cold, dacryoadenitis, dementia, dermatitis, dermatomyositis, diabetes, diabetic neuropathy, diabetic retinopathy, diabetic nephropathy, diabetic ulcer, digestive system disease, eczema, emphysema, encephalitis, endocarditis, endometritis, enteritis, enterocolitis, epicondylitis, epididymitis, fasciitis, fibromyalgia, fibrosis, fibrositis, gastritis, gastroenteritis, gingivitis, glomerulonephritis, glossitis, heart disease, heart valve dysfunction, hepatitis, hidradenitis suppurativa, Huntington's disease, hyperlipidemic pancreatitis, hypertension, ileitis, infection, inflammatory bowel disease, inflammatory cardiomegaly, insulin resistance, interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), a migraine, multiple sclerosis, myelitis, myocarditis, myositis, nephritis, non-alcoholic steatohepatitis, obesity, omphalitis, oophoritis, orchitis, osteochondritis, osteopenia, osteomyelitis, osteoporosis, osteitis, otitis, pancreatitis, Parkinson's disease, parotitis, pelvic inflammatory disease, pemphigus vularis, pericarditis, peripheral neuropathy, peritonitis, pharyngitis, phlebitis, pleuritis, pneumonitis, polycystic nephritis, proctitis, prostatitis, psoriasis, pulpitis, pyelonephritis, pylephlebitis, renal failure, reperfusion injury, retinitis, rheumatic fever, rhinitis, salpingitis, sarcoidosis, sialadenitis, sinusitis, spastic colon, stenosis, stomatitis, stroke, surgical complication, synovitis, tendonitis, tendinosis, tenosynovitis, thrombophlebitis, tonsillitis, trauma, traumatic brain injury, transplant rejection, trigonitis, tuberculosis, tumor, urethritis, ursitis, uveitis, vaginitis, vasculitis, and vulvitis. See also, Eric R. First, Application of Botulinum Toxin to the Management of Neurogenic Inflammatory Disorders, U.S. Pat. No. 6,063,768, which is hereby incorporated by reference in its entirety.

In one embodiment, a chronic inflammation comprises a tissue inflammation. Tissue inflammation is a chronic inflammation that is confined to a particular tissue or organ. In aspect of this embodiment, a tissue inflammation comprises, e.g., a skin inflammation, a muscle inflammation, a tendon inflammation, a ligament inflammation, a bone inflammation, a cartilage inflammation, a lung inflammation, a heart inflammation, a liver inflammation, a pancreatic inflammation, a kidney inflammation, a bladder inflammation, a stomach inflammation, an intestinal inflammation, a neuronal inflammation, and a brain inflammation.

In another embodiment, a chronic inflammation comprises a systemic inflammation. Although the processes involved are identical to tissue inflammation, systemic inflammation is not confined to a particular tissue but in fact overwhelms the body, involving the endothelium and other organ systems. When it is due to infection, the term sepsis is applied, with the terms bacteremia being applied specifically for bacterial sepsis and viremia specifically to viral sepsis. Vasodilation and organ dysfunction are serious problems associated with widespread infection that may lead to septic shock and death.

In another embodiment, a chronic inflammation comprises an arthritis. Arthritis includes a group of conditions involving damage to the joints of the body due to the inflammation of the synovium including, without limitation osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

In another embodiment, a chronic inflammation comprises an autoimmune disorder. Autoimmune diseases can be broadly divided into systemic and organ-specific autoimmune disorders, depending on the principal clinico-pathologic features of each disease. Systemic autoimmune diseases include, without limitation, systemic lupus erythematosus (SLE), Sjögren's syndrome, Scleroderma, rheumatoid arthritis, and polymyositis. Local autoimmune diseases may be endocrinologic (Diabetes Mellitus Type 1, Hashimoto's thyroiditis, Addison's disease etc.), dermatologic (pemphigus vulgaris), hematologic (autoimmune haemolytic anemia), neural (multiple sclerosis) or can involve virtually any circumscribed mass of body tissue. Types of autoimmune disorders include, without limitation, acute disseminated encephalomyelitis (ADEM), Addison's disease, an allergy or sensitivity, amyotrophic lateral sclerosis, anti-phospholipid antibody syndrome (APS), arthritis, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD), diabetes mellitus type 1 (IDDM), endometriosis, fibromyalgia, Goodpasture's syndrome, Graves' disease, Guillain-Barré syndrome (GBS), Hashimoto's thyroiditis, hidradenitis suppurativa, idiopathic thrombocytopenic purpura, inflammatory bowel disease, interstitial cystitis, lupus (including discoid lupus erythematosus, drug-induced lupus erythematosus. lupus nephritis, neonatal lupus, subacute cutaneous lupus erythematosus and systemic lupus erythematosus), morphea, multiple sclerosis (MS), myasthenia gravis, myopathies, narcolepsy, neuromyotonia, pemphigus vulgaris, pernicious anaemia, primary biliary cirrhosis, recurrent disseminated encephalomyelitis (multiphasic disseminated encephalomyelitis), rheumatic fever, schizophrenia, scleroderma, Sjögren's syndrome, tenosynovitis, vasculitis, and vitiligo. See Pamela D. Van Schaack & Kenneth L. Tong, Treatment of Autoimmune Disorder with a Neurotoxin, U.S. Patent Publication 2006/138059, which is hereby incorporated by reference in its entirety.

In another embodiment, a chronic inflammation comprises a myopathy. Myopathies are caused when the immune system inappropriately attacks components of the muscle, leading to inflammation in the muscle. A myopathy includes an inflammatory myopathy and an auto-immune myopathy. Myopathies include, without limitation, dermatomyositis, inclusion body myositis, and polymyositis.

In another embodiment, a chronic inflammation comprises a vasculitis. Vasculitis is a varied group of disorders featuring inflammation of a vessel wall including lymphatic vessels and blood vessels like veins (phlebitis), arteries (arteritis) and capillaries due to leukocyte migration and resultant damage. The inflammation may affect any size blood vessel, anywhere in the body. It may affect either arteries and/or veins. The inflammation may be focal, meaning that it affects a single location within a vessel; or it may be widespread, with areas of inflammation scattered throughout a particular organ or tissue, or even affecting more than one organ system in the body. Vasculitis include, without limitation, Buerger's disease (thromboangiitis obliterans), cerebral vasculitis (central nervous system vasculitis), Churg-Strauss arteritis, cryoglobulinemia, essential cryoglobulinemic vasculitis, giant cell (temporal) arteritis, Golfer's vasculitis, Henoch-Schonlein purpura, hypersensitivity vasculitis (allergic vasculitis), Kawasaki disease, microscopic polyarteritis/polyangiitis, polyarteritis nodosa, polymyalgia rheumatica (PMR), rheumatoid vasculitis, Takayasu arteritis, Wegener's granulomatosis, and vasculitis secondary to connective tissue disorders like systemic lupus erythematosus (SLE), rheumatoid arthritis (RA), relapsing polychondritis, Behget's disease, or other connective tissue disorders, vasculitis secondary to viral infection.

In another embodiment, a chronic inflammation comprises a skin disorder. Skin disorders include, without limitation, an acne, including acne vulgaris, a bullous phemigoid, a dermatitis, including atopic dermatitis and chronic actinic dermatitis, an eczema like atopic eczema, contact eczema, xerotic eczema, seborrhoeic dermatitis, dyshidrosis, discoid eczema, venous eczema, dermatitis herpetiformis, neurodermatitis, and autoeczematization, and statis dermatitis, hidradenitis suppurativa, lichen planus, psoriasis including plaqure psoriasis, nail psoriasis, guttate psoriasis, scalp psoriasis, inverse psoriasis, pustular psoriasis, erythrodermis psoriasis, and psoriatic arthritis, rosacea and scleroderma including morphea.

In another embodiment, a chronic inflammation comprises a gastrointestinal disorder. A gastrointestinal disorder includes, without limitation, irritable bowel disease, an inflammatory bowel disease including Crohn's disease and an ulcerative colitis like ulcerative proctitis, left-sided colitis, pancolitis and fulminant colitis.

In another embodiment, a chronic inflammation comprises a cardiovascular disease. When LDL cholesterol becomes embedded in arterial walls, it can invoke an immune response. Chronic inflammation eventually can damage the arteries, which can cause them to burst. Cardiovascular disease is any of a number of specific diseases that affect the heart itself and/or the blood vessel system, especially the veins and arteries leading to and from the heart. There are more than 60 types of cardiovascular disorders including, without limitation, a hypertension, endocarditis, myocarditis, heart valve dysfunction, congestive heart failure, myocardial infarction, a diabetic cardiac conditions, blood vessel inflammation like arteritis, phlebitis, vasculitis; arterial occlusive disease like arteriosclerosis and stenosis, inflammatory cardiomegaly, a peripheral arterial disease; an aneurysm; an embolism; a dissection; a pseudoaneurysm; a vascular malformation; a vascular nevus; a thrombosis; a thrombphlebitis; a varicose veins; a stroke. Symptoms of a cardiovascular disorder affecting the heart include, without limitation, chest pain or chest discomfort (angina), pain in one or both arms, the left shoulder, neck, jaw, or back, shortness of breath, dizziness, faster heartbeats, nausea, abnormal heartbeats, feeling fatigued. Symptoms of a cardiovascular disorder affecting the brain include, without limitation, sudden numbness or weakness of the face, arm, or leg, especially on one side of the body, sudden confusion or trouble speaking or understanding speech, sudden trouble seeing in one or both eyes, sudden dizziness, difficulty walking, or loss of balance or coordination, sudden severe headache with no known cause. Symptoms of a cardiovascular disorder affecting the legs, pelvis and/or arm include, without limitation, claudication, which is a pain, ache, or cramp in the muscles, and cold or numb feeling in the feet or toes, especially at night.

In another embodiment, a chronic inflammation comprises a cancer. Inflammation orchestrates the microenvironment around tumors, contributing to proliferation, survival, and migration. For example, fibrinous inflammation results from a large increase in vascular permeability which allows fibrin to pass through the blood vessels. If an appropriate procoagulative stimulus is present, such as cancer cells, a fibrinous exudate is deposited. This is commonly seen in serous cavities, where the conversion of fibrinous exudate into a scar can occur between serous membranes, limiting their function. In another example, a cancer is an inflammatory cancer like a NF-κB-driven inflammatory cancer.

In another embodiment, a chronic inflammation comprises a pharmacologically induced inflammation. Certain drugs or exogenic chemical compounds are known to affect inflammation. For example, Vitamin A deficiency causes an increase in an inflammatory response. Certain illicit drugs such as cocaine and ecstasy may exert some of their detrimental effects by activating transcription factors intimately involved with inflammation (e.g. NF-κB).

In another embodiment, a chronic inflammation comprises an infection. An infectious organism can escape the confines of the immediate tissue via the circulatory system or lymphatic system, where it may spread to other parts of the body. If an organism is not contained by the actions of acute inflammation, it may gain access to the lymphatic system via nearby lymph vessels. An infection of the lymph vessels is known as lymphangitis, and infection of a lymph node is known as lymphadenitis. A pathogen can gain access to the bloodstream through lymphatic drainage into the circulatory system. Infections include, without limitation, bacterial cystitis, bacterial encephalitis, pandemic influenza, viral encephalitis, and viral hepatitis (A, B and C).

In another embodiment, a chronic inflammation comprises a tissue or organ injury. Tissue or organ injuries include, without limitation, a burn, a laceration, a wound, a puncture, or a trauma.

In another embodiment, a chronic inflammation comprises a transplant rejection. Transplant rejection occurs when a transplanted organ or tissue is not accepted by the body of the transplant recipient because the immune system of the recipient attacks the transplanted organ or tissue. An adaptive immune response, transplant rejection is mediated through both T cell mediated and humoral immune (antibodies) mechanisms. A transplant rejection can be classified as a hyperacute rejection, an acute rejection, or a chronic rejection. Chronic rejection of a transplanted organ or tissue is where the rejection is due to a poorly understood chronic inflammatory and immune response against the transplanted tissue. Also included in the term “transplant rejection” is a graft-versus-host disease (GVHD). GVHD is a common complication of allogeneic bone marrow transplantation in which functional immune cells in the transplanted marrow recognize the recipient as “foreign” and mount an immunologic attack. It can also take place in a blood transfusion under certain circumstances. GVHD is divided into acute and chronic forms. Acute and chronic GVHD appear to involve different immune cell subsets, different cytokine profiles, somewhat different host targets, and respond differently to treatment.

In another embodiment, a chronic inflammation comprises a Th1-mediated inflammatory disease. In a well-functioning immune system, an immune response should result in a well-balanced pro-inflammatory Th1 response and anti-inflammatory Th2 response that is suited to address the immune challenge. Generally speaking, once a pro-inflammatory Th1 response is initiated, the body relies on the anti-inflammatory response invoked by a Th2 response to counteract this Th1 response. This counteractive response includes the release of Th2 type cytokines such as, e.g., IL-4, IL-5, and IL-13 which are associated with the promotion of IgE and eosinophilic responses in atopy, and also IL-10, which has an anti-inflammatory response. A Th1-mediated inflammatory disease involves an excessive pro-inflammatory response produced by Th1 cells that leads to chronic inflammation. The Th1-mediated disease may be virally, bacterially or chemically (e.g. environmentally) induced. For example, a virus causing the Th1-mediated disease may cause a chronic or acute infection, which may cause a respiratory disorder or influenza.

In another embodiment, a chronic inflammation comprises a chronic neurogenic inflammation. Chronic neurogenic Inflammation refers to an inflammatory response initiated and/or maintained through the release of inflammatory molecules like SP or CGRP which released from peripheral sensory nerve terminals (i.e., an efferent function, in contrast to the normal afferent signaling to the spinal cord in these nerves). Chronic neurogenic inflammation includes both primary inflammation and secondary neurogenic inflammation. As used herein, the term “primary” neurogenic inflammation refers to tissue inflammation (inflammatory symptoms) that is initiated by, or results from, the release of substances from primary sensory nerve terminals (such as C and A-delta fibers). As used herein, the term “secondary” neurogenic inflammation” refers to tissue inflammation initiated by non-neuronal sources (e.g., extravasation from vascular bed or tissue interstitium-derived, such as from mast cells or immune cells) of inflammatory mediators, such as peptides or cytokines, stimulating sensory nerve terminals and causing a release of inflammatory mediators from the nerves. The net effect of both forms (primary and secondary) of chronic neurogenic inflammation is to have an inflammatory state that is maintained by the sensitization of the peripheral sensory nerve fibers. The physiological consequence of the resulting chronic neurogenic inflammation depends on the tissue in question, producing, such as, e.g., cutaneous pain (allodynia, hyperalgesia), joint pain and/or arthritis, visceral pain and dysfunction, pulmonary dysfunction (asthma, COPD), and bladder dysfunction (pain, overactive bladder).

Neuroinflammation includes both acute neuroinflammation and chronic neuroinflammation. Acute neuroinflammation usually follows injury to the central nervous system immediately, and is characterized by rapid activation of microglia, release of inflammatory molecules, endothelial cell activation, platelet deposition, and tissue edema. Chronic neuroinflammation is the sustained activation of glial cells and recruitment of other immune cells into the brain. However, over time, chronic inflammation causes the degradation of tissue and of the BBB and reactive oxygen species generated and inflammatory signals released by microglia recruit peripheral immune cells to assist in mounting a neuroimmune response.

Chronic inflammation is typically associated with neurodegenerative diseases. A neurodegenerative disease is caused by the progressive loss of structure or function of neurons, in the process known as neurodegeneration. Such neuronal damage may ultimately involve cell death. Neurodegeneration can be found in the brain at many different levels of neuronal circuitry, ranging from molecular to systemic. Because there is no known way to reverse the progressive degeneration of neurons, these diseases are considered to be incurable. The two major contributing factors to neurodegeneration are oxidative stress and neuroinflammation. Non-limiting examples of a neurodegenerative disease associated with neuroinflammation include Alzheimer's disease, amyotrophic lateral sclerosis, aneurysm, anxiety, aphasia, Asperger's syndrome, ataxia, attention deficit hyperactivity disorder, bipolar disorder, brain cancer, cancer-related or chemotherapy-related cognitive impairment, cerebral ischemia, cerebral palsy, chronic fatigue syndrome, CNS neuropathy, Creutzfeldt-Jakob disease, dementia, depression, encephalitis, epilepsy, fibromyalgia, functional neurological disorder, Guillain-Barre syndrome, headaches, idiopathic intracranial hypertension, migraine, multiple sclerosis, Parkinson's disease, pediatric neuroinflammatory disorder, peri-operative neurocognitive disorder, post-operative cognitive decline, post-traumatic stress disorder, SARS-CoV-2 infection, schizophrenia, stroke, substance abuse derived neuroinflammation, traumatic brain injury, Tourette syndrome.

A composition or compound is administered to an individual. An individual is typically a human being. Typically, any individual who is a candidate for a conventional chronic inflammation and/or a neuroinflammation treatment is a candidate for a chronic inflammation and/or a neuroinflammation treatment disclosed herein. Pre-operative evaluation typically includes routine history and physical examination including biomarker evaluation in addition to thorough informed consent disclosing all relevant risks and benefits of the procedure.

A pharmaceutical composition disclosed herein may comprise a fibrate in a therapeutically effective amount. As used herein, the term “effective amount” is synonymous with “therapeutically effective amount”, “effective dose”, or “therapeutically effective dose” and when used in reference to treating a chronic inflammation and/or a neuroinflammation refers to the minimum dose of a fibrate disclosed herein necessary to achieve the desired therapeutic effect and includes a dose sufficient to reduce a symptom associated with a chronic inflammation and/or a neuroinflammation. The effectiveness of a fibrate disclosed herein in treating a chronic inflammation and/or a neuroinflammation can be determined by observing an improvement in an individual based upon one or more clinical symptoms, and/or physiological indicators associated with the condition. An improvement in a chronic inflammation and/or a neuroinflammation also can be indicated by a reduced need for a concurrent therapy.

The appropriate effective amount of a fibrate disclosed herein to be administered to an individual for a particular chronic inflammation and/or a neuroinflammation can be determined by a person of ordinary skill in the art by taking into account factors, including, without limitation, the type of chronic inflammation and/or a neuroinflammation, the location of the chronic inflammation and/or a neuroinflammation, the cause of the chronic inflammation and/or a neuroinflammation, the severity of the chronic inflammation and/or a neuroinflammation, the degree of relief desired, the duration of relief desired, the particular fibrate used, the pharmacokinetic properties of the particular fibrate used including liberation, absorption, distribution, metabolism, and excretion, the pharmacodynamic properties of the particular fibrate used including mechanism of action, dose-response relationship, desired activity, undesirable side effects, therapeutic window and duration of action, the nature of the other compounds to be included in the composition, the particular formulation, the particular route of administration, the particular characteristics, history and risk factors of the patient, such as, e.g., age, weight, general health and the like, or any combination thereof. It is known by a person of ordinary skill in the art that an effective amount of a fibrate disclosed herein can be extrapolated from in-vitro assays and in-vivo administration studies using animal models prior to administration to humans. In addition, variations in the necessary effective amount are to be expected in view of the differing efficiencies of the various routes of administration. For instance, oral administration of a fibrate disclosed herein generally would be expected to require higher dosage levels than intravenous administration. Similarly, systemic administration of a fibrate disclosed herein would be expected to require higher dosage levels than a local administration. Variations in these dosage levels can be adjusted using standard empirical routines of optimization, which are well-known to a person of ordinary skill in the art. One skilled in the art will also recognize that the condition of the individual can be monitored throughout the course of a method or use disclosed herein and that the effective amount of a fibrate disclosed herein that is administered can be adjusted accordingly. Thus, the precise therapeutically effective dosage levels and patterns are preferably determined by the attending healthcare professional in consideration of the above-identified factors.

In some embodiments, a therapeutically effective amount of a fibrate disclosed herein reduces a symptom associated with a chronic inflammation and/or a neuroinflammation by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a fibrate disclosed herein reduces a symptom associated with a chronic inflammation and/or a neuroinflammation by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a fibrate disclosed herein reduces a symptom associated with a chronic inflammation and/or a neuroinflammation by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In some embodiments, a therapeutically effective amount of a fibrate disclosed herein generally is in the range of about 0.01 mg/kg to about 10 mg/kg. In aspects of these embodiments, an effective amount of a fibrate disclosed herein may be, e.g., at least 0.01 mg/kg, at least 0.05 mg/kg, at least 0.1 mg/kg, at least 0.5 mg/kg, at least 1.0 mg/kg, at least 2.0 mg/kg, at least 3.0 mg/kg, at least 4.0 mg/kg, at least 5.0 mg/kg, at least 6.0 mg/kg, at least 7.0 mg/kg, at least 8.0 mg/kg, at least 9.0 mg/kg, or at least 10 mg/kg. In other aspects of these embodiments, an effective amount of a fibrate disclosed herein may be, e.g., at most 0.01 mg/kg, at most 0.05 mg/kg, at most 0.1 mg/kg, at most 0.5 mg/kg, at most 1.0 mg/kg, at most 2.0 mg/kg, at most 3.0 mg/kg, at most 4.0 mg/kg, at most 5.0 mg/kg, at most 6.0 mg/kg, at most 7.0 mg/kg, at most 8.0 mg/kg, at most 9.0 mg/kg, or at most 10 mg/kg. In yet other aspects of these embodiments, an effective amount of a fibrate disclosed herein may be in the range of, e.g., about 0.01 mg/kg to about 0.05 mg/kg, about 0.01 mg/kg to about 0.1 mg/kg, about 0.01 mg/kg to about 0.5 mg/kg, about 0.01 mg/kg to about 1.0 mg/kg, about 0.01 mg/kg to about 2.0 mg/kg, about 0.01 mg/kg to about 3.0 mg/kg, about 0.01 mg/kg to about 4.0 mg/kg, about 0.01 mg/kg to about 5.0 mg/kg, about 0.01 mg/kg to about 6.0 mg/kg, about 0.01 mg/kg to about 7.0 mg/kg, about 0.01 mg/kg to about 8.0 mg/kg, about 0.01 mg/kg to about 9.0 mg/kg, about 0.01 mg/kg to about 10 mg/kg, about 0.1 mg/kg to about 0.5 mg/kg, about 0.1 mg/kg to about 1.0 mg/kg, about 0.1 mg/kg to about 2.0 mg/kg, about 0.1 mg/kg to about 3.0 mg/kg, about 0.1 mg/kg to about 4.0 mg/kg, about 0.1 mg/kg to about 5.0 mg/kg, about 0.1 mg/kg to about 6.0 mg/kg, about 0.1 mg/kg to about 7.0 mg/kg, about 0.1 mg/kg to about 8.0 mg/kg, about 0.1 mg/kg to about 9.0 mg/kg, about 0.1 mg/kg to about 10 mg/kg, about 0.5 mg/kg to about 1.0 mg/kg, about 0.5 mg/kg to about 2.0 mg/kg, about 0.5 mg/kg to about 3.0 mg/kg, about 0.5 mg/kg to about 4.0 mg/kg, about 0.5 mg/kg to about 5.0 mg/kg, about 0.5 mg/kg to about 6.0 mg/kg, about 0.5 mg/kg to about 7.0 mg/kg, about 0.5 mg/kg to about 8.0 mg/kg, about 0.5 mg/kg to about 9.0 mg/kg, about 0.5 mg/kg to about 10 mg/kg, about 1.0 mg/kg to about 2.0 mg/kg, about 1.0 mg/kg to about 3.0 mg/kg, about 1.0 mg/kg to about 4.0 mg/kg, about 1.0 mg/kg to about 5.0 mg/kg, about 1.0 mg/kg to about 6.0 mg/kg, about 1.0 mg/kg to about 7.0 mg/kg, about 1.0 mg/kg to about 8.0 mg/kg, about 1.0 mg/kg to about 9.0 mg/kg, or about 1.0 mg/kg to about 10 mg/kg.

In some embodiments, a therapeutically effective amount of a fibrate disclosed herein generally is in the range of about 0.01 mg/kg/day to about 10 mg/kg/day. In aspects of these embodiments, an effective amount of a fibrate disclosed herein may be, e.g., at least 0.01 mg/kg/day, at least 0.05 mg/kg/day, at least 0.1 mg/kg/day, at least 0.5 mg/kg/day, at least 1.0 mg/kg/day, at least 2.0 mg/kg/day, at least 3.0 mg/kg/day, at least 4.0 mg/kg/day, at least 5.0 mg/kg/day, at least 6.0 mg/kg/day, at least 7.0 mg/kg/day, at least 8.0 mg/kg/day, at least 9.0 mg/kg/day, or at least 10 mg/kg/day. In other aspects of these embodiments, an effective amount of a fibrate disclosed herein may be, e.g., at most 0.01 mg/kg/day, at most 0.05 mg/kg/day, at most 0.1 mg/kg/day, at most 0.5 mg/kg/day, at most 1.0 mg/kg/day, at most 2.0 mg/kg/day, at most 3.0 mg/kg/day, at most 4.0 mg/kg/day, at most 5.0 mg/kg/day, at most 6.0 mg/kg/day, at most 7.0 mg/kg/day, at most 8.0 mg/kg/day, at most 9.0 mg/kg/day, or at most 10 mg/kg/day. In yet other aspects of these embodiments, an effective amount of a fibrate disclosed herein may be in the range of, e.g., about 0.01 mg/kg/day to about 0.05 mg/kg/day, about 0.01 mg/kg/day to about 0.1 mg/kg/day, about 0.01 mg/kg/day to about 0.5 mg/kg/day, about 0.01 mg/kg/day to about 1.0 mg/kg/day, about 0.01 mg/kg/day to about 2.0 mg/kg/day, about 0.01 mg/kg/day to about 3.0 mg/kg/day, about 0.01 mg/kg/day to about 4.0 mg/kg/day, about 0.01 mg/kg/day to about 5.0 mg/kg/day, about 0.01 mg/kg/day to about 6.0 mg/kg/day, about 0.01 mg/kg/day to about 7.0 mg/kg/day, about 0.01 mg/kg/day to about 8.0 mg/kg/day, about 0.01 mg/kg/day to about 9.0 mg/kg/day, about 0.01 mg/kg/day to about 10 mg/kg/day, about 0.1 mg/kg/day to about 0.5 mg/kg/day, about 0.1 mg/kg/day to about 1.0 mg/kg/day, about 0.1 mg/kg/day to about 2.0 mg/kg/day, about 0.1 mg/kg/day to about 3.0 mg/kg/day, about 0.1 mg/kg/day to about 4.0 mg/kg/day, about 0.1 mg/kg/day to about 5.0 mg/kg/day, about 0.1 mg/kg/day to about 6.0 mg/kg/day, about 0.1 mg/kg/day to about 7.0 mg/kg/day, about 0.1 mg/kg/day to about 8.0 mg/kg/day, about 0.1 mg/kg/day to about 9.0 mg/kg/day, about 0.1 mg/kg/day to about 10 mg/kg/day, about 0.5 mg/kg/day to about 1.0 mg/kg/day, about 0.5 mg/kg/day to about 2.0 mg/kg/day, about 0.5 mg/kg/day to about 3.0 mg/kg/day, about 0.5 mg/kg/day to about 4.0 mg/kg/day, about 0.5 mg/kg/day to about 5.0 mg/kg/day, about 0.5 mg/kg/day to about 6.0 mg/kg/day, about 0.5 mg/kg/day to about 7.0 mg/kg/day, about 0.5 mg/kg/day to about 8.0 mg/kg/day, about 0.5 mg/kg/day to about 9.0 mg/kg/day, about 0.5 mg/kg/day to about 10 mg/kg/day, about 1.0 mg/kg/day to about 2.0 mg/kg/day, about 1.0 mg/kg/day to about 3.0 mg/kg/day, about 1.0 mg/kg/day to about 4.0 mg/kg/day, about 1.0 mg/kg/day to about 5.0 mg/kg/day, about 1.0 mg/kg/day to about 6.0 mg/kg/day, about 1.0 mg/kg/day to about 7.0 mg/kg/day, about 1.0 mg/kg/day to about 8.0 mg/kg/day, about 1.0 mg/kg/day to about 9.0 mg/kg/day, or about 1.0 mg/kg/day to about 10 mg/kg/day.

Dosing can be single dosage or cumulative (serial dosing), and can be readily determined by one skilled in the art. For instance, treatment of a chronic inflammation and/or a neuroinflammation may comprise a one-time administration of an effective dose of a pharmaceutical composition disclosed herein. Alternatively, treatment of a chronic inflammation and/or a neuroinflammation may comprise multiple administrations of an effective dose of a pharmaceutical composition carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a pharmaceutical composition disclosed herein can be administered to an individual once daily for an indefinite period of time, or until the individual no longer requires therapy. A person of ordinary skill in the art will recognize that the condition of the individual can be monitored throughout the course of treatment and that the effective amount of a pharmaceutical composition disclosed herein that is administered can be adjusted accordingly.

In one embodiment, upon administration to an individual, a pharmaceutical composition comprising a fibrate disclosed herein results in a bio-distribution of the fibrate different than a bio-distribution of the fibrate included in the same pharmaceutical composition, except without the one or more digestion enhancers disclosed herein.

In some embodiments, a pharmaceutical composition comprises one or more fibrates, one or more glycerolipids, and one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 50% to about 95% by weight of one or more glycerolipids, and about 0.1% to about 40% by weight of one or more digestion enhancers. In some embodiments, a pharmaceutical composition comprises about 25 mg/mL to about 300 mg/mL of one or more fibrates, about 50% to about 95% by weight of one or more glycerolipids, and about 0.1% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 50% to about 95% by weight of one or more glycerolipids, and about 0.1% to about 10% by weight of one or more digestion enhancers. In some embodiments, a pharmaceutical composition comprises about 25 mg/mL to about 300 mg/mL of one or more fibrates, about 50% to about 95% by weight of one or more glycerolipids, and about 0.1% to about 10% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 50% to about 95% by weight of one or more glycerolipids, and about 5% to about 40% by weight of one or more digestion enhancers. In some embodiments, a pharmaceutical composition comprises about 25 mg/mL to about 300 mg/mL of one or more fibrates, about 50% to about 95% by weight of one or more glycerolipids, and about 5% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 50% to about 85% by weight of one or more glycerolipids, and about 10% to about 40% by weight of one or more digestion enhancers. In some embodiments, a pharmaceutical composition comprises about 25 mg/mL to about 300 mg/mL of one or more fibrates, about 50% to about 85% by weight of one or more glycerolipids, and about 10% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises a fenofibrate, one or more glycerolipids, and one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 50% to about 95% by weight of one or more glycerolipids, and about 0.1% to about 40% by weight of one or more digestion enhancers. In some embodiments, a pharmaceutical composition comprises about 25 mg/mL to about 300 mg/mL of a fenofibrate, about 50% to about 95% by weight of one or more glycerolipids, and about 0.1% to about 40% by weight of one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 50% to about 95% by weight of one or more glycerolipids, and about 0.1% to about 10% by weight of one or more digestion enhancers. In some embodiments, a pharmaceutical composition comprises about 25 mg/mL to about 300 mg/mL of a fenofibrate, about 50% to about 95% by weight of one or more glycerolipids, and about 0.1% to about 10% by weight of one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 50% to about 95% by weight of one or more glycerolipids, and about 5% to about 40% by weight of one or more digestion enhancers. In some embodiments, a pharmaceutical composition comprises about 25 mg/mL to about 300 mg/mL of a fenofibrate, about 50% to about 95% by weight of one or more glycerolipids, and about 5% to about 40% by weight of one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 50% to about 85% by weight of one or more glycerolipids, and about 10% to about 40% by weight of one or more digestion enhancers. In some embodiments, a pharmaceutical composition comprises about 25 mg/mL to about 300 mg/mL of a fenofibrate, about 50% to about 85% by weight of one or more glycerolipids, and about 10% to about 40% by weight of one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises one or more fibrates, one or more hard fats, one or more liquid fats, and one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 40% to about 50% by weight of one or more hard fats, about 30% to about 50% by weight of one or more liquid fats, and about 0.1% to about 40% by weight of one or more digestion enhancers. In some embodiments, a pharmaceutical composition comprises about 25 mg/mL to about 300 mg/mL of one or more fibrates, about 40% to about 50% by weight of one or more hard fats, about 30% to about 50% by weight of one or more liquid fats, and about 0.1% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 40% to about 50% by weight of one or more hard fats comprising glycerolipids, about 30% to about 50% by weight of one or more liquid fats comprising partially hydrolyzed glycerolipids including a mixture of mono-, di- and triglycerides, and about 0.1% to about 40% by weight of one or more digestion enhancers. In some embodiments, a pharmaceutical composition comprises about 25 mg/mL to about 300 mg/mL of one or more fibrates, about 40% to about 50% by weight of one or more hard fats comprising glycerolipids, about 30% to about 50% by weight of one or more liquid fats comprising partially hydrolyzed glycerolipids including a mixture of mono-, di- and triglycerides, and about 0.1% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 40% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 40% by weight of one or more digestion enhancers. In some embodiments, a pharmaceutical composition comprises about 25 mg/mL to about 300 mg/mL of one or more fibrates, about 40% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of one or more fibrates, about 40% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 40% by weight of one or more digestion enhancers. In some embodiments, a pharmaceutical composition comprises about 25 mg/mL to about 300 mg/mL of one or more fibrates, about 40% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 40% by weight of one or more digestion enhancers. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises a fenofibrate, one or more hard fats, one or more liquid fats, and one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 40% to about 50% by weight one or more hard fats, about 30% to about 50% by weight one or more liquid fats, and about 0.1% to about 40% by weight one or more digestion enhancers. In some embodiments, a pharmaceutical composition comprises about 25 mg/mL to about 300 mg/mL of a fenofibrate, about 40% to about 50% by weight one or more hard fats, about 30% to about 50% by weight one or more liquid fats, and about 0.1% to about 40% by weight one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 40% to about 50% by weight one or more hard fats comprising glycerolipids, about 30% to about 50% by weight one or more liquid fats comprising partially hydrolyzed glycerolipids including a mixture of mono-, di- and triglycerides, and about 0.1% to about 40% by weight one or more digestion enhancers. In some embodiments, a pharmaceutical composition comprises about 25 mg/mL to about 300 mg/mL of a fenofibrate, about 40% to about 50% by weight one or more hard fats comprising glycerolipids, about 30% to about 50% by weight one or more liquid fats comprising partially hydrolyzed glycerolipids including a mixture of mono-, di- and triglycerides, and about 0.1% to about 40% by weight one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 40% to about 50% by weight one or more hard fats comprising C₁₀-C₂₄ triglycerides, about 30% to about 50% by weight one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 40% by weight one or more digestion enhancers. In some embodiments, a pharmaceutical composition comprises about 25 mg/mL to about 300 mg/mL of a fenofibrate, about 40% to about 50% by weight one or more hard fats comprising C₁₀-C₂₄ triglycerides, about 30% to about 50% by weight one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 40% by weight one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises about 0.1% to about 25% by weight of a fenofibrate, about 40% to about 50% by weight one or more hard fats comprising a mixture of saturated C₁₀-C₁₈ triglycerides and/or saturated C₁₂-C₁₈ triglycerides, about 30% to about 50% by weight one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 40% by weight one or more digestion enhancers. In some embodiments, a pharmaceutical composition comprises about 25 mg/mL to about 300 mg/mL of a fenofibrate, about 40% to about 50% by weight one or more hard fats comprising a mixture of saturated C₁₀-C₁₈ triglycerides and/or saturated C₁₂-C₁₈ triglycerides, about 30% to about 50% by weight one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 40% by weight one or more digestion enhancers. In some embodiments, the one or more digestion enhancers comprises one or more bile acids, one or more one or more free C₁₄₋₂₄ fatty acids, one or more one or more free C₁₄₋₂₄ fatty acid surfactants, or any combination thereof.

In some embodiments, a pharmaceutical composition comprises one or more fibrates, one or more hard fats, one or more liquid fats, and one or more bile acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid.

In some embodiments, a pharmaceutical composition comprises about 0.1% to about 25% by weight of one or more fibrates, about 40% to about 50% by weight of one or more hard fats, about 30% to about 50% by weight of one or more liquid fats, and about 0.1% to about 10% by weight of one or more bile acids. In some embodiments, a pharmaceutical composition comprises about 25 mg/mL to about 300 mg/mL of one or more fibrates, about 40% to about 50% by weight of one or more hard fats, about 30% to about 50% by weight of one or more liquid fats, and about 0.1% to about 10% by weight of one or more bile acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid.

In some embodiments, a pharmaceutical composition comprises about 0.1% to about 25% by weight of one or more fibrates, about 40% to about 50% by weight of one or more hard fats comprising glycerolipids, about 30% to about 50% by weight of one or more liquid fats comprising partially hydrolyzed glycerolipids including a mixture of mono-, di- and triglycerides, and about 0.1% to about 10% by weight of one or more bile acids. In some embodiments, a pharmaceutical composition comprises about 25 mg/mL to about 300 mg/mL of one or more fibrates, about 40% to about 50% by weight of one or more hard fats comprising glycerolipids, about 30% to about 50% by weight of one or more liquid fats comprising partially hydrolyzed glycerolipids including a mixture of mono-, di- and triglycerides, and about 0.1% to about 10% by weight of one or more bile acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid.

In some embodiments, a pharmaceutical composition comprises about 0.1% to about 25% by weight of one or more fibrates, about 40% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 10% by weight of one or more bile acids. In some embodiments, a pharmaceutical composition comprises about 25 mg/mL to about 300 mg/mL of one or more fibrates, about 40% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 10% by weight of one or more bile acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid.

In some embodiments, a pharmaceutical composition comprises about 0.1% to about 25% by weight of one or more fibrates, about 40% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 10% by weight of one or more bile acids. In some embodiments, a pharmaceutical composition comprises about 25 mg/mL to about 300 mg/mL of one or more fibrates, about 40% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 10% by weight of one or more bile acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid.

In some embodiments, a pharmaceutical composition comprises a fenofibrate, one or more hard fats, one or more liquid fats, and one or more bile acids. In some embodiments, the one or more bile acids in the above embodiments comprises cholic acid.

In some embodiments, a pharmaceutical composition comprises about 0.1% to about 25% by weight of a fenofibrate, about 40% to about 50% by weight of one or more hard fats, about 30% to about 50% by weight of one or more liquid fats, and about 0.1% to about 10% by weight of one or more bile acids. In some embodiments, a pharmaceutical composition comprises about 25 mg/mL to about 300 mg/mL of a fenofibrate s, about 40% to about 50% by weight of one or more hard fats, about 30% to about 50% by weight of one or more liquid fats, and about 0.1% to about 10% by weight of one or more bile acids. In some embodiments, the one or more bile acids in the above embodiments comprises cholic acid.

In some embodiments, a pharmaceutical composition comprises about 0.1% to about 25% by weight of a fenofibrate, about 40% to about 50% by weight of one or more hard fats comprising glycerolipids, about 30% to about 50% by weight of one or more liquid fats comprising partially hydrolyzed glycerolipids including a mixture of mono-, di- and triglycerides, and about 0.1% to about 10% by weight of one or more bile acids. In some embodiments, a pharmaceutical composition comprises about 25 mg/mL to about 300 mg/mL of a fenofibrate, about 40% to about 50% by weight of one or more hard fats comprising glycerolipids, about 30% to about 50% by weight of one or more liquid fats comprising partially hydrolyzed glycerolipids including a mixture of mono-, di- and triglycerides, and about 0.1% to about 10% by weight of one or more bile acids. In some embodiments, the one or more bile acids in the above embodiments comprises cholic acid.

In some embodiments, a pharmaceutical composition comprises about 0.1% to about 25% by weight of a fenofibrate, about 40% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 10% by weight of one or more bile acids. In some embodiments, a pharmaceutical composition comprises about 25 mg/mL to about 300 mg/mL of a fenofibrate, about 40% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, and about 0.1% to about 10% by weight of one or more bile acids. In some embodiments, the one or more bile acids in the above embodiments comprises cholic acid.

In some embodiments, a pharmaceutical composition comprises about 0.1% to about 25% by weight of a fenofibrate, about 40% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 10% by weight of one or more bile acids. In some embodiments, a pharmaceutical composition comprises about 25 mg/mL to about 300 mg/mL of a fenofibrate, about 40% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, and about 0.1% to about 10% by weight of one or more bile acids. In some embodiments, the one or more bile acids in the above embodiments comprises cholic acid.

In some embodiments, a pharmaceutical composition comprises one or more fibrates, one or more hard fats, one or more liquid fats, one or more bile acids and one or more free C₁₄₋₂₄ fatty acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of one or more fibrates, about 25% to about 50% by weight of one or more hard fats, about 30% to about 50% by weight of one or more liquid fats, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of one or more fibrates, about 25% to about 50% by weight of one or more hard fats, about 30% to about 50% by weight of one or more liquid fats, about 0.1% to about 10% by weight of one or more bile acids, and about 5% to about 25% by weight of one or more free C₁₄₋₂₄ fatty acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of one or more fibrates, about 25% to about 50% by weight of one or more hard fats comprising glycerolipids, about 30% to about 50% by weight of one or more liquid fats comprising partially hydrolyzed glycerolipids including a mixture of mono-, di- and triglycerides, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of one or more fibrates, about 25% to about 50% by weight of one or more hard fats comprising glycerolipids, about 30% to about 50% by weight of one or more liquid fats comprising partially hydrolyzed glycerolipids including a mixture of mono-, di- and triglycerides, about 0.1% to about 10% by weight of one or more bile acids, and about 5% to about 25% by weight of one or more free C₁₄₋₂₄ fatty acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of one or more fibrates, about 25% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of one or more fibrates, about 25% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, and about 5% to about 25% by weight of one or more free C₁₄₋₂₄ fatty acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of one or more fibrates, about 25% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of one or more fibrates, about 25% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, and about 5% to about 25% by weight of one or more free C₁₄₋₂₄ fatty acids. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises a fenofibrate, one or more hard fats, one or more liquid fats, one or more bile acids and one or more free C₁₄₋₂₄ fatty acids. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of a fenofibrate, about 25% to about 50% by weight of one or more hard fats, about 30% to about 50% by weight of one or more liquid fats, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of a fenofibrate, about 25% to about 50% by weight of one or more hard fats, about 30% to about 50% by weight of one or more liquid fats, about 0.1% to about 10% by weight of one or more bile acids, and about 5% to about 25% by weight of one or more free C₁₄₋₂₄ fatty acids. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of a fenofibrate, about 25% to about 50% by weight of one or more hard fats comprising glycerolipids, about 30% to about 50% by weight of one or more liquid fats comprising partially hydrolyzed glycerolipids including a mixture of mono-, di- and triglycerides, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of a fenofibrate, about 25% to about 50% by weight of one or more hard fats comprising glycerolipids, about 30% to about 50% by weight of one or more liquid fats comprising partially hydrolyzed glycerolipids including a mixture of mono-, di- and triglycerides, about 0.1% to about 10% by weight of one or more bile acids, and about 5% to about 25% by weight of one or more free C₁₄₋₂₄ fatty acids. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of a fenofibrate, about 25% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of a fenofibrate, about 25% to about 50% by weight of one or more hard fats comprising C₁₀-C₂₄ triglycerides, about 30% to about 50% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, and about 5% to about 25% by weight of one or more free C₁₄₋₂₄ fatty acids. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises about 0.1% to about 25% by weight of a fenofibrate, about 25% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, and about 5% to about 25% by weight of one or more free C₁₄₋₂₄ fatty acids. In some embodiments, a pharmaceutical composition comprises about 25 mg/mL to about 300 mg/mL of a fenofibrate, about 25% to about 50% by weight of one or more hard fats comprising a mixture of saturated C₁₀-C₁₈ triglycerides and/or saturated C₁₂-C₁₈ triglycerides, about 30% to about 50% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, and about 5% to about 25% by weight of one or more free C₁₄₋₂₄ fatty acids. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, a pharmaceutical composition comprises one or more fibrates, one or more hard fats, one or more liquid fats, one or more bile acids, one or more free C₁₄₋₂₄ fatty acids, and one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of one or more fibrates, about 25% to about 40% by weight of one or more hard fats, about 30% to about 45% by weight of one or more liquid fats, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of one or more fibrates, about 25% to about 40% by weight of one or more hard fats, about 30% to about 45% by weight of one or more liquid fats, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of one or more fibrates, about 25% to about 40% by weight of one or more hard fats comprising glycerolipids, about 30% to about 45% by weight of one or more liquid fats comprising partially hydrolyzed glycerolipids including a mixture of mono-, di- and triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of one or more fibrates, about 25% to about 40% by weight of one or more hard fats comprising glycerolipids, about 30% to about 45% by weight of one or more liquid fats comprising partially hydrolyzed glycerolipids including a mixture of mono-, di- and triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of one or more fibrates, about 25% to about 40% by weight of one or more hard fats comprising C₁₀-C₂₄ triglycerides, about 30% to about 45% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of one or more fibrates, about 25% to about 40% by weight of one or more hard fats comprising C₁₀-C₂₄ triglycerides, about 30% to about 45% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, and about 10% to about 25% by weight of one or more free C₁₄₋₂₄ fatty acids, about 0.1% to about 5% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of one or more fibrates, about 25% to about 40% by weight of one or more hard fats comprising a mixture of saturated C₁₀-C₁₈ triglycerides and/or saturated C₁₂-C₁₈ triglycerides, about 30% to about 45% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of one or more fibrates, about 25% to about 40% by weight of one or more hard fats comprising a mixture of saturated C₁₀-C₁₈ triglycerides and/or saturated C₁₂-C₁₈ triglycerides, about 30% to about 45% by weight of one or more liquid fats comprising partially hydrolyzed triglycerides including a mixture of C₁₆-C₁₈ mono-, C₁-C₁₈ di- and C₁₆-C₁₈ triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, one or more hard fats, one or more liquid fats, one or more bile acids, one or more free C₁₄₋₂₄ fatty acids, and one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of a fenofibrate, about 25% to about 40% by weight of one or more hard fats, about 30% to about 45% by weight of one or more liquid fats, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of a fenofibrate, about 25% to about 40% by weight of one or more hard fats, about 30% to about 45% by weight of one or more liquid fats, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of a fenofibrate, about 25% to about 40% by weight of one or more hard fats comprising glycerolipids, about 30% to about 45% by weight of one or more liquid fats comprising partially hydrolyzed glycerolipids including a mixture of mono-, di- and triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of a fenofibrate, about 25% to about 40% by weight of one or more hard fats comprising glycerolipids, about 30% to about 45% by weight of one or more liquid fats comprising partially hydrolyzed glycerolipids including a mixture of mono-, di- and triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of a fenofibrate, about 25% to about 40% by weight of one or more hard fats comprising C₁₀-C₂₄ triglycerides, about 30% to about 45% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of a fenofibrate, about 25% to about 40% by weight of one or more hard fats comprising C₁₀-C₂₄ triglycerides, about 30% to about 45% by weight of one or more liquid fats comprising partially hydrolyzed triglycerides including a mixture of C₁₀-C₂₄ mono-, C₁₀-C₂₄ di- and C₁₀-C₂₄ triglycerides, about 0.1% to about 10% by weight of one or more bile acids, and about 10% to about 25% by weight of one or more free C₁₄₋₂₄ fatty acids, about 0.1% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 0.1% to about 25% by weight of a fenofibrate, about 25% to about 40% by weight of one or more hard fats comprising a mixture of saturated C₁₀-C₁₈ triglycerides and/or saturated C₁₂-C₁₈ triglycerides, about 30% to about 45% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, a pharmaceutical composition comprises about 25 mg/mL to about 300 mg/mL of a fenofibrate, about 25% to about 40% by weight of one or more hard fats comprising a mixture of saturated C₁₀-C₁₈ triglycerides and/or saturated C₁₂-C₁₈ triglycerides, about 30% to about 45% by weight of one or more liquid fats comprising partially hydrolyzed triglycerides including a mixture of C₁₆-C₁₈ mono-, C₁₆-C₁₈ di- and C₁₆-C₁₈ triglycerides, about 0.1% to about 10% by weight of one or more bile acids, about 10% to about 25% by weight of one or more free C₁₄₋₂₄ fatty acids, and about 0.1% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of one or more fibrates, about 26% to about 36% by weight of one or more hard fats, about 26% to about 36% by weight of one or more liquid fats, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of one or more fibrates, about 26% to about 36% by weight of one or more hard fats, about 26% to about 36% by weight of one or more liquid fats, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of one or more fibrates, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of one or more fibrates, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of one or more fibrates, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of one or more fibrates, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 2% to about 8% by weight of one or more bile acids, and about 14% to about 20% by weight of one or more free C₁₄₋₂₄ fatty acids, about 0.8% to about 4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of one or more fibrates, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of one or more fibrates, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, one or more hard fats, one or more liquid fats, one or more bile acids, one or more free C₁₄₋₂₄ fatty acids, and one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of a fenofibrate, about 26% to about 36% by weight of one or more hard fats, about 26% to about 36% by weight of one or more liquid fats, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of a fenofibrate, about 26% to about 36% by weight of one or more hard fats, about 26% to about 36% by weight of one or more liquid fats, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of a fenofibrate, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of a fenofibrate, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of a fenofibrate, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of a fenofibrate, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 2% to about 8% by weight of one or more bile acids, and about 14% to about 20% by weight of one or more free C₁₄₋₂₄ fatty acids, about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 10% to about 20% by weight of a fenofibrate, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, a pharmaceutical composition comprises about 10 mg/mL to about 400 mg/mL of a fenofibrate, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides, about 2% to about 8% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of one or more fibrates, about 28% to about 34% by weight of one or more hard fats, about 28% to about 34% by weight of one or more liquid fats, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of one or more fibrates, about 28% to about 34% by weight of one or more hard fats, about 28% to about 34% by weight of one or more liquid fats, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of one or more fibrates, about 28% to about 34% by weight of one or more hard fats comprising glycerolipids, about 28% to about 34% by weight of one or more liquid fats comprising partially hydrolyzed glycerolipids including a mixture of mono-, di- and triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of one or more fibrates, about 28% to about 34% by weight of one or more hard fats comprising glycerolipids, about 28% to about 34% by weight of one or more liquid fats comprising partially hydrolyzed glycerolipids including a mixture of mono-, di- and triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of one or more fibrates, about 28% to about 34% by weight of one or more hard fats comprising C₁₀-C₂₄ triglycerides, about 28% to about 34% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of one or more fibrates, about 28% to about 34% by weight of one or more hard fats comprising C₁₀-C₂₄ triglycerides, about 28% to about 34% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 7% by weight of one or more bile acids, and about 14% to about 20% by weight of one or more free C₁₄₋₂₄ fatty acids, about 0.8% to about 3% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of one or more fibrates, about 28% to about 34% by weight of one or more hard fats comprising a mixture of saturated C₁₀-C₁₈ triglycerides and/or saturated C₁₂-C₁₈ triglycerides, about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of one or more fibrates, about 28% to about 34% by weight of one or more hard fats comprising a mixture of saturated C₁₀-C₁₈ triglycerides and/or saturated C₁₂-C₁₈ triglycerides, about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of a fenofibrate, about 28% to about 34% by weight of one or more hard fats, about 28% to about 34% by weight of one or more liquid fats, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of a fenofibrate, about 28% to about 34% by weight of one or more hard fats, about 28% to about 34% by weight of one or more liquid fats, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of a fenofibrate, about 28% to about 34% by weight of one or more hard fats comprising glycerolipids, about 28% to about 34% by weight of one or more liquid fats comprising partially hydrolyzed glycerolipids including a mixture of mono-, di- and triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of a fenofibrate, about 28% to about 34% by weight of one or more hard fats comprising glycerolipids, about 28% to about 34% by weight of one or more liquid fats comprising partially hydrolyzed glycerolipids including a mixture of mono-, di- and triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof.

In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of a fenofibrate, about 28% to about 34% by weight of one or more hard fats comprising C₁₀-C₂₄ triglycerides, about 28% to about 34% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of a fenofibrate, about 28% to about 34% by weight of one or more hard fats comprising C₁₀-C₂₄ triglycerides, about 28% to about 34% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 7% by weight of one or more bile acids, and about 14% to about 20% by weight of one or more free C₁₄₋₂₄ fatty acids, about 0.8% to about 3% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 18% by weight of a fenofibrate, about 28% to about 34% by weight of one or more hard fats comprising a mixture of saturated C₁₀-C₁₈ triglycerides and/or saturated C₁₂-C₁₈ triglycerides, about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, a pharmaceutical composition comprises about 25 mg/mL to about 300 mg/mL of a fenofibrate, about 28% to about 34% by weight of one or more hard fats comprising a mixture of saturated C₁₀-C₁₈ triglycerides and/or saturated C₁₂-C₁₈ triglycerides, about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 7% by weight of one or more bile acids, about 14% to about 20% by weight of one or more free C₁₄₋₂₄ fatty acids, and about 0.8% to about 3% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of one or more fibrates, about 30% to about 32% by weight of one or more hard fats, about 30% to about 32% by weight of one or more liquid fats, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of one or more fibrates, about 30% to about 32% by weight of one or more hard fats, about 30% to about 32% by weight of one or more liquid fats, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄. 18 fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of one or more fibrates, about 30% to about 32% by weight of one or more hard fats comprising glycerolipids, about 30% to about 32% by weight of one or more liquid fats comprising partially hydrolyzed glycerolipids including a mixture of mono-, di- and triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of one or more fibrates, about 30% to about 32% by weight of one or more hard fats comprising glycerolipids, about 30% to about 32% by weight of one or more liquid fats comprising partially hydrolyzed glycerolipids including a mixture of mono-, di- and triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of one or more fibrates, about 30% to about 32% by weight of one or more hard fats comprising C₁₀-C₂₄ triglycerides, about 30% to about 32% by weight of one or more liquid fats comprising partially hydrolyzed triglycerides including a mixture of C₁₀-C₂₄ mono-, C₁₀-C₂₄ di- and C₁₀-C₂₄ triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of one or more fibrates, about 30% to about 32% by weight of one or more hard fats comprising C₁₀-C₂₄ triglycerides, about 30% to about 32% by weight of one or more liquid fats comprising partially hydrolyzed triglycerides including a mixture of C₁₀-C₂₄ mono-, C₁₀-C₂₄ di- and C₁₀-C₂₄ triglycerides, about 4% to about 6% by weight of one or more bile acids, and about 16% to about 18% by weight of one or more free C₁₄₋₂₄ fatty acids, about 1% to about 1.4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of one or more fibrates, about 30% to about 32% by weight of one or more hard fats comprising a mixture of saturated C₁₀-C₁₈ triglycerides and/or saturated C₁₂-C₁₈ triglycerides, about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of one or more fibrates, about 30% to about 32% by weight of one or more hard fats comprising a mixture of saturated C₁₀-C₁₈ triglycerides and/or saturated C₁₂-C₁₈ triglycerides, about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more bile acids in the above embodiments comprises cholic acid. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more free C₁₄₋₂₄ fatty acids in the above embodiments comprises one or more free C₁₄-18 fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more fibrates in the above embodiments comprises a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of a fenofibrate, about 30% to about 32% by weight of one or more hard fats, about 30% to about 32% by weight of one or more liquid fats, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of a fenofibrate, about 30% to about 32% by weight of one or more hard fats, about 30% to about 32% by weight of one or more liquid fats, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of a fenofibrate, about 30% to about 32% by weight of one or more hard fats comprising glycerolipids, about 30% to about 32% by weight of one or more liquid fats comprising partially hydrolyzed glycerolipids including a mixture of mono-, di- and triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of a fenofibrate, about 30% to about 32% by weight of one or more hard fats comprising glycerolipids, about 30% to about 32% by weight of one or more liquid fats comprising partially hydrolyzed glycerolipids including a mixture of mono-, di- and triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of a fenofibrate, about 30% to about 32% by weight of one or more hard fats comprising C₁₀-C₂₄ triglycerides, about 30% to about 32% by weight of one or more liquid fats comprising partially hydrolyzed triglycerides including a mixture of C₁₀-C₂₄ mono-, C₁₀-C₂₄ di- and C₁₀-C₂₄ triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of a fenofibrate, about 30% to about 32% by weight of one or more hard fats comprising C₁₀-C₂₄ triglycerides, about 30% to about 32% by weight of one or more liquid fats comprising partially hydrolyzed triglycerides including a mixture of C₁₀-C₂₄ mono-, C₁₀-C₂₄ di- and C₁₀-C₂₄ triglycerides, about 4% to about 6% by weight of one or more bile acids, and about 16% to about 18% by weight of one or more free C₁₄₋₂₄ fatty acids, about 1% to about 1.4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 14% to about 16% by weight of a fenofibrate, about 30% to about 32% by weight of one or more hard fats comprising a mixture of saturated C₁₀-C₁₈ triglycerides and/or saturated C₁₂-C₁₈ triglycerides, about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, a pharmaceutical composition comprises about 25 mg/mL to about 200 mg/mL of a fenofibrate, about 30% to about 32% by weight of one or more hard fats comprising a mixture of saturated C₁₀-C₁₈ triglycerides and/or saturated C₁₂-C₁₈ triglycerides, about 30% to about 32% by weight of one or more liquid fats comprising partially hydrolyzed triglycerides including a mixture of C₁₆-C₁₈ mono-, C₁₆₋₁₈ di- and C₁₆-C₁₈ triglycerides, about 4% to about 6% by weight of one or more bile acids, about 16% to about 18% by weight of one or more free C₁₄₋₂₄ fatty acids, and about 1% to about 1.4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 4% to about 8% by weight of a fenofibrate, about 26% to about 36% by weight of one or more hard fats, about 26% to about 36% by weight of one or more liquid fats, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 4% to about 8% by weight of a fenofibrate, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 4% to about 8% by weight of a fenofibrate, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 4% to about 8% by weight of a fenofibrate, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₀-C₁₈ di- and C₁₆-C₁₈ triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 8% to about 12% by weight of a fenofibrate, about 26% to about 36% by weight of one or more hard fats, about 26% to about 36% by weight of one or more liquid fats, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 8% to about 12% by weight of a fenofibrate, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 8% to about 12% by weight of a fenofibrate, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 8% to about 12% by weight of a fenofibrate, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₀-C₁₈ di- and C₁₆-C₁₈ triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 16% by weight of a fenofibrate, about 26% to about 36% by weight of one or more hard fats, about 26% to about 36% by weight of one or more liquid fats, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 16% by weight of a fenofibrate, about 26% to about 36% by weight of one or more hard fats comprising glycerolipids, about 26% to about 36% by weight of one or more liquid fats comprising partially hydrolyzed glycerolipids including a mixture of mono-, di- and triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄-1₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 16% by weight of a fenofibrate, about 26% to about 36% by weight of one or more hard fats comprising C₁₀-C₂₄ triglycerides, about 26% to about 36% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises about 12% to about 16% by weight of a fenofibrate, about 26% to about 36% by weight of one or more hard fats comprising a mixture of saturated C₁₀-C₁₈ triglycerides and/or saturated C₁₂-C₁₈ triglycerides, about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 6% by weight of one or more bile acids, about 14% to about 20% by weight of one or more free C₁₄₋₂₄ fatty acids, and about 0.8% to about 4% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 30% to about 40% by weight of one or more hard fats, about 30% to about 40% by weight of one or more liquid fats, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 30% to about 40% by weight of one or more hard fats comprising glycerolipids, about 30% to about 40% by weight of one or more liquid fats comprising partially hydrolyzed glycerolipids including a mixture of mono-, di- and triglycerides, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 30% to about 40% by weight of one or more hard fats comprising C₁₀-C₂₄ triglycerides, about 30% to about 40% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 30% to about 40% by weight of one or more hard fats comprising a mixture of saturated C₁₀-C₁₈ triglycerides and/or saturated C₁₂-C₁₈ triglycerides, about 30% to about 40% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats, about 50% to about 60% by weight of one or more liquid fats, about 3% to about 6% by weight of one or more bile acids, about 10% to about 20% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats comprising glycerolipids, about 50% to about 60% by weight of one or more liquid fats comprising partially hydrolyzed glycerolipids including a mixture of mono-, di- and triglycerides, about 3% to about 6% by weight of one or more bile acids, about 10% to about 20% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats comprising C₁₀-C₂₄ triglycerides, about 50% to about 60% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 10% to about 20% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats comprising a mixture of saturated C₁₀-C₁₈ triglycerides and/or saturated C₁₂-C₁₈ triglycerides, about 50% to about 60% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 6% by weight of one or more bile acids, about 10% to about 20% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats, about 10% to about 20% by weight of one or more liquid fats, about 3% to about 6% by weight of one or more bile acids, about 50% to about 60% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats comprising glycerolipids, about 10% to about 20% by weight of one or more liquid fats comprising partially hydrolyzed glycerolipids including a mixture of mono-, di- and triglycerides, about 3% to about 6% by weight of one or more bile acids, about 50% to about 60% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats comprising C₁₀-C₂₄ triglycerides, about 10% to about 20% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 50% to about 60% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

In some embodiments, a pharmaceutical composition comprises a fenofibrate, about 15% to about 20% by weight of one or more hard fats comprising a mixture of saturated C₁₀-C₁₈ triglycerides and/or saturated C₁₂-C₁₈ triglycerides, about 10% to about 20% by weight of one or more liquid fats comprising partially hydrolyzed triglycerides including a mixture of C₁₆-C₁₈ mono-, C₁₆-C₁₈ di- and C₁₆-C₁₈ triglycerides, about 3% to about 6% by weight of one or more bile acids, about 50% to about 60% by weight of one or more free C₁₄₋₂₄ fatty acids, and about 0.5% to about 5% by weight of one or more free C₁₄₋₂₄ fatty acid surfactants. In some embodiments, the one or more bile acids in the above embodiments comprises cholic acid. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid and the one or more free C₁₄₋₂₄ fatty acids in the above embodiments comprises one or more free C₁₄₋₁₈ fatty acids, preferably oleic acid, steric acid, linoleic acid, or any combination thereof. In some embodiments, the one or more bile acids in the above embodiments comprises cholic acid and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof. In some embodiments, the one or more bile acids in the above embodiments comprises cholic acid, the one or more free C₁₄₋₂₄ fatty acids in the above embodiments comprises one or more free C₁₄₋₁₈ fatty acids, preferably oleic acid, steric acid, linoleic acid, or any combination thereof, and the one or more free C₁₄₋₂₄ fatty acid surfactants in the above embodiments comprises one or more free C₁₄₋₁₈ fatty acid surfactants, preferably an oleate alkali metal or alkali earth metal salt, a stearate alkali metal or alkali earth metal salt, a linoleate alkali metal or alkali earth metal salt, or any combination thereof.

According to an aspect of the present specification there is provided a pharmaceutical composition comprising one or more fibrates, one or more glycerolipids, one or more bile acids, one or more free C₁₄-C₂₄ fatty acids, one or more free C₁₄-C₂₄ fatty acid surfactants, or any combination thereof for use in the treatment of inflammation. In a preferred embodiment, a pharmaceutical composition is not an emulsion or self-emulsifying formulation. In a preferred embodiment, an inflammation is a neuroinflammation. The terms inflammation and neuroinflammation in the context of this application are associated with disease which are caused by chronic inflammation such as for example autoimmune disease or Parkinson disease.

In a preferred embodiment, the one or more fibrates comprise a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof. In a preferred embodiment, the one or more fibrates comprise or consist of fenofibrate or fenofibric acid. In a preferred embodiment, the one or more fibrates are preferably in an amount of between 0.1 to 25%, preferably between 1 to 20% by weight, or preferably at between 4-16% by of the composition.

In a preferred embodiment, the one or more glycerolipids are provided in the amount of between 50-95% by weight, preferably between 55-85% by weight or even more preferably between 55-75% by weight of the composition. In a preferred embodiment, the one or more glycerolipids comprise or consist of one or more hard fats and one or more liquid fats. In a preferred embodiment, the one or more hard fats and the one or more liquid fats are in a hard fat to liquid fat ratio of 2:1 or 1:2 and preferably at a ratio of 1:1. In a preferred embodiment, the one or more hard fats is a glycerolipid that is solid at 18° C. In a preferred embodiment of the invention said liquid fat is a glycerolipid that is liquid at 18° C.

In a preferred embodiment, the one or more hard fats comprises or consist of one or more triglycerides. In a preferred embodiment, the one or more hard fat is provided at between 25% to 40% by weight of the composition, or preferably between 28% to 36% by weight of the composition. In a preferred embodiment, the one or more triglycerides include a mixture of a saturated and/or unsaturated C₁₀-C₂₄ triglyceride, C₁₀-C₁₈ triglyceride or C₁₂-C₁₈ triglyceride, or more preferable include a mixture of saturated and/or unsaturated C₁₀-C₁₈ triglycerides and/or a mixture of saturated and/or unsaturated C₁₂-C₁₈ triglycerides. In a preferred embodiment, the one or more triglycerides is CAS 85665-33-4 or 67701-26-2 or UNII Code (FDA) 8334LX7S21 and sold under the tradename GELUCIRE® 43/01.

In a preferred embodiment, the one or more liquid fats comprises or consist of a mixture of mono, di- and triglycerides. In a preferred embodiment, the mixture of mono-, di- and triglycerides comprises or consist of a mixture of C₁₆-C₁₈ mono-, C₁₆-C₁₈ di- and C₁₆-C₁₈ triglycerides. In a preferred embodiment, the one or more liquid fat includes glycerol monomyristoleate, glycerol monopalmitoleate, glycerol monosapienate, glycerol monooleate, glycerol monoelaidate, glycerol monovaccenate, glycerol monolinoleate, glycerol monolinoelaidate, glycerol monolinolenate, glycerol monostearidonate, glycerol monoeicosenoate, glycerol monomeadate, glycerol monoarachidonate, glycerol monoeicosapentaenoate, glycerol monoerucate, glycerol monodocosahexaenoate, and glycerol mononervonate. In a preferred embodiment, the one or more liquid fats comprise or consist of partially hydrolyzed triglycerides include a mixture of C₁₆-C₁₈ mono-, C₁₆-C₁₈ di- and C₁₆-C₁₈ triglycerides. In a preferred embodiment, the one or more liquid fats comprise or consist of partially hydrolyzed triglycerides include a mixture of C₁₆-C₁₈ monoglycerides comprising or consist of glycerol monolinoleate, C₁₆-C₁₈ di- and C₁₆-C₁₈ triglycerides. In a preferred embodiment comprise or consist of liquid fats includes or consist of glycerol monolinoleate and/or glycerol monooleate. In an alternative preferred embodiment, the one or more liquid fats comprises or consist of mono, di- and triglycerides of linoleic and/or oleic acid. In a preferred embodiment, the one or more liquid fats is CAS 68424-61-3 or 26545-74-4 or 91744-20-6 or UNII Code (FDA) 4763AX184L or 1 DAF35W3S2 and sold under the trade name MAISINE 35 CC. In an alternative preferred embodiment of the invention said one or more liquid fat is provided at between 30 to 45% by weight, or preferably between 28-36% by weight of the composition.

In a preferred embodiment, the one or more bile acids are selected from the group comprising or consisting of cholic acid, chenodeoxycholic acid, dafachronic acid, deoxycholic acid, glycocholic acid, glycohenodeoxycholic acid, lithocholic acid, taurochenodeoxycholic acid, taurocholic acid, any stereoisomer thereof, and any combination thereof. In a preferred embodiment, the one or more bile acids comprises or consist of cholic acid. In a preferred embodiment, the one or more bile acids are provided between 0.1-10% by weight, preferably between 3% to 7% by weight or even more preferably between 5% to 6% by weight of the composition.

In an alternative preferred embodiment, the one or more free C₁₄-C₂₄ fatty acids include palmitic acid (hexadecenoic acid), palmitolinolenic acid, palmitidonic acid, palmitovaccenic acid, palmitoleic acid, sapienic acid, 4-Hexadecenoic acid, stearic acid (octadecenoic acid), α-linolenic acid, stearidonic acid, α-eleostearic acid, β-eleostearic acid, pumicic acid, 7,10,13-octadecatrienoic acid, 12-octadecenoic acid, linoleic acid, linolelaidic acid. γ-linolenic acid, calendic acid, pinolenic acid, vaccinic acid, ruminic acid, oleic acid, elaidic acid, petroselinic acid, arachidic acid (eicosanoic acid), dihomo-α-linolenic acid, eicosic acidtraenoic acid, eicosapentaenoic acid, 9,12,15-eicosatrienoic acid, β-eicosic acidtraenoic acid, dihomo-linoleic acid, dihomo-γ-linolenic acid, arachidonic acid, paullinic acid, 7,10,13-eicosatrienoic acid, gondoic acid, 8,11-eicosadienoic acid, meadic acid, gadoleic acid, 8-eicosenoic acid, behenic acid (docosanoic acid), clupanodonic acid, docosahexaenoic acid, adrenic acid, osbondic acid, erucic acid, lignoceric acid (tetracosanic acid), 9,12,15,18,21-Tetracosapentaenoic acid, 6,9,12,15,18,21-Tetracosahexaenoic acid, and nervonic acid. In a preferred embodiment, the one or more free C₁₄-C₂₄ fatty acids comprises or consists of an oleic acid, a steric acid, a linoleic acid, or any combination thereof. In a preferred embodiment, the one or more free C₁₄-C₂₄ fatty acids are provided in an amount of between 10% to 25% by weight of the composition, preferably between 14% to 20% by weight of the composition, or even more preferably between 16% to 19% by weight of the composition.

In a preferred embodiment, the one or more free C₁₄-C₂₄ fatty acid surfactants include sodium palmitate (hexadecanoate), sodium palmitolinolenate, sodium palmitidonate, sodium palmitovaccenate, sodium palmitoleate, sodium sapienate, sodium 4-Hexadecenoate, sodium stearate (octadecenoate), sodium α-linolenate, sodium stearidonate, sodium α-eleostearate, sodium β-eleostearate, sodium pumicate, sodium 7,10,13-octadecatrienoate, sodium 12-octadecenoate, sodium linoleate, sodium linolelaidate. Sodium γ-linolenate, sodium calendate, sodium pinolenate, sodium vaccinate, sodium ruminate, sodium oleate, sodium elaidate, sodium petroselinate, sodium arachidate (eicosanoate), sodium dihomo-α-linolenate, sodium eicosatetraenoate, sodium eicosapentaenoate, sodium 9,12,15-eicosatrienoate, sodium β-eicosatetraenoate, sodium dihomo-linoleate, sodium dihomo-γ-linolenate, sodium arachidonate, sodium paullinate, sodium 7,10,13-eicosatrienoate, sodium gondoate, Sodium 8,11-eicosadienoate, sodium meadate, sodium gadoleate, sodium 8-eicosenoate, sodium behenate (docosanoate), sodium clupanodonate, sodium docosahexaenoate, sodium adrenate, sodium osbondate, sodium erucate, sodium lignocerate (tetracosanate), sodium 9,12,15,18,21-Tetracosapentaenoate, sodium 6,9,12,15,18,21-Tetracosahexaenoate, and sodium nervonate. In a preferred embodiment, the one or more free C₁₄-C₂₄ fatty acid surfactants is sodium stearate. In a preferred embodiment, the one or more free C₁₄-C₂₄ fatty acid surfactants are provided in an amount of between 0.1% to 5% by weight of the composition, or preferably between 0.8% to 3% by weight of the composition, or even more preferably between 1% to 1.4% by weight of the composition. In a preferred embodiment, the one or more free C₁₄-C₂₄ fatty acid surfactants comprise or consist of one or more free C₁₄-C₁₈ fatty acid surfactants. In a preferred embodiment, the one or more free C₁₄-C₁₈ fatty acid surfactants are oleate alkali metal or alkali earth metal salt, a stearate alkali metal or alkali earth metal salt, a linoleate alkali metal or alkali earth metal salt, or any combination thereof and preferably sodium stearate, sodium linoleate, sodium oleate, or a combination thereof.

In a preferred embodiment, the pharmaceutical composition comprises 0.1% to 25% by weight of one or more fibrates, 25% to 40% by weight of one or more hard fats, 30% to 45% by weight of one or more liquid fats, 0.1% to 10% by weight of one or more bile acids, 10% to 25% of one or more free C₁₄-C₂₄ fatty acids, and 0.1% to 5% of one or more free C₁₄-C₂₄ fatty acid surfactants.

In a preferred embodiment, the pharmaceutical composition comprises 1% to 20% by weight of a fenofibrate, 25% to 40% by weight of one or more hard fats, 30% to about 45% by weight of one or more liquid fats, 3% to about 7% by weight of one or more bile acids, 14% to 20% by weight of one or more free C₁₄-C₂₄ fatty acids, and 0.8% to about 3% by weight of one or more free C₁₄-C₂₄ fatty acid surfactants.

In a preferred embodiment, the pharmaceutical composition comprises 4% to 16% by weight of a fenofibrate, 28% to 36% by weight of one or more hard fats, 28% to about 36% by weight of one or more liquid fats, 5% to about 6% by weight of one or more bile acids, 16% to 19% by weight of one or more free C₁₄-C₂₄ fatty acids, and 1% to about 1.4% by weight of one or more free C₁₄-C₂₄ fatty acid surfactants. In a preferred embodiment of the invention said one or more bile acids comprises cholic acid.

Aspects of the present specification can also be described by the following embodiments:
1. A pharmaceutical composition, the pharmaceutical composition comprising a) one or more fibrates, b) one or more glycerolipids, and c) one or more digestion enhancers.
2. The pharmaceutical composition of embodiment 1, wherein the one or more fibrates comprise a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof.
3. The pharmaceutical composition of embodiment 1 or 2, wherein the one or more fibrates are in an amount of about 0.05% to about 1%, about 0.05% to about 2.5%, about 0.05% to about 5%, about 0.05% to about 7.5%, about 0.05% to about 10%, about 0.05% to about 12.5%, about 0.05% to about 15%, about 0.05% to about 17.5%, about 0.05% to about 20%, about 0.05% to about 22.5%, about 0.05% to about 25%, about 0.05% to about 30%, about 0.05% to about 40%, about 0.05% to about 50%, about 0.1% to about 1%, about 0.1% to about 2.5%, about 0.1% to about 5%, about 0.1% to about 7.5%, about 0.1% to about 10%, about 0.1% to about 12.5%, about 0.1% to about 15%, about 0.1% to about 17.5%, about 0.1% to about 20%, about 0.1% to about 22.5%, about 0.1% to about 25%, about 0.1% to about 30%, about 0.1% to about 40%, about 0.1% to about 50%, about 1% to about 2.5%, about 1% to about 5%, about 1% to about 7.5%, about 1% to about 10%, about 1% to about 12.5%, about 1% to about 15%, about 1% to about 17.5%, about 1% to about 20%, about 1% to about 22.5%, about 1% to about 25%, about 1% to about 30%, about 1% to about 40%, about 1% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50% by weight.
4. The pharmaceutical composition of any one of embodiments 1-3, wherein the one or more fibrates are in an amount of about 10 mg/mL to about 25 mg/mL, about 10 mg/mL to about 50 mg/mL, about 10 mg/mL to about 75 mg/mL, about 10 mg/mL to about 100 mg/mL, about 10 mg/mL to about 125 mg/mL, about 10 mg/mL to about 150 mg/mL, about 10 mg/mL to about 200 mg/mL, about 10 mg/mL to about 250 mg/mL, about 10 mg/mL to about 300 mg/mL, about 25 mg/mL to about 50 mg/mL, about 25 mg/mL to about 75 mg/mL, about 25 mg/mL to about 100 mg/mL, about 25 mg/mL to about 125 mg/mL, about 25 mg/mL to about 150 mg/mL, about 25 mg/mL to about 200 mg/mL, about 25 mg/mL to about 250 mg/mL, about 25 mg/mL to about 300 mg/mL, about 50 mg/mL to about 100 mg/mL, about 50 mg/mL to about 150 mg/mL, about 50 mg/mL to about 200 mg/mL, about 50 mg/mL to about 250 mg/mL, about 50 mg/mL to about 300 mg/mL, about 75 mg/mL to about 100 mg/mL, about 75 mg/mL to about 150 mg/mL, about 75 mg/mL to about 200 mg/mL, about 75 mg/mL to about 250 mg/mL, about 75 mg/mL to about 300 mg/mL, about 100 mg/mL to about 150 mg/mL, about 100 mg/mL to about 200 mg/mL, about 100 mg/mL to about 250 mg/mL, about 100 mg/mL to about 300 mg/mL, about 125 mg/mL to about 150 mg/mL, about 125 mg/mL to about 200 mg/mL, about 125 mg/mL to about 250 mg/mL, about 125 mg/mL to about 300 mg/mL, about 150 mg/mL to about 200 mg/mL, about 150 mg/mL to about 250 mg/mL, about 150 mg/mL to about 300 mg/mL, about 200 mg/mL to about 250 mg/mL, about 200 mg/mL to about 300 mg/mL, or about 250 mg/mL to about 300 mg/mL.
5. The pharmaceutical composition of any one of embodiments 1-4, wherein the one or more glycerolipids are in an amount of at least 40% by weight, at least 45% by weight, at least 50% by weight, at least 55% by weight, at least 60% by weight, at least 65% by weight, at least 70% by weight, at least 75% by weight, at least 80% by weight, at least 85% by weight, at least 90% by weight, or at least 95% by weight and/or at most 40% by weight, at most 45% by weight, at most 50% by weight, at most 55% by weight, at most 60% by weight, at most 65% by weight, at most 70% by weight, at most 75% by weight, at most 80% by weight, at most 85% by weight, at most 90% by weight, or at most 95% by weight or between about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 55% to about 60%, about 55% to about 65%, about 55% to about 70%, about 55% to about 75%, about 55% to about 80%, about 55% to about 85%, about 55% to about 90%, about 55% to about 95%, about 60% to about 65%, about 60% to about 70%, about 60% to about 75%, about 60% to about 80%, about 60% to about 85%, about 60% to about 90%, about 60% to about 95%, about 65% to about 70%, about 65% to about 75%, about 65% to about 80%, about 65% to about 85%, about 65% to about 90%, about 65% to about 95%, about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95% by weight.
6. The pharmaceutical composition of any one of embodiments 1-5, wherein the one or more glycerolipids comprises one or more hard fats and one or more liquid fats.
7. The pharmaceutical composition of embodiment 6, wherein the one or more hard fats include one or more triglycerides.
8. The pharmaceutical composition of embodiment 7, wherein the one or more triglycerides include a mixture of saturated C₁₀-C₁₈ triglycerides, a mixture of saturated C₁₀-C₂₀ triglycerides, a mixture of saturated C₁₀-C₂₂ triglycerides, a mixture of saturated C₁₀-C₂₄ triglycerides, a mixture of saturated C₁₂-C₁₈ triglycerides, a mixture of saturated C₁₂-C₂₀ triglycerides, a mixture of saturated C₁₂-C₂₂ triglycerides, a mixture of saturated C₁₂-C₂₄ triglycerides, a mixture of saturated C₁₄-C₁₈ triglycerides, a mixture of saturated C₁₄-C₂₀ triglycerides, a mixture of saturated C₁₄-C₂₂ triglycerides, a mixture of saturated C₁₄-C₂₄ triglycerides, a mixture of saturated C₁₆-C₁₈ triglycerides, a mixture of saturated C₁₆-C₂₀ triglycerides, a mixture of saturated C₁₆-C₂₂ triglycerides, a mixture of saturated C₁₆-C₂₄ triglycerides, a mixture of saturated C₁₈-C₂₀ triglycerides, a mixture of saturated C₁₈-C₂₂ triglycerides, a mixture of saturated C₁₈-C₂₄ triglycerides, a mixture of saturated C₂₀-C₂₂ triglycerides, or a mixture of saturated C₂₂-C₂₄ triglycerides.
9. The pharmaceutical composition of embodiment 7, wherein the one or more triglycerides include a mixture of unsaturated C₁₀-C₁₈ triglycerides, a mixture of unsaturated C₁₀-C₂₀ triglycerides, a mixture of unsaturated C₁₀-C₂₂ triglycerides, a mixture of unsaturated C₁₀-C₂₄ triglycerides, a mixture of unsaturated C₁₂-C₁₈ triglycerides, a mixture of unsaturated C₁₂-C₂₀ triglycerides, a mixture of unsaturated C₁₂-C₂₂ triglycerides, a mixture of unsaturated C₁₂-C₂₄ triglycerides, a mixture of unsaturated C₁₄-C₁₈ triglycerides, a mixture of unsaturated C₁₄-C₂₀ triglycerides, a mixture of unsaturated C₁₄-C₂₂ triglycerides, a mixture of unsaturated C₁₄-C₂₄ triglycerides, a mixture of unsaturated C₁₆-C₁₈ triglycerides, a mixture of unsaturated C₁₆-C₂₀ triglycerides, a mixture of unsaturated C₁₆-C₂₂ triglycerides, a mixture of unsaturated C₁₆-C₂₄ triglycerides, a mixture of unsaturated C₁₈-C₂₀ triglycerides, a mixture of unsaturated C₁₈-C₂₂ triglycerides, a mixture of unsaturated C₁₈-C₂₄ triglycerides, a mixture of unsaturated C₂₀-C₂₂ triglycerides, or a mixture of unsaturated C₂₂-C₂₄ triglycerides.
10. The pharmaceutical composition of embodiment 7, wherein the one or more triglycerides include a mixture of saturated and unsaturated C₁₀-C₁₈ triglycerides, a mixture of saturated and unsaturated C₁₀-C₂₀ triglycerides, a mixture of saturated and unsaturated C₁₀-C₂₂ triglycerides, a mixture of saturated and unsaturated C₁₀-C₂₄ triglycerides, a mixture of saturated and unsaturated C₁₂-C₁₈ triglycerides, a mixture of saturated and unsaturated C₁₂-C₂₀ triglycerides, a mixture of saturated and unsaturated C₁₂-C₂₂ triglycerides, a mixture of saturated and unsaturated C₁₂-C₂₄ triglycerides, a mixture of saturated and unsaturated C₁₄-C₁₈ triglycerides, a mixture of saturated and unsaturated C₁₄-C₂₀ triglycerides, a mixture of saturated and unsaturated C₁₄-C₂₂ triglycerides, a mixture of saturated and unsaturated C₁₄-C₂₄ triglycerides, a mixture of saturated and unsaturated C₁₆-C₁₈ triglycerides, a mixture of saturated and unsaturated C₁₆-C₂₀ triglycerides, a mixture of saturated and unsaturated C₁₆-C₂₂ triglycerides, a mixture of saturated and unsaturated C₁₆-C₂₄ triglycerides, a mixture of saturated and unsaturated C₁₈-C₂₀ triglycerides, a mixture of saturated and unsaturated C₁₈-C₂₂ triglycerides, a mixture of saturated and unsaturated C₁₈-C₂₄ triglycerides, a mixture of saturated and unsaturated C₂₀-C₂₂ triglycerides, or a mixture of saturated and unsaturated C₂₂-C₂₄ triglycerides.
11. The pharmaceutical composition of any one of embodiments 7-10, wherein the one or more hard fats are in an amount of at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, and/or at most 10% by weight, at most 15% by weight, at most 20% by weight, at most 25% by weight, at most 30% by weight, at most 35% by weight, at most 40% by weight, at most 45% by weight, at most 50% by weight, at most 55% by weight, at most 60% by weight, or between about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, about 55% to about 60% by weight.
12. The pharmaceutical composition of any one of embodiments 6-11, wherein the one or more liquid fats include one or more partially hydrolyzed glycerolipids, one or more monoglycerides, or a combination thereof.
13. The pharmaceutical composition of embodiment 12, wherein the one or more partially hydrolyzed glycerolipids comprise a mixture of mono-, di- and triglycerides.
14. The pharmaceutical composition of embodiment 12, wherein the one or more partially hydrolyzed glycerolipids comprise a mixture of unsaturated C₁₀-C₁₈ monoglycerides, C₁₀-C₁₈ diglycerides, and C₁₀-C₁₈ triglycerides, a mixture of unsaturated C₁₀-C₂₀ monoglycerides, C₁₀-C₂₀ diglycerides, and C₁₀-C₂₀ triglycerides, a mixture of unsaturated C₁₀-C₂₂ monoglycerides, C₁₀-C₂₂ diglycerides, and C₁₀-C₂₂ triglycerides, a mixture of unsaturated C₁₀-C₂₄ monoglycerides, C₁₀-C₂₄ diglycerides, and C₁₀-C₂₄ triglycerides, a mixture of unsaturated C₁₂-C₁₈ monoglycerides, C₁₂-C₁₈ diglycerides, and C₁₂-C₁₈ triglycerides, a mixture of unsaturated C₁₂-C₂₀ monoglycerides, C₁₂-C₂₀ diglycerides, and C₁₂-C₂₀ triglycerides, a mixture of unsaturated C₁₂-C₂₂ monoglycerides, C₁₂-C₂₂ diglycerides, and C₁₂-C₂₂ triglycerides, a mixture of unsaturated C₁₂-C₂₄ monoglycerides, C₁₂-C₂₄ diglycerides, and C₁₂-C₂₄ triglycerides, a mixture of unsaturated C₁₄-C₁₈ monoglycerides, C₁₄-C₁₈ diglycerides, and C₁₄-C₁₈ triglycerides, a mixture of unsaturated C₁₄-C₂₀ monoglycerides, C₁₄-C₂₀ diglycerides, and C₁₄-C₂₀ triglycerides, a mixture of unsaturated C₁₄-C₂₂ monoglycerides, C₁₄-C₂₂ diglycerides, and C₁₄-C₂₂ triglycerides, a mixture of unsaturated C₁₄-C₂₄ monoglycerides, C₁₄-C₂₄ diglycerides, and C₁₄-C₂₄ triglycerides, a mixture of unsaturated C₁₆-C₁₈ monoglycerides, C₁₆-C₁₈ diglycerides, and C₁₆-C₁₈ triglycerides, a mixture of unsaturated C₁₆-C₂₀ monoglycerides, C₁₆-C₂₀ diglycerides, and C₁₆-C₂₀ triglycerides, a mixture of unsaturated C₁₆-C₂₂ monoglycerides, C₁₆-C₂₂ diglycerides, and C₁₆-C₂₂ triglycerides, a mixture of unsaturated C₁₆-C₂₄ monoglycerides, C₁₆-C₂₄ diglycerides, and C₁₆-C₂₄ triglycerides, a mixture of unsaturated C₁₈-C₂₀ monoglycerides, C₁₈-C₂₀ diglycerides, and C₁₈-C₂₀ triglycerides, a mixture of unsaturated C₁₈-C₂₂ monoglycerides, C₁₈-C₂₂ diglycerides, and C₁₈-C₂₂ triglycerides, a mixture of unsaturated C₁₈-C₂₄ monoglycerides, C₁₈-C₂₄ diglycerides, and C₁₈-C₂₄ triglycerides, a mixture of unsaturated C₂₀-C₂₂ monoglycerides, C₂₀-C₂₂ diglycerides, C₂₀-C₂₂ triglycerides, or a mixture of unsaturated C₂₂-C₂₄ monoglycerides, C₂₂-C₂₄ diglycerides, and C₂₂-C₂₄ triglycerides.
15. The pharmaceutical composition of embodiment 12, wherein the one or more partially hydrolyzed glycerolipids comprise a mixture of saturated C₁₀-C₁₈ monoglycerides, C₁₀-C₁₈ diglycerides, and C₁₀-C₁₈ triglycerides, a mixture of saturated C₁₀-C₂₀ monoglycerides, C₁₀-C₂₀ diglycerides, and C₁₀-C₂₀ triglycerides, a mixture of saturated C₁₀-C₂₂ monoglycerides, C₁₀-C₂₂ diglycerides, and C₁₀-C₂₂ triglycerides, a mixture of saturated C₁₀-C₂₄ monoglycerides, C₁₀-C₂₄ diglycerides, and C₁₀-C₂₄ triglycerides, a mixture of saturated C₁₂-C₁₈ monoglycerides, C₁₂-C₁₈ diglycerides, and C₁₂-C₁₈ triglycerides, a mixture of saturated C₁₂-C₂₀ monoglycerides, C₁₂-C₂₀ diglycerides, and C₁₂-C₂₀ triglycerides, a mixture of saturated C₁₂-C₂₂ monoglycerides, C₁₂-C₂₂ diglycerides, and C₁₂-C₂₂ triglycerides, a mixture of saturated C₁₂-C₂₄ monoglycerides, C₁₂-C₂₄ diglycerides, and C₁₂-C₂₄ triglycerides, a mixture of saturated C₁₄-C₁₈ monoglycerides, C₁₄-C₁₈ diglycerides, and C₁₄-C₁₈ triglycerides, a mixture of saturated C₁₄-C₂₀ monoglycerides, C₁₄-C₂₀ diglycerides, and C₁₄-C₂₀ triglycerides, a mixture of saturated C₁₄-C₂₂ monoglycerides, C₁₄-C₂₂ diglycerides, and C₁₄-C₂₂ triglycerides, a mixture of saturated C₁₄-C₂₄ monoglycerides, C₁₄-C₂₄ diglycerides, and C₁₄-C₂₄ triglycerides, a mixture of saturated C₁₆-C₁₈ monoglycerides, C₁₆-C₁₈ diglycerides, and C₁₆-C₁₈ triglycerides, a mixture of saturated C₁₆-C₂₀ monoglycerides, C₁₆-C₂₀ diglycerides, and C₁₆-C₂₀ triglycerides, a mixture of saturated C₁₆-C₂₂ monoglycerides, C₁₆-C₂₂ diglycerides, and C₁₆-C₂₂ triglycerides, a mixture of saturated C₁₆-C₂₄ monoglycerides, C₁₆-C₂₄ diglycerides, and C₁₆-C₂₄ triglycerides, a mixture of saturated C₁₈-C₂₀ monoglycerides, C₁₈-C₂₀ diglycerides, and C₁₈-C₂₀ triglycerides, a mixture of saturated C₁₈-C₂₂ monoglycerides, C₁₈-C₂₂ diglycerides, and C₁₈-C₂₂ triglycerides, a mixture of saturated C₁₈-C₂₄ monoglycerides, C₁₈-C₂₄ diglycerides, and C₁₈-C₂₄ triglycerides, a mixture of saturated C₂₀-C₂₂ monoglycerides, C₂₀-C₂₂ diglycerides, C₂₀-C₂₂ triglycerides, or a mixture of saturated C₂₂-C₂₄ monoglycerides, C₂₂-C₂₄ diglycerides, and C₂₂-C₂₄ triglycerides.
16. The pharmaceutical composition of embodiment 12, wherein the one or more partially hydrolyzed glycerolipids comprise a mixture of saturated and unsaturated C₁₀-C₁₈ monoglycerides, C₁₀-C₁₈ diglycerides, and C₁₀-C₁₈ triglycerides, a mixture of saturated and unsaturated C₁₀-C₂₀ monoglycerides, C₁₀-C₂₀ diglycerides, and C₁₀-C₂₀ triglycerides, a mixture of saturated and unsaturated C₁₀-C₂₂ monoglycerides, C₁₀-C₂₂ diglycerides, and C₁₀-C₂₂ triglycerides, a mixture of saturated and unsaturated C₁₀-C₂₄ monoglycerides, C₁₀-C₂₄ diglycerides, and C₁₀-C₂₄ triglycerides, a mixture of saturated and unsaturated C₁₂-C₁₈ monoglycerides, C₁₂-C₁₈ diglycerides, and C₁₂-C₁₈ triglycerides, a mixture of saturated and unsaturated C₁₂-C₂₀ monoglycerides, C₁₂-C₂₀ diglycerides, and C₁₂-C₂₀ triglycerides, a mixture of saturated and unsaturated C₁₂-C₂₂ monoglycerides, C₁₂-C₂₂ diglycerides, and C₁₂-C₂₂ triglycerides, a mixture of saturated and unsaturated C₁₂-C₂₄ monoglycerides, C₁₂-C₂₄ diglycerides, and C₁₂-C₂₄ triglycerides, a mixture of saturated and unsaturated C₁₄-C₁₈ monoglycerides, C₁₄-C₁₈ diglycerides, and C₁₄-C₁₈ triglycerides, a mixture of saturated and unsaturated C₁₄-C₂₀ monoglycerides, C₁₄-C₂₀ diglycerides, and C₁₄-C₂₀ triglycerides, a mixture of saturated and unsaturated C₁₄-C₂₂ monoglycerides, C₁₄-C₂₂ diglycerides, and C₁₄-C₂₂ triglycerides, a mixture of saturated and unsaturated C₁₄-C₂₄ monoglycerides, C₁₄-C₂₄ diglycerides, and C₁₄-C₂₄ triglycerides, a mixture of saturated and unsaturated C₁₆-C₁₈ monoglycerides, C₁₆-C₁₈ diglycerides, and C₁₆-C₁₈ triglycerides, a mixture of saturated and unsaturated C₁₆-C₂₀ monoglycerides, C₁₆-C₂₀ diglycerides, and C₁₆-C₂₀ triglycerides, a mixture of saturated and unsaturated C₁₆-C₂₂ monoglycerides, C₁₆-C₂₂ diglycerides, and C₁₆-C₂₂ triglycerides, a mixture of saturated and unsaturated C₁₆-C₂₄ monoglycerides, C₁₆-C₂₄ diglycerides, and C₁₆-C₂₄ triglycerides, a mixture of saturated and unsaturated C₁₈-C₂₀ monoglycerides, C₁₈-C₂₀ diglycerides, and C₁₈-C₂₀ triglycerides, a mixture of saturated and unsaturated C₁₈-C₂₂ monoglycerides, C₁₈-C₂₂ diglycerides, and C₁₈-C₂₂ triglycerides, a mixture of saturated and unsaturated C₁₈-C₂₄ monoglycerides, C₁₈-C₂₄ diglycerides, and C₁₈-C₂₄ triglycerides, a mixture of saturated and unsaturated C₂₀-C₂₂ monoglycerides, C₂₀-C₂₂ diglycerides, C₂₀-C₂₂ triglycerides, or a mixture of saturated and unsaturated C₂₂-C₂₄ monoglycerides, C₂₂-C₂₄ diglycerides, and C₂₂-C₂₄ triglycerides.
17. The pharmaceutical composition of any one or embodiments 12-16, wherein the one or more partially hydrolyzed glycerolipids are in an amount of at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, and/or at most 10% by weight, at most 15% by weight, at most 20% by weight, at most 25% by weight, at most 30% by weight, at most 35% by weight, at most 40% by weight, at most 45% by weight, at most 50% by weight, at most 55% by weight, at most 60% by weight, or between about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, about 55% to about 60% by weight.
18. The pharmaceutical composition of any one of embodiments 12-17, wherein the one or more monoglycerides comprise unsaturated C₁₀-C₁₈ monoglycerides, unsaturated C₁₀-C₂₀ monoglycerides, unsaturated C₁₀-C₂₂ monoglycerides, unsaturated C₁₀-C₂₄ monoglycerides, unsaturated C₁₂-C₁₈ monoglycerides, unsaturated C₁₂-C₂₀ monoglycerides, unsaturated C₁₂-C₂₂ monoglycerides, unsaturated C₁₂-C₂₄ monoglycerides, unsaturated C₁₄-C₁₈ monoglycerides, unsaturated C₁₄-C₂₀ monoglycerides, unsaturated C₁₄-C₂₂ monoglycerides, unsaturated C₁₄-C₂₄ monoglycerides, unsaturated C₁₆-C₁₈ monoglycerides, unsaturated C₁₆-C₂₀ monoglycerides, unsaturated C₁₆-C₂₂ monoglycerides, unsaturated C₁₆-C₂₄ monoglycerides, unsaturated C₁₈-C₂₀ monoglycerides, unsaturated C₁₈-C₂₂ monoglycerides, unsaturated C₁₈-C₂₄ monoglycerides, unsaturated C₂₀-C₂₂ monoglycerides, or unsaturated C₂₂-C₂₄ monoglycerides.
19. The pharmaceutical composition of any one of embodiments 12-17, wherein the one or more monoglycerides comprise saturated C₁₀-C₁₈ monoglycerides, saturated C₁₀-C₂₀ monoglycerides, saturated C₁₀-C₂₂ monoglycerides, saturated C₁₀-C₂₄ monoglycerides, saturated C₁₂-C₁₈ monoglycerides, saturated C₁₂-C₂₀ monoglycerides, saturated C₁₂-C₂₂ monoglycerides, saturated C₁₂-C₂₄ monoglycerides, saturated C₁₄-C₁₈ monoglycerides, saturated C₁₄-C₂₀ monoglycerides, saturated C₁₄-C₂₂ monoglycerides, saturated C₁₄-C₂₄ monoglycerides, saturated C₁₆-C₁₈ monoglycerides, saturated C₁₆-C₂₀ monoglycerides, saturated C₁₆-C₂₂ monoglycerides, saturated C₁₆-C₂₄ monoglycerides, saturated C₁₈-C₂₀ monoglycerides, saturated C₁₈-C₂₂ monoglycerides, saturated C₁₈-C₂₄ monoglycerides, saturated C₂₀-C₂₂ monoglycerides, or saturated C₂₂-C₂₄ monoglycerides.
20. The pharmaceutical composition of any one of embodiments 12-17, wherein the one or more monoglycerides comprise a mixture of saturated and unsaturated C₁₀-C₁₈ monoglycerides, a mixture of saturated and unsaturated C₁₀-C₂₀ monoglycerides, a mixture of saturated and unsaturated C₁₀-C₂₂ monoglycerides, a mixture of saturated and unsaturated C₁₀-C₂₄ monoglycerides, a mixture of saturated and unsaturated C₁₂-C₁₈ monoglycerides, a mixture of saturated and unsaturated C₁₂-C₂₀ monoglycerides, a mixture of saturated and unsaturated C₁₂-C₂₂ monoglycerides, a mixture of saturated and unsaturated C₁₂-C₂₄ monoglycerides, a mixture of saturated and unsaturated C₁₄-C₁₈ monoglycerides, a mixture of saturated and unsaturated C₁₄-C₂₀ monoglycerides, a mixture of saturated and unsaturated C₁₄-C₂₂ monoglycerides, a mixture of saturated and unsaturated C₁₄-C₂₄ monoglycerides, a mixture of saturated and unsaturated C₁₆-C₁₈ monoglycerides, a mixture of saturated and unsaturated C₁₆-C₂₀ monoglycerides, a mixture of saturated and unsaturated C₁₆-C₂₂ monoglycerides, a mixture of saturated and unsaturated C₁₆-C₂₄ monoglycerides, a mixture of saturated and unsaturated C₁₈-C₂₀ monoglycerides, a mixture of saturated and unsaturated C₁₈-C₂₂ monoglycerides, a mixture of saturated and unsaturated C₁₈-C₂₄ monoglycerides, a mixture of saturated and unsaturated C₂₀-C₂₂ monoglycerides, or a mixture of saturated and unsaturated C₂₂-C₂₄ monoglycerides.
21. The pharmaceutical composition of any one or embodiments 18-20, wherein the one or more monoglycerides are in an amount of at least 10% by weight, at least 15% by weight, at least 20% by weight, at least 25% by weight, at least 30% by weight, at least 35% by weight, at least 40% by weight, at least 45% by weight, at least 50% by weight, at least 55% by weight, at least 60% by weight, and/or at most 10% by weight, at most 15% by weight, at most 20% by weight, at most 25% by weight, at most 30% by weight, at most 35% by weight, at most 40% by weight, at most 45% by weight, at most 50% by weight, at most 55% by weight, at most 60% by weight, or between about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 20% to about 25%, about 20% to about 30%, about 20% to about 35%, about 20% to about 40%, about 20% to about 45%, about 20% to about 50%, about 20% to about 55%, about 20% to about 60%, about 25% to about 30%, about 25% to about 35%, about 25% to about 40%, about 25% to about 45%, about 25% to about 50%, about 25% to about 55%, about 25% to about 60%, about 30% to about 35%, about 30% to about 40%, about 30% to about 45%, about 30% to about 50%, about 30% to about 55%, about 30% to about 60%, about 35% to about 40%, about 35% to about 45%, about 35% to about 50%, about 35% to about 55%, about 35% to about 60%, about 40% to about 45%, about 40% to about 50%, about 40% to about 55%, about 40% to about 60%, about 45% to about 50%, about 45% to about 55%, about 45% to about 60%, about 50% to about 55%, about 50% to about 60%, about 55% to about 60% by weight.
22. The pharmaceutical composition of any one or embodiments 6-21, wherein the one or more hard fats and the one or more liquid fats are in a hard fat to liquid fat ratio of about 5:1 to about 4:1, about 5:1 to about 3:1, about 5:1 to about 2:1, about 5:1 to about 1:1, about 4:1 to about 3:1, about 4:1 to about 2:1, about 4:1 to about 1:1, about 3:1 to about 2:1, about 3:1 to about 1:1, or about 2:1 to about 1:1.
23. The pharmaceutical composition of any one or embodiments 6-21, wherein the one or more hard fats and the one or more liquid fats are in a hard fat to liquid fat ratio of about 1:5 to about 1:4, about 1:5 to about 1:3, about 1:5 to about 1:2, about 1:5 to about 1:1, about 1:4 to about 1:3, about 1:4 to about 1:2, about 1:4 to about 1:1, about 1:3 to about 1:2, about 1:3 to about 1:1, or about 1:2 to about 1:1.
24. The pharmaceutical composition of any one of embodiments 1-23, wherein the one or more digestion enhancers are in an amount of at least 1% by weight, at least 2.5% by weight, at least 5% by weight, at least 7.5% by weight, at least 10% by weight, at least 12.5% by weight, at least 15% by weight, at least 17.5% by weight, at least 20% by weight, at least 22.5% by weight, at least 25% by weight, at least 30% by weight, at least 35% by weight, at least 40% by weight, at least 45% by weight, at least 50% by weight, at least 55% by weight, at least 60% by weight, at least 65% by weight, at least 70% by weight, at least 75% by weight, at least 75% by weight and/or at most 1% by weight, at most 2.5% by weight, at most 5% by weight, at most 7.5% by weight, at most 10% by weight, at most 12.5% by weight, at most 15% by weight, at most 17.5% by weight, at most 20% by weight, at most 22.5% by weight, at most 25% by weight, at most 30% by weight, at most 35% by weight, at most 40% by weight, at most 45% by weight, at most 50% by weight, at most 55% by weight, at most 60% by weight, at most 65% by weight, at most 70% by weight, at most 75% by weight, or at most 80% by weight, or between about 1% to about 2.5% by weight, about 1% to about 5% by weight, about 1% to about 10% by weight, about 1% to about 15% by weight, about 1% to about 20% by weight, about 1% to about 25% by weight, about 2.5% to about 5% by weight, about 2.5% to about 10% by weight, about 2.5% to about 15% by weight, about 2.5% to about 20% by weight, about 2.5% to about 25% by weight, about 5% to about 10% by weight, about 5% to about 15% by weight, about 5% to about 20% by weight, about 5% to about 25% by weight, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 30% to about 35%, about 30% to about 40%, about 30% to about 45%, about 30% to about 50%, about 30% to about 55%, about 30% to about 60%, about 30% to about 65%, about 30% to about 70%, about 35% to about 40%, about 35% to about 45%, about 35% to about 50%, about 35% to about 55%, about 35% to about 60%, about 35% to about 65%, about 35% to about 70%, about 40% to about 45%, about 40% to about 50%, about 40% to about 55%, about 40% to about 60%, about 40% to about 65%, about 40% to about 70%, about 40% to about 75%, about 40% to about 80%, about 45% to about 50%, about 45% to about 55%, about 45% to about 60%, about 45% to about 65%, about 45% to about 70%, about 45% to about 75%, about 45% to about 80%, about 50% to about 55%, about 50% to about 60%, about 50% to about 65%, about 50% to about 70%, about 50% to about 75%, about 50% to about 80%, about 55% to about 60%, about 55% to about 65%, about 55% to about 70%, about 55% to about 75%, about 55% to about 80%, about 60% to about 65%, about 60% to about 70%, about 60% to about 75%, about 60% to about 80%, about 65% to about 70% by weight, about 65% to about 75%, about 65% to about 80%, about 70% to about 75%, about 70% to about 80%, or about 75% to about 80% by weight.
25. The pharmaceutical composition of embodiment 24, wherein the one or more digestion enhancers include one or more bile acids, one or more phospholipids, one or more free C₁₄₋₂₄ fatty acids, one or more free C₁₄₋₂₄ fatty acid surfactants, or any combination thereof.
26. The pharmaceutical composition of embodiment 25, wherein the one or more bile acids includes cholic acid, chenodeoxycholic acid, dafachronic acid, deoxycholic acid, glycocholic acid, glycohenodeoxycholic acid, lithocholic acid, taurochenodeoxycholic acid, taurocholic acid, any stereoisomer thereof, and any combination thereof.
27. The pharmaceutical composition of embodiment 25 or 26, wherein the one or more bile acids are in an amount of at least 0.1% by weight, at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, or at least 10.0% by weight and/or at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, or at most 10.0% by weight or between about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.
28. The pharmaceutical composition of any one of embodiments 25-27, wherein the one or more phospholipids include one or more phosphoglycerides, one or more phosphosphingolipids, one or more lecithins, or any combination thereof.
29. The pharmaceutical composition of embodiment 28, wherein the one or more phosphoglycerides include a phosphatidic acid (phosphatidate) (PA), a phosphatidylethanolamine (PE), a phosphatidylcholine (PC), a phosphatidylserine (PS), a cardiolipin, a phosphoinositide, or any combination thereof.
30. The pharmaceutical composition of embodiment 28, wherein the one or more phosphosphingolipids include phosphatidylethanolamine (PE), phosphatidylcholine (PC), ceramide phosphorylethanolamine (Cer-PE), ceramide phosphorylcholine (Cer-PC), or any combination thereof.
31. The pharmaceutical composition of embodiment 25 or 28-30, wherein the one or more phospholipids are in an amount of at least 0.1% by weight, at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, or at least 10.0% by weight and/or at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, or at most 10.0% by weight or between about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.
32. The pharmaceutical composition of any one of embodiments 25-31, wherein the one or more free C₁₄₋₂₄ fatty acids include unsaturated free C₁₄-C₁₆ fatty acids, unsaturated free C₁₄-C₁₈ fatty acids, unsaturated free C₁₄-C₂₀ fatty acids, unsaturated free C₁₄-C₂₂ fatty acids, unsaturated free C₁₄-C₂₄ fatty acids, unsaturated free C₁₆-C₁₈ fatty acids, unsaturated free C₁₆-C₂₀ fatty acids, unsaturated free C₁₆-C₂₂ fatty acids, unsaturated free C₁₆-C₂₄ fatty acids, unsaturated free C₁₈-C₂₀ fatty acids, unsaturated free C₁₈-C₂₂ fatty acids, unsaturated free C₁₈-C₂₄ fatty acids, unsaturated free C₂₀-C₂₂ fatty acids, or unsaturated free C₂₂-C₂₄ fatty acids.
33. The pharmaceutical composition of any one of embodiments 25-31, wherein the one or more free C₁₄₋₂₄ fatty acids include ω-3 unsaturated free C₁₈-C₂₂ fatty acids, ω-5 unsaturated free C₁₈-C₂₂ fatty acids, ω-6 unsaturated free C₁₈-C₂₂ fatty acids, ω-7 unsaturated free C₁₈-C₂₂ fatty acids, ω-9 unsaturated free C₁₈-C₂₂ fatty acids, ω-10 unsaturated free C₁₈-C₂₂ fatty acids, ω-11 unsaturated free C₁₈-C₂₂ fatty acids, or ω-12 unsaturated free C₁₈-C₂₂ fatty acids.
34. The pharmaceutical composition of any one of embodiments 25-31, wherein the one or more free C₁₄₋₂₄ fatty acids include saturated free C₁₄-C₁₆ fatty acids, saturated free C₁₄-C₁₈ fatty acids, saturated free C₁₄-C₂₀ fatty acids, saturated free C₁₄-C₂₂ fatty acids, saturated free C₁₄-C₂₄ fatty acids, saturated free C₁₆-C₁₈ fatty acids, saturated free C₁₆-C₂₀ fatty acids, saturated free C₁₆-C₂₂ fatty acids, saturated free C₁₆-C₂₄ fatty acids, saturated free C₁₈-C₂₀ fatty acids, saturated free C₁₈-C₂₂ fatty acids, saturated free C₁₈-C₂₄ fatty acids, saturated free C₂₀-C₂₂ fatty acids, or saturated free C₂₂-C₂₄ fatty acids.
35. The pharmaceutical composition of any one of embodiments 25-31, wherein the one or more free C₁₄₋₂₄ fatty acids include a mixture of saturated and unsaturated free C₁₄-C₁₆ fatty acids, a mixture of saturated and unsaturated free C₁₄-C₁₈ fatty acids, a mixture of saturated and unsaturated free C₁₄-C₂₀ fatty acids, a mixture of saturated and unsaturated free C₁₄-C₂₂ fatty acids, a mixture of saturated and unsaturated free C₁₄-C₂₄ fatty acids, a mixture of saturated and unsaturated free C₁₆-C₁₈ fatty acids, a mixture of saturated and unsaturated free C₁₆-C₂₀ fatty acids, a mixture of saturated and unsaturated free C₁₆-C₂₂ fatty acids, a mixture of saturated and unsaturated free C₁₆-C₂₄ fatty acids, a mixture of saturated and unsaturated free C₁₈-C₂₀ fatty acids, a mixture of saturated and unsaturated free C₁₈-C₂₂ fatty acids, a mixture of saturated and unsaturated free C₁₈-C₂₄ fatty acids, a mixture of saturated and unsaturated free C₂₀-C₂₂ fatty acids, or a mixture of saturated and unsaturated free C₂₂-C₂₄ fatty acids.
36. The pharmaceutical composition of any one of embodiments 25-31, wherein the one or more free C₁₄₋₂₄ fatty acids include palmitic acid (hexadecenoic acid), palmitolinolenic acid, palmitidonic acid, palmitovaccenic acid, palmitoleic acid, sapienic acid, 4-Hexadecenoic acid, stearic acid (octadecenoic acid), α-linolenic acid, stearidonic acid, α-eleostearic acid, β-eleostearic acid, pumicic acid, 7,10,13-octadecatrienoic acid, 12-octadecenoic acid, linoleic acid, linolelaidic acid. γ-linolenic acid, calendic acid, pinolenic acid, vaccinic acid, ruminic acid, oleic acid, elaidic acid, petroselinic acid, arachidic acid (eicosanoic acid), dihomo-α-linolenic acid, eicosic acidtraenoic acid, eicosapentaenoic acid, 9,12,15-eicosatrienoic acid, β-eicosic acidtraenoic acid, dihomo-linoleic acid, dihomo-γ-linolenic acid, arachidonic acid, paullinic acid, 7,10,13-eicosatrienoic acid, gondoic acid, 8,11-eicosadienoic acid, meadic acid, gadoleic acid, 8-eicosenoic acid, behenic acid (docosanoic acid), clupanodonic acid, docosahexaenoic acid, adrenic acid, osbondic acid, erucic acid, lignoceric acid (tetracosanic acid), 9,12,15,18,21-Tetracosapentaenoic acid, 6,9,12,15,18,21-Tetracosahexaenoic acid, and nervonic acid.
37. The pharmaceutical composition of any one of embodiments 25-36, wherein the one or more free C₁₄₋₂₄ fatty acids are in an amount of at least 1% by weight, at least 2.5% by weight, at least 5% by weight, at least 7.5% by weight, at least 10% by weight, at least 12.5% by weight, at least 15% by weight, at least 17.5% by weight, at least 20% by weight, at least 22.5% by weight, at least 25% by weight, at least 30% by weight, at least 35% by weight, at least 40% by weight, at least 50% by weight, at least 60% by weight, at least 70% by weight, at least 75% by weight and/or at most 1% by weight, at most 2.5% by weight, at most 5% by weight, at most 7.5% by weight, at most 10% by weight, at most 12.5% by weight, at most 15% by weight, at most 17.5% by weight, at most 20% by weight, at most 22.5% by weight, at most 25% by weight, at most 30% by weight, at most 35% by weight, at most 40% by weight, at most 50% by weight, at most 60% by weight, at most 70% by weight, at most 75% by weight or between about 1% to about 2.5% by weight, about 1% to about 5% by weight, about 1% to about 10% by weight, about 1% to about 15% by weight, about 1% to about 20% by weight, about 1% to about 25% by weight, about 2.5% to about 5% by weight, about 2.5% to about 10% by weight, about 2.5% to about 15% by weight, about 2.5% to about 20% by weight, about 2.5% to about 25% by weight, about 2.5% to about 30% by weight, about 5% to about 10% by weight, about 5% to about 15% by weight, about 5% to about 20% by weight, about 5% to about 25% by weight, about 5% to about 30% by weight, about 10% to about 15% by weight, about 10% to about 20% by weight, about 10% to about 25% by weight, about 10% to about 30% by weight, about 10% to about 40% by weight, about 10% to about 45% by weight, about 10% to about 50% by weight, about 10% to about 60% by weight, about 10% to about 70% by weight, about 15% to about 20% by weight, about 15% to about 25% by weight, about 15% to about 30% by weight, about 15% to about 40% by weight, about 15% to about 45% by weight, about 15% to about 50% by weight, about 15% to about 60% by weight, about 15% to about 70% by weight, about 20% to about 25% by weight, about 20% to about 30% by weight, about 20% to about 40% by weight, about 20% to about 45% by weight, about 20% to about 50% by weight, about 20% to about 60% by weight, about 20% to about 70% by weight, about 30% to about 40% by weight, about 30% to about 50% by weight, about 30% to about 60% by weight, about 30% to about 70% by weight, about 30% to about 75% by weight, about 35% to about 40% by weight, about 35% to about 50% by weight, about 35% to about 60% by weight, about 35% to about 70% by weight, about 35% to about 75% by weight, about 40% to about 50% by weight, about 40% to about 60% by weight, about 40% to about 70% by weight, about 40% to about 75% by weight, about 50% to about 60% by weight, about 50% to about 70% by weight, or about 60% to about 70% by weight.
38. The pharmaceutical composition of any one of embodiments 25-37, wherein the one or more free C₁₄₋₂₄ fatty acid surfactants include unsaturated free C₁₄-C₁₆ fatty acid surfactants, unsaturated free C₁₄-C₁₈ fatty acid surfactants, unsaturated free C₁₄-C₂₀ fatty acid surfactants, unsaturated free C₁₄-C₂₂ fatty acid surfactants, unsaturated free C₁₄-C₂₄ fatty acid surfactants, unsaturated free C₁₆-C₁₈ fatty acid surfactants, unsaturated free C₁₆-C₂₀ fatty acid surfactants, unsaturated free C₁₆-C₂₂ fatty acid surfactants, unsaturated free C₁₆-C₂₄ fatty acid surfactants, unsaturated free C₁₈-C₂₀ fatty acid surfactants, unsaturated free C₁₈-C₂₂ fatty acid surfactants, unsaturated free C₁₈-C₂₄ fatty acid surfactants, unsaturated free C₂₀-C₂₂ fatty acid surfactants, or unsaturated free C₂₂-C₂₄ fatty acid surfactants.
39. The pharmaceutical composition of any one of embodiments 25-37, wherein the one or more free C₁₄₋₂₄ fatty acid surfactants include ω-3 unsaturated free C₁₈-C₂₂ fatty acid surfactants, ω-5 unsaturated free C₁₈-C₂₂ fatty acid surfactants, ω-6 unsaturated free C₁₈-C₂₂ fatty acid surfactants, ω-7 unsaturated free C₁₈-C₂₂ fatty acid surfactants, ω-9 unsaturated free C₁₈-C₂₂ fatty acid surfactants, ω-10 unsaturated free C₁₈-C₂₂ fatty acid surfactants, ω-11 unsaturated free C₁₈-C₂₂ fatty acid surfactants, or ω-12 unsaturated free C₁₈-C₂₂ fatty acid surfactants.
40. The pharmaceutical composition of any one of embodiments 25-37, wherein the one or more free C₁₄₋₂₄ fatty acid surfactants include saturated free C₁₄-C₁₆ fatty acid surfactants, saturated free C₁₄-C₁₈ fatty acid surfactants, saturated free C₁₄-C₂₀ fatty acid surfactants, saturated free C₁₄-C₂₂ fatty acid surfactants, saturated free C₁₄-C₂₄ fatty acid surfactants, saturated free C₁₆-C₁₈ fatty acid surfactants, saturated free C₁₆-C₂₀ fatty acid surfactants, saturated free C₁₆-C₂₂ fatty acid surfactants, saturated free C₁₆-C₂₄ fatty acid surfactants, saturated free C₁₈-C₂₀ fatty acid surfactants, saturated free C₁₈-C₂₂ fatty acid surfactants, saturated free C₁₈-C₂₄ fatty acid surfactants, saturated free C₂₀-C₂₂ fatty acid surfactants, or saturated free C₂₂-C₂₄ fatty acid surfactants.
41. The pharmaceutical composition of any one of embodiments 25-37, wherein the one or more free C₁₄₋₂₄ fatty acid surfactants include a mixture of saturated and unsaturated free C₁₄-C₁₆ fatty acid surfactants, a mixture of saturated and unsaturated free C₁₄-C₁₈ fatty acid surfactants, a mixture of saturated and unsaturated free C₁₄-C₂₀ fatty acid surfactants, a mixture of saturated and unsaturated free C₁₄-C₂₂ fatty acid surfactants, a mixture of saturated and unsaturated free C₁₄-C₂₄ fatty acid surfactants, a mixture of saturated and unsaturated free C₁₆-C₁₈ fatty acid surfactants, a mixture of saturated and unsaturated free C₁₆-C₂₀ fatty acid surfactants, a mixture of saturated and unsaturated free C₁₆-C₂₂ fatty acid surfactants, a mixture of saturated and unsaturated free C₁₆-C₂₄ fatty acid surfactants, a mixture of saturated and unsaturated free C₁₈-C₂₀ fatty acid surfactants, a mixture of saturated and unsaturated free C₁₈-C₂₂ fatty acid surfactants, a mixture of saturated and unsaturated free C₁₈-C₂₄ fatty acid surfactants, a mixture of saturated and unsaturated free C₂₀-C₂₂ fatty acid surfactants, or a mixture of saturated and unsaturated free C₂₂-C₂₄ fatty acid surfactants.
42. The pharmaceutical composition of any one of embodiments 25-37, wherein the one or more free C₁₄₋₂₄ fatty acid surfactants include sodium palmitate (hexadecanoate), sodium palmitolinolenate, sodium palmitidonate, sodium palmitovaccenate, sodium palmitoleate, sodium sapienate, sodium 4-Hexadecenoate, sodium stearate (octadecenoate), sodium α-linolenate, sodium stearidonate, sodium α-eleostearate, sodium β-eleostearate, sodium pumicate, sodium 7,10,13-octadecatrienoate, sodium 12-octadecenoate, sodium linoleate, sodium linolelaidate. Sodium γ-linolenate, sodium calendate, sodium pinolenate, sodium vaccinate, sodium ruminate, sodium oleate, sodium elaidate, sodium petroselinate, sodium arachidate (eicosanoate), sodium dihomo-α-linolenate, sodium eicosatetraenoate, sodium eicosapentaenoate, sodium 9,12,15-eicosatrienoate, sodium p-eicosatetraenoate, sodium dihomo-linoleate, sodium dihomo-γ-linolenate, sodium arachidonate, sodium paullinate, sodium 7,10,13-eicosatrienoate, sodium gondoate, Sodium 8,11-eicosadienoate, sodium meadate, sodium gadoleate, sodium 8-eicosenoate, sodium behenate (docosanoate), sodium clupanodonate, sodium docosahexaenoate, sodium adrenate, sodium osbondate, sodium erucate, sodium lignocerate (tetracosanate), sodium 9,12,15,18,21-Tetracosapentaenoate, sodium 6,9,12,15,18,21-Tetracosahexaenoate, and sodium nervonate.
43. The pharmaceutical composition of any one of embodiments 25-42, wherein the one or more free C₁₄₋₂₄ fatty acid surfactants are in an amount of at least 0.5% by weight, at least 1.0% by weight, at least 1.5% by weight, at least 2.0% by weight, at least 2.5% by weight, at least 3.0% by weight, at least 3.5% by weight, at least 4.0% by weight, at least 4.5% by weight, at least 5.0% by weight, at least 5.5% by weight, at least 6.0% by weight, at least 6.5% by weight, at least 7.0% by weight, at least 7.5% by weight, at least 8.0% by weight, at least 8.5% by weight, at least 9.0% by weight, at least 9.5% by weight, at least 10.0% by weight and/or at most 0.1% by weight, at most 0.5% by weight, at most 1.0% by weight, at most 1.5% by weight, at most 2.0% by weight, at most 2.5% by weight, at most 3.0% by weight, at most 3.5% by weight, at most 4.0% by weight, at most 4.5% by weight, at most 5.0% by weight, at most 5.5% by weight, at most 6.0% by weight, at most 6.5% by weight, at most 7.0% by weight, at most 7.5% by weight, at most 8.0% by weight, at most 8.5% by weight, at most 9.0% by weight, at most 9.5% by weight, at most 10.0% by weight or between about 0.1% to about 0.5%, about 0.1% to about 1.0%, about 0.1% to about 2.0%, about 0.1% to about 3.0%, about 0.1% to about 4.0%, about 0.1% to about 5.0%, about 0.1% to about 6.0%, about 0.1% to about 7.0%, about 0.1% to about 8.0%, about 0.1% to about 9.0%, about 0.1% to about 10.0%, about 0.5% to about 1.0%, about 0.5% to about 2.0%, about 0.5% to about 3.0%, about 0.5% to about 4.0%, about 0.5% to about 5.0%, about 0.5% to about 6.0%, about 0.5% to about 7.0%, about 0.5% to about 8.0%, about 0.5% to about 9.0%, about 0.5% to about 10.0%, about 1.0% to about 2.0%, about 1.0% to about 3.0%, about 1.0% to about 4.0%, about 1.0% to about 5.0%, about 1.0% to about 6.0%, about 1.0% to about 7.0%, about 1.0% to about 8.0%, about 1.0% to about 9.0%, about 1.0% to about 10.0%, about 2.0% to about 3.0%, about 2.0% to about 4.0%, about 2.0% to about 5.0%, about 2.0% to about 6.0%, about 2.0% to about 7.0%, about 2.0% to about 8.0%, about 2.0% to about 9.0%, about 2.0% to about 10.0%, about 3.0% to about 4.0%, about 3.0% to about 5.0%, about 3.0% to about 6.0%, about 3.0% to about 7.0%, about 3.0% to about 8.0%, about 3.0% to about 9.0%, about 3.0% to about 10.0%, about 4.0% to about 5.0%, about 4.0% to about 6.0%, about 4.0% to about 7.0%, about 4.0% to about 8.0%, about 4.0% to about 9.0%, about 4.0% to about 10.0%, about 5.0% to about 6.0%, about 5.0% to about 7.0%, about 5.0% to about 8.0%, about 5.0% to about 9.0%, about 5.0% to about 10.0%, about 6.0% to about 7.0%, about 6.0% to about 8.0%, about 6.0% to about 9.0%, about 6.0% to about 10.0%, about 7.0% to about 8.0%, about 7.0% to about 9.0%, about 7.0% to about 10.0%, about 8.0% to about 9.0%, about 8.0% to about 10.0%, or about 9.0% to about 10.0% by weight.
44. The pharmaceutical composition of any one of embodiments 1-43, wherein the pharmaceutical composition is not an emulsion or self-emulsifying formulation.
45. A pharmaceutical composition for use in treating an inflammation, the pharmaceutic composition being defined by any one of embodiments 1-44.
46. A pharmaceutical composition for use in treating a neuroinflammation, the pharmaceutic composition being defined by any one of embodiments 1-44.
47. A method of treating an individual with an inflammation, the method comprising the step of administering to the individual in need thereof a pharmaceutical composition as defined by any one of embodiments 1-44, wherein administration results in a reduction in a symptom associated with the inflammation, thereby treating the individual.
48. A method of treating an individual with a neuroinflammation, the method comprising the step of administering to the individual in need thereof a pharmaceutical composition as defined by any one of embodiments 1-44, wherein administration results in a reduction in a symptom associated with the inflammation, thereby treating the individual.
49. Use of a pharmaceutical composition as defined by any one of embodiments 1-44 in the manufacture of a medicament for the treatment of an inflammation.
50. Use of a pharmaceutical composition as defined by any one of embodiments 1-44 in the manufacture of a medicament for the treatment of a neuroinflammation.

Aspects of the present specification can also be described by the following embodiments:
1. A pharmaceutical composition comprising a fenofibrate, about 30% to about 40% by weight of one or more hard fats, about 30% to about 40% by weight of one or more liquid fats, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants.
2. The pharmaceutical composition according to embodiment 1, comprising about 10% to about 20% by weight of a fenofibrate; about 26% to about 36% by weight of one or more hard fats comprising a mixture of saturated C₁₀-C₁₈ triglycerides and/or saturated C₁₂-C₁₈ triglycerides; about 26% to about 36% by weight of one or more liquid fats comprising partially hydrolyzed triglycerides including a mixture of C₁₆-C₁₈ mono-, C₁₆₋₁₈ di- and C₁₆-C₁₈ triglycerides; about 3% to about 7% by weight of one or more bile acids; about 12% to about 22% by weight of one or more free C₁₄₋₂₄ fatty acids; and about 0.5% to about 4% by weight of one or more free C₁₄₋₂₄ fatty acid surfactants.
3. The pharmaceutical composition according to embodiments 1 or 2, comprising about 12% to about 18% by weight of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
4. The pharmaceutical composition according to any one of embodiments 1-4 comprising about 14% to about 16% by weight of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
5. The pharmaceutical composition according to embodiment 1 comprising about 25 mg/mL to about 300 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
6. The pharmaceutical composition according to embodiments 1 or 5, comprising about 35 mg/mL to about 200 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
7. The pharmaceutical composition according to embodiments 1, 5 or 6 comprising about 70 mg/mL to about 150 mg/mL of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
8. A pharmaceutical composition comprises a fenofibrate, about 15% to about 20% by weight of one or more hard fats, about 50% to about 60% by weight of one or more liquid fats, about 3% to about 6% by weight of one or more bile acids, about 10% to about 20% by weight of one or more free C₁₄₋₂₄ fatty acids, and about 0.5% to about 5% by weight of one or more free C₁₄₋₂₄ fatty acid surfactants.
9. A pharmaceutical composition comprises a fenofibrate, about 15% to about 20% by weight of one or more hard fats comprising C₁₀-C₂₄ triglycerides, about 10% to about 20% by weight of one or more liquid fats comprising partially hydrolyzed triglycerides including a mixture of C₁₀-C₂₄ mono-, C₁₀-C₂₄ di- and C₁₀-C₂₄ triglycerides, about 3% to about 6% by weight of one or more bile acids, about 50% to about 60% by weight of one or more free C₁₄₋₂₄ fatty acids, and about 0.5% to about 5% by weight of one or more free C₁₄₋₂₄ fatty acid surfactants.
10. The pharmaceutical composition according to embodiments 8 or 9, wherein the one or more hard fats comprise glycerolipids, preferably one or more hard fats comprising C₁₀-C₂₄ triglycerides, and more preferably one or more hard fats comprising a mixture of saturated C₁₀-C₁₈ triglycerides and/or saturated C₁₂-C₁₈ triglycerides.
11. The pharmaceutical composition according to any one of embodiments 8-10, wherein the one or more liquid fats comprise partially hydrolyzed glycerolipids including a mixture of mono-, di- and triglycerides, preferably one or more liquid fats comprising partially hydrolyzed triglycerides including a mixture of C₁₀-C₂₄ mono-, C₁₀-C₂₄ di- and C₁₀-C₂₄ triglycerides, and more preferably one or more liquid fats comprising partially hydrolyzed triglycerides including a mixture of C₁₆-C₁₈ mono-, C₁₆-C₁₈ di- and C₁₆-C₁₈ triglycerides.
12. The pharmaceutical composition according to any one of embodiments 8-11, wherein the fenofibrate is present in an amount between about 10% to about 20% by weight of the composition or the fenofibrate is present in a concentration of between about 25 mg/mL to about 300 mg/mL, preferably in a concentration of between about 35 mg/mL to about 200 mg/mL, and more preferably in a concentration of between about 70 mg/mL to about 150 mg/mL.
13. The pharmaceutical composition according to any one of embodiments 1-12, wherein the one or more bile acids comprise a cholic acid.
14. The pharmaceutical composition according to any one of embodiments 1-13, wherein the one or more free C₁₄₋₂₄ fatty acids comprise one or more free C₁₄₋₁₈ fatty acids.
15. The pharmaceutical composition according to embodiment 14, wherein the one or more free C₁₄₋₁₈ fatty acids comprise an oleic acid, a steric acid, a linoleic acid, or any combination thereof.
16. The pharmaceutical composition according to any one of embodiments 1-15, wherein the one or more free C₁₄₋₂₄ fatty acid surfactants comprise one or more free C₁₄₋₁₈ fatty acid surfactants.
17. The pharmaceutical composition according to embodiment 16, wherein the one or more free C₁₄₋₁₈ fatty acid surfactants comprise an oleate alkali metal or alkali earth metal salt, a stearate alkali metal or alkali earth metal salt, a linoleate alkali metal or alkali earth metal salt, or any combination thereof.
18. The pharmaceutical composition according to embodiments 16 or 17, wherein the one or more free C₁₄₋₁₈ fatty acid surfactants comprise a sodium oleate, a sodium stearate, a sodium linoleate, or any combination thereof.
19. A pharmaceutical composition for use in treating an inflammation, the pharmaceutic composition being defined by any one of embodiments 1-18.
20. The pharmaceutical composition for use according to embodiment 19, wherein the inflammation is neuroinflammation.
21. A method of treating an individual with an inflammation, the method comprising the step of administering to the individual in need thereof a pharmaceutical composition as defined by any one of embodiments 1-18, wherein administration results in a reduction in a symptom associated with the inflammation, thereby treating the individual.
22. The method according to embodiment 21, wherein the inflammation is neuroinflammation.
23. Use of a pharmaceutical composition as defined by any one of embodiments 1-18 in the manufacture of a medicament for the treatment of an inflammation.
24. The use according to embodiment 23, wherein the inflammation is neuroinflammation.
25. A pharmaceutical composition for use in treating neuroinflammation, the pharmaceutical composition comprising a fenofibrate, about 30% to about 40% by weight of one or more hard fats, about 30% to about 40% by weight of one or more liquid fats, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants.
26. The pharmaceutical composition for use according to embodiment 25, comprising about 10% to about 20% by weight of a fenofibrate; about 26% to about 36% by weight of one or more hard fats comprising a mixture of saturated C₁₀-C₁₈ triglycerides and/or saturated C₁₂-C₁₈ triglycerides; about 26% to about 36% by weight of one or more liquid fats comprising partially hydrolyzed triglycerides including a mixture of C₁₆-C₁₈ mono-, C₁₆₋₁₈ di- and C₁₆-C₁₈ triglycerides; about 3% to about 7% by weight of one or more bile acids; about 12% to about 22% by weight of one or more free C₁₄₋₂₄ fatty acids; and about 0.5% to about 4% by weight of one or more free C₁₄₋₂₄ fatty acid surfactants.
27. The pharmaceutical composition for use according to embodiments 25 or 26, comprising about 12% to about 18% by weight of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
28. The pharmaceutical composition for use according to any one of embodiments 25-27 comprising about 14% to about 16% by weight of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
29. The pharmaceutical composition for use according to embodiment 25 comprising about 25 mg/mL to about 300 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
30. The pharmaceutical composition for use according to embodiments 25 or 29, comprising about 35 mg/mL to about 200 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
31. The pharmaceutical composition for use according to embodiments 25, 29 or 30 comprising about 70 mg/mL to about 150 mg/mL of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
32. The pharmaceutical composition for use according to any one of embodiments 25-31, wherein the one or more bile acids comprise a cholic acid.
33. The pharmaceutical composition for use according to any one of embodiments 25-32, wherein the one or more free C₁₄₋₂₄ fatty acids comprise one or more free C₁₄₋₁₈ fatty acids.
34. The pharmaceutical composition for use according to embodiment 33, wherein the one or more free C₁₄₋₁₈ fatty acids comprise an oleic acid, a steric acid, a linoleic acid, or any combination thereof.
35. The pharmaceutical composition for use according to any one of embodiments 25-34, wherein the one or more free C₁₄₋₂₄ fatty acid surfactants comprise one or more free C₁₄₋₁₈ fatty acid surfactants.
36. The pharmaceutical composition for use according to embodiment 35, wherein the one or more free C₁₄₋₁₈ fatty acid surfactants comprise an oleate alkali metal or alkali earth metal salt, a stearate alkali metal or alkali earth metal salt, a linoleate alkali metal or alkali earth metal salt, or any combination thereof.
37. The pharmaceutical composition for use according to embodiments 35 or 36, wherein the one or more free C₁₄₋₁₈ fatty acid surfactants comprise a sodium oleate, a sodium stearate, a sodium linoleate, or any combination thereof.
38. A method of treating an individual with a neuroinflammation, the method comprising the step of administering to the individual in need thereof a pharmaceutical composition, the pharmaceutical composition comprising a fenofibrate, about 30% to about 40% by weight of one or more hard fats, about 30% to about 40% by weight of one or more liquid fats, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants
39. The method according to embodiment 38, wherein the pharmaceutical composition comprises about 10% to about 20% by weight of a fenofibrate; about 26% to about 36% by weight of one or more hard fats comprising a mixture of saturated C₁₀-C₁₈ triglycerides and/or saturated C₁₂-C₁₈ triglycerides; about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 12% to about 22% by weight of one or more free C₁₄₋₂₄ fatty acids; and about 0.5% to about 4% by weight of one or more free C₁₄₋₂₄ fatty acid surfactants, wherein administration results in a reduction in a symptom associated with the inflammation, thereby treating the individual.
40. The method according to embodiments 38 or 39, wherein the pharmaceutical composition comprises about 12% to about 18% by weight of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
41. The method according to any one of embodiments 38-40, wherein the pharmaceutical composition comprises about 14% to about 16% by weight of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
42. The method according to embodiment 38, wherein the pharmaceutical composition comprises about 25 mg/mL to about 300 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
43. The method according to embodiments 38 or 42, wherein the pharmaceutical composition comprises about 35 mg/mL to about 200 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
44. The method according to embodiments 38, 42 or 43, wherein the pharmaceutical composition comprises about 70 mg/mL to about 150 mg/mL of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
45. The method according to any one of embodiments 38-44, wherein the one or more bile acids comprise a cholic acid.
46. The method according to any one of embodiments 38-45, wherein the one or more free C₁₄₋₂₄ fatty acids comprise one or more free C₁₄₋₁₈ fatty acids.
47. The method according to embodiment 46, wherein the one or more free C₁₄₋₁₈ fatty acids comprise an oleic acid, a steric acid, a linoleic acid, or any combination thereof.
48. The method according to any one of embodiments 38-47, wherein the one or more free C₁₄₋₂₄ fatty acid surfactants comprise one or more free C₁₄₋₁₈ fatty acid surfactants.
49. The method according to embodiment 48, wherein the one or more free C₁₄₋₁₈ fatty acid surfactants comprise an oleate alkali metal or alkali earth metal salt, a stearate alkali metal or alkali earth metal salt, a linoleate alkali metal or alkali earth metal salt, or any combination thereof.
50. The method according to embodiments 48 or 49, wherein the one or more free C₁₄₋₁₈ fatty acid surfactants comprise a sodium oleate, a sodium stearate, a sodium linoleate, or any combination thereof.
51. Use of a pharmaceutical composition in the manufacture of a medicament for the treatment of a neuroinflammation, the pharmaceutical composition comprising a fenofibrate, about 30% to about 40% by weight of one or more hard fats, about 30% to about 40% by weight of one or more liquid fats, about 3% to about 6% by weight of one or more bile acids, about 15% to about 25% by weight of one or more free C₁₄₋₂₄ fatty acids, and about 0.5% to about 5% by weight of one or more free C₁₄₋₂₄ fatty acid surfactants
52. The use according to embodiment 51, wherein the composition comprises about 10% to about 20% by weight of a fenofibrate; about 26% to about 36% by weight of one or more hard fats comprising a mixture of saturated C₁₀-C₁₈ triglycerides and/or saturated C₁₂-C₁₈ triglycerides; about 26% to about 36% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 12% to about 22% by weight of one or more free C₁₄₋₂₄ fatty acids; and about 0.5% to about 4% by weight of one or more free C₁₄₋₂₄ fatty acid surfactants.
53. The use according to embodiments 51 or 52, wherein the composition comprises about 12% to about 18% by weight of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
54. The use according to any one of embodiments 51-53, wherein the composition comprises about 14% to about 16% by weight of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
55. The use according to embodiment 51, wherein the composition comprises about 25 mg/mL to about 300 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
56. The use according to embodiments 51 or 55, wherein the composition comprises about 35 mg/mL to about 200 mg/mL of a fenofibrate; about 28% to about 34% by weight of one or more hard fats comprising a mixture of saturated C₁₀-C₁₈ triglycerides and/or saturated C₁₂-C₁₈ triglycerides; about 28% to about 34% by weight of one or more liquid fats comprising partially hydrolyzed triglycerides including a mixture of C₁₆-C₁₈ mono-, C₁₆-C₁₈ di- and C₁₆-C₁₈ triglycerides; about 3% to about 7% by weight of one or more bile acids; about 14% to about 20% by weight of one or more free C₁₄₋₂₄ fatty acids; and about 0.8% to about 3% by weight of one or more free C₁₄₋₂₄ fatty acid surfactants.
57. The use according to embodiments 51, 55 or 56, wherein the composition comprises about 70 mg/mL to about 150 mg/mL of a fenofibrate; about 30% to about 32% by weight of one or more hard fats comprising a mixture of saturated C₁₀-C₁₈ triglycerides and/or saturated C₁₂-C₁₈ triglycerides; about 30% to about 32% by weight of one or more liquid fats comprising partially hydrolyzed triglycerides including a mixture of C₁₆-C₁₈ mono-, C₁₆-C₁₈ di- and C₁₆-C₁₈ triglycerides; about 4% to about 6% by weight of one or more bile acids; about 16% to about 18% by weight of one or more free C₁₄₋₂₄ fatty acids; and about 1% to about 1.4% by weight of one or more free C₁₄₋₂₄ fatty acid surfactants.
58. The use according to any one of embodiments 51-57, wherein the one or more bile acids comprise a cholic acid.
59. The use according to any one of embodiments 51-58, wherein the one or more free C₁₄₋₂₄ fatty acids comprise one or more free C₁₄₋₁₈ fatty acids.
60. The use according to embodiment 59, wherein the one or more free C₁₄₋₁₈ fatty acids comprise an oleic acid, a steric acid, a linoleic acid, or any combination thereof.
61. The use according to any one of embodiments 51-60, wherein the one or more free C₁₄₋₂₄ fatty acid surfactants comprise one or more free C₁₄₋₁₈ fatty acid surfactants.
62. The use according to embodiment 61, wherein the one or more free C₁₄₋₁₈ fatty acid surfactants comprise an oleate alkali metal or alkali earth metal salt, a stearate alkali metal or alkali earth metal salt, a linoleate alkali metal or alkali earth metal salt, or any combination thereof.
63. The use according to embodiments 61 or 62, wherein the one or more free C₁₄₋₁₈ fatty acid surfactants comprise a sodium oleate, a sodium stearate, a sodium linoleate, or any combination thereof.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, pharmaceutical compositions, or methods and uses disclosed herein.

### Example 1 Assessing Critical Micellar Concentration Parameters

One of the key features of the disclosed pharmaceutical compositions is that they are incapable of self-emulsifying. A compound can self-emulsify to form micelles when its concentration reaches its critical micellar concentration (CMC). The lipid components GELUCIRE® 43/01 and MAISINE™ CC have no recorded CMC values, cannot form micelles, and as such are not self-emulsifying compounds. Similarly, while cholic acid is reported to have a CMC of 14.7 mM, below the 0.25 mM or less of cholic acid used in the disclosed pharmaceutical compositions. Likewise sodium stearate is reported to have a CMC of 71 μM, below the 35 μM or less of sodium stearate used in the disclosed pharmaceutical compositions. As such, cholic acid and sodium stearate cannot self-emulsify because their concentrations are insufficient to form micelles. On the other hand, oleic acid is reported to have a CMC of 2.3 mM which is near the up to 2 mM of oleic acid is used in the disclosed pharmaceutical compositions. As such, it was not clear whether oleic acid could act as a self-emulsifying agent as it could conceivably form micelles at the concentrations used in the disclosed pharmaceutical compositions.

To assess whether oleic acid could self-emulsify at the concentrations used in the disclosed pharmaceutical compositions, experiments was performed that were designed to model dissolution testing according to International Council for Harmonisation (ICH) guidelines. One liter of a dissolution media comprising phosphate buffered saline (PBS) and 2.5% curcumin was warmed to 37° C. and constant stirring at 300 rpm maintained. Curcumin was added as a coloring agent and was very effective at visualizing the distribution of lipids through the dissolution media. Miscibility experiments were performed using two concentrations of oleic acid nominally above the CMC concentration for this fatty acid. In one series of experiments, 1 g of 100% oleic acid was added to the dissolution media. The control compound was 1 g of 100% MAISINE™ CC. In a second series of experiments 1 g of 75% oleic acid (diluted with 250 mg of MAISINE™ CC) was added to the dissolution media. The control compound was 1 g of 75% MAISINE™ CC. The miscibility of oleic acid and MAISINE™ CC was assessed by observing the dissolution media for 10 minutes with constant stirring and then 10 minutes without stirring. Photographs were taken at time 0, 1, 2, 3, 4, 5 and 10 minutes during the stirring phase and 2, 4 and 10 minutes after stirring ceased.

Both oleic acid preparations quickly distributed throughout the PBS in small but clearly defined lipid droplets which were still observable after 10 minutes of stirring. On cessation of stirring, the lipid droplets either adhered to the stirrer or floated to the top of the PBS, with the bulk of the PBS clearing. In contrast, the MAISINE™ CC preparations rapidly dispersed in the dissolution media and this dispersion was maintained after the 10-minute period of no stirring. These results demonstrate that 1 gram of 100% oleic acid and 75% oleic acid were not miscible in the dissolution media, despite being at a concentration that is high enough to theoretically reach the critical micellar concentration. These experiment, modelling ICH dissolution conditions, demonstrate that oleic acid did not form emulsions and shown that this fatty acid cannot self-emulsify into micelles. Moreover, oleic acid is even less likely to self-emulsify, when combined with other lipid components which have no CMC concentration, such as MAISINE™ CC and GELUCIRE® 43/01.

### Example 2 Assessing Cholic Acid Parameters

During the course of developing the disclosed pharmaceutical compositions, it became clear that there was a direct correlation between having cholic acid in the formulation and improved performance. For many drugs, cholic acid is required for solubility in the lipid matrix. As such we wanted to assess the effect of having increased concentrations of cholic in the lipid matrix.

Unless stated otherwise X-ray powder diffraction patterns for the samples were acquired on a Bruker D8 diffractometer using Cu Ka radiation (40 kV, 40 mA) and a 6-26 goniometer. The incident beam passes through a 2.0 mm divergence slit followed by a 0.2 mm antiscatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively. Samples were run under ambient conditions over an angular range of 2° to 42° 26 (using a step size of 0.05° 26 and a step time of 0.5 seconds) as flat plate specimens using powder as received. The sample was prepared on a polished, zero-background (510) silicon wafer by gently pressing onto the flat surface or packed into a cut cavity. The sample was rotated in its own plane. Small D8 disc recess holders were used to prepare sample. Pharmaceutical composition analyzed comprised 36.5% MAISINE™ CC, 36.5% GELUCIRE® 43/01, 20.3% oleic acid, 6.1% cholic acid, 1.4% sodium stearate, and 0.6% fenofibrate. In addition, a vehicle was also analyzed and comprised 36.5% MAISINE™ CC, 36.5% GELUCIRE® 43/01, 20.3% oleic acid, 6.1% cholic acid, and 1.4% sodium stearate. Controls for these experiments included GELUCIRE® 43/01, cholic acid, and fenofibrate. Controls for MAISINE™ CC, oleic acid, and sodium stearate were not performed as these compounds are liquid at room temperature, and as such, cannot be analyzed using X-ray powder diffraction.

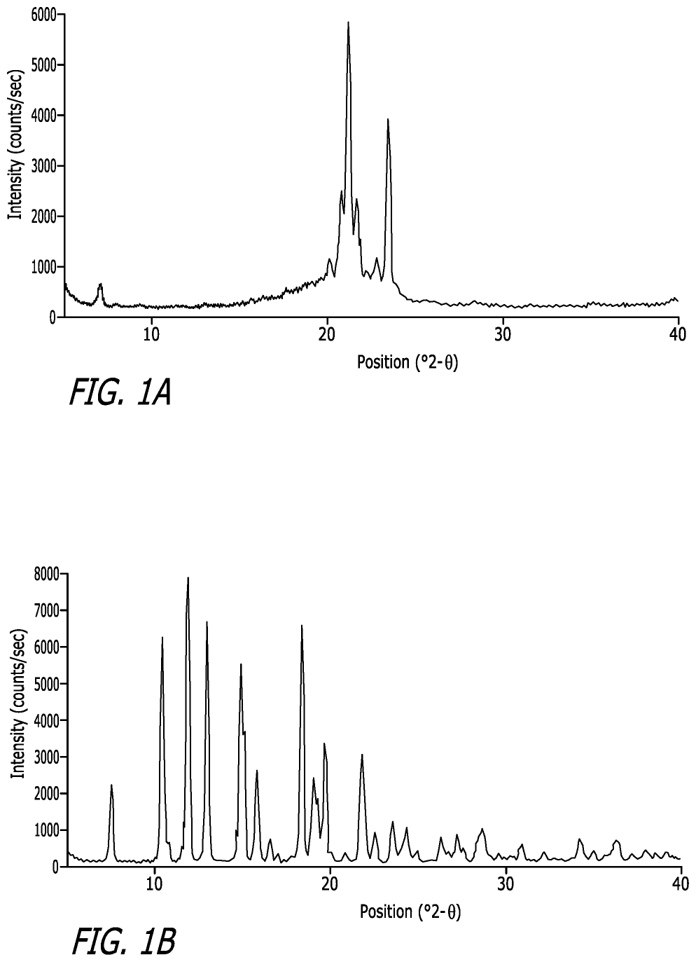

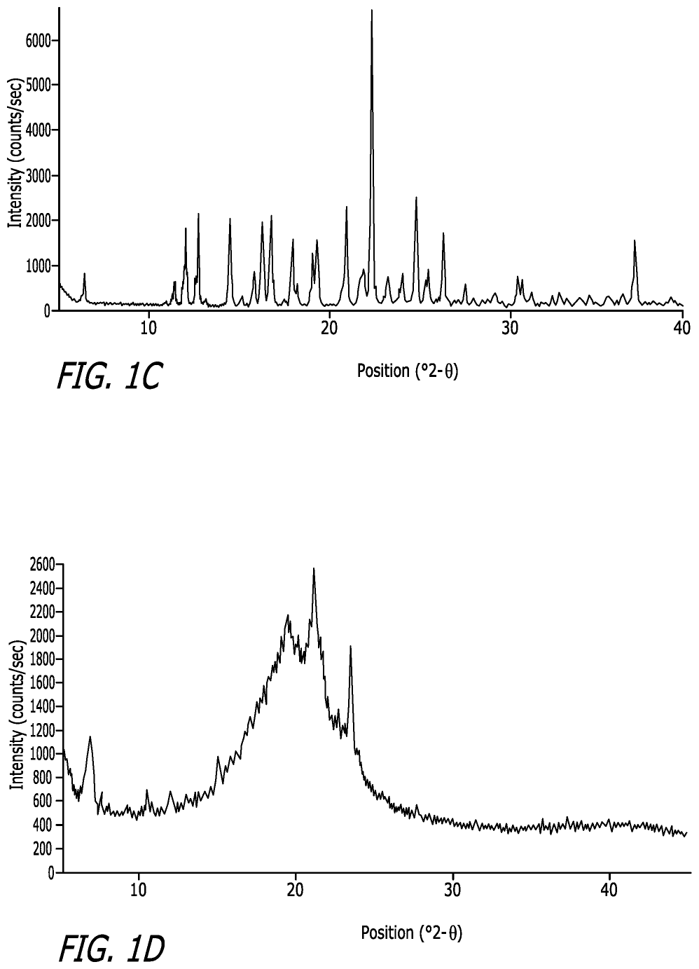

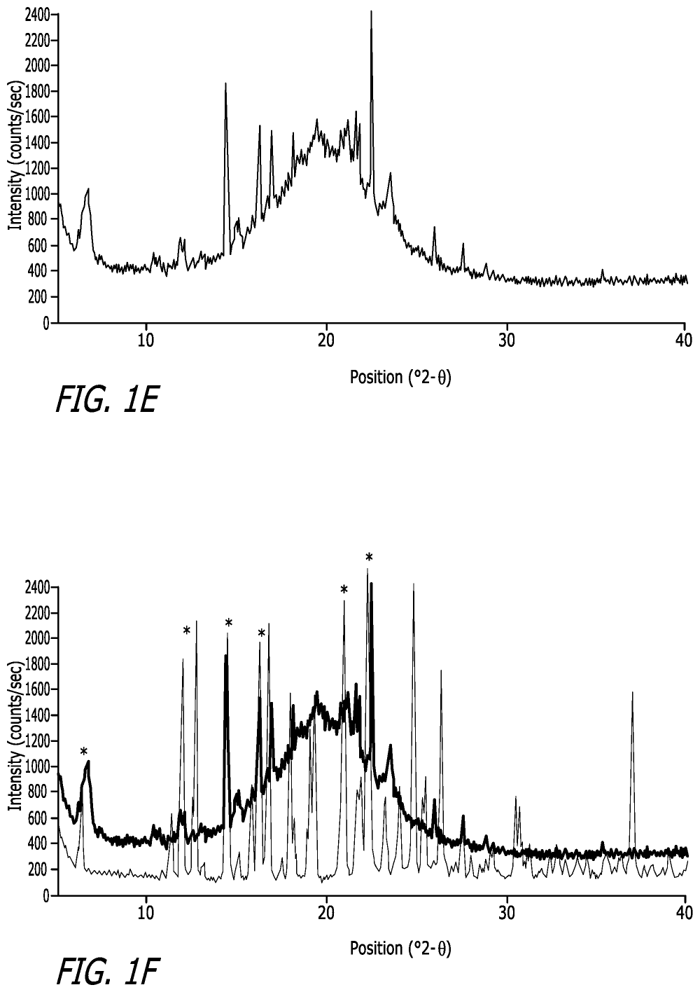

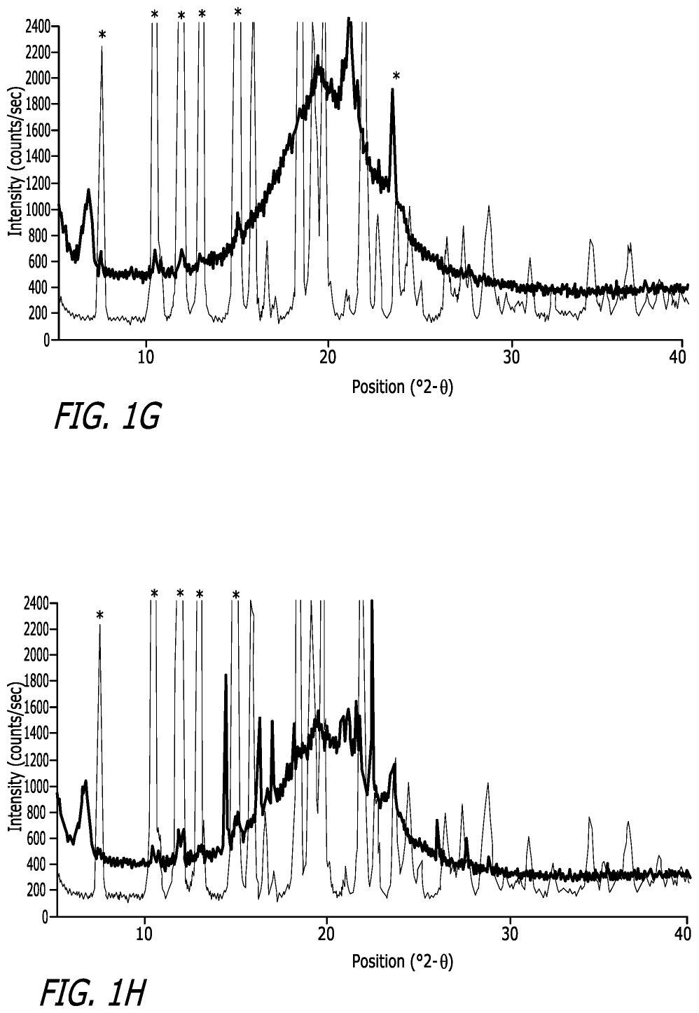

Representative PXRD spectra of GELUCIRE® 43/01 (FIG. 1A), cholic acid (FIG. 1B), and fenofibrate (FIG. 1C), as well as the vehicle (FIG. 1D) and the pharmaceutical composition (FIG. 1E), each show a distinctive pattern of peaks. Interestingly, when control PXRD spectra are superimposed over the vehicle and pharmaceutical composition PXRD spectra, there is a high degree of coincidental peaks. For example, FIG. 1F, is a PXRD spectra of the pharmaceutical composition superimposed with a PXRD spectra of fenofibrate. As shown by the asterisks, the pharmaceutical composition exhibits a peak profile coincident with fenofibrate, indicating that the pharmaceutical composition contains crystalline fenofibrate. This finding is important as it illustrates that the pharmaceutical composition is not a solid solution (or molecular dispersion) transitional phase instead comprises some solid fenofibrate micro to nano-sized particles suspended within the lipid matrix formed during the cooling of the formulation from a fully solubilized state.

Even more surprising were the results obtained by analyzing the peak profile of cholic acid. FIG. 1G shows a PXRD spectra of the vehicle superimposed with a PXRD spectra of cholic acid. As shown by the asterisks, the vehicle exhibited a peak profile coincident with cholic acid, indicating that the pharmaceutical composition contains crystalline cholic acid. Similarly, FIG. 1H shows a PXRD spectra of the pharmaceutical composition superimposed with a PXRD spectra of cholic acid. As shown by the asterisks, the pharmaceutical composition exhibits a peak profile coincident with cholic acid, indicating that the pharmaceutical composition contains crystalline cholic acid. This finding was completely unexpected as it shows that pharmaceutical composition comprises solid cholic acid particles suspended within the lipid matrix. Additionally, as these particles were not observed during the formulation of the pharmaceutical composition, it was concluded that the crystalline cholic acid are present as micro to nano-sized particles formed during the cooling of the formulation.

Taken together, the results of this analysis demonstrate that a pharmaceutical composition disclosed hereon is a mixed phase solid suspension of solid crystalline fenofibrate and solid crystalline cholic acid embedded within a solid lipid matrix.

Such mixed phase solid suspensions provide a unique formulation that offers several advantageous. For example, in a single-phase composition, all the components are dissolved and digestion of the lipid matrix by pancreatic juices results in uniform processing of the composition into mixed micelles that are absorbed by the enterocytes. However, in mixed phase solid suspensions, while the dissolved components behave as in a single-phase composition, the solid crystalline compounds can solvent and interact with the aqueous environment of the small intestine to form bile salts. This dual processing scheme affords more opportunity for more complex processes to occur, that is faster, and produces more micellar formations.

### Example 3 Fenofibrate Formulations

Pharmaceutical compositions comprising fenofibrate were formulated according to Tables 1 and 2 below. MAISINE™ CC, GELUCIRE® 43/01, and the fatty acid component or the surfactant component were combined and heated to 60° C. to produce a clear yellow solution. While maintaining the temperature at 60° C., fenofibrate was then added under constant stirring until a clear solution was produced. The resulting composition produced a clear yellow solution which was then allowed to cool to room temperature (18-20° C.) at which time stirring ceased and the composition was transferred to suitable containers where it solidified. The resulting solid composition remelted at 40° C. to give a clear yellow solution with no precipitate formation and again solidified on cooling.

TABLE 1
Fenofibrate Formulations

| Component | FENF1 | | FENF2 | | FENF3 | | FENF4 | |
|---|---|---|---|---|---|---|---|---|
| MAISINE ™ CC | 2,020 mg | (44.6%) | 2,100 mg | (46.4%) | 300 mg | (32.6%) | 3.65 g | (36.5%) |
| GELUCIRE ® 43/01 | 2.020 mg | (44.6%) | 2,250 mg | (49.6%) | 300 mg | (32.6%) | 3.65 g | (36.5%) |
| Cholic Acid | 179 mg | (4.0%) | 150 mg | (3.3%) | — | | 0.61 g | (6.1%) |
| Linoleic acid | — | | — | | 110 mg | (12.0%) | — | |
| Oleic Acid | 279 mg | (6.1%) | — | | — | | 2.03 g | (20.3%) |
| Sodium Stearate | — | | — | | 10 mg | (1.1%) | 0.14 g | (1.4%) |
| Fenofibrate | 30 mg | (0.7%) | 30 mg | (0.7%) | 200 mg | (21.7%) | 0.06 g | (0.6%) |

TABLE 2
Fenofibrate Formulations

| Component | FENF5 | | FENF6 | | FENF7 | | FENF8 | |
|---|---|---|---|---|---|---|---|---|
| MAISINE ™ CC | 4.11 g | (41.1%) | 4.42 g | (44.2%) | 3.31 g | (33.1%) | 4.44 g | (44.4%) |
| GELUCIRE ® 43/01 | 2.81 g | (28.1%) | 4.42 g | (44.2%) | 4.42 g | (44.2%) | 4.44 g | (44.4%) |
| Cholic Acid | 0.9 g | (9.0%) | — | | — | | 0.04 g | (0.4%) |
| Linoleic acid | — | | — | | — | | — | |
| Oleic Acid | 2.01 g | (20.1%) | 1.1 g | (11.0%) | 2.21 g | (22.1%) | 0.66 g | (6.6%) |
| Sodium Stearate | 0.14 g | (1.4%) | — | | — | | — | |
| Fenofibrate | 0.07 g | (0.7%) | 0.07 g | (0.7%) | 0.07 g | (0.7%) | 0.07 g | (0.7%) |

Previous studies have shown that fenofibrate is soluble in a glycerolipid admixture of MAISINE™ CC and GELUCIRE® 43/01. To assess whether the addition of one or more digestion enhancers to glycerolipid admixture comprising fenofibrate could improve the bioavailability of this therapeutic compound, both the pharmacokinetics and pharmacodynamics of these formulations were assessed. Pharmacokinetic analysis will ascertain how an organism affects the therapeutic compound being examined, whereas pharmacodynamics will determine how the therapeutic compound affects the organism.

In one series of experiments, the pharmacokinetic profiles of FENF1 and FENF2 were evaluated against Comparator Formulation 1 (CMF1) comprising 2,250 mg (49.6%) of MAISINE™ CC, 2,250 mg (49.6%) of GELUCIRE® 43/01, and 30 mg (0.7%) of fenofibrate, and Comparator Formulation 2 (CMF2) a suspension comprising 1% carboxymethyl cellulose and 0.7% of fenofibrate. C57BL/6 male mice, each with an average weight of between 20 g to 25 g, were divided into four groups of nine animals. Animals from each group were oral dosed by gavage as follows: Group 1 animals received a single dose of FENF1 administered at 30 mg/kg; Group 2 animals received a single dose of FENF2 administered at 30 mg/kg; Group 3 animals received a single dose of CMF1 administered at 30 mg/kg; and Group 4 animals received a single dose of CMF2 administered at 30 mg/kg. Samples of whole blood were taken just prior to administration, 0 h, and at the following 7 post-administration time points: 0.5 hr, 1 hr, 2 hr, 4 hr, 8 hr, 12 hr, and 24 hr. Collected whole blood samples were processed by adding 200 μL of a tolbutamide internal standard solution (1 mg/mL tolbutamide in DMSO/water/acetonitrile) to 50 μL of whole blood and precipitating blood proteins from this admixture using standard procedures and the resulting supernatants were stored for analysis. Samples of brain were taken at 3 post-administration time points (n=3 per time point): 2 hr, 4 hr, and 24 hr. Collected brain samples were processed by homogenizing 1 g of brain in 1 mL of water, adding 200 μL of tolbutamide internal standard solution and 5 μL of 1:1 acetonitrile/water to 50 μL aliquots of sample brain homogenate, centrifuging this admixture, and adding 100 μL water to 50 μL of the resulting supernatant which was then stored for analysis. Processed blood and brain supernatants were sent for bioanalysis, utilizing UHPLC-MS/MS with a reverse phase C18 column (50×21 mm), 1.7 mm 50° C. with a mobile phase gradient between: 0.1% formic acid in water and 0.1% formic acid in acetonitrile, a flow rate of 0.4 mL/min and injection volume 2 mL. Blood samples were analyzed for various pharmacokinetic parameters including the amount of fenofibrate administer (dose), the peak concentration of fenofibrate and fenofibric acid achieved after administration (Cmax), the time required for fenofibrate and fenofibric acid to reach its Cmax (Tmax), the time required for the concentration of fenofibrate and fenofibric acid to reach half its Cmax (T½), the integral of the concentration-time curve between from time zero to the time of last quantifiable measurement taken during the experiments (AUClast), and the integral of the concentration-time curve from time zero to infinity (AUCinf). Since brain samples were limited to three time points only the pharmacokinetic parameters of amount of fenofibrate administer (dose) and the peak concentration of fenofibrate and fenofibric acid achieved after administration (Cmax) could be reliably defined.

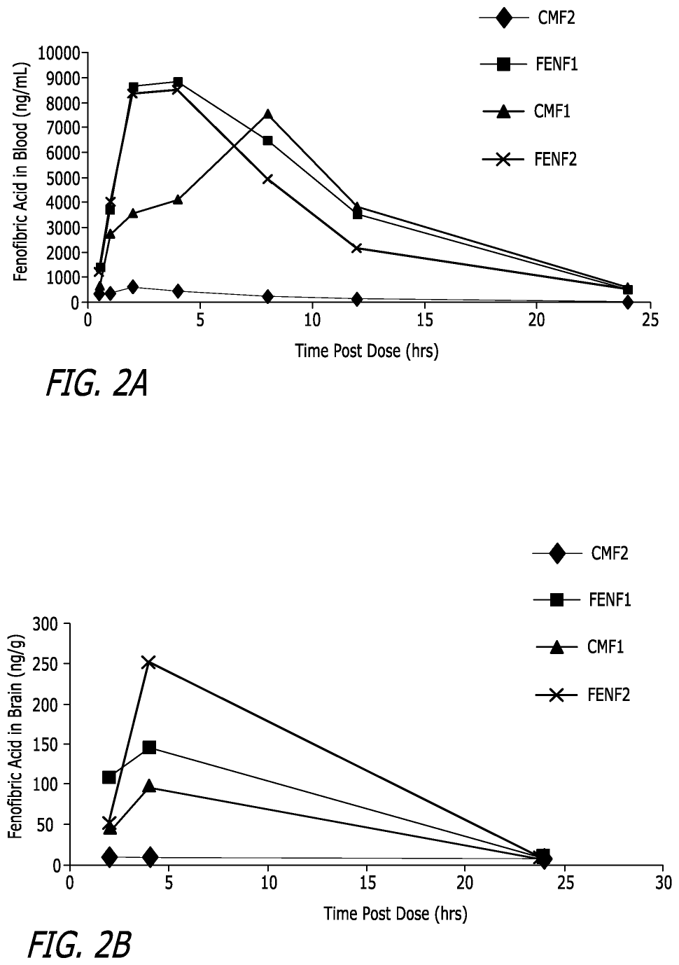

As shown in FIG. 2A and Table 3, FENF1 and FENF2, fenofibrate formulated with lipids and with digestion enhancers, exhibited superior pharmacokinetic properties for fenofibric acid relative to CMF1 and CMF2. In comparison to CMF1 (previous lipid formulation without digestion enhancers), FENF1 and FENF2 demonstrated higher absorption levels of fenofibric acid in blood as indicated by increased Cmax, with a shorter time to maximum concentration as shown by decreased Tmax, and lower clearance over time as indicated by a larger AUC versus CMF1. In comparison to CMF2 (non-lipid comparator formulation), FENF1 and FENF2 demonstrated substantially higher absorption levels of fenofibric acid in blood as indicated by increased Cmax, with lower clearance over time as indicated by a larger AUC versus CMF2. FENF1 had a higher Cmax and AUC than either FFEN2, CMF1 or CMF2 suggesting that FENF1 produces higher concentrations of fenofibric acid in blood with and the slowest clearance over time indicating greater drug exposure relative to the other formulations. Overall, FENF1 and FENF2 demonstrate pharmacokinetic improvements compared to the comparator formulations. CMF1 and CMF2.

TABLE 3
Mean Pharmacokinetic Parameters of Blood
Fenofibric Acid by Formulation

| Parameter | FENF1 | FENF2 | CMF1 | CMF2 |
|---|---|---|---|---|
| T1/2 (hr) | 4.4 | 5.1 | 4.4 | 3.3 |
| Tmax (hr) | 4.0 | 4.0 | 8.0 | 2.0 |
| Cmax (ng/mL) | 8875.7 | 8557.3 | 7586.0 | 611.9 |
| AUClast (hr*ng/mL) | 100664.4 | 82082.5 | 84882.6 | 4598.6 |
| AUCinf (hr*ng/mL) | 103969.7 | 85871.7 | 88625.7 | 4636.0 |

As shown in FIG. 2B and Table 4, FENF1 and FENF2 substantially increased the absorption level of fenofibric acid in brain over both CMF1 and CMF2. In comparison to CMF1, FENF1 and FENF2 exhibited higher absorption levels of fenofibric acid in brain as indicated by increased Cmax and larger AUC versus CMF1, with FENF1 being absorbed 1.5 times more than CMF1, and FENF2 being absorbed 2.5 times more than CMF1. In comparison to CMF2, FENF1 and FENF2 demonstrated substantially higher absorption levels of fenofibric acid in brain as indicated by increased Cmax and larger AUC versus CMF2, with FENF1 being absorbed 14 times more than CMF1, and FENF2 being absorbed 24 times more than CMF2. In addition, with respect to FENF2, the overall amount of fenofibric acid entering the brain as well as its proportion relative to blood concentration is great than FENF1 as well as CMF1 and CMF2. These results supports the pharmacokinetic advantages associated with FENF1 and FENF2, fenofibrate formulated with lipids and with digestion enhancers, by demonstrating enhanced concentration levels of fenofibric acid in brain, that are not achieved with previous lipid formulations without digestion enhancers (CMF1) and non-lipid comparators (CMF2).

TABLE 4
Fenofibric Acid Blood and Brain Ratios of Mean Cmax by Formulation

| Sample | FENF1 | | FENF2 | | CMF1 | | CMF2 | |
|---|---|---|---|---|---|---|---|---|
| Blood | 8875.7 | ng/mL | 8557.3 | ng/mL | 7586.0 | ng/mL | 611.9 | ng/mL |
| Brain | 145.9 | ng/g | 253.9 | ng/g | 98.8 | ng/g | 10.4 | ng/g |

| | | | | |
|---|---|---|---|---|
| Brain:Blood Ratio | 0.016 | 0.030 | 0.013 | 0.17 |

Additionally, as shown in Table 5, fenofibrate formulated with lipids and with digestion enhancers, exhibited superior pharmacokinetic properties for fenofibrate itself relative to CMF1 and CMF2. Although no significant differences in measured pharmacokinetic parameters of fenofibrate was observed in blood, enhanced pharmacokinetic parameters of fenofibrate in brain were detected. For example, in comparison to CMF1, FENF1 and FENF2 exhibited higher absorption levels of fenofibrate in brain as indicated by increased Cmax versus CMF1, with FENF1 being absorbed 3.8 times more than CMF1, and FENF2 being absorbed 2.6 times more than CMF1. In comparison to CMF2, FENF1 and FENF2 exhibited higher absorption levels of fenofibrate in brain as indicated by increased Cmax versus CMF2, with FENF1 being absorbed 3.4 times more than CMF1, and FENF2 being absorbed 2.3 times more than CMF2. FENF1 and FENF2 produced higher concentrations of fenofibrate in both blood and brain compared to CMF2, providing pharmacokinetic support that the lipid formulations of fenofibrate achieve higher levels of drug in brain compared to non-lipid-based comparators. One explanation for these finding is that the lipid-formulations of FENF1 and FENF2 slow down the conversion rate of fenofibrate to fenofibric acid, thereby protecting fenofibrate in blood so that this compound can be transported into the brain. Although the levels of fenofibrate in the blood and brain were lower than those observed for fenofibric acid, this was expected based on the conversion rate of fenofibrate to fenofibric acid which can be slowed, but not prevented.

TABLE 5
Fenofibrate Blood and Brain Ratios of Mean Cmax by Formulation

| Sample | FENF1 | | FENF2 | | CMF1 | | CMF2 | |
|---|---|---|---|---|---|---|---|---|
| Blood | 5.9 | ng/mL | 9.3 | ng/mL | 12.8 | ng/mL | 5.1 | ng/mL |
| Brain | 33.9 | ng/g | 23.0 | ng/g | 8.9 | ng/g | 10.0 | ng/g |

| | | | | |
|---|---|---|---|---|
| Brain:Blood Ratio | 5.7 | 2.5 | 0.7 | 2.0 |

Taken together, these results show that FENF1 and FENF2 formulations exhibited significantly improved pharmacokinetic properties of fenofibrate relative to either the CMF1 or CMF2 formulations. Overall, this data supports the proposition that the inclusion of digestion enhancers increases the extent and speed of absorption of drug, in both blood and brain, over previous lipid formulations (CMF1) and non-lipid-based comparator formulations of fenofibrate (CMF2).

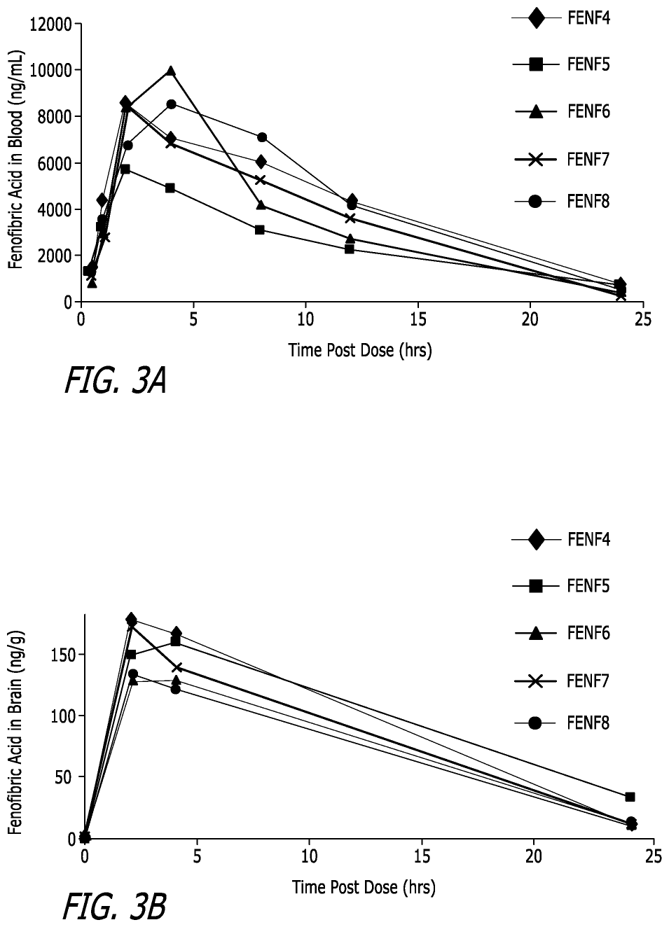

To extend this analysis, the pharmacokinetic properties of five further fenofibrate formulations were evaluated. In these experiments, C57BL/6 male mice were administered 30 mg/kg of fenofibrate formulated as FENF4, FENF5, FENF6, FENF7, or FENF8 using the experimental design described above. As shown in FIG. 3A and Table 6, FENF4, FENF6, FENF7, and FENF8, each a formulation of fenofibrate with lipids and with digestion enhancers, all demonstrated pharmacokinetic properties of fenofibric acid in blood that were comparable to the pharmacokinetic properties observed for FENF1 and FENF2. For example, absorption levels of fenofibric acid in blood as indicated by Cmax for FENF4, FENF6, FENF7, and FENF8 ranged between 8436.0 ng/mL to 10,010.0 ng/mL, levels comparable to the Cmax value of 8875.7 ng/mL for FENF1 and 8557.3 ng/mL for FENF2. Similarly, time to maximum fenofibric acid concentration as indicated by Tmax for FENF4, FENF6, FENF7, and FENF8 ranged between 3.6 hr to 4.8 hr, a time comparable to the Tmax of 4.0 for both FENF1 and FENF2. In terms of individual formulations, FENF6 demonstrated the highest Cmax (10,010.0 ng/mL), FENF7 demonstrated the lowest Tmax (3.6 hrs), and FENF4 demonstrated the lowest T½ (2.0 hrs). Overall, with the exception of FENF5, all fenofibrate formulations comprising lipids and digestion enhancers demonstrated a similar absorption profile of fenofibric acid relative to FENF1 and FENF2. Compared to all other formulations, FENF4 demonstrated the best overall balance of pharmacokinetic parameters having both low T½ and Tmax values and high Cmax and AUC values, illustrating this formulation reaches maximum concentration in the shortest amount of time with the greatest amount of drug exposure over time in the blood.

TABLE 6
Mean Pharmacokinetic Parameters of Blood
Fenofibric Acid by Formulation

| Parameter | FENF4 | FENF5 | FENF6 | FENF7 | FENF8 |
|---|---|---|---|---|---|
| T1/2 (hr) | 2.0 | 2.0 | 4.0 | 2.0 | 4.0 |
| Tmax (hr) | 4.8 | 6.8 | 4.5 | 3.6 | 3.9 |
| Cmax (ng/mL) | 8575.0 | 5693.0 | 10010.0 | 8436.0 | 8574.0 |
| AUClast | 100600.0 | 60233.0 | 85889.0 | 87304.0 | 104221.0 |
| (hr*ng/mL) | | | | | |
| AUCinf | 105125.0 | 66248.0 | 88335.0 | 88764.0 | 106732.0 |
| (hr*ng/mL) | | | | | |

As shown in Table 7, FENF4, FENF5, FENF6, FENF7, and FENF8 all demonstrated pharmacokinetic properties of fenofibrate in blood that were superior to pharmacokinetic properties observed for FENF1 and FENF2. For example, absorption levels of fenofibrate in blood as indicated by Cmax for FENF4, FENF6, FENF7, and FENF8 ranged between 119.0 ng/mL to 548.0 ng/mL, levels significantly higher than the Cmax values of 5.9 ng/mL for FENF1 and 9.3 ng/mL for FENF2. In terms of individual formulations, FENF4 demonstrated the highest Cmax (548.0 ng/mL), FENF4, FENF5, and FENF8 all demonstrated the lowest Tmax (1.9 hrs), and FENF5 demonstrated the lowest T½ (0.5 hrs). Overall, all fenofibrate formulations comprising lipids and digestion enhancers demonstrated a superior absorption profile of fenofibrate relative to FENF1 and FENF2. Compared to all other formulations, FENF4 again demonstrated the best overall balance of pharmacokinetic parameters having both low T½ and Tmax values and high Cmax and AUC values, illustrating this formulation reaches maximum concentration in the shortest amount of time with the greatest amount of drug exposure over time in the brain.

TABLE 7
Mean Pharmacokinetic Parameters of
Blood Fenofibrate by Formulation

| Parameter | FENF4 | FENF5 | FENF6 | FENF7 | FENF8 |
|---|---|---|---|---|---|
| T1/2 (hr) | 1.0 | 0.5 | 1.3 | 1.3 | 0.5 |
| Tmax (hr) | 1.9 | 1.9 | 2.0 | 2.0 | 1.9 |
| Cmax (ng/mL) | 548.0 | 154.8 | 131.8 | 119.0 | 195.0 |
| AUClast (hr*ng/mL) | 1520.0 | 580.0 | 282.9 | 269.0 | 576.0 |
| AUCinf (hr*ng/mL) | 1538.0 | 933.0 | NR | NR | NR |

NR: No result, insufficient data to reliably calculate pharmacokinetic parameter.

Additionally, as shown in FIG. 3B and Table 8, FENF4, FENF5, FENF6, FENF7, and FENF8 all demonstrated pharmacokinetic properties of fenofibric acid that were comparable to pharmacokinetic properties observed for FENF1 and FENF2. For example, absorption levels of fenofibric acid in brain as indicated by Cmax for FENF4, FENF5, FENF6, FENF7, and FENF8 ranged between 130.0 ng/g to 178.0 ng/g, levels comparable to the Cmax values of 145.9 ng/g ng/mL for FENF1 and 253.9 ng/ng for FENF2. In terms of individual formulations, FENF4 demonstrated the highest Cmax (178.0 ng/g). Overall, all fenofibrate formulations comprising lipids and digestion enhancers demonstrated a comparable absorption profile of fenofibric acid relative to FENF1 and FENF2. Compared to all other formulations, FENF4 again demonstrated the best overall balance of pharmacokinetic parameters having a high Cmax values, illustrating this formulation reaches maximum concentration in the shortest amount of time with the greatest amount of drug exposure over time in the brain.

TABLE 8
Fenofibric Acid Blood and Brain Ratios of Mean Cmax by Formulation

| Sample | FENF4 | | FENF5 | | FENF6 | | FENF7 | | FENF8 | |
|---|---|---|---|---|---|---|---|---|---|---|
| Blood | 8575.0 | ng/mL | 5693.0 | ng/mL | 10010.0 | ng/mL | 8436.0 | ng/mL | 8574.0 | ng/mL |
| Brain | 178.0 | ng/g | 159.3 | ng/g | 130.0 | ng/g | 173.0 | ng/g | 133.0 | ng/g |

| | | | | | |
|---|---|---|---|---|---|
| Brain:Blood Ratio | 0.02 | 0.03 | 0.01 | 0.02 | 0.02 |

Additionally, as shown in Table 9, FENF4, FENF5, FENF6, FENF7, and FENF8 all demonstrated pharmacokinetic properties of fenofibrate that were superior to pharmacokinetic properties observed for FENF1 and FENF2. For example, absorption levels of fenofibrate in brain as indicated by Cmax for FENF4, FENF5, FENF6, FENF7, and FENF8 ranged between 87.9 ng/g to 276.4 ng/g, levels significantly higher than the Cmax values of 33.9 ng/g ng/mL for FENF1 and 23.0 ng/ng for FENF2. In terms of individual formulations, FENF6 demonstrated the highest Cmax (276.4 ng/g). Overall, all fenofibrate formulations comprising lipids and digestion enhancers demonstrated a superior absorption profile of fenofibrate relative to FENF1 and FENF2.

TABLE 9
Fenofibrate Blood and Brain Ratios of Mean Cmax by Formulation

| Sample | FENF4 | | FENF5 | | FENF6 | | FENF7 | | FENF8 | |
|---|---|---|---|---|---|---|---|---|---|---|
| Blood | 548.0 | ng/mL | 154.8 | ng/mL | 131.8 | ng/mL | 119.0 | ng/mL | 195.0 | ng/mL |
| Brain | 195.8 | ng/g | 199.5 | ng/g | 276.4 | ng/g | 257.7 | ng/g | 87.9 | ng/g |

| | | | | | |
|---|---|---|---|---|---|
| Brain:Blood Ratio | 0.36 | 1.29 | 2.10 | 2.17 | 0.45 |

Taken together, these results show that FENF4, FENF5, FENF6, FENF7, and FENF8 formulations exhibited significantly improved pharmacokinetic properties of fenofibric acid and fenofibrate that were largely equivalent to, and in many instances better than, FENF1 and FENF2. Of these formulations, FENF4 appeared to have the best pharmacokinetic properties of fenofibrate with demonstration of high Cmax, low Tmax and high AUC. Overall, this data supports the proposition that the inclusion of digestion enhancers increase the extent and speed of absorption of drug over a glycerolipid admixture alone, and over a non-lipid comparator.

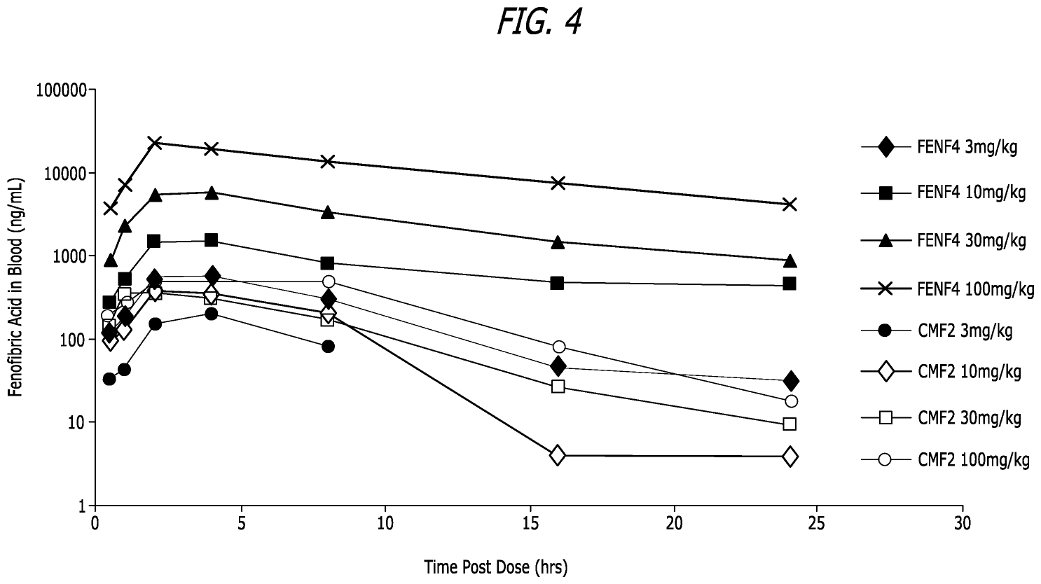

As FENF4 exhibited the best pharmacokinetic properties of all tested formulations, these studies were repeated using four different doses of fenofibrate. In these dose response experiments C57BL/6 male mice were administered 3 mg/kg, 10 mg/kg, 30 mg/kg, or 100 mg/kg of fenofibrate formulated as FENF4 using the experimental design described above. As comparator controls, C57BL/6 male mice were administered 3 mg/kg, 10 mg/kg, 30 mg/kg, or 100 mg/kg of fenofibrate formulated as CMF2 using the experimental design described above. As shown in FIG. 4, animals exhibited a significantly greater absorption level in blood of fenofibric acid formulated as FENF4 relative to the CMF2 controls. In fact, the lowest dose of FENF4 (3 mg/kg of fenofibrate) demonstrated equivalent absorption rates of fenofibric acid in blood as compared to the highest dose of CMF2 (100 mg/kg of fenofibrate), suggesting that only a low dose of FENF4 is required to meet systemic equivalence with a high dose of standard, non-lipid fenofibrate.

Additionally, as shown in FIG. 4 and Table 9, FENF4 revealed a dose-response effect on the absorption rate of fenofibric acid in blood with increasing doses producing progressively higher blood concentrations of fenofibric acid in animals and higher AUC. For example, the 3 mg/kg dose of FENF4 had a Cmax of 569.2 ng/mL of fenofibric acid in the blood, with the 10 mg/kg dose of FENF4 had a Cmax of 569.2 ng/mL of fenofibric acid in blood (representing a 2.6 times increase in Cmax versus the 3 mg/kg dose), the 30 mg/kg dose of FENF4 had a Cmax of 5690.6 ng/mL of fenofibric acid in the blood (representing a 3.8 times increase in Cmax versus the 10 mg/kg dose and a 10 times increase in Cmax versus the 3 mg/kg dose), and the 100 mg/kg dose of FENF4 had a Cmax of 22679.5 ng/mL of fenofibric acid in the blood (representing a 4 times increase in Cmax versus the 30 mg/kg dose, a 15.1 time increase in Cmax versus the 10 mg/kg dose, and a 39.8 times increase in Cmax versus the 3 mg/kg dose).

TABLE 9
Mean Pharmacokinetic Parameters of
Blood Fenofibric Acid by FENF4 Dose

| | FENF4 Dosage | | | |
|---|---|---|---|---|
| Parameter | 3 mg/kg | 10 mg/kg | 30 mg/kg | 100 mg/kg |
| T1/2 (hr) | 4.8 | 17.7 | 8.5 | 9.3 |
| Tmax (hr) | 4.0 | 4.0 | 4.0 | 2.0 |
| Cmax (ng/mL) | 569.2 | 1498.9 | 5690.6 | 22679.5 |
| AUClast (hr*ng/mL) | 4986.6 | 17591.5 | 62571.5 | 259542.1 |
| AUCinf (hr*ng/mL) | 5196.1 | 28729.8 | 73503.6 | 316198.0 |

As shown in Table 10, FENF4 revealed a similar dose-response effect on the absorption rate of fenofibrate in blood with increasing doses producing progressively higher blood concentrations of fenofibrate in animals. For example, the 3 mg/kg dose of FENF4 had a Cmax of 10.3 ng/mL of fenofibrate in the blood, with the 10 mg/kg dose of FENF4 had a Cmax of 20.6 ng/mL of fenofibrate in blood (representing a 2.0 times increase versus the 3 mg/kg dose), the 30 mg/kg dose of FENF4 had a Cmax of 35.1 ng/mL of fenofibrate in the blood (representing a 1.7 times increase versus the 10 mg/kg dose and a 3.4 times increase versus the 3 mg/kg dose), and the 100 mg/kg dose of FENF4 had a Cmax of 117.9 ng/mL of fenofibrate in the blood (representing a 3.4 times increase in Cmax versus the 30 mg/kg dose, a 5.7 times increase in Cmax versus the 10 mg/kg dose, and a 11.4 times increase in Cmax versus the 3 mg/kg dose).

TABLE 10
Mean Pharmacokinetics Parameter of
Blood Fenofibrate by FENF4 Dose

| | FENF4 Dosage | | | |
|---|---|---|---|---|
| Parameter | 3 mg/kg | 10 mg/kg | 30 mg/kg | 100 mg/kg |
| T1/2 (hr) | NR | 1.7 | 9.4 | NR |
| Tmax (hr) | 0.5 | 0.5 | 1.0 | 0.5 |
| Cmax (ng/mL) | 10.3 | 20.6 | 35.1 | 117.9 |
| AUClast (hr*ng/mL) | NR | 32.6 | 175.2 | 507.9 |
| AUCinf (hr*ng/mL) | NR | 40.2 | 227.8 | NR |

NR: No result, insufficient data to reliably calculate pharmacokinetic parameter.

As shown in Table 11, FENF4 revealed that the dose-response effect on the absorption rate of fenofibric acid and fenofibrate was maintained in brain, with increasing doses of fenofibrate formulated as FENF4 producing progressively higher brain concentrations of both fenofibric acid and fenofibrate in animals.

TABLE 11
Mean Cmax of Brain Fenofibric Acid
and Fenofibrate by FENF4 Dose

| | FENF4 Dosage | | | |
|---|---|---|---|---|
| Sample | 3 mg/kg | 10 mg/kg | 30 mg/kg | 100 mg/kg |
| Brain Fenofibric Acid | NR | 9 ng/g | 162 ng/g | 324 ng/g |
| Brain Fenofibrate | 6 ng/g | 9 ng/g | 182 ng/g | 324 ng/g |

NR: No result, insufficient data to reliably calculate pharmacokinetic parameter.

Taken together, FENF4 demonstrated an improved pharmacokinetic profile, compared to the non-lipid based CMF2 comparator formulation. In terms of fenofibric acid exposure in the blood, 3 mg/kg of FENF4 was equivalent to 100 mg/kg CMF2. In addition, fenofibrate formulated as FENF4 exhibited a dose-response curve for both fenofibric acid and fenofibrate with increasing doses of fenofibrate formulated as FENF4 producing progressively higher blood and brain concentrations of both fenofibric acid and fenofibrate in animals. With substantially improved pharmacokinetics over CMF1 and CMF2 with regards to both blood and brain exposure of fenofibrate and fenofibric acid, the 30 mg/kg dose of the FENF4 formulation was selected for pharmacodynamic analysis.

FENF4 was further evaluated for its pharmacodynamic properties. To evaluate the efficacy of FENF4 in treating an inflammatory response, lipopolysaccharide (LPS) challenge assays were performed in an animal model to measure the animal's ability to respond to an inflammatory stimulus by mounting an acute phase inflammatory response. Bacterial LPS is an endotoxin, a potent inducer of the acute phase response and systemic inflammation. This response is induced by the production of cytokines from activated monocytes and neutrophils in response to inflammatory stimuli, such as, e.g., IL-1β, IL-6; TNFα; MCP-1; IL-10; and MCP-2.

In one series of experiments, FENF4 were evaluated against CMF2 comprising carboxymethylcellulose and fenofibrate. C57BL/6 male mice, each with an average weight of between 15 g to 25 g, were divided into eight groups of ten animals. For each group, half of the animals were administered a test or control composition 5 days prior to a LPS challenge, with dosing on Days −4, −3, −2, −1 and 1 while the remaining half were administered its test or control composition on Day 1 prior to a LPS challenge. Animals were intraperitoneally administered a single, daily, sub-lethal dose of LPS (1 mg/kg) for four consecutive days with the initial LPS dose being administered at a time that correlated with Tmax for the test composition (about 4 hours post dosing). Animals from each group were as follows: Group A animals received a single dose of saline administered intraperitoneally for four consecutive days in lieu of LPS and served as an LPS unchallenged control for inflammation; Group B animals received a single dose of non-lipid vehicle administered orally and served as a CMF2 vehicle control; Group C animals received a single dose of lipid vehicle administered orally and served as a FENF4 vehicle control; Group D animals received a single dose of CMF2 administered orally at 30 mg/kg for five consecutive days prior to the initial LPS challenge; Group E animals received a single dose of CMF2 administered orally at 30 mg/kg only on the same day as the LPS challenge; Group F animals received a single dose of CMF2 administered orally at 100 mg/kg for five consecutive days prior to the initial LPS challenge; Group G animals received a single dose of CMF2 administered orally at 100 mg/kg only on the same day as the LPS challenge; Group H animals received a single dose of FENF4 administered orally at 30 mg/kg for five consecutive days prior to the initial LPS challenge; Group I animals received a single dose of FENF4 administered orally at 30 mg/kg only on the same day as the LPS challenge; and Group J animals received a single dose of Dexamethasone administered orally at 3 mg/kg and served as a positive control for treatment.

Samples of whole blood were taken by venipuncture via lateral tail vein at Day 1, Day 2, Day 3, Day 4 immediately prior to and 4 hours after the LPS challenge and Day 5 immediately prior to sacrifice. Collected whole blood samples were processed by centrifugation at 1500 g for 10 min at 4° C. to extract plasma and the plasma aliquoted into 100 μL samples and stored at −80° C. for cytokine analysis. The brain of sacrificed animals was taken and processed by rinsing with saline, blotting dry and sectioning in half from the frontal anterior to the posterior to create two brain portions. The first brain portion was prepared for cytokine analysis by weighing the brain tissue and then snap freezing the tissue and storing at −80° C. for subsequent cytokine analysis. Frozen brain samples were processed by lysing samples by adding 5 μL of cell lysis buffer per mg of sample, homogenizing using a sonic probe, and incubating at room temperature with gentle agitation for 30 minutes. Resultant brain homogenates were centrifuged for 10 minutes and the decanted supernatant diluted 1:1 with PBS. Cytokines levels were evaluated using a customized multiplex assay (Biotechne Ltd., UK) using a Magpix system (Luminex). Cytokines evaluated in both plasma and brain tissue were IL-1β, IL-6, TNFα, MCP-1, IL-10, and MCP-2.

The second brain portion was prepared for assessment of microglia activity by placing the brain tissue into a sterile container with 10% formalin for 48 hours before being immersed in ethanol for storage prior to immunohistochemistry. Fixed brain samples were processed in paraffin wax and 5 μM sections were transversely cut to encompass the cortex and hippocampus before being mounted on slides and stained using anti-IBA-1 antibodies and anti-CD68 antibodies. Cell counts and biomarker density were assessed via digital image analysis with cells staining positive IBA-1 (IBA-1⁺ cells) being indicative of microglia activation and cells staining positive for CD68 (CD68⁺ cells) being indicative of microglia accumulation. Statistical analyses for both the microglia and cytokine experiments were as follows: Inter-group deviations were statistically analyzed by a one-way analysis of variance (ANOVA). In the case of a significant difference in the mean values among the different levels of treatment, comparisons versus the vehicle group were carried out using the Dunnett's test. In case the equal variance test failed, a Kruskal-Wallis one-way analysis of variance on ranks followed by a Dunn's test was proposed. There was no failure of the equal variance test and other variance tests were not required. Dosing regimens of the Lipid formulation of Fenofibrate were also compared to the standard Fenofibrate formulation using unpaired t-test. For all tests, a p value <0.05 was considered statistically significant.

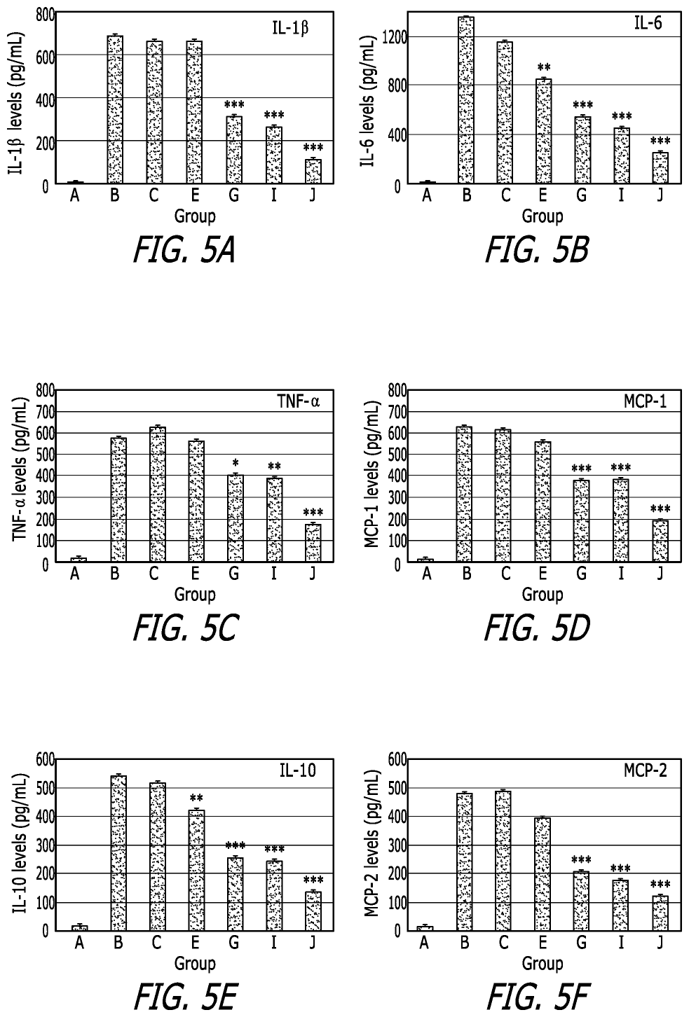

Analysis of blood cytokine levels in these experiments revealed that FENF4 exhibited significant anti-inflammatory activity being three-fold more effective than CMF2. FIGS. 5A-5F show blood cytokine levels in animals undergoing 5 days of an LPS challenge with treatment initiated on Day 1 prior to the initial LPS challenge. As expected, administration of the LPS unchallenged control resulted in no appreciable increase in any of the six cytokines assayed (see Group A in FIGS. 5A-5F). Similarly, administration of vehicle controls for CMF2 (see Group B in FIGS. 5A-5F) and FENF4 (see Group C in FIGS. 5A-5F) resulted, as expected, in significant increases of all cytokines measured. Likewise, administration of positive treatment control showed that dexamethasone significant reduced levels of all six cytokines assayed versus vehicle controls (p<0.001; see Group J in FIGS. 5A-5F). At the lower 30 mg/kg dose of fenofibrate, CMF2 exhibited marginal effects, with only IL-6 and IL-10 levels being lowered significantly versus its vehicle control (p<0.01; see Group E in FIGS. 5B & 5E); IL-1β, TNFα; MCP-1; and MCP-2 levels exhibited no statistically relevant lowering (see Group E in FIGS. 5A, 5C, 5D & 5F). At the higher 100 mg/kg dose of fenofibrate, CMF2 demonstrated significant lowering of all measured cytokines with IL-1β, IL-6, MCP-1, IL-10, and MCP-2 levels being reduced to a greater statistically significant level versus its vehicle control (p<0.001; see Group G in FIGS. 5A, 5B & 5D-5F), relative to TNFα (p<0.05; see Group G in FIG. 5C). FENF4 exhibited significant levels of reduction for all six cytokines assayed and the observed reduction was equivalent to both the higher 100 mg/kg dose of fenofibrate used in CMF2. For example, FENF4 demonstrated significant lowering of all measured cytokines with IL-1β, IL-6, MCP-1, IL-10, and MCP-2 levels being reduced to a greater statistically significant level versus its vehicle control (p<0.001; see Group I in FIGS. 5A, 5B & 5D-5F). relative to TNFα (p<0.01; see Group I in FIG. 5C). These results illustrate that a 30 mg/kg dose of fenofibrate formulated with lipids and with digestion enhancers (FENF4) achieved the same therapeutic potential for reducing systemic inflammation as the high 100 mg/kg dose of fenofibrate formulated in a non-lipid vehicle (CMF2).

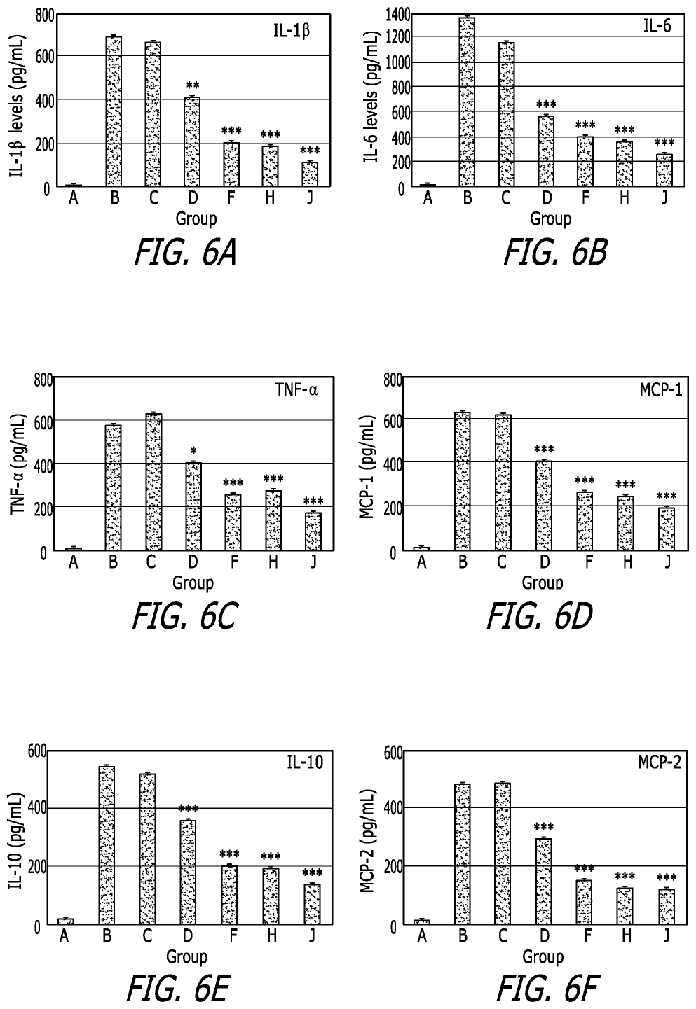

FIGS. 6A-6F shows blood cytokine levels in animals undergoing 5 days of pre-treatment before initiation of an LPS challenge. As expected, administration of the LPS unchallenged control resulted in no appreciable increase in any of the six cytokines assayed (see Group A in FIGS. 6A-6F). Similarly, administration of vehicle controls for CMF2 (see Group B in FIGS. 6A-5F) and FENF4 (see Group C in FIGS. 6A-6F) resulted, as expected, in significant increases of all cytokines measured. Administration of positive treatment control showed that dexamethasone significant reduced levels of all six cytokines assayed versus vehicle controls (p<0.001; see Group J in FIGS. 6A-6F). At the lower 30 mg/kg dose of fenofibrate, CMF2 exhibited substantial effects, with IL-6, MCP-1, IL-10, and MCP-2 levels being reduced to a greater statistically significant level versus its vehicle control (p<0.001; see Group D in FIGS. 6B & 6D-6F) relative to IL-11P (p<0.01; see Group D in FIGS. 6A) and TNFα (p<0.05; see Group D in FIG. 6C). At the higher 100 mg/kg dose of fenofibrate, CMF2 demonstrated significantly reduced levels of all six cytokines assayed versus vehicle controls (p<0.001; see Group F in FIGS. 6A-6F). As seen previously, a 30 mg/kg dose of fenofibrate formulated as FENF4 exhibited significant levels of reduction for all six cytokines assayed and the observed reduction was equivalent to both the higher 100 mg/kg dose of fenofibrate used in CMF2. Specifically, FENF4 demonstrated significantly reduced levels of all six cytokines assayed versus vehicle controls (p<0.001; see Group H in FIGS. 6A-6F).

Taken together, the FENF4 formulation of fenofibrate performed significantly better than the CMF2 formulation at the equivalent 30 mg/kg dose of fenofibrate and was equally as effective as the CMF2 formulation containing three times the amount of fenofibrate (100 mg/kg dose of CMF2). These results reveal that a 30 mg/kg dose of fenofibrate formulated with lipids and with digestion enhancers (FENF4) appeared to be three-fold more effective than non-lipid formulated CMF2 at inhibiting an inflammatory response. Interestingly, these results also indicate that the amount of anti-inflammatory activity achieved by FENF4 seemed enhanced with a 5-day pre-treatment dosing regimen versus dosing on the same day as LPS challenge commencement.

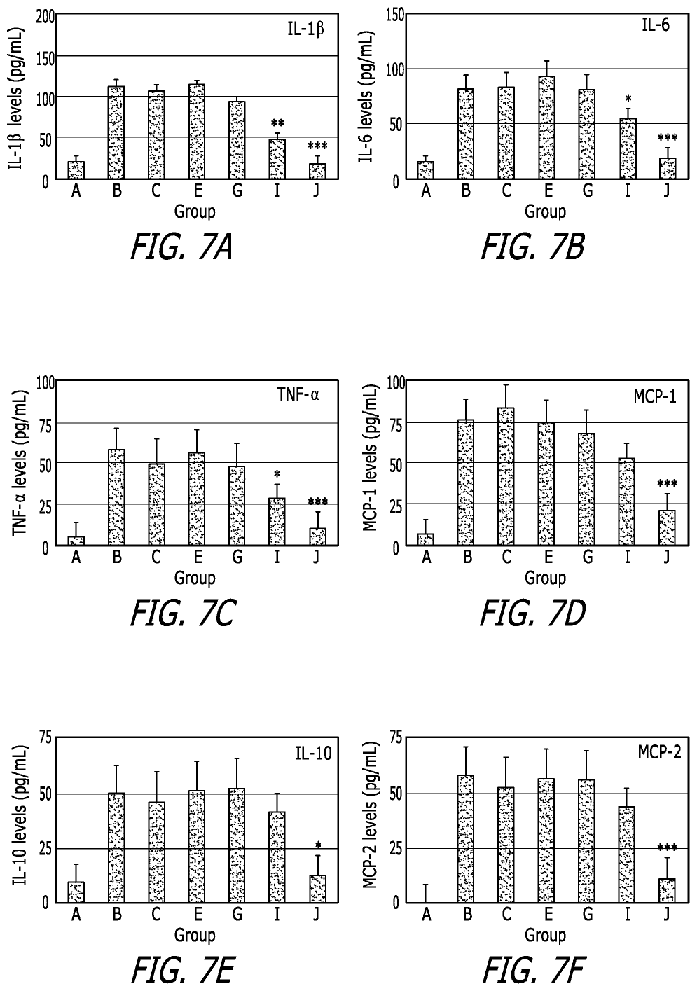

Analysis of brain cytokine levels in these experiments revealed that FENF4 exhibited significant anti-inflammatory activity, with such activity being entirely absent in CMF2. FIGS. 7A-7F shows brain cytokine levels in animals undergoing 5 days of an LPS challenge with treatment initiated on day one prior to the initial LPS challenge. As expected, administration of the LPS unchallenged control resulted in no appreciable increase in any of the six cytokines assayed (see Group A in FIGS. 7A-7F). Similarly, administration of vehicle controls for CMF2 (see Group B in FIGS. 7A-7F) and FENF4 (see Group C in FIGS. 7A-7F) resulted, as expected, in significant increases of all cytokines measured. Administration of positive treatment control showed that dexamethasone significant reduced levels of all six cytokines assayed versus vehicle controls (p<0.001; see Group J in FIGS. 7A-7F). At both the lower 30 mg/kg dose of fenofibrate and higher 100 mg/kg dose of fenofibrate, CMF2 demonstrated no significant reduction in any of the six cytokines assayed (see Group E and Group G in FIGS. 7A-7F). Only, a 30 mg/kg dose of fenofibrate formulated with lipids and with digestion enhancers (FENF4) exhibited significant levels of reduction for some of the cytokines assayed. Specifically, FENF4 demonstrated significant lowering of IL-1β levels versus its vehicle control (p<0.01; see Group I in FIG. 7A) as well as IL-6 and TNFα levels (p<0.05; see Group I in FIGS. 7B & 7C).

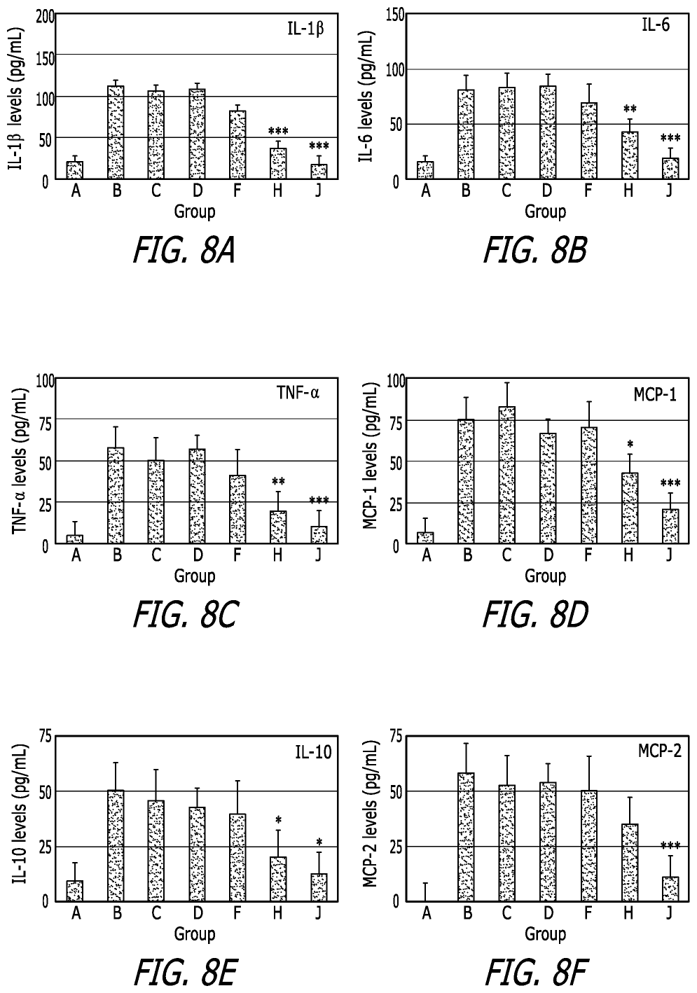

FIGS. 8A-8F shows brain cytokine levels in animals undergoing 5 days of pre-treatment before initiation of an LPS challenge. As expected, administration of the LPS unchallenged control resulted in no appreciable increase in any of the six cytokines assayed (see Group A in FIGS. 8A-8F). Similarly, administration of vehicle controls for CMF2 (see Group B in FIGS. 8A-8F) and FENF4 (see Group C in FIGS. 8A-8F) resulted, as expected, in significant increases of all cytokines measured. Likewise, administration of positive treatment control showed that dexamethasone significant reduced levels of all six cytokines assayed versus vehicle controls (p<0.001; see Group J in FIGS. 8A-8F). At both the lower 30 mg/kg dose of fenofibrate and higher 100 mg/kg dose of fenofibrate, CMF2 demonstrated no significant reduction in in any of the six cytokines assayed (see Group D and Group F in FIGS. 8A-8F). Only, a 30 mg/kg dose of fenofibrate formulated with lipids and with digestion enhancers (FENF4) exhibited significant levels of reduction for most of the cytokines assayed. Specifically, FENF4 demonstrated significant lowering of IL-1β levels versus its vehicle control (p<0.001; see Group H in FIG. 8A), IL-6 and TNFα levels (p<0.01; see Group H in FIGS. 8B & 8C) as well as MCP-1 and IL-10 (p<0.01; see Group H in FIGS. 8D & 8E).

Taken together, the FENF4 formulation of fenofibrate performed significantly better than the CMF2 formulation at either dose. In fact, only FENF4 administration had any effect on cytokine levels in the brain compared to CMF2, suggesting that only FENF4 is effective at being able to reduce levels of brain related inflammation. Of note, levels of both MCP-1 and MCP-2 appeared more resistant to FENF4 administration relative to the other cytokines suggesting that monocytokine chemotaxis was not affected by FENF4 administration. Overall, FENF4 was effective at inhibiting the production of a wide range of cytokines in both the blood and the brain. In addition, both the broad anti-cytokine effect of FENF4 in the brain and the lack of chemotaxis effects, suggests that macrophage infiltration was not affected and that resident microglial cells could be the site of activity for FENF4 activity in the brain. As seen with blood cytokine levels, these brain results also indicate that the amount of anti-inflammatory activity achieved by FENF4 seemed enhanced with a 5-day pre-treatment dosing regimen versus dosing on the same day as LPS challenge commencement. This result suggests that prophylactic treatment with FENF4 could provide additional anti-inflammatory efficacy which will be considered in the design of clinical studies to follow.

To further extend the pharmacodynamic analysis of FENF4, experiments designed to ascertain the dose-response relationship of fenofibrate and the beneficial effect associated with this compound will be conducted. Additionally, the experiments described in this example can be repeated for other fibrates, such as, bezafibrate, ciprofibrate, clofibrate, and gemfibrozil.

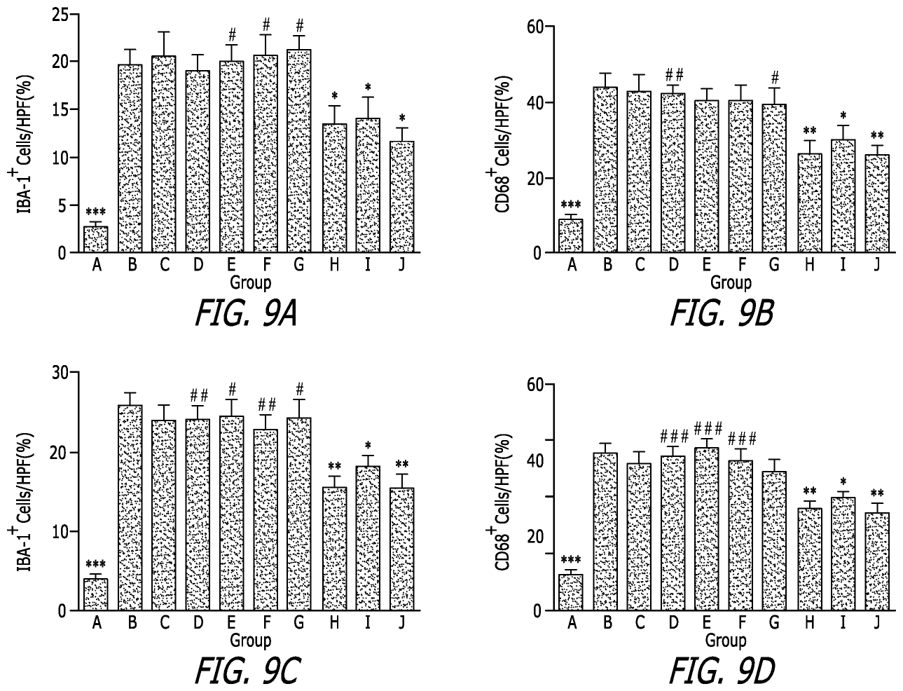

Analysis of the percent level of IBA-1⁺ cells revealed that, regardless of the treatment protocol, FENF4 exhibited significant reduction of microglia activation in both hippocampal and cortical brain tissue, whereas CMF2 did not have any impact on the reduction of microglia activation. For example, FIG. 9A shows microglia activation in hippocampal brain tissue in animals undergoing 5 days of an LPS challenge with treatment initiated either on day one of the initial LPS challenge, or five days prior to the LPS challenge. As expected, administration of the LPS unchallenged control resulted in a no appreciable increase in the percent level of IBA-1⁺ cell indicating that hippocampal tissue did not undergo significant microglia activation (see Group A in FIG. 9A). In contrast, administration of vehicle controls for CMF2 (see Group B in FIG. 9A) and FENF4 (see Group C in FIG. 9A) to LPS challenged animals resulted, as expected, in significant increases in the percent level of IBA-1⁺ cell indicating that hippocampal tissue underwent significant microglia activation. Administration of positive treatment control to LPS challenged animals showed that dexamethasone significantly reduced the percent level of IBA-1⁺ cell indicating microglia activation versus vehicle controls (p<0.05; see Group J versus Groups B and C in FIG. 9A) indicating that dexamethasone significantly suppressed microglia activation in hippocampal tissue. At both the lower 30 mg/kg dose of fenofibrate and higher 100 mg/kg dose of fenofibrate, LPS challenged animals demonstrated no reduction in the percent level of IBA-1⁺ cell versus CMF2 vehicle controls (see Groups D, E, F and G versus B in FIG. 9A) indicating that CMF2 was ineffective at either dose in suppressing microglia activation in hippocampal tissue. In contrast, administration of the 30 mg/kg dose of fenofibrate formulated as FENF4 revealed that LPS challenged animals exhibited significant reduction in the percent level of IBA-1⁺ cell as compared to the FENF4 vehicle control (p<0.05; see Groups H and I versus Group C in FIG. 9A) indicating that FENF4 significantly suppressed microglia activation in hippocampal tissue. Additionally, FENF4 administration significant reduction in the percent level of IBA-1⁺ cells as compared to the 30 mg/kg of CMF2 administered for five consecutive days prior to the LPS challenge (p=0.01 for Group H or I versus Group E in FIG. 9A) or the 100 mg/kg of CMP2 administered by either dosing regimen (p=0.05 for Group H or I versus Group F or G in FIG. 9A). Overall, these results indicate that FENF4 significantly suppressed microglia activation in hippocampal tissue.

Referring to FIG. 9C, similar microglia activation results were observed in the analysis of cortical brain tissue. At both the lower 30 mg/kg dose of fenofibrate and higher 100 mg/kg dose of fenofibrate, LPS challenged animals demonstrated no reduction in the percent level of IBA-1⁺ cell versus CMF2 vehicle controls (see Groups D, E, F and G versus B in FIG. 9C) indicating that CMF2 was ineffective at either dose in suppressing microglia activation in cortical tissue. In contrast, administration of the 30 mg/kg dose of fenofibrate formulated as FENF4 revealed that LPS challenged animals exhibited significant reduction in the percent level of IBA-1⁺ cell as compared to the FENF4 vehicle control (see Groups H and I versus Group C in FIG. 9C). Furthermore, animals administered FENF4 five days prior to the LPS challenge demonstrated a more statistically significant reduction in microglia activation than animals administered FENF4 on Day 1 of the LPS challenge (see Group H versus Group I in FIG. 9C). Additionally, FENF4 administration significant reduction in the percent level of IBA-1⁺ cells as compared to either 30 mg/kg of CMF2 administered by either dosing regimen (p=0.01 for Group H or I versus Group D; p=0.05 for Group H or I versus Group E in FIG. 9C) or the 100 mg/kg of CMP2 administered by either dosing regimen (p=0.01 for Group H or I versus Group F; p=0.05 for Group H or I versus Group G in FIG. 9C). Overall, these results indicate that FENF4 significantly suppressed microglia activation in cortical tissue and are consistent with the findings that FENF4 suppressed microglia activation in hippocampal tissue.

Analysis of the percent level of CD68⁺ cells also revealed that, regardless of the treatment protocol, FENF4 e exhibited a significant reduction in microglia accumulation in both hippocampal and cortical brain tissue, whereas CMF2 did not have any impact on the reduction of microglia accumulation. For example, FIG. 9B shows microglia accumulation in hippocampal brain tissue in animals undergoing 5 days of an LPS challenge with treatment initiated either on day one of the initial LPS challenge or five days prior to the LPS challenge. As expected, administration of the LPS unchallenged control resulted in a no significant increase in the percent level of CD68⁺ cells indicating that hippocampal tissue did not undergo significant microglia accumulation (see Group A in FIG. 9B). In contrast, administration of vehicle controls for CMF2 (see Group B in FIG. 9B) and FENF4 (see Group C in FIG. 9B) to LPS challenged animals resulted, as expected, in significant increases in the percent level of CD68⁺ cells indicating that hippocampal tissue underwent significant microglia accumulation. Administration of positive treatment control to LPS challenged animals showed that dexamethasone significantly reduced the percent level of CD68⁺ cells indicating microglia accumulation versus vehicle controls (p<0.05; see Group J versus Groups B and C in FIG. 9B) indicating that dexamethasone significantly suppressed microglia accumulation in hippocampal tissue. At both the lower 30 mg/kg dose of fenofibrate and higher 100 mg/kg dose of fenofibrate, LPS challenged animals demonstrated no significant reduction in the percent level of CD68⁺ cells versus CMF2 vehicle controls (see Groups D, E, F and G versus B in FIG. 9B) indicating that CMF2 was ineffective at either dose in suppressing microglia accumulation in hippocampal tissue. In contrast, administration of the 30 mg/kg dose of fenofibrate formulated as FENF4 revealed that LPS challenged animals exhibited significant reduction in the percent level of CD68⁺ cells as compared to the FENF4 vehicle control (see Groups H and I versus Group C in FIG. 9B). Furthermore, animals administered FENF4 five days prior to the LPS challenge demonstrated a more statistically significant reduction in microglia accumulation than animals administered FENF4 on Day 1 of the LPS challenge (see Group H versus Group I in FIG. 9B). Additionally, FENF4 administration significant reduction in the percent level of CD68⁺ cells as compared to either the 30 mg/kg or 100 mg/kg CMP2 administered only on the same day of the LPS challenge (p=0.01 for Group H or I versus Group D; p=0.05 for Group H or I versus Group G in FIG. 9B). Overall, these results indicate that FENF4 significantly suppressed microglia accumulation in hippocampal tissue and are consistent with the findings that FENF4 suppressed microglia activation in hippocampal tissue.

Referring to FIG. 9D, similar microglia accumulation results were observed for cortical brain tissue. At both the lower 30 mg/kg dose of fenofibrate and higher 100 mg/kg dose of fenofibrate, LPS challenged animals demonstrated no significant reduction in the percent level of CD68⁺ cell versus CMF2 vehicle controls (see Groups D, E, F and G versus B in FIG. 9D) indicating that CMF2 was ineffective at either dose in suppressing microglia accumulation in cortical tissue. In contrast, administration of the 30 mg/kg dose of fenofibrate formulated as FENF4 revealed that LPS challenged animals exhibited significant reduction in the percent level of CD68⁺ cell as compared to the FENF4 vehicle control (see Groups H and I versus Group C in FIG. 9D). Furthermore, animals administered FENF4 five days prior to the LPS challenge demonstrated a more statistically significant reduction in microglia accumulation than animals administered FENF4 on Day 1 of the LPS challenge (see Group H versus Group I in FIG. 9D). Additionally, FENF4 administration significant reduction in the percent level of CD68⁺ cells as compared to either 30 mg/kg administered only on the same day of or for five consecutive days prior to the LPS challenge or 100 mg/kg CMP2 administered only on the same day of the LPS challenge (p=0.001 for Group H or I versus Group D or E; p=0.01 for Group H or I versus Group F in FIG. 9D). Overall, these results indicate that FENF4 significantly suppressed microglia accumulation in cortical tissue and are consistent with the findings that FENF4 suppressed microglia activation in cortical tissue.

Taken together, the FENF4 formulation of fenofibrate performed significantly better than the CMF2 formulation at either dose in terms of reducing both microglia activation and accumulation, as only FENF4 reduced microglia activation and accumulation in both hippocampal and cortical brain slices. These results are consistent with the cytokine analysis of FENF4 in brain tissue that also illustrate the anti-inflammatory properties of FENF4, that are not observed with CMF2. Comparable to the analysis of brain cytokine levels, these results associated with microglia activation and accumulation also indicate that the amount of anti-inflammatory activity achieved by FENF4 seemed enhanced with a 5-day pre-treatment dosing regimen versus treatment on day of the LPS challenge with FENF4. This result suggests that prophylactic treatment with FENF4 could provide additional anti-inflammatory efficacy which will be considered in the design of clinical studies to follow.

Overall, results from the present experiments demonstrate that lipid fenofibrate with digestion enhancers (FENF4) significantly increases the pharmacokinetic and pharmacodynamic profile of fenofibrate for the successful treatment of inflammation and neuroinflammation, compared to standard non-lipid-based comparators (CMF2). Furthermore, the increased ability of FENF4 to deliver both fenofibric acid as well as fenofibrate to the brain, allows anti-inflammatory effects to be elicited that are mediated by PPARα activation, COX2 inhibition and CB2 activation, thereby increasing the ability of this lipid fenofibrate formulation to treat disorders of the CNS where the underlying pathological process is neuroinflammatory in nature.

### Example 4 Clinical Studies Demonstrate Efficacy of Fenofibrate

In the preclinical murine pharmacokinetic studies conducted, FENF4 had the best fenofibrate exposure in blood of all the compositions (see Table 7) while maintaining good exposure levels for fenofibrate in the brain (Table 9) and fenofibric acid in both the blood and the brain (Table 6 and Table 8). As a result, the FENF4 composition was selected to be developed for human clinical pharmacokinetic studies. The preclinical murine pharmacokinetic studies used a dose strength of 30 mg/kg of FENF4 having a concentration of 6 mg/mL. To select the appropriate dose for human clinical studies, two factors were considered. First, since the mean weigh of a mouse is approximately 25 grams and the mean weight of a human is about 75 kg (a 3,000-fold difference), a higher dose strength would be required for the clinical studies due to the much higher weight of an average human. Additionally, it is known that dose strengths for fenofibrate in its current clinical usage of lowering blood lipids, range between 90 mg and 200 mg. Consequently, accounting for the weight differential and to bracket these known oral doses, unit dose strengths of between 12.5 mg to 125 mg were prepared in the FENF4 composition to assess human exposure for fenofibrate and fenofibric acid in subsequent clinical studies.

The study design for the clinical trials used two capsules to achieve the target single dose strength. Capsules prepared for use in clinical studies were made as follows. A Lipid Vehicle was prepared by mixing MAISINE™ CC, cholic acid, oleic acid, and sodium stearate under heating and with stirring using the amounts according to Table 12. The mixture was then cooled to 60° C. after which GELUCIRE® 43/01 was added under stirring to produce a clear yellow solution. The Lipid Vehicle was then cooled to 30° C. for immediate use in preparing FENF4 capsules or storage at 4° C. for future use.

TABLE 12
Composition of Lipid Vehicle

| | Composition | |
|---|---|---|
| Component | (% w/w) | Quality Standard |
| MAISINE CC ™ | 36.2 | Ph. Eur./USP |
| Cholic Acid | 6.0 | Ph. Eur./USP |
| Oleic Acid | 20.1 | Ph. Eur./USP |
| Sodium Stearate | 1.3 | Ph. Eur./USP |
| GELUCIRE ® 43/01 | 36.2 | Ph. Eur./USP |

For preparation of capsule comprising the FENF4 composition, two intermediate compositions were prepared. A High-Dose Fenofibrate Intermediate Composition was prepared by mixing MAISINE™ CC, cholic acid, oleic acid, and sodium stearate under heating and with stirring. The mixture was then cooled to 60° C. after which GELUCIRE® 43/01 and fenofibrate were added under stirring to produce a clear yellow solution. The mixture is then cooled to 30° C. for immediate use in preparing FENF4 capsules or storage at 4° C. for future use. The final concentration of fenofibrate in the High-Dose Fenofibrate Intermediate Composition is 137.4 mg/mL. A Low-Dose Fenofibrate Intermediate Composition was prepared by mixing MAISINE™ CC, cholic acid, oleic acid, and sodium stearate under heating and with stirring. The mixture was then cooled to 60° C. after which GELUCIRE® 43/01 and fenofibrate were added under stirring to produce a clear yellow solution. The mixture is then cooled to 30° C. for immediate use in preparing FENF4 capsules or storage at 4° C. for future use. The final concentration of fenofibrate in the Low-Dose Fenofibrate Intermediate Composition is 13.7 mg/mL.

To prepare FENF4 capsules (SWEDISH ORANGE®, hydroxypropyl methylcellulose (HPMC), size 00 hard capsule) containing a 0.91 mL fill comprising 12.5 mg to 125 mg of fenofibrate, the appropriate volume of either the High-Dose Fenofibrate Intermediate Composition or the High-Dose Fenofibrate Intermediate Composition were added directly to a capsule or diluted with the appropriate volume of the Lipid Vehicle in order to reach the targeted dosing amount of fenofibrate. For example, to prepare a FENF4 capsule comprising 125 mg of fenofibrate, 0.91 mL of the High-Dose Fenofibrate Intermediate Composition is added directly to the capsule as the fill. For FENF4 capsules comprising 70 mg or 35 mg of fenofibrate, the appropriate amount of the High-Dose Fenofibrate Intermediate Composition was mixed under heating and stirring with the appropriate amount of the Lipid Vehicle to prepare a 0.91 mL fill containing either 70 mg or 35 mg of fenofibrate, which, upon cooling to 30° C., was then dispensed into a capsule. To prepare a FENF4 capsule comprising 12.5 mg of fenofibrate, 0.91 mL of the Low-Dose Fenofibrate Intermediate Composition is added directly to the capsule as the fill. The fill weight of each individual capsule was recorded to ensure equal fill volume (i.e., 0.91 mL). Table 13 shows three exemplary examples of fills used in the clinical studies.

TABLE 13
Composition of Fill in FENF4 Capsules

| | 125 mg | 70 mg | 35 mg | 12.5 mg | |
|---|---|---|---|---|---|
| | Capsule¹ | Capsule² | Capsule³ | Capsule⁴ | Quality |
| Component | (% w/w) | (% w/w) | (% w/w) | (% w/w) | Standard |
| Fenofibrate | 14.9 | 8.3 | 4.2 | 1.4 | CoA |
| MAISINE | 30.8 | 33.2 | 34.7 | 35.7 | Ph. Eur./USP |
| CC ™ | | | | | |
| Cholic Acid | 5.1 | 5.5 | 5.8 | 6.0 | Ph. Eur./USP |
| Oleic Acid | 17.1 | 18.4 | 19.3 | 19.9 | Ph. Eur./USP |
| Sodium | 1.2 | 1.2 | 1.3 | 1.3 | Ph. Eur./USP |
| Stearate | | | | | |
| GELUCIRE ® | 30.8 | 33.2 | 34.7 | 35.7 | Ph. Eur./USP |
| 43/01 | | | | | |

¹Composition 125 mg is a 125 mg capsule with a 0.91 mL fill containing 137.4 mg/mL of fenofibrate.
²Composition 70 mg is a 70 mg capsule with a 0.91 mL fill containing 76.9 mg/mL of fenofibrate.
³Composition 35 mg is a 35 mg capsule with a 0.91 mL fill containing 38.5 mg/mL of fenofibrate.
⁴Composition 12.5 mg is a 12.5 mg capsule with a 0.91 mL fill containing 13.7 mg/mL of fenofibrate.

A Phase I clinical study, divided in two parts, was conducted to evaluate the safety and systemic availability of a disclosed pharmaceutical composition comprising fenofibrate (FENF4). The first part was a single dose, randomized, open-label, placebo-controlled study using a sentinel dosing design conducted to ensure optimal safety. Eight (8) healthy male and female volunteers were recruited and randomly divided into two groups, a Treatment Group of six individuals and a Control Group of two individuals. Individuals in the Treatment Group received a single dose of FENF4 while in a fasted state which was administered as 2 capsules, each capsule comprising 70 mg of fenofibrate, along with 240 mL water. Individuals in the Control Group received a single dose of Lipid Vehicle while in a fasted state which was administered as 2 capsules along with 240 mL water and served as a placebo. With respect to the Treatment Group, blood samples were taken from Day 1 to Day 4 from all individuals for measurement of fenofibric acid and fenofibrate in plasma and subsequent pharmacokinetic (PK) analysis. In addition, CSF samples were taken on Day 1 at 3 hours and 7 hours post-dose from all individuals in the Treatment Group for measurement of fenofibric acid and fenofibrate. With respect to the Control Group, blood samples were taken at a single post-dose timepoint from all individuals in order to confirm correct administration and protocol compliance.

The second part was a single dose, randomized, single-blinded, placebo-controlled study using a sentinel dosing design conducted to ensure optimal safety. Twenty-two (22) healthy male and female volunteers were recruited and randomly divided into three groups, 1) Treatment Group 1 which consisted of eight individuals, each of which received a single dose of FENF4 while in a fasted state and administered as 2 capsules, each capsule comprising 35 mg of fenofibrate, along with 240 mL water; 2) Treatment Group 2 which consisted of six individuals, each of which received a single dose of FENF4 while in a fasted state and administered as 2 capsules, each capsule comprising 70 mg of fenofibrate, along with 240 mL water; and 3) Treatment Group 3 which consisted of eight individuals, each of which received a single dose of FENF4 while in a fasted state and administered as 2 capsules, each capsule comprising 125 mg of fenofibrate, along with 240 mL water. With respect to each Treatment Group, blood samples were taken from Day 1 to Day 4 from all individuals for measurement of fenofibric acid and fenofibrate in plasma and subsequent pharmacokinetic (PK) analysis. In addition, CSF samples were taken on Day 1 at 3 hours and 7 hours post-dose from all individuals in each Treatment Group for measurement of fenofibric acid and fenofibrate.

FENF4 compositions revealed a dose-response effect on the absorption rate of fenofibrate in blood with increasing doses producing progressively higher blood concentrations of fenofibrate in individuals with dose proportional increases in Cmax and AUC₀₋ₗₐₛₜ (Table 14). For example, the 70 mg dose of FENF4 had a Cmax of 2.3 ng/mL of fenofibrate in the blood, with the 140 mg dose of FENF4 having a Cmax of 2.7 ng/mL of fenofibrate in blood (representing a 1.2 times increase in Cmax versus the 70 mg dose), and the 250 mg dose of FENF4 having a Cmax of 5.1 ng/mL of fenofibrate in the blood (representing a 1.9 times increase in Cmax versus the 140 mg dose and a 2.2 times increase in Cmax versus the 70 mg dose). Compared to all other formulations, FENF4 at the 250 mg dose demonstrated the best overall balance of pharmacokinetic parameters for fenofibrate, having low Tmax values and high Cmax and AUC values, illustrating that of the unit dose strengths tested, this formulation reaches maximum concentration of fenofibrate in the shortest amount of time with the greatest amount of drug exposure over time in the blood.

TABLE 14
Mean Pharmacokinetics Parameter of
Blood Fenofibrate by FENF4 Dose

| | FENF4 Dosage | | |
|---|---|---|---|
| Parameter | 70 mg | 140 mg | 250 mg |
| Tmax (hr) | 2.5 | 2.0 | 2.0 |
| Cmax (ng/mL) | 2.3 | 2.7 | 5.1 |
| AUC₀₋ₗₐₛₜ (hr*ng/mL) | 6.7 | 8.2 | 10.3 |

FENF4 compositions revealed a dose-response effect on the absorption rate of fenofibric acid in blood with increasing doses producing progressively higher blood concentrations of fenofibric acid in individuals with dose proportional increases in Cₘₐₓ, AUC₀₋ₗₐₛₜ, and AUC_{0-inf} (Table 15). For example, the 70 mg dose of FENF4 had a Cmax of 3,604 ng/mL of fenofibric acid in the blood, with the 140 mg dose of FENF4 having a Cmax of 8,529 ng/mL of fenofibric acid in blood (representing a 2.4 times increase in Cmax versus the 70 mg dose), and the 250 mg dose of FENF4 having a Cmax of 13,689 ng/mL of fenofibric acid in the blood (representing a 1.6 times increase in Cmax versus the 140 mg dose and a 3.8 times increase in Cmax versus the 70 mg dose). Compared to all other formulations, FENF4 at the 250 mg dose demonstrated the best overall balance of pharmacokinetic parameters for fenofibric acid, having low Tmax values and high Cmax and AUC values, illustrating that of the unit dose strengths tested, this formulation reaches maximum concentration of fenofibric acid in the shortest amount of time with the greatest amount of drug exposure over time in the blood.

TABLE 15
Mean Pharmacokinetics Parameter of
Blood Fenofibric Acid by FENF4 Dose

| | FENF4 Dosage | | |
|---|---|---|---|
| Parameter | 70 mg | 140 mg | 250 mg |
| T1/2 (hr) | 13.6 | 14.2 | 18.1 |
| Tmax (hr) | 2.0 | 2.0 | 2.0 |
| Cmax (ng/mL) | 3604 | 8529 | 13689 |
| AUClast (hr*μg/mL) | 60.3 | 103 | 179 |
| AUCinf (hr*μg/mL) | 62.2 | 106 | 191 |

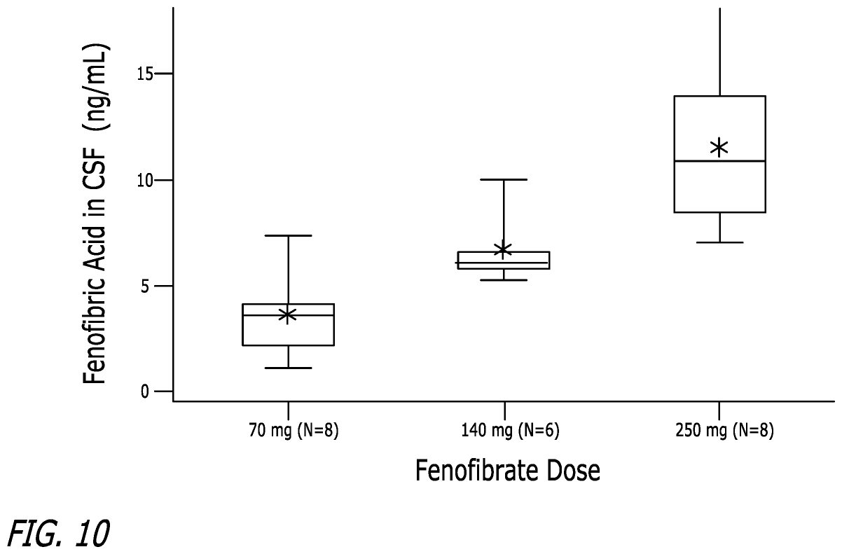

As shown in FIG. 10, at 7 hours post-dose, with increasing fenofibrate doses from 70 mg to 250 mg, the geometric mean concentrations of fenofibric acid in the CSF likewise increased. For example, the 70 mg fenofibrate dose exhibited 3.08 ng/mL of fenofibric acid in the CSF, the 140 mg fenofibrate dose exhibited 6.52 ng/mL of fenofibric acid in the CSF (representing a 2.1 times increase in the geometric mean concentration versus the 70 mg dose), while the 250 mg fenofibrate dose exhibited 11.0 ng/mL of fenofibric acid in the CSF (representing a 1.7 times increase in the geometric mean concentration versus the 140 mg dose and a 3.6 times increase in the geometric mean concentration versus the 70 mg dose). In addition, the ratio of the geometric mean concentration of fenofibric acid present in the CSF relative to the ratio of the geometric mean concentration of fenofibric acid present in the plasma was similar across all FENF4 doses, ranging from 0.00131 to 0.00149 at 7 hours post-dose. Fenofibrate concentrations in the CSF were below LLOQ (less than 0.5 ng/mL) for all individuals at all timepoints measured for all fenofibrate doses examined. This study provides the first data for the transportation of fenofibric acid across the blood brain barrier in humans. As such, these results are indicative of FENF4 composition being able to safely deliver fenofibric acid into the brain using the lymphatic system, thereby bypassing the blood-brain barrier. Furthermore, the results from this study will inform on the dose selection for the subsequent clinical studies.

In a second Phase I clinical study will be conducted to evaluate the safety and systemic availability of a disclosed pharmaceutical composition comprising fenofibrate (FENF4) compared to a standard fenofibrate formulation (STND) as the comparator. In a first clinical study, approximately 32 healthy volunteers will be recruited and equally divided into four groups. The first group will receive a single dose of FENF4 administered in an amount below the anticipated efficacious dose; the second group will receive a single dose of FENF4 administered in an amount at the anticipated efficacious dose; the third group will receive a single dose of FENF4 administered in an amount above the anticipated efficacious dose; and the fourth group will receive a single dose of STND (or placebo) administered in an effective amount currently approved by a regulatory agency. Cerebral spinal fluid (CSF) samples will be collected in each individual to determine levels of fenofibrate and fenofibric acid in CSF. The pharmacokinetic results of this first clinical study are expected to demonstrate that administration of FENF4 is safe. In addition, these results are expected to reveal the presence of fenofibrate in the brain in individual receiving FENF4 but not in the brain (or at lower levels in the brain) of individuals administered STND. Such results would be indicative of FENF4 being able to safely deliver fenofibrate into the brain using the lymphatic system, thereby bypassing the blood-brain barrier. Results from this study will inform dose selection for the subsequent clinical study.

In a third Phase I clinical study, in approximately 32 healthy volunteers will be recruited and equally divided into five groups. The first group will receive multiple doses of FENF4 administered at one dose level; the second group will receive multiple doses of FENF4 administered at a second dose level; the third group will receive multiple doses of FENF4 administered at a third dose level; the fourth group will receive multiple doses of STND (or placebo) administered at an effective dose currently approved by a regulatory agency. Cerebral spinal fluid (CSF) samples will be collected in each individual (excluding the cohort administer STND) and assayed to determine levels of fenofibrate and fenofibric acid in CSF. The results of this second clinical study are expected to demonstrate that administration of FENF4 is safe. In addition, the FENF4 results are expected to reveal the presence of fenofibrate in the brain in individual receiving FENF4. Furthermore, a separate cohort of participants will be recruited for this study to undergo a LPS challenge. This will recruit approximately 16 subjects dosed in a ratio of 1:1 to receive multiple days of dosing of either FENF4 (at one dose level) or STND/placebo (at one dose level), prior to administration of LPS. CSF and/or TSPO PET imaging will be taken following LPS administration. The results are expected to show that FENF4 administration reduced the inflammatory brain response primed by the LPS challenge. Cognition would also be measured to demonstrate remediation of cognitive deficit following the LPS challenge when treated with FENF4 versus STND/placebo. Thus, the results of this study will demonstrate that FENF4 administration was effective at minimizing the harmful effects of a LPS challenge.

Phase II clinical studies will be conducted to demonstrate dose finding, safety, and the efficacy of FENF4 to protect patients against perioperative neurocognitive disorder (PND; or an alternative clinical indication driven by neuroinflammation. In this double-blind study, 120 to 150 patients will be recruited based on biomarker stratification indicating susceptibility to PND and equally divided into three groups. The first group will receive a dose level of FENF4 administered in an amount determined to be an efficacious dose in the Phase I clinical studies and administered daily 7 days pre-surgery and 14 days post-surgery; the second group will receive two doses of FENF4 administered in an amount determined to be an efficacious dose in the Phase I clinical studies and administered daily 7 days pre-surgery and 14 days post-surgery; and the third group will receive a single dose of STND/placebo administered in an effective amount currently approved by a regulatory agency and administered daily 7 days pre-surgery and 14 days post-surgery. Patients will be followed for up to 12 weeks post-surgery. The results of this Phase 11 clinical study are expected to demonstrate that administration of FENF4 is safe. Furthermore, the Phase 11 clinical study results are expected to provide information regarding biomarker validation and provide proof of mechanism that FENF4 reduces neuroinflammation by preventing/reducing PND incidence.

Phase III clinical studies will be conducted in to demonstrate safety and efficacy of FENF4 to protect patients against perioperative neurocognitive disorder (PND; or an alternative clinical indication driven by neuroinflammation). In this double-blind study, approximately 300 patients will be recruited based on biomarker stratification indicating susceptibility to PND, and equally divided into two groups. The first group will receive a dose level of FENF4 administered at an amount determined to be an efficacious dose in the Phase 11 clinical study and administered daily 7 days pre-surgery and 14 days post-surgery; the second group will receive a dose level of STND/placebo administered in an effective amount currently approved by a regulatory agency and administered daily 7 days pre-surgery and 14 days post-surgery. Patients will be followed for 12 weeks post-surgery. The results of this Phase III clinical study are expected to demonstrate that administration of FENF4 is safe. Furthermore, the clinical study results are expected to finalize biomarker validation, provide statistically significant results that FENF4 reduces neuroinflammation by preventing/reducing PND incidence, and provide evidence to conduct label extension studies targeting CNS disorders where neuroinflammation is a predominant cause.

In closing, foregoing descriptions of embodiments of the present invention have been presented for the purposes of illustration and description. It is to be understood that, although aspects of the present invention are highlighted by referring to specific embodiments, one skilled in the art will readily appreciate that these described embodiments are only illustrative of the principles comprising the present invention and such examples are not limiting thereto. As such, the specific embodiments are not intended to be exhaustive or to limit the invention to the precise forms disclosed. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

In addition, groupings of alternative embodiments, elements, steps and/or limitations of the present invention are not to be construed as limitations. Each such grouping may be referred to and claimed individually or in any combination with other groupings disclosed herein. It is anticipated that one or more alternative embodiments, elements, steps and/or limitations of a grouping may be included in, or deleted from, the grouping for reasons of convenience and/or patentability. When any such inclusion or deletion occurs, the specification is deemed to contain the grouping as modified, thus fulfilling the written description of all Markush groups used in the appended claims. In addition, all methods described herein can be performed in any suitable order unless otherwise indicated herein or otherwise clearly contradicted by context. Therefore, it should be understood that embodiments of the disclosed subject matter are in no way limited to a particular element, compound, composition, component, article, apparatus, methodology, use, protocol, step, and/or limitation described herein, unless expressly stated as such.

While aspects of the invention have been described with reference to at least one exemplary embodiment, it is to be clearly understood by those skilled in the art that the invention is not limited thereto. Furthermore, those of ordinary skill in the art will recognize that certain changes, modifications, permutations, alterations, additions, subtractions, and sub-combinations thereof can be made in accordance with the teachings herein without departing from the spirit of the present invention. It is intended that the following appended claims and claims hereafter introduced are interpreted to include all such changes, modifications, permutations, alterations, additions, subtractions, and sub-combinations as are within their true spirit and scope. Accordingly, the scope of the present invention is not to be limited to that precisely as shown and described by this specification. Rather, the scope of the invention is to be interpreted only in conjunction with the appended claims and it is made clear, here, that the inventor(s) believe that the claimed subject matter is the invention.

Certain embodiments of the present invention are described herein, including the best mode known to the inventors for conducting the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

The words, language, and terminology used in this specification is for the purpose of describing particular embodiments, elements, steps and/or limitations only and is not intended to limit the scope of the present invention, which is defined solely by the claims. In addition, such words, language, and terminology are to be understood not only in the sense of their commonly defined meanings, but to include by special definition in this specification structure, material or acts beyond the scope of the commonly defined meanings. Thus, if an element, step, or limitation can be understood in the context of this specification as including more than one meaning, then its use in a claim must be understood as being generic to all possible meanings supported by the specification and by the word itself.

The definitions and meanings of the elements, steps or limitations recited in a claim set forth below are, therefore, defined in this specification to include not only the combination of elements, steps or limitations which are literally set forth, but all equivalent structure, material or acts for performing substantially the same function in substantially the same way to obtain substantially the same result. In this sense it is therefore contemplated that an equivalent substitution of two or more elements, steps and/or limitations may be made for any one of the elements, steps or limitations in a claim set forth below or that a single element, step, or limitation may be substituted for two or more elements, steps and/or limitations in such a claim. Although elements, steps or limitations may be described above as acting in certain combinations and even initially claimed as such, it is to be expressly understood that one or more elements, steps and/or limitations from a claimed combination can in some cases be excised from the combination and that the claimed combination may be directed to a sub-combination or variation of a sub-combination. As such, notwithstanding the fact that the elements, steps and/or limitations of a claim are set forth below in a certain combination, it must be expressly understood that the invention includes other combinations of fewer, more, or different elements, steps and/or limitations, which are disclosed in above combination even when not initially claimed in such combinations. Furthermore, insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements. Accordingly, the claims are thus to be understood to include what is specifically illustrated and described above, what is conceptually equivalent, what can be obviously substituted and also what essentially incorporates the essential idea of the invention.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, all lists of values should be considered as inclusive of intermediate values unless the context indicates the contrary.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a comparable manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators— such as, e.g., “first,” “second,” “third,” etc.—for identified elements are used to distinguish between the elements, and do not indicate or imply a required or limited number of such elements, and do not indicate a particular position or order of such elements unless otherwise specifically stated.

When used in the claims, whether as filed or added per amendment, the open-ended transitional term “comprising”, variations thereof such as, e.g., “comprise” and “comprises”, and equivalent open-ended transitional phrases thereof like “including”, “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

Any claims intended to be treated under 35 U.S.C. § 112(f) will begin with the words “means for,” but use of the term “for” in any other context is not intended to invoke treatment under 35 U.S.C. § 112(f). Accordingly, Applicant reserves the right to pursue additional claims after filing this application, in either this application or in a continuing application.

It should be understood that the methods and the order in which the respective elements of each method are performed are purely exemplary. Depending on the implementation, they may be performed in any order or in parallel, unless indicated otherwise in the present disclosure.

Finally, all patents, patent publications, and other references cited and identified in the present specification are individually and expressly incorporated herein by reference in their entirety to the same extent as if each individual publication or patent application were specifically and individually indicated to be incorporated by reference. These publications are provided solely for their disclosure prior to the filing date of the present application. The reference to any prior art in this specification is not, and should not be taken as, an acknowledgement or any form of suggestion that the prior art forms part of the common general knowledge from any country. In addition, where a definition or use of a term in an incorporated reference is inconsistent or contrary to the definition of that term provided herein, the definition of that term provided herein applies and the definition of that term in the reference does not apply. Lastly, nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

1-63. (canceled)

64. A method of treating an individual with an inflammation, the method comprising the step of administering to the individual in need thereof a pharmaceutical composition, the pharmaceutical composition comprising a) one or more fibrates, b) one or more glycerolipids, and c) one or more digestion enhancers for use in the treatment of inflammation, wherein said digestion enhancer comprise one or more bile acids, one or more one or more free C14-24 fatty acids, one or more one or more free C14-24 fatty acid surfactants, or any combination thereof.

65. The method according to claim 64, wherein the composition comprises 0.1% to 25% by weight of one or more fibrates, 50% to 95% by weight of one or more glycerolipids, and 0.1% to 40% by weight of one or more digestion enhancers.

66. The method according to claim 64, wherein the composition comprises 25 mg/mL to 300 mg/mL of one or more fibrates, 50% to 95% by weight of one or more glycerolipids, and 0.1% to 40% by weight of one or more digestion enhancers.

67. The method according to claim 64, wherein the one or more fibrates comprise; a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof.

68. The method according to claim 64, wherein the one or more glycerolipids comprises one or more hard fats and one or more liquid fats.

69. The method according to claim 68, wherein the composition comprises 0.1% to 25% by weight of one or more fibrates, 40% to 50% by weight of one or more hard fats, 30% to 50% by weight of one or more liquid fats, and 0.1% to about 40% by weight of one or more digestion enhancers.

70. The method according to claim 68, wherein the composition comprises 25 mg/mL to 300 mg/mL of a fibrate, 40% to 50% by weight of one or more hard fats, 30% to 50% by weight of one or more liquid fats, and 0.1% to 40% by weight of one or more digestion enhancers.

71. The method according to claim 68, wherein the one or more hard fats comprise C10-C24 triglycerides and the one or more liquid fats comprise partially hydrolyzed triglycerides including a mixture of C10-C24 mono-, C10-C24 di- and C10-C24 triglycerides.

72. The method according to claim 68, wherein the one or more hard fats comprise a mixture of saturated C10-C18 triglycerides and/or saturated C12-C18 triglycerides and the one or more liquid fats comprise partially hydrolyzed triglycerides including a mixture of C16-C18 mono-, C16-C18 di- and C16-C18 triglycerides.

73. The method according to claim 68, wherein the one or more liquid fats are selected from the list consisting of: glycerol monomyristoleate, glycerol monopalmitoleate, glycerol monosapienate, glycerol monooleate, glycerol monoelaidate, glycerol monovaccenate, glycerol monolinoleate, glycerol monolinoelaidate, glycerol monolinolenate, glycerol monostearidonate, glycerol monoeicosenoate, glycerol monomeadate, glycerol monoarachidonate, glycerol monoeicosapentaenoate, glycerol monoerucate, glycerol monodocosahexaenoate, and glycerol mononervonate.

74. The method according to claim 64, wherein the one or more bile acids includes cholic acid, chenodeoxycholic acid, dafachronic acid, deoxycholic acid, glycocholic acid, glycohenodeoxycholic acid, lithocholic acid, taurochenodeoxycholic acid, taurocholic acid, any stereoisomer thereof, and any combination thereof.

75. The method according to claim 64, wherein the one or more free C14-24 fatty acids include palmitic acid (hexadecenoic acid), palmitolinolenic acid, palmitidonic acid, palmitovaccenic acid, palmitoleic acid, sapienic acid, 4-Hexadecenoic acid, stearic acid (octadecenoic acid), α-linolenic acid, stearidonic acid, α-eleostearic acid, β-eleostearic acid, pumicic acid, 7,10,13-octadecatrienoic acid, 12-octadecenoic acid, linoleic acid, linolelaidic acid. γ-linolenic acid, calendic acid, pinolenic acid, vaccinic acid, ruminic acid, oleic acid, elaidic acid, petroselinic acid, arachidic acid (eicosanoic acid), dihomo-α-linolenic acid, eicosic acidtraenoic acid, eicosapentaenoic acid, 9,12,15-eicosatrienoic acid, β-eicosic acidtraenoic acid, dihomo-linoleic acid, dihomo-γ-linolenic acid, arachidonic acid, paullinic acid, 7,10,13-eicosatrienoic acid, gondoic acid, 8,11-eicosadienoic acid, meadic acid, gadoleic acid, 8-eicosenoic acid, behenic acid (docosanoic acid), clupanodonic acid, docosahexaenoic acid, adrenic acid, osbondic acid, erucic acid, lignoceric acid (tetracosanic acid), 9,12,15,18,21-Tetracosapentaenoic acid, 6,9,12,15,18,21-Tetracosahexaenoic acid, and nervonic acid.

76. The method according to claim 64, wherein the one or more free C14-24 fatty acid surfactants include sodium palmitate (hexadecanoate), sodium palmitolinolenate, sodium palmitidonate, sodium palmitovaccenate, sodium palmitoleate, sodium sapienate, sodium 4-Hexadecenoate, sodium stearate (octadecenoate), sodium α-linolenate, sodium stearidonate, sodium α-eleostearate, sodium β-eleostearate, sodium pumicate, sodium 7,10,13-octadecatrienoate, sodium 12-octadecenoate, sodium linoleate, sodium linolelaidate. Sodium γ-linolenate, sodium calendate, sodium pinolenate, sodium vaccinate, sodium ruminate, sodium oleate, sodium elaidate, sodium petroselinate, sodium arachidate (eicosanoate), sodium dihomo-α-linolenate, sodium eicosatetraenoate, sodium eicosapentaenoate, sodium 9,12,15-eicosatrienoate, sodium β-eicosatetraenoate, sodium dihomo-linoleate, sodium dihomo-γ-linolenate, sodium arachidonate, sodium paullinate, sodium 7,10,13-eicosatrienoate, sodium gondoate, Sodium 8,11-eicosadienoate, sodium meadate, sodium gadoleate, sodium 8-eicosenoate, sodium behenate (docosanoate), sodium clupanodonate, sodium docosahexaenoate, sodium adrenate, sodium osbondate, sodium erucate, sodium lignocerate (tetracosanate), sodium 9,12,15,18,21-Tetracosapentaenoate, sodium 6,9,12,15,18,21-Tetracosahexaenoate, and sodium nervonate.

77. The method according to claim 64, wherein the one or more glycerolipids comprises one or more hard fats and one or more liquid fats and the one or more digestion enhancers comprising one or more bile acids, one or more free C14-24 fatty acids, and one or more free C14-24 fatty acid surfactants.

78. The method according to claim 64, wherein the pharmaceutical composition comprises 0.1% to 25% by weight of one or more fibrates, 25% to 40% by weight of one or more hard fats, 30% to 45% by weight of one or more liquid fats, 0.1% to 10% by weight of one or more bile acids, 10% to 25% of one or more free C14-24 fatty acids, and 0.1% to 5% of one or more free C14-24 fatty acid surfactants,

79. The method according to claim 78, wherein the one or more fibrates are selected from the group consisting of: a bezafibrate, a ciprofibrate, a clinofibrate, a clofibrate, a clofibride, an etofibrate, a fenofibrate, a fenofibric acid, a gemfibrozil, a nafenopin, a ronifibrate, a simfibrate, or any combination thereof, the one or more bile acids comprises cholic acid, the one or more free C14-24 fatty acids is selected from the group consisting of: oleic acid, steric acid and linoleic acid, or any combination thereof, the one or more free C14-18 fatty acid surfactants are oleate alkali metal or alkali earth metal salt, a stearate alkali metal or alkali earth metal salt, a linoleate alkali metal or alkali earth metal salt, or any combination thereof.

80. The method according to claim 78, wherein the one or more hard fats comprise C10-C24 triglycerides and the one or more liquid fats comprise partially hydrolyzed triglycerides including a mixture of C10-C24 mono-, C10-C24 di- and C10-C24 triglycerides.

81. The method according to claim 80, wherein the one or more hard fats comprise a mixture of saturated C10-C18 triglycerides and/or saturated C12-C18 triglycerides and the one or more liquid fats comprising partially hydrolyzed triglycerides including a mixture of C16-C18 mono-, C16-C18 di- and C16-C18 triglycerides.

82. The method according to claim 64, wherein the pharmaceutical composition is not an emulsion or self-emulsifying formulation.

83. The method according to claim 64, wherein the inflammation is a neuroinflammation.
